SEQUENCE LISTING (314 sequences)

SEQ ID NO: 1 - protein, 506 aa, Bacillus sp.
MSTPLQQPHQKSKKTSQMITTRKFKLAIVSDNRNEAYSFIRNEIRNQNKALNAAYNHLYFEHIATEKLKHSDAEYQKHLTKYREVATNKYQDYLKAKEKVNASKDDEKLQKRVDKAREAYNKAQEKVYKIEKEFNKKSMETYQKVVGLSKQTRIGKLLKSQFTLHYDTEDRITSTVLSHFNNDMKTGVLRGDRSLRTYKNSHPLLVRARSMKVYEENGDYFIKWVKGIVFKIVISAGSKQKANIGELKSVLINILNGHYKVCDSSISLNKDLILNLSLNIPVSKENVFVPGRVVGVDLGLKIPAYVSLNDTPYIKKGIGNIDDFLRVRTQLQSQRKRLQKTLECTSGGKGRSKKLKGLDRLKAKEKNFVNTYNHFLSKKIIQFAVKNNAGVIHLEELQFDKLKHKSLLRNWSYYQLQTMIEYKAEREGIEVKYVDASYTSQTCSKCGHYEEGQRVLQDTFTCKNKECKGYVHKVNADFNASQNIAKSTDIIRCTEMAKNNDIEKNA

SEQ ID NO: 2 - protein, 506 aa, Bacillus sp.
MSTPLQQPHQKSKKTSQMITTRKFKLAIVSDNRNEAYSFIRNEIRNQNKALNAAYNHLYFEHIATEKLKHSDEEYQKHLTKYREVATNKYQDYLKVKEKVNASKDDEKLQKRVDKAREAYNKAQEKVYKIEKEFNKKSMETYQKVVGLSKQTRIGKLLKSQFTLHYDTEDRITSTVLSHFNNDMKTGVLRGDRSLRTYKNSHPLLVRARSMKVYEENGDYFIKWVKGIVFKIVISAGSKQKANIGELKSVLINILNGHYKVCDSSISLNKDLILNLSLNIPVSKENVFVPGRVVGVDLGLKIPAYVSLNDNPYIKKGIGNIDDFLNVRTQLQNQRKRLQKTLECTSGGKGRSKKLKGLDRLKAKEKNFVNTYNHFLSKKIIQFAVKNNAGVIHLEELQFDKLKHKSLLRNWSYYQLQTMIEYKAEREGIEVKYVDASYTSQTCSKCGHYEEGQRVLQDTFTCKNKECKGYVHKVNADFNASQNIAKSTDIIRCTEMAKNNDIEKNA

SEQ ID NO: 3 - protein, 506 aa, Bacillus sp.
MSTPLQQPHQKSKKTSQMITTRKFKLAIVSDNRNEAYSFIRNEIRNQNKALNAAYNHLYFEHIATEKLKHSDAEYQKHLTKYREVATNKYQDYLKAKEKVNASKDDEKLQKRVDKAREAYNKAQEKVYKIEKEFNKKSMETYQKVVGLSKQTRIGKLLKSQFTLHYDTEDRITSTVLSHFNNDMKTGVLRGDRSLRTYKNSHPLLVRARSMKVYEENGDYFIKWVKGIVFKIVISAGSKQKANIGELKSVLINILNGHYKVCDSSISLNKDLILNLSLNIPVSKENVFVPGRVVGVDLGLKIPAYVSLNDTPYIKKGIGNIDDFLNVRTQLQNQRKRLQKTLECTSGGKGRSKKLKGLDRLKAKEKNFVNTYNHFLSKKIIQFAVKNNAGVIHLEELQFDKLKHKSLLRNWSYYQLQTMIEYKAEREGIEVKYVDASYTSQTCSKCGHYEEGQRVLQDTFTCKNKECKGYVHKVNADFNASQNIAKSTDIIRCNEMAKNNDIEKNA

SEQ ID NO: 4 - protein, 506 aa, Bacillus sp.
MSTPLQQPHQKSKKTSQMITTRKFKLAIVSDNRNEAYSFIRNEIRNQNKALNAAYNHLYFEHIATEKLKHSDEEYQKHLTKYREVATNKYQDYLKVKEKVNDSKDDEKLQKRVDKAREAYNKAQEKVYKIEKEFNKKSMETYQKVVGLSKQTRIGKLLKSQFTLHYDTEDRITSTVISHFNNDMKTGVLRGDRSLRTYKNSHPLLVRARSMKVYEENGDYFIKWVKGIVFKIVISAGSKQKANIGELKSVLINILNGHYKVCDSSISLNKDLILNLSLNIPVSKENVFVPGRVVGVDLGLKIPAYVSLNDTPYIKKGIGNIDDFLKVRTQLQSQRKRLQKTLECTSGGKGRNKKLKGLDRLKAKEKNFVNTYNHFLSKKIIQFAVKNNAGVIHLEELQFDKLKHKSLLRNWSYYQLQTMIEYKAEREGIEVKYVDASYTSQTCSKCGHYEEGQRVLQDTFTCKNKECKGYVHKVNADFNASQNIAKSTDIIRCTEMAKNNDIEKNA

SEQ ID NO: 5 - protein, 506 aa, Bacillus sp.
MSTPLQQPHQKSKKTSQMITTRKFKLAIVSDNRNEAYSFIRNEIRNQNKALNAAYNHLYFEHIATEKLKHSDEEYQKHLTKYREVSTNKYQDYLKVKEKVNASKDDEKLQKRVDKAREAYNKAQEKVYKIEKEFNKKSMETYQKVVGLSKQTRIGKLLKSQFTLHYDTEDRITSTVLSHFNNDMKTGVLRGDRSLRTYKNSHPLLVRARSMKVYEENGDYFIKWVKGIVFKIVISAGSKQKANIGELKSVLINILNGHYKVCDSSISLNKDLILNLSLNIPVSKENVFVPGRVVGVDLGLKIPAYVSLNDTPYIKKGIGNIDDFLNVRTQLQNQRKRLQKTLECTSGGKGRSKKLKGLDRLKAKEKNFVNTYNHFLSKKIIQFAVKNNAGVIHLEELQFDKLKNKSLLRNWSYYQLQTMIEYKAEREGIEVKYVDASYTSQTCSKCGHYEKGQRVLQDTFTCKNKECKGYAHKVNADFNASQNIAKSTDIIRCTEMAKNNDIEKNA

SEQ ID NO: 6 - protein, 450 aa, Bacillus sp.
MSIAVKVMKYQIVCPVNVEWKAFETYLRTLSYQSRTIGNRTIQKIWEFDNLSLNHFKETGEYPSAQQLYGCTQKTISGYIYNQLREEYQDINKANMSTTIQKSLKNWNSRKKEIWRGEMSIPSFRSDLPIDIHGNSIQLIKEKSGDYFANVSLFSSKFIKENDLPNGKILVKLSTRKQNSMKVILDRLINSTYSKGACMLHKHKNKWYLSITYKATKKEGHKFDEELIMGIDMGKINVLYFAYNKGLVRGAISGEEIEAFRKKIEYRRISLLRQGKYCSENRIGKGRKKRIKPIEVLNDKITKFRNATNHKYANYIVQQCLKYNCGTIQLEDLQGISKEQTFLKNWTYFDLQEKIKNQANQYGIKVVKIDPSYTSQRCSECGYIHKNNRQDQSTFECQHCSFKVHADYNAAKNISIYNIEKVIQKQLELQEKLNLTKYKEQYIEQMENIN

SEQ ID NO: 7 - protein, 506 aa, Bacillus sp.; identical to SEQ ID NO: 5

SEQ ID NO: 8 - protein, 450 aa, Bacillus sp.
MSLAVKVMKYQIVCPVNVEWKVFETYLRTLSYQSRTIGNRTIQKIWEFDNLSLKHFKETGEYPSAQQLYGCTQKTISGYIYDQLKEEYKDINKANMSTTIQKTLKNWNSRKKEIWRGEMSIPSFRSDLPIDIHGNSIQLIKEKGGDYIASVSLFSSKFIKENDLPNGKILMKLSTRKQNSMKVILDRLINSTYSKGACMLHKHKKKWYLSITYKSNIKEELKFDEDLIMGIDMGKINVLYFAFNKSLVRGAISGEEIEAFRKKIEHRRISLLRQGKYCSGNRIGKGREKRIKPIDVLNDKVAKFRNATNHKYANYIVQQCLKYNCGTIQLEDLKGISKEQTFLKNWTYFDLQEKIKNQANQYGVKVVKIDPSYTSQRCSECGYIHKDNRQDQSTFECLQCSFKAHADYNAAKNISVYTIEKVIQKQLELQEKLNLTKYKEQYIEQIKNIN

SEQ ID NO: 9 - protein, 486 aa, Bacillus sp.
MITSRKIRLSIVSDNATEAYNFIRKEMREQNKALNVALNHLYFNSIARQKILLADEAYQQKLKDAINSQEKSFNTLKELERKQCIEEDFEKKQTLKERLIKAKNAYEKAKEKVSHLRKSRSKDSFQEYKNIIGQVEQTHLRDIISSQFNLHSDTKDRLTMIANQDFKNDIAEVLSGNCSLRTYKKNNPLYIRGRNTVLYKEGNEFFIKWIKGIVFKCILGVKNQNKTELYKTLECVLAGNYKICDSSMNFNQQNKLIMNLTLNMPDKDENRKVPGRIAGIDLGLKIPAYFAVNDAPYIRKALGKIEDFLKVRTSIQSQKRSLERALQSSKGGKGRKKKLRALDQFKGKEKRYVTTYNHFISKKIISLAIQYGVEQINLELLTLKETQKKSLLRNWSYYQLQQFIEYKAKREGILIKYVDPFNTSQTCSKCDHYEGGQREKQANFLCKSCGFEENADFNAARNIAKSKNYITRKEESEYYKRNNEIA

SEQ ID NO: 10 - protein, 451 aa, Bacillus sp.
MSVMIKIMKYQILCPTNVDWSTFEKNLRDLTYQVRTISNRTIQQLWEFDALSYDYFKETGTSPTVQDLYKCTQKKIDGYIYHVLQSKYPDVHKGNMSTTLQKIIKTWKSRRSEIRKGEMSIPSFRNQLPIDLHNNSVEVTKEKNGDYIAGLSLFSRDFHKENSDVPKGKIFVKLATKKQKSMKVILDRLISGTYSKGACMIHKYKNKWYLSITYKFNSITENKFDENLIMGIDMGGVNTVYFAFNEGFVRSNIRSDEIRAFNERIRQRRINLLNQSKYCSDSRTGKGRAKRLQPIDVISNKIAQFRNSTNHKYANFIVKQCLKYNCGRIQIEHLKGISKDDKVLKDWTYYDLQEKIKKQAQAYGIEVITIAPAYTSQRCSQCGYISNENRCTQAVFECKQCEYKTHADYNAAKNISTYDIENIINKQLAVQSRLHSKKYMEDHIEELGYSG

SEQ ID NO: 11 - protein, 599 aa, Gordonia sp.
MAITVHTMGAHYRWEIPEQLRAQLWLAHNLREDLVTLQHEYEARTKEVWSSFPDVAATEDRLTAAELEAEALAEKVKSERIRQRTKRVTGPVADQLAAARKTAKEARIARRAAIAAVRDTAKAQLNELSDELKAAHKRLYAEYCQSGHLYWATFNATLDHHKTAVKRMQQLRAAGRPAQMRHHRFDGTGTIAVQLQRQSGQPQRTPALIGDPEGKYRNVFYSPWVDPDQWDTMTRSEQRKAGRVTVTMRCGSTDDGPAWIAVPVQQHRMLPADADITGAQLTVTRKGSATHVKIAVTARVGEPEPITDGPTVAVHLGWRDTDTGTVVAHWRASEPLDVPFDMRDIMPTDPGGRTGTVVIPTRIVDRIESAAGIASQRGDAQNEMKRQLVEWLTEVGPQPHPTRDGEEISAADAARWRNPGRFAALAMAWRDNPPNRGEQIAYVLESWRATDKGLWNRQEGGRRKALGHRTDIYRQVAALIADQAGVVVVDDTSIADIAARPTELPNEVEARIARRRGSAAPGELREAVRSAAVRDGVGVEVVAHKGLSRTHAACGHENPADDRYMTVGVVCDGCGRTYDQDLSATLLMLQRATSTAAAT

SEQ ID NO: 12 - protein, 553 aa, Micrococcus sp.
MTTTTGTDLVPRLRAFKHRLDPNPAQATLLAQYAGAARVAYNMLTAHNRAALAASAARRTELAETGLAGPELAARMKAERAADPTLRVASYQSYSTTHLTPLIRRHREAAAAIAAGADPAEAWTDERYAEPWMHTVPRRVLVSGLQNAAKATENWMASASGTRAGARVGLPRFKKKGRSRDSFTIPAPEVIGAAGTPYKRGEPRRGVITDHRHLRLASLGTIRTYDKTSRLVRACRRGAQIRSMTISQAGGRWYASILVADPTPIRTGPSRRQRANDAVGVDLGVKHLAALSTGEVIDNGRPGARQAARLTRLQRAYARTQPGSNRRERVRRQIAALHHGIALRRAGLLHQVSTRLAMDFAVVALEDLNVAGMTRSARGTLEAPGRNVAAKSGLNRAILDAGLGMLRRQLDYKTSWAGSQVKMIDRFAPSSKACSRCGTVKSTLSLAERTFECEACHLVIDRDVNAAINIRAWAVQEERGAGVGLARGRRESRNGRGAAVSGPPSGGAAGQGRGSVKPAPQGVGMSSRATGWSSQPPSTEGESAERGASALAR

SEQ ID NO: 13 - protein, 553 aa, Micrococcus sp.
MTTTTGTDLAPRLRAFKHRLDPNPAQATLLAQYAGAARVAYNMLTAHNRAALAAGAARRTELAETGLAGPELAARMKAERAADPTLRVASYQSYSTAHLTPLIRRHREAAAAIAAGADPAEAWTDERYAEPWMHTVPRRVLVSGLQNAAKATENWMASASGTRAGARVGLPRFKKKGRSRDSFTIPAPEVIGAAGTPYKRGEPRRGVITDHRHLRLASLGTIRTYDKTSRLVRACRRGAQIRSMTISQAGGRWYASILVADPTPIRTGPSRRQRANGAVGVDLGVKHLAALSTGEVIDNGRPGARQAARLTRLQRAHARTQPGSNRRERVRRQIAALQHGIALRRAGLLHQVSTRLATDFAVVALEDLNVAGMTRSARGTLEAPGRNVAAKSGLNRAILDAGLGMLRRQLDYKTSWAGSQVKMIDRFAPSSKACSRCGTVKSTLSLAERTFECEACHLVIDRDVNAAINIRAWAVQEERGAGVELARGRRESRNGRGAAVSGPPSGGAARQGRGSVKPAPQGVGMSSRATGWSSQPPSTEGESAERGASALAR

SEQ ID NO: 14 - protein, 505 aa, Paeniglutamicibacter sp.
MSVQAAMATTVIHRAYRLTLDPTPQQAQKLSQWAGAARAMYNHAIAAKQASHRSWLQEVAFATYEQELTEEQARKSIKVPIPTAYGFNAWLTETRNTHHDAAEKGLLLPGRDGREHEPWLHAVNRSALMGAMRHADDAWTNWIDSLTGTRAGRKIGYPRFKKRGVARDSFTITHDRKSPGIRLATTRRLRIPTFGEIRIHDHAKRLHRKLHTGTVEVTSVTVSRHGPRWYASLTVEETIPTPRLSKRKRAAGIIGVDLGVKITAALSNGDLIPNPRVKASHAKKLARLQKALAKSQKGSRNRAQLVQKIGCLTHLEARQREGHAHNLANRLVHTWAIIGIEDLNVAGMTRSSRGTIEKPGKNVRAKAGLNRSILDVAPAQIRHLLDYKTAWSGTQLVVIDRWAPTSKKCSTCGAVKAKLTLAERTFECEACGLVLDRDINAARNIAALAAVAPSTEETQNARRAAPRKPVPSTVKQRAAMKREDPPGSSPPSNGRTFHTVLVQVS

SEQ ID NO: 15 - protein, 515 aa, Streptomyces sp.
MEREVLRAFKFALDPTPAQAEALARHAGAARWAFNYALAVKVSAHQRWRAEVAGLVAQGIEEAEARRRVKVPVPSKPQIQKRLNEVKGDSRIDGRLPEGTFGPERPCPWWYEVNTYAFQSAFIDADRAWKNWLDSLRGVRAGRKVGYPRFKKKGRSRDSFRLHHDVKRPGIRLATYRRLRLPTIGEVRLHDSGKRLGRLVDRSLAVVQSVTVSRAGHRWYASVLCKVTMTVPDQPSRRQRERGTIGVDLGVKTLAALSQPLDPTDPDSDLIANPRHLARAQQRLLKAQRALSRTEKGSRRRDRARRKVARLHHEVALRRESALHAVTKRLATAFAVVAVEDLHVAGMTASARGTLEKPGRRVRQKAGLNRAVLDAAPGEFRRQLTYKTSWYGSKLAVCDRWFPSSKTCSACGWQNPHLTLTDRTFHCPDCGLTIDRDLNAARNIARHATVADAPPVAPGRGETQNARRAPVRPDDRKAARHGAMKREDTRPLGQVPPQRSNPLASPPTQKQATLF

SEQ ID NO: 16 - protein, 553 aa, Micrococcus sp.
MTTTTGTDLAPRLRAFKHRLDPNPAQATLLAQYAGAARVAYNMLIAHNRAALAAGAARRTELAESGLAGPELAARMKAERAADPTLRVASYQSYSTAHLTPLIRRHREAAAAIAAGADPAEAWTDERYAEPWMHTVPRRVLVSGLQNAAKATENWMASASGTRAGARVGLPRFKKKGRSRDSFTIPAPEVIGAAGTPYKRGEPRRGVITDHRHLRLASLGTIRTYDKTSRLVRACRRGAQIRSMTISQAGGRWYASILVADPTPIRTGPSRRQRANGAVGVDLGVKHLAALSTGEVIDNGRPGARQAARLARLQRAYARTQPGSNRRERVRRQIAALHHGIALRRAGLLHQVSTRLATDFAVVALEDLNVAGMTRSARGTLEAPGRNVAAKSGLNRAILDAGLGMLRRQLDYKTSWAGSQVKMIDRFAPSSKACSRCGTVKSTLSLAERTFECEACHLVIDRDVNAAINIRAWAVQEERGAGVELARGRRESRNGRGAAVSGPPSGGAAGQGRGSVKPAPQGVGMSSRATGWSSQPPSTEGESAERGASALAR

SEQ ID NO: 17 - protein, 557 aa, Bacillus sp.
MTTREILRAYRVPLDPTDAQTAALASHAGASRAAFNWALGAKVHAHRMWSACVADLTYTRYGHLDADQALAAAKKDASRYYRIPTSQTNEKAFDRDPDYAWRTEVNRRSWVSGMRQADTAWQNWLDSLTGRRAGRRVGYPRFKSKGRCRDSFTLAHDVKRPSIRPDGYRRLTLPKKISVTGSIRLKGNIRHLARRIRRGVARIQSATISRAGNGWSVSILALETLDIPDHPTPRQQAAGAVGVDVGVHHLMAFSDATIIDNPRHLRAAQKRLTRAQRALSRSKWRLPNGDLIDTPKRGQRVTPTTGRVKARARLAREHAAVAQHRASTLHAITKQLATSHAVVAVDDLNVAVMTRSARGSIDKPGRNVAAKAGLNRSILDASFAEMRRQLTYKTSWYRSQLLPSGCFVPTSRTCSTCGAEKANLPRSERVYHCENCATVLDRDVNAAKNVLRTALASHDAPGMEESQNARGGRGETSVARRSAKREDPPRGGPPRPCNRVRSSPPDGEAKVRGKLPPPTAKRGNTAPRKGRPRARGDEPNKPNRGALLKMSAPRTRR

SEQ ID NO: 18 - protein, 451 aa, Bacillus sp.
MGVTIKIMKYQILCPMNVDWTIFEKHLRNLTYQVRTISNRTIQQLWEFDALSFDYFKERGTSPTVQDLYGCTQKKIDGYIYHTLQSKYPDIHKGNMSTTLQKIIKTWKSRRNEIRKGEMSIPSFRNRIPIDLHNNSVDITKEKNGDYIAGISLFSRDFHKENGDVPKGKIFVKLATQKQKSMKVILDRLMNQTYSKGACMIHKYKNKWYLSITYKFNALKENKFDKELIMGIDMGGINTVYFAFNKGLIRSNIKSDEIKMFNERIRQRRINLLKQSKYCSNSRTGKGRTKRLQPIDVLSNKIAKFRNSTNHKYANYIIKQCLKHNCGRIQMELLKGISKNDKVLKDWTYFDLQEKIKNQAEFHGIEVIKVAPAYTSQRCSQCGYICKENRCTQAMFECKQCGYKTHADYNAAKNIATYDIENIINKQLAVQSKLHSKKCIEEYIEELGYLD

SEQ ID NO: 19 - protein, 451 aa, Bacillus sp.
MGVTIKIMKYQILCPMNVDWTIFEKHLRNLTYQVRTISNRTIQQLWEFDALSFDYFKERGTYPTVQDLYGCTQKKIDGYIYHTLQSKYPDIHKGNMSTTLQKIIKTWKSRRNEIRKGEMSIPSFRNRIPIDLHNNSVDIIKEKNGDYIAGISLFSRDFHKENGDVPKGKIFVKLGTQKQKSMKVILDRLINQTYSKGACMIHKYKNKWYLSITYKFNAIKENKFDKELIMGIDMGGINTVYFAFNEGFIRSNIKSDEIKMFNERIRQRRINLLKQSKYCSNSRTGKGRTKRLQPIDVLSNKIAKFRNSTNHKYANYIVKQCLKHNCGRIQMELLKGISKNDKVLKDWTYFDLQEKIKNQAEIYGIEVIKVVPAYTSQRCSQCGYICKENRCTQAMFECKQCGYKTHADYNAAKNISTYDIENIINKQLAVQSKLHSKKCMEEYIEELGYLD

SEQ ID NO: 20 - protein, 451 aa, Bacillus sp.
MGVTIKIMKYQILCPMNVDWTIFEKHLRNLTYQVRTISNRTIQQLWEFDALSFDYFKARGTYPTVQDLYGCTQKKIDGYIYHTLQSKYPDIHKGNMSTTLQKIIKTWKSRRNEIRKGEMSIPSFRNRIPIDLHNNSVDITKEKNGDYIAGISLFSRDFHKENDDVPKGKIFIKLATQKQKSMKVILDRLINQTYSKGACMIHKYKNKWYLSITYKFNAIKENKFDKELIMGIDMGGINTVYFAFNEGFIRSNIKSDEIKTFNERIRQRRINLLKQSKYCSNSRTGKGRTKRLQPIDVLSNKIAKFRNSTNHKYANYIIKQCLKHNCGRIQMELLKGISKNDKVLKDWTYFDLQEKIKNQAEIHGIEVIKIAPAYTSQRCSQCGYICKENRCTQAMFECKQCGYKTHADYNAAKNISTYDIENIINKQLAVQSKLHSKKCMAEYMEELGYLD

SEQ ID NO: 21 - protein, 377 aa, Bacillus sp.
MLVNKAYKFRIYPNKEQEILIAKTIGCSRFVFNHFLGMWNDTYKETGKGLTYNSCSAQLPQLKIELEWLKEVDSIAIQSALKNLVDAYNRFFKKQNDKPRFKSKKNDVQSYKTKHTNGNIAIVNNKIKLPKLGFVTFAKSREVDGRIMNATVRRNSSGKYFVAILTEVEIQPLKKADSAIGIDLGITDFAILSDGHKIDNNKFTSKMEKKLKREQRKLSKRALLAKNKGIHLLDAQNYQKQKCKVARLHERVINQRDDFLNKLSTEIIKNHDIICIEDLNTKGMLRNHKLAKSISDVSWSAFVSKLEYKATWYGKTIVKVSRWFPSSQICSDCGHHDGKKSLEIRGWTCPICHANHDRDFNASKNILAEGLRTLALV

SEQ ID NO: 22 - protein, 445 aa, Bacillus sp.
MGVTIKIMKYQILCPLNVDWTIFEKHLRNLTYQVRTISNRTIQQLWEFDALSFDYFKERGTYPTVQDLYGCTQKKIDGYIYHTLQSKYPDIHKGNMSTTLQKIIKTWKSRRNEIRKGEMSIPSFRNRIPIDLHNNSVDITKEKNGDYIAGISLFSRDFHKENDDVPKGKIFVKLATQKQKSMKVILDRLINQTYSKGACMIHKYKNKWYLSITYKFNAIKENKFDKELIMGIDLGGINTVYSAFNEGFIRSNIKSDEIIRQRRINLLKQSKYCSNSRTGKGRTKRLQPIDVLSNKIAKFRNSTNHKYANYIVKQCLKHNCGRIQMELLKGISKNDRILKDWTYFDLQEKIKNQAEIHGIEVIKVAPAYTSQRCSQCGYICKENRCTQATFECKQCGYKTHADYNAAKNISTYDIENIINKQLAVQSKLHSKKCMEEYIEELGYLD

SEQ ID NO: 23 - protein, 451 aa, Bacillus sp.
MGVTIKIMKYQILCPMNVDWTIFEKHLRNLTYQVRTISNRTIQQLWEFDALSFDYFKAIGTYPTVQDLYGCTQKKIDGYIYHTLQSKYPDIHKGNMSTTLQKIIKTWKSRRNEIRKGEMSIPSFRNRIPIDLHNNSVDIKKEKNGDYIAGISLFSRDFHKENSDVPKGKIFVKLATKKQKSMKIILDRLMNQTYSKGACMIHKYKNKWYLSITYKFNAIKENNFDKELIMGIDMGGINTVYFAFNEGFIRSNIKSDEIKAFNEKIRQRRINLLKQSNYCSNSRTGKGRAKRLQPIDVLSNKIAKFRNSTNHKYANYIIKQCLKHNCGRIQIELLKGISKNDKVLKDWTYFDLQEKIKNQAEVHGIEVIKVASAYTSQRCSQCGYICKENRCTQAVFECKQCGYKTHADYNAAKNIATYDIENIINKQLAVQSKLHSKKCMEEYIEELGYLD

SEQ ID NO: 24 - protein, 506 aa, Bacillus sp.; identical to SEQ ID NO: 1

SEQ ID NO: 25 - protein, 377 aa, Bacillus sp.
MLVNKAYKFRLYPTKEQKTLIAKTIGCSRFVFNHFLGQWNDTYKETGKGLTYNSSSAELTKLKKELVRLKEVDSIALQSSLKNLADSYSRFFKKQNNAPRFKSKRNRVQSYTTKETNGNIAVVGNKMKLPKLGLVRFAKSREVHGRVLNATVRRTPSGKYFVSILAEVDVLPMEKAESSIGIDLGITDFAIFSDGRMIDNNKFTAKMEKKLKREQRKLSRRALHAKQNGINLLDAKNYQKQKRKVARLHERVLNQRDDFLNKLSTEIIKNHDLICIENLNTKGMLRNHKLAKSISDVSWSAFVSKLEYKATWYGKAIMKVSKWFPSSQICSDCGHQDCKKSLAIREWTCPICHQHHDRDINASKNILAEGLRTLALA

SEQ ID NO: 26 - protein, 445 aa, Bacillus sp.
MGVTIKIMKYQILCPLNVDWTIFEKHLRNLTYQVRTISNRTIQQLWEFDALSFDYFKERGTYPTVQDLYGCTQKKIDGYIYHTLQSKYPDIHKGNMSTTLQKIIKTWKSRRNEIRKGEMSIPSFRNRIPIDLHNNSVDITKEKNGDYIAGISLFSRDFHKENDDVPKGKIFVKLATQKQKSMKVILDRLINQTYSKGACMIHKYKNKWYLSITYKFNAIKENKFDKELIMGIDLGGINTVYFAFNEGFIRSNIKSDEIIRQRRINLLKQSKYCSNSRTGKGRTKRLQPIDVLSNKIAKFRNSTNHKYANYIVKQCLKHNCGRIQMELLKGISKNDRILKDWTYFDLQEKIKNQAEIHGIEVIKVAPAYTSQRCSQCGYICKENRCTQATFECKQCGYKTHADYNAAKNISTYDIENIINKQLAVQSKLHSKKCMEEYIEELGYLD

SEQ ID NO: 27 - protein, 448 aa, Bacillus sp.
MTFFGVTIKIMKYQILCPLNVDWTIFEKHLRNLTYQVRTISNRTIQQLWEFDALSFDYFKERGTYPTVQDLYGCTQKKIDGYIYHTLQSKYPDIHKGNMSTTLQKIIKTWKSRRNEIRKGEMSIPSFRNRIPIDLHNNSVDITKEKNGDYIAGISLFSRDFHKENDDVPKGKIFVKLATQKQKSMKVILDRLINQTYSKGACMIHKYKNKWYLSITYKFNAIKENKFDKELIMGIDLGGINTVYSAFNEGFIRSNIKSDEIIRQRRINLLKQSKYCSNSRTGKGRTKRLQPIDVLSNKIAKFRNSTNHKYANYIVKQCLKHNCGRIQMELLKGISKNDRILKDWTYFDLQEKIKNQAEIHGIEVIKVAPAYTSQRCSQCGYICKENRCTQATFECKQCGYKTHADYNAAKNISTYDIENIINKQLAVQSKLHSKKCMEEYIEELGYLD

SEQ ID NO: 28 - protein, 451 aa, Bacillus sp.
MGVTIKIMKYQILCPMNVDWTIFEKHLRNLTYQVRTISNRTIQQLWEFDALSFDYFKERGTYPTVQDLYGCTQKKIDGYIYHTLQSKYPDIHKGNMSTTLQKIIKTWKSRRNEIRKGEMSIPSFRNRIPIDLHNNSVDITKEKNGDYIAGISLFSRDFHKENDDVPKGKIFVKLATQKQKSMKVILDRLINQTYSKGACMIHKYKNKWYLSITYKFNAIKENKFDKELIMGIDLGGINTVYFAFNEGFIRSNIKSDEIKMFNERIRQRRINLLKQSKYCSNSRTGKGRTKRLQPIDVLSNKIAKFRNSTNHKYANYIVKQCLKHNCGRIQMELLKGISKNDRILKDWTYFDLQEKIKNQAEIHGIEVIKVAPAYTSQRCSQCGYICKENRCTQATFECKQCGYKTHADYNAAKNISTYDIENIINKQLAVQSKLHSKKCMEEYIEELGYLD

SEQ ID NO: 29 - protein, 471 aa, Bacillus sp.
MIIARKIKLIIIGEDRDTQYKFIREERYKQNKALNVAMNHLYFLHVAKEKIRLLDNKFLQDEKKLQEGIKKLYAEKKVIKDGKKRNELEKKIEKQTNELKKLRSKGNKEADKILQEAIKINLSSTTREVISKQFDLISDTKDRITQKVYQDFKSDLKNGLLSGERVLRTYKKNNPLLIRGRALNFYREGKDVMIKWFGGIIFKCMLGQHKNNAQELKATLNKVLEGSYKVCDSSISVGKELILNISLDIGEVNSNVSCKKGRVLGVDLGMKVPAYMSINDKPYIRKSLGSLDDFLRIRVQMQKRRRNLHKTLVSVKGGKGREKKLQALDRLKEKNFATTYNHFLSYNIVKFAKDNLAEQINMEFLALAGEDKNIILRNWSYYQLQQFVEDKAKREGIDVKYVDPYRTSQMCSKCRNYEPGQRESQEKFICKSCHLEINADYNASQNIAHSTKYITNKNQSEYFKKLQHTTE

SEQ ID NO: 30 - protein, 451 aa, Bacillus sp.
  MOD_RES (160)..(160): Any amino acid
MGVTIKIMKYQILCPMNVDWTIFEKHLRNLTYQVRTISNRTIQQLWEFDALSFDYFKERGTYPTVQDLYGCTQKKIDGYIYHTLQSKYPDIHKGNMSTTLQKIIKTWKSRRNEIRKGEMSIPSFRNRIPIDLHNNSVDIIKEKNGDYIAGISLFSRDFHXENGDVPKGKIFVKLGTQKQKSMKVILDRLINQTYSKGACMIHKYKNKWYLSITYKFNAIKENKFDKELIMGIDMGGINTVYFAFNEGFIRSNIKSDEIKMFNERIRQRRINLLKQSKYCSNSRTGKGRTKRLQPIDVLSNKIAKFRNSTNHKYANYIVKQCLKHNCGRIQMELLKGISKNDKVLKDWTYFDLQEKIKNQAEIYGIEVIKVVPAYTSQRCSQCGYICKENRCTQAMFECKQCGYKTHADYNAAKNISTYDIENIINKQLAVQSKLHSKKCMEEYIEELGYLD

SEQ ID NO: 31 - protein, 451 aa, Bacillus sp.; identical to SEQ ID NO: 19

SEQ ID NO: 32 - protein, 451 aa, Bacillus sp.; identical to SEQ ID NO: 19

SEQ ID NO: 33 - protein, 471 aa, Bacillus sp.; identical to SEQ ID NO: 29

SEQ ID NO: 34 - protein, 451 aa, Bacillus sp.
MGVTIKIMKYQILCPMNVDWTIFEKQLRNLTYQVRTISNRTIQQLWEFDALSFDYFKERGTYPTVQDLYGCTQKKIDGYIYHTLQSKYPDIHKGNMSTTLQKIIKTWKSRRNEIRKGEMSIPSFRTRIPIDLHNNSVDIIKEKNGDYIAGISLFSRDFHKENGDVPKGKIFVKLGTQKQKSMKVILDRLINQTYSKGACMIHKYKNKWYLSITYKFNAIKENKFDKELIMGIDMGGINTVYFAFNEGFIHSNIKSDEIKMFNERIRQRRINLLKQSKYCSNSRTGKGRTKRLQPIDVLSNKIAKFRNSTNHKYANYIVKQCLKHNCGRIQMELLKGISKNDKVLKDWTYFDLQEKIKNQAEIYGIEVIKVVPAYTSQRCSQCGYICKENRCTQAMFECKQCGYKTHADYNAAKNISTYDIENIINKQLAVQSKLHSKKCMEEYIEELGYLD

SEQ ID NO: 35 - protein, 196 aa, Bacillus sp.
MDVGLKELFVASNGMKERNINKDAKVKKLLKRKKSAQRDMSRRFKNGVKIQSAGYEKAKAEHLRLSRKITNIRNNHIHQATATLVKTKPMRIVVEDLSISNLLKNKKLSKALSFQKLNLFFQCLSYKCEKYGIEYVKADKWVASSKICSCCGVKYDHSVQSEGQWSLKIREWRCVRCNSHHDRDVNAAINLSRWVK

SEQ ID NO: 36 - protein, 445 aa, Bacillus sp.; identical to SEQ ID NO: 22

SEQ ID NO: 37 - protein, 451 aa, Bacillus sp.
MGVTIKIMKYQILCPMNVDWTIFEKQLRNLTYQVRTISNRTIQQLWEFDALSFDYFKERGTYPTVQDLYGCTQKKIDGYIYHTLQSKYPDIHKGNMSTTLQKIIKTWKSRRNEIRKGEMSIPSFRNRIPIDLHNNSVDIIKEKNGDYIAGISLFSRDFHKENGDVPKGKIFVKLGTQKQKSMKVILDRLINQTYSKGACMIHKYKNKWYLSITYKFNAIKENKFDKELIMGIDMGGINTVYFAFNEGFIRSNIKSDEIKMFNERIRQRRINLLKQSKYCSNSRTGKGRTKRLQPIDVLSNKIAKFRNSTNHKYANYIVKQCLKHNCGRIQMELLKGISKNDKVLKDWTYFDLQEKIKNQAEIYGIEVIKVVPAYTSQRCSQCGYICKENRCTQAMFECKQCGYKTHADYNAAKNISTYDIENIINKQLAVQSKLHSKKCMEEYIEELGYLD

SEQ ID NO: 38 - protein, 362 aa, Bacillus sp.
MLKAFKYRIYPNNEQRIFFAKTFGCVRFVYNKMLADRIESYQESQDKPDKSIKYPTPAQYKVEFPFLKEVDSLALANAQMNLNKAYANFFRDKSVGFPKFKSKKDRHRSYTTNNQKGTVCIEKGYIKLPKLKTLVKIKQHRQFFGLMKSVTISQTPTGKYFVSVLVEEKEQLSPKTDEKVGVDLGLKDFAILSNGTKYENPKWLRRLEKRLAFLQRSLSRKKKGSNNLNKARLQVARLHEKIANQRNDFLHKISNEITNENQVIVIEDLKVKNMQKNHKLTRAISEVSWSKFREYLAYKTAWKGRDLIVAPKNYASSQLCSCCGYKNKEVKNLNLREWTCPECNSHHNRDINASINLLKLAM

SEQ ID NO: 39 - protein, 451 aa, Bacillus sp.
MGVTIKIMKYQILCPMNVDWTIFEKHLRNLTYQVRTISNRTIQQLWEFDVLSFDYFKERGTYPTVQDLYGCTQKKIDGYIYHTLQSKYPDIHKGNMSTTLQKIIKTWKSRRNEIRKGEISIPSFRNRIPIDLHNNSVDITKEKNGDYIAGISLFSRDFHKENDDVPKGKIFVKLATQKQKSMKVILDRLINQTYSKGACMIHKYKNKWYLSIIYKFNAIKENKFDKELIMGIDMGGINTVYFAFNEGFIRNNIKSDEIKAFNERIRQRRINLLNQSKYCSNSRTGKGRTKRLQPIDVLSNKIAKFRNSTNHKYANYIIKQCLKYNCGRIQMELLKGISKNDKVLKDWTYFDLQEKIKNQAEIHGIEVIKVAPAYTSQRCSQCGYICKENRCTQAMFECKECGYKTHADYNAAKNIATYDIENIINKQLAVQSKLHSKKCMEEYIEELGYLD

SEQ ID NO: 40 - protein, 451 aa, Bacillus sp.; identical to SEQ ID NO: 19

SEQ ID NO: 41 - protein, 450 aa, Bacillus sp.
MSIAVKVMKYQIVCPVNIEWKTFEIYLRTLSYHFRTIGNRTIQKLWEYDNQSLKHFKDTGQYPSAQQLYGCTQKTISGYIYDQLKEEYQDINKANMSTTLQKTIRTWNSRKKEIWSGEMSIPSFRNNLPIDIHGNSIQIIKEKSGDYIASVSLFSSKFIKENDLPNGKILVKLSTRKQNSMKVILDRIIDSTYAKGACMLHKHKKKWYLSITYKSNIKEELKFDEDLIMGIDMGKINVLYFAFNKGLVRGAISGEEIEAFRKKIEHRRISLLRQGKYCSGNRIGKGREKRIKPIDVLNDKVAKFRNATNHKYANYIVQQCLKYNCGTIQLEDLKGISKEQTFLKNWTYFDLQEKIKNQANQYGVKVVKIDPSYTSQRCSECGYIHKNNRQDQSTFECQQCSFKVHADYNAAKNISLYNIEKVIQKQLELQEKLNQTKYKEQYIEQMKNIN

SEQ ID NO: 42 - protein, 450 aa, Bacillus sp.; identical to SEQ ID NO: 41

SEQ ID NO: 43 - protein, 444 aa, Bacillus sp.
MKYQILCPMNVDWTIFEKHLRNLTYQVRTISNRTIQQLWEFDALSFDYFKERGTYPTVQDLYGCTQKKIDGYIYHTLQSKYPDIHKGNMSTTLQKIIKTWKSRRNEIRKGEMSIPSFRNRIPIDLHNNSVDIIKEKNGDYIAGISLFSRDFHKENGDVPKGKIFVKLGTQKQKSMKVILDRLINQTYSKGACMIHKYKNKWYLSITYKFNAIKENKFDKELIMGIDMGGINTVYFAFNEGFIRSNIKSDEIKMFNERIRQRRINLLKQSKYCSNSRTGKGRTKRLQPIDVLSNKIAKFRNSTNHKYANYIVKQCLKHNCGRIQMELLKGISKNDKVLKDWTYFDLQEKIKNQAEIYGIEVIKVVPAYTSQRCSQCGYICKENRCTQAMFECKQCGYKTHADYNAAKNISTYDIENIINKQLAVQSKLHSKKCMEEYIEELGYLD

SEQ ID NO: 44 - protein, 451 aa, Bacillus sp.
MGVTIKIMKYQILCPMNVDWTIFEKHLRHLTYQVRTISNRTIQQLWEFDTLSFDYFKERGTYPTVQDLYGCTQKKIDGYIYHTLQSKYPDIHKGNMSTTLQKIIKTWKSRRNEIRKGEMSIPSFRNRIPIDLHNNSVDITKEKNGDYIAGISLFSRDFHKENGDVPKGKIFVKLATQKQKSMKVILDRLINQTYSKGACMIHKYKNKWYLSITYKFNAIKENKFDKELVMGIDMGGINTVYFAFNEGFIRSNIKSDEIKMFNERIRQRRINLLKQSKYCSNSRTGKGRTKRLQPIDVLSNKIAKFRNSTNHKYANYIVKQCLKHNCGRIQMELLKGISKNDKILKDWTYFDLQEKIKNQAEIHGIEVIKVAPAYTSQRCSQCGYICKENRCTQATFECKQCGYKTHADYNAAKNISTYDIENIINKQLAVQSKLHSKKCMEEYIEELGYLD

SEQ ID NO: 45 - protein, 451 aa, Bacillus sp.
MRVTIKIMKYQILCPMNVDWTIFEKHLRNLTYQVRTISNRTIQQLWEFDALSFDYFKEKGTYPTVQDLYGCTQKKIDGYIYHTLQSKYLDIHKGNMSTTLQKIIKTWKSRRNEIRKGEMSIPSFRNRIPIDLHNNSVDITKEKNGDYIAGISLFSRDFHKENGDVPKGKIFVKLATQRQKSMKVILDRLINQTYSKGACMIHKYKNKWYLSITYKFNAIKENKFDKELIMGIDMGGINTVYFAFNEGFIRSNIKSDEIKAFNERIRQRRINLLKQSKYCSNSRTGKGREKRLQPIDVLSNKIAKFRNSTNHKYANYIIKQCLKHNCGRIQMELLKGISKNDKVLKDWTYFDLQEKIQNQAEIHGIEVIKVAPAYTSQRCSQCGYICKENRCTQAVFECKQCGYKTHADYNAAKNIATYDIENIINKQLAVQSKLHSKKCMEEYIEELGYLD

SEQ ID NO: 46 - protein, 506 aa, Bacillus sp.; identical to SEQ ID NO: 4

SEQ ID NO: 47 - protein, 506 aa, Bacillus sp.
MSTPLQQAHQKSKKTSQMITTRKFKLAIVSDNRNEAYSFIRNEIRNQNKALNAAYNHLYFEHIATEKLKHSDAEYQKHLTKYREVATNKYQDYLKAKEKVNASKDDEKLEKRVDKAREAYNKAQEKVYKIEKEFNKKSKETYQKVVGLSKQTRIGKLLKSQFTLHYDTEDRITSTVLSHFNNDMKTGVLRGDRSLRTYKNSHPLLVRARSMKVYEENGDYFIKWVKGIVFKIVISAGSKQKANIGELKSVLINILNGHYKVCDSSISLNKDLILNLSLNIPVSKENVFVPGRVVGVDLGLKIPAYVSLNDTPYIKKGIGNIDDFLKVRTQLQNQRKRLQKTLECTSGGKGRSKKLKGLDRLKAKEKNFVNTYNHFLSKKIIQFAVKNNAGVIHLEELQFDKLKHKSLLRNWSYYQLQTMIEYKAEREGIKVKYVDASYTSQTCSKCDHYEKGQRVLQDTFTCKNKECKGYDHKVNADFNASQNIAKSTDIIRCTEMAKNNDIEKNA

SEQ ID NO: 48 - protein, 451 aa, Bacillus sp.; identical to SEQ ID NO: 28

SEQ ID NO: 49 - protein, 451 aa, Bacillus sp.; identical to SEQ ID NO: 45

SEQ ID NO: 50 - protein, 506 aa, Bacillus sp.; identical to SEQ ID NO: 5

SEQ ID NO: 51 - protein, 405 aa, Bacillus sp.
MARKTVEQRNKELAKENKVLRHYGLKLRALPTPFQEEKIAKTIGSARFSYNFYLNEKIEIYKLTNLTLDYSSFKKSFNGLKQHPAFDWLKEVDKFSLESALEQVDDAFERFFKGQTKFPKFKSKHKTKQSYTTKETNGNIAFDLENQVVKLPKMGKVPVKLSKKHLTMFQKNEFTGKIKSATVTRHSSGQYYISLKCEEIILLEEKIDVTTIPTDGIIGCDLGLTYFLIDSNGQKIENPRYLKENLKKLAKLQRGLKHKKIGSSNFQKLKQKIAKLHLHISNMRKDFLHKVSRKLVNENQVIILEDLNVKGMIKNPKLARSIADVGWGMFKTFVSYKANWANKILILINRFFPSSKQCNGCKEKNTLLSLSDRLWMCPSCGTHHDRDDNAALNIKEEGIRLLLNA

SEQ ID NO: 52 - protein, 252 aa, Bacillus sp.
MIHKYKNKWYLSITYKFNAIKENKFDKELIMGIDMGGINTVYFAFNEGFIRSNIKSDEIKAFNERIRQRRINLLKQSKYCSNSRTGKGREKRLQPIDVLSNKIAKFRNSTNHKYANYIIKQCLKHNCGRIQMELLKGISKNDKVLKDWTYFDLQEKIQNQAEIHGIEVIKVAPAYTSQRCSQCGYICKENRCTQAVFECKQCGYKTHADYNAAKNIATYDIENIINKQLAVQSKLHSKKCMEEYIEELGYLD

SEQ ID NO: 53 - protein, 451 aa, Bacillus sp.; identical to SEQ ID NO: 45

SEQ ID NO: 54 - protein, 445 aa, Bacillus sp.; identical to SEQ ID NO: 26

SEQ ID NO: 55 - protein, 451 aa, Bacillus sp.; identical to SEQ ID NO: 37

SEQ ID NO: 56 - protein, 451 aa, Bacillus sp.
MGVTIKIMKYQILCPMNVDWTIFEKHLRNLTYQVRTISNRTIQQLWEFDALSFDYFKARGTYPTVQDLYGCTQKKIDGYIYHRLQSKYPDIHKGNMSTTIQKIIKTWKSRRNEIRKGEMSIPSFRNRIPIDLHNNSVDITKEKNGDYIAGISLFSRDFHKENGDVPKGKIFVRLATQKQKSMKVILDRLMNQTYSKGACMIHKYKNKWYLLITYKFNAVKENIFDKELIMGIDMGGINTVYFAFNEGFIRSNIKSDEIKMFNERIRQRRINLLKQSKYCSNSRTGKGRTKRLQPIDLLSNKIAKFRNSTNHKYANYIIKQCLKHNCGRIQMELLKGISKNDKVLKDWTYFDLQEKIKNQAEIHGIEVIKVAPAYTSQRCSQCGYICKENRCTQAMFECKQCGYKTHADYNAAKNIATYDIENIINKQLAVQSKLHSKKYMEEYIEELGYLD

SEQ ID NO: 57 - protein, 451 aa, Bacillus sp.; identical to SEQ ID NO: 37

SEQ ID NO: 58 - protein, 445 aa, Bacillus sp.; identical to SEQ ID NO: 26

SEQ ID NO: 59 - protein, 445 aa, Bacillus sp.; identical to SEQ ID NO: 22

SEQ ID NO: 60 - protein, 451 aa, Bacillus sp.
MRVTIKIMKYQILCPLNVDWTIFEKHLRNLTYQVRTISNRTIQQLWEFDALSFDYFKEKGTYPTVQDLYGCTQKKIDGYIYHTLQSKYPDIHKGNMSTTLQKIIKTWKSRRNEIRKGEMSIPSFRNRIPIDLHNNSVDITKEKNGDYIAGISLFSRDFHKENGDVPKGKIFVKLATQKQKSMKVILDRLINQTYSKGACMIHKYKNKWYLSITYKFNAIKENKFDKDLIMGIDMGGINTVYFAFNEGFIRSNIKSDEIKAFNERIRQRRINLLKQSKYCSNSRTGKGREKRLQPIDVLSNKIAKFRNSTNHKYANYIIKQCLKHNCGRIQMELLKGISKNDKVLKDWTYFDLQEKIQNQTEIHGIEVIKVAPAYTSQRCSQCGYICKENRCTQAVFECKQCGYKTHADYNAAKNIATYDIENIINKQLAVQSKLHSKKCMEEYIEELGYLD

SEQ ID NO: 61 - protein, 451 aa, Bacillus sp.; identical to SEQ ID NO: 23

SEQ ID NO: 62 - protein, 451 aa, Bacillus sp.; identical to SEQ ID NO: 19

SEQ ID NO: 63 - protein, 451 aa, Bacillus sp.; identical to SEQ ID NO: 39

SEQ ID NO: 64 - protein, 445 aa, Bacillus sp.; identical to SEQ ID NO: 26

SEQ ID NO: 65 - protein, 451 aa, Bacillus sp.; identical to SEQ ID NO: 19

SEQ ID NO: 66 - protein, 451 aa, Bacillus sp.; identical to SEQ ID NO: 23

SEQ ID NO: 67 - protein, 451 aa, Bacillus sp.
MGVTIKIMKYQILCPMNVDWTIFEKHLRNLTYQVRTISNRTIQQLWEFDVLSFDYFKERGTYPTVQDLYGCTQKKIDGYIYHTLQSKYPDIHKGNMSTTLQKIIKTWKSRRNEIRKGEISIPSFRNRIPIDLHNNSVDITKEKNGDYIAGISLFSRDFHKENGDVPKGKIFVKLATQKQKSMKVILDRLINQTYSKGACMIHKYKNKWYLSIIYKFNAIKENKFDKELIMGIDMGGINTIYFTFNEGFIRNNIKSDEIKAFNERIRQRRINLLNQSKYCSNSRTGKGRTKRLQPIDVLSNKIAKFRNSTNHKYANYIIKQCLKYNCGRIQMELLKGISKNDKVLKDWTYFDLQEKIKNQAEIHGIEVIKVAPAYTSQRCSQCGYICKENRCTQAMFECKQCGYKTHADYNAAKNIATYDIENIINKQLAAQSKLHSKKCMEEYIEELRYLD

SEQ ID NO: 68 - protein, 451 aa, Bacillus sp.; identical to SEQ ID NO: 18

SEQ ID NO: 69 - protein, 496 aa, Bacillus sp.
MILTRKVKLVIVSDNRDEGYKLIRNEIREQHKALNLAYNHLYFEHNAIQILKQNDEDYKQKRNKLQELINKKYEEHQKAKNLERKEALREAYNNKKQELYKFERECNEEARKAYQQVVGFTQQTRVRNLINRECNLMSDTKDGITSKVTQDYKNDCKAGLLIGKRSLRNYKKDNPLLVRGRSLKFYKEDGDYFIKWNKGTVFKCILHIRKKNVAELQSVLENVLLGAYKICDSSIGFNNKDMILNLSLNIPDKETYDYIPGRVVGVDLGLKIPAYVSLSDKVYVRKGIGGIDDFLRVRTQMQKRRRQLQESLAAVKGGKGREKKLKALDHLKGKEANFAKTYNHFLSTQIVTFAVKNQAGQINMEFLEFDKMKNKSLLRNWSYYQLQMMVEYKAKREGIIIKYVDAYLTSQTCSKCDYYEEGQREKQEKFICKSCAFEVNADYNASQNIAKSARYISDSTEREYHKKKQEDLKEILGENDIINEQLSLFDNHDDIA

SEQ ID NO: 70 - protein, 496 aa, Bacillus sp.; identical to SEQ ID NO: 69

SEQ ID NO: 71 - protein, 496 aa, Bacillus sp.; identical to SEQ ID NO: 69

SEQ ID NO: 72 - protein, 451 aa, Bacillus sp.
MGVTIKIMKYQILCPMNVDWTIFEKQLRNLTYQVRTISNRTIQQLWEFDALSFDYFKERGTYPTVQDLYGCTQKKIDGYIYHTLQSKYPDIHKGNMSTTLQKIIKTWKSRRNEIRKGEMSIPSFRNRIPIDLHNNSVDIIKEKNGDYIAGISLFSRDFHKENGDVPKGKIFVKLGTQKQKSMKVILDRLINQTYSKGACMIHKYKNKWYLSITYKFNAIKENKFDKELIMGIDMGGINTVYFAFNEGFIHSNIKSDEIKMFNERIRQRRINLLKQSKYCSNSRTGKGRTKRLQPIDVLSNKIAKFRNSTNHKYANYIVKQCLKHNCGRIQMELLKGISKNDKVLKDWTYFDLQEKIKNQAEIYGIEVIKVVPAYTSQRCSQCGYICKENRCTQAMFECKQCGYKTHADYNAAKNISTYDIENIINKQLAVQSKLHSKKCMEEYIEELGYLD

SEQ ID NO: 73 - protein, 451 aa, Bacillus sp.; identical to SEQ ID NO: 39

SEQ ID NO: 74 - protein, 451 aa, Bacillus sp.; identical to SEQ ID NO: 28

SEQ ID NO: 75 - protein, 473 aa, Bacillus sp.
MIIARKIKLIIIGEDRDTQYKFIREERYKQNKALNVAMNHLYFLHVAKEKIRLLDNKFLQDEKKLQEGIKKLYAEKKVIKDGKKRNELEKKIEKQTNELKKLRSKGNKEADKILQEAIKINLSSTTREVISKQFDLISDTKDRITQKVYQDFKSDLKNGLLSGERVLRTYKKNNPLLIRGRALNFYREGKDVMIKWFGGIIFKCMLGQHKNNAQELKATLNKVLEGSYKVCDSSISVGKELILNISLDIGEVNSNVSCKKGRVLGVDLGMKVPAYMSINDKPYIRKSLGSLDDFLRIRVQMQKRRRNLHKTLVSVKGGKGREKKLQALDRLKEKEKNFATTYNHFLSYNIVKFAKDNLAEQINMEFLALAGEDKNIILRNWSYYQLQQFVEDKAKREGIDVKYVDPYRTSQMCSKCRNYEPGQRESQEKFICKSCHLEINADYNASQNIAHSTKYITNKNQSEYFKKLQHTTE

SEQ ID NO: 76 - protein, 451 aa, Bacillus sp.; identical to SEQ ID NO: 19

SEQ ID NO: 77 - protein, 451 aa, Bacillus sp.; identical to SEQ ID NO: 37

SEQ ID NO: 78 - protein, 451 aa, Bacillus sp.; identical to SEQ ID NO: 37

SEQ ID NO: 79 - protein, 449 aa, Bacillus sp.
MDKKASKVMKYEIIKPVDVDWDVFGSVLRELQFESKNLMNKTIQLCWEWQGFSSDYKKENGEYPKLENHTKYKAITGFVYDRMKTQYNKFNTGNMSVSIKSATDKWKSDLKDILRGEKSIPSYKKNVPIDIHGNSIKIKEVNKNGAILQLSLISSTYKKELGIKNGFFNVLIKIGDNSQHEIVKRLFEGDYKISASKIVKHKYKNKWFLNLTYSFFLEKRELNPDNIMGIDVGVVNALYMAFNESLSRYSIEGGEITKFRKGVEARRKSLLRQGKYCGEGRKGRGRATRIKPIEKLSQRVDNFKDSCNHKYSKYVIDMALKHNCGTIQMEDLTGIAEGEKKSSFLGNWTYFDLQEKITYKAKEHGIKVVKIKPKYTSQRCSKCGFISKDSRPDQATFECIKCNFKTSADYNAARNIAMKDIEKIIAEQLKVQEKAKKLTKKQLANILDD

SEQ ID NO: 80 - protein, 451 aa, Bacillus sp.
MGVTIKIMKYQILCPMNVDWTIFEKHLRNLTYQVRTISNRTIQQLWEFDALSFDYFKERGTYPTVQDLYGCTQKKIDGYIYHTLQSKYPDIHKGNMSTTLQKIIKTWKSRRNEIRKGEMSIPSFRNRIPIDLHNNSVDIIKEKNGDYIAGISLFSRDFHKENGDVPKGKIFVKLGTQKQKSMKVILDRLINQTYSKGACMIHKYKNKWYLSITYKFNAIKENKFDKELIMGIDMGGINTVYFAFNEGFIRSNIKSDEIKMFNERIRQRRINLLKQSKYCSNSRTGKGRTKRLQPIDVLSNKIAKFRNSTNHKYANYIVKQCLKHNCGRIQMELLKGISKNDKVLKDWTYFDLQEKIKNQAEIYGIEVIKVVPAYTSQRCSQCGYICKENRCTQAMFECKQCGYKTHADYNAAKNISTYDIENIINEQLAVQSKLHSKKCMEEYIEELGYLD

SEQ ID NO: 81 - protein, 444 aa, Bacillus sp.; identical to SEQ ID NO: 43

SEQ ID NO: 82 - protein, 448 aa, Bacillus sp.
MTFLGVTIKIMKYQILCPLNVDWTIFEKHLRNLTYQVRTISNRTIQQLWEFDALSFDYFKERGTYPTVQDLYGCTQKKIDGYIYHTLQSKYPDIHKGNMSTTLQKIIKTWKSRRNEIRKGEMSIPSFRNRIPIDLHNNSVDITKEKNGDYIAGISLFSRDFHKENDDVPKGKIFVKLATQKQKSMKVILDRLINQTYSKGACMIHKYKNKWYLSITYKFNAIKENKFDKELIMGIDLGGINTVYSAFNEGFIRSNIKSDEIIRQRRINLLKQSKYCSNSRTGKGRTKRLQPIDVLSNKIAKFRNSTNHKYANYIVKQCLKHNCGRIQMELLKGISKNDRILKDWTYFDLQEKIKNQAEIHGIEVIKVAPAYTSQRCSQCGYICKENRCTQATFECKQCGYKTHADYNAAKNISTYDIENIINKQLAVQSKLHSKKCMEEYIEELGYLD

SEQ ID NO: 83 - protein, 444 aa, Bacillus sp.
MKYQILCPMNVDWTIFEKHLRNLTYQVRTISNRTIQQLWEFDALSFDYFKERGTYPTVQDLYGCTQKKIDGYIYHTLQSKYPDIHKGNMSTTLQKIIKTWKSRRNEIRKGEMSIPSFRNRIPIDLHNNSVDITKEKNGDYIAGISLFSRDFHKENDDVPKGKIFVKLATQKQKSMKVILDRLINQTYSKGACMIHKYKNKWYLSITYKFNAIKENKFDKELIMGIDLGGINTVYFAFNEGFIRSNIKSDEIKMFNERIRQRRINLLKQSKYCSNSRTGKGRTKRLQPIDVLSNKIAKFRNSTNHKYANYIVKQCLKHNCGRIQMELLKGISKNDRILKDWTYFDLQEKIKNQAEIHGIEVIKVAPAYTSQRCSQCGYICKENRCTQATFECKQCGYKTHADYNAAKNISTYDIENIINKQLAVQSKLHSKKCMEEYIEELGYLD

SEQ ID NO: 84 - protein, 448 aa, Bacillus sp.; identical to SEQ ID NO: 82

SEQ ID NO: 85 - protein, 486 aa, Bacillus sp.
MITSRKIRLSIVSDNSTEAYNFIRKEMREQNKALNVSMNHLYFNSIARQKILLADEAYQQKLNDAINSQEKSFAALKEIEQKQCIEDSEKKQVLKERLIKTKNAYEKAKEKVNNLRKSRSKDSFQEYKNIIGQVEQTHLRDIISSQFNLHSDTKDRLTMITNQDFKNDIAEVLSGDRSLRTYKKNNPLYIRSRNTVLYKEGNEFFIKWIKGIVFKCILGVKNQNKTELYKTLECVLAGIYKICDSSMNFNQQNKLILNLTLDMPDKSEHRKVPERIAGIDLGLKIPAYFAVNDVSYIRQAIGKIEDFLKVRTSIQSQKRSLEHALQSSKGGKGRKKKLKALGQFKEKEKSYITTYNHFISKKIISLAIQYGVVQINLELLTLKETQKKSLLRNWSYYQLQQFIEYKAERAGILVKYVDPFHTSQTCSKCGHYEDGQREKQDTFCCKSCGFTENADYNAARNIAASTRYITNKEESEYYRKNNNEIA

SEQ ID NO: 86 - protein, 264 aa, Bacillus sp.
MKVILDRLINQTYSKGACMIHKYKNKWYLSITYKFNAIKENKFEKELIMGIDLGGINTVYSAFNEGFIRSNIKSDEIIRQRRINLLKQSKYCSNSRTGKGRTKRLQPIDVLSNKIAKFRNSTNHKYANYIVKQCLKHNCGRIQMELLKGISKNDRILKDWTYFDLQEKIKNQAEIHGIEVIKVAPAYTSQRCSQCGYICKENRCTQATFECKQCGYKTHADYNAAKNISTYDIENIINKQLAVQSKLHSKKCMEEYIEELGYLD

SEQ ID NO: 87 - protein, 438 aa, Bacillus sp.
MKYQILCPLNVDWTIFEKHLRNLTYQVRTISNRTIQQLWEFDALSFDYFKERGTYPTVQDLYGCTQKKIDGYIYHTLQSKYPDIHKGNMSTTLQKIIKTWKSRRNEIRKGEMSIPSFRNRIPIDLHNNSVDITKEKNGDYIAGISLFSRDFHKENDDVPKGKIFVKLATQKQKSMKVILDRLINQTYSKGACMIHKYKNKWYLSITYKFNAIKENKFDKELIMGIDLGGINTVYSAFNEGFIRSNIKSDEIIRQRRINLLKQSKYCSNSRTGKGRTKRLQPIDVLSNKIAKFRNSTNHKYANYIVKQCLKHNCGRIQMELLKGISKNDRILKDWTYFDLQEKIKNQAEIHGIEVIKVAPAYTSQRCSQCGYICKENRCTQATFECKQCGYKTHADYNAAKNISTYDIENIINKQLAVQSKLHSKKCMEEYIEELGYLD

SEQ ID NO: 88 - protein, 450 aa, Bacillus sp.
MSTVVKVMKYQIVCPVNIEWKVFETYLRTLTYQVRTIGNRTIQKLWEFDNQSLNHFKENGVYPSTQQLYGCTQKTISGYIYDQLKFEYLDINKANMSTTLQKTIKTWNSRKKEIWSGEMSIPSFRNNLPIDIHGNSIQLTKEKSGDYIANLSLFSSSFIRENELSSGKILVKLSTRKQNSMKVILDRLIDGTYAKGASMLHKYKNKWYLSVTYKANAIEESKFDEDFIMGIDMGKVNVLYFAFNKGFIRGSISGEEIEAFRKKIEHRRISLLRQGKYCSENRIGKGRKKRIKPIDVINDKVARFRNSTNHKYANYIVQQCLKYKCGCIQLENLQGISKEQTFLKNWTYFDLQEKIENQANQYGIKVVKIDPSYTSQRCSECGYIHKNNRQDQSTFECQQCNLKVHADYNAAKNISIYNIEKLIQKQLKLQEKLNSKKYTEQYIEQIENIN

SEQ ID NO: 89 - protein, 487 aa, Bacillus sp.
MITSRKIRLSIVSDNSTEAYNFIRKEMREQNKALNVAMNHLYFNYVARQKISFADKAYQHKLNKAIEAQQKSFILLKELEQKQIVETDSSKKSILKERLIKSKTTYEKTKEKLSSLRKARNKELFQEYNNMIGQLEDTHLRDIVSSQFNLLSDTKDRLTKIAYQDFKNDITEVLSGNRSLRTYKKNNPLYIRGRTLNLFKESEEFFIKWTKKIVFKCVLGVKYQNKTELYKTLECILTGEYELCDSSMNFNQQNKLILNLALNIPEKSENRKVPGRIAGIDLGLKIPAYFAVNDVPYIRKALGKIEDFLKVRTNIQSQKRSLQRALQSSKGGKGRKKKLKALDQFKEKEKNYITTYNHFISKKIISLAVQYGVEQINLELLTLKETQKKSLLRNWSYYQLQQFIEYKANREEILIKYVDPFHTSQTCSKCGHYEDGQREKQDTFCCKSCGFIDNADYNAARNIAASTRYITNKEESEYYRKNNNEIA

SEQ ID NO: 90 - protein, 451 aa, Bacillus sp.
MGVTIKIMKYQILCPMNVDWTIFEKHLRNLTYQVRTISNRTIQQLWEFDALSFDYFKERGTYPTVQDLYGCTQKKIDGYIYHTLQSKYPDIHKGNMSTTLQKIIKTWKSRRNEIRKGKMSIPSFRNRIPIDLHNNSVDITKEKNGDYIAGISLFSRDFHKENDDVPKGKIFVKLATQKQKSMKVILDRLINQTYSKGACMIHKYKNKWYLSITYKFNAIKENKFDKELIMGIDLGGINTVYFAFNEGFIRSNIKSDEIKMFNERIRQRRINLLKQSKYCSNGRTGKGRTKRLQPIDVLSNKIAKFRNSTNHKYANYIVKQCLKHNCGRIQMELLKGISKNDRILKDWTYFDLQEKIKNQAEIHGIEVIKVAPAYTSQRCSQCGYICKENRCTQATFECKQCGYKTHADYNAAKNISTYDIENIINKQLAVQSKLHSKKCMEEYIEELGYLD

SEQ ID NO: 91 - protein, 451 aa, Bacillus sp.; identical to SEQ ID NO: 90

SEQ ID NO: 92 - protein, 496 aa, Bacillus sp.
MILTRKIQLVIISENQKEGYSLIRNEIQEQYKALNLSYNHLYFEHNAIQKLKWNDEDYKQKRSKLQELVNKKYEEYQKVKNLEKKEALREAYNKKKQELYKFEKECNEEARKVYQQVVGFTQQTRVRNLINRECNLMSDTKDGITSKVTQDYKNDCKAGLLIGKRSLRNYKKDNPLLVRGRSLKFYKENGEYFIKWNKGTIFKCILHIRKKNIVELQSVLENVLQGAYKVCDSSIAFNNRDMILNLTLNIPNKETQDYIPGRVVGVDLGLKIPAYVSLSDKVYVRKGIGSIDDFLRVRTQMQKRRRQLQESLSAVKGGKGRKKKLKALEHLKEKEANFAKTYNHFLSTQIVTFAVKNHAGQINMEFLEFDKMKNKSLLRNWSYYQLQMMIEYKAKREGIIIKYVDAYLTSQTCSKCDYYEEGQRETQERFMCKSCGFEVNADYNASQNIAKSTRYISDSTESEYHKKKQEALKGILGENDTINEQLSLFNNCDDIA

SEQ ID NO: 93 - protein, 445 aa, Bacillus sp.; identical to SEQ ID NO: 26

SEQ ID NO: 94 - protein, 506 aa, Bacillus sp.; identical to SEQ ID NO: 5

SEQ ID NO: 95 - protein, 451 aa, Bacillus sp.
MGVTIKIMKYQILCPMNVDWIIFEKHLRNLTYQIRTISNRTIQQLWEFDALSFDYFKERGTYPTVQDLYGCTQKKIDGYIYHTLQSKYPDIHKGNMSTTLQKIIKTWKSRRNEIRKGEMSIPSFRNRIPIDLHNNSVDITKEKNGDYIAGISLFSRDFHKENDDVPKGKIFVKLATQKQKSMKVILDRLINQTYSKGACMIHKYKNKWYLSITYKFNAIKEKKFDKELIMGIDMGGINTVYFAFNEGFIRSNIKSDEIKEFNERIRQRRINLLNQSKYCSNSRTGKGRTKRLQPIDVLSNKIAKFRNSTNHKYANYIIKQCLKYNCGRIQMELLKGISKNDKVLKDWTYFDLQEKIKNQAEIHGIEVIKVAPAYTSQRCSQCGYICKENRCTQAMFECKQCGYKTHADYNAAKNIATYDIENIINKQLAAQSKLHSKKCMEEYIEELGYLD

SEQ ID NO: 96 - protein, 445 aa, Bacillus sp.; identical to SEQ ID NO: 22

SEQ ID NO: 97 - protein, 438 aa, Bacillus sp.; identical to SEQ ID NO: 87

SEQ ID NO: 98 - protein, 276 aa, Bacillus sp.
MPQFKSKKNKIKSYTTKCTNHNIAIVGHTIKLPKLGFVRFAKSREVSGRILNATIRRNPSGRHFVSILTETEVQPVEKTGSSVGIDVGLKDYAILSDGITYKNPKFFRTLEEKLAKAQRILSRRTKGSSNWNKQRVKVARIHEHIANARADYLHKLSTEIIKNHDVIGMEDLQVRNMLKNRKLAKAIGEVSWSQFRTMLEYKAKWYGKKVVMVSKTFASSQLCSNCEYKNKDVKNLTIREWDCPSCGAHHDRDRNAALNLKNEAIRLLTVGTTGIA

SEQ ID NO: 99 - protein, 405 aa, Bacillus sp.
MARTTVKKRNEELAKENKVLRHYGIKLRAYPTPTQEEKIAKTMGCARFAFNFYLHEKQEVYQLTRETLDYYTFRKAFNGLKQHPAFNWLKEVDKFSLESALEQVDDAYDHFFKGQNKFPKFKSKHTSKQSYTTKETNGNIALDVEQQMVKLPKVGKISVKLSKKHRTMFQKNGFTAKIKSATVTRHSSGQYYVSLKCEEIMPLEKSVDVTAIPTNEIIGCDLGLTHFLIDSNGQKIENPRYLKKNLEKLAKFQRRLKYKQIGSANYRKHAQKIAKLHLHISNLRKDFLHKISRKLVNENQVIILEDLNVKGMIRNKKLARSIADVGWGMFKTFVSYKANWANKLLILIDRFFPSSKQCNGCKETNPLLSLSDRVWMCPSCGTHHDRDDNAAFNIKEEGIRLLLNA

SEQ ID NO: 100 - protein, 451 aa, Bacillus sp.; identical to SEQ ID NO: 37

SEQ ID NO: 101 - protein, 487 aa, Bacillus sp.
MITSRKIRLAIISDNSTETYNFIRQEMREQNKALNVAMNHLYFNYVARQKISFADKAYQQKLNKAIEAQQKSFILLKELEQKQIVETDSSKKSLLKERLIKTKTTYEKTKEKLSNLRKARNKELFQEYNNIIGQLEETHLRDIVSSQFNLLSDTKDSLTKIAYQDFKNDIVEVLSGDRSLRTYKKNNPLYIRGRSLNLFKESDEFFIKWTKGIVFKCVLGVKYQNKTELYKTLECILAGEYKICDSSMNFNQQKKLILNLVLNIPEKSENRKVPGRIAGIDLGLKIPAYFAVNDVPYIRKAIGNIEDFLKVRTNIHSQQRSLQRALQSSKGGKGRKKKLKALDQFKEKEKNYITTYNHFISKKIITLAIRYGVEQINLELLTLKDTQKKSLLRNWSYYQLQQFIEYKANREGILIKYVDPFHTSQTCSKCSHYEDGQREKQDTFCCKSCGFKDNADYNAARNIAASTKYIIKKEESEYYRQNNNEIA

SEQ ID NO: 102 - protein, 451 aa, Bacillus sp.; identical to SEQ ID NO: 19

SEQ ID NO: 103 - protein, 377 aa, Bacillus sp.; identical to SEQ ID NO: 21

SEQ ID NO: 104 - protein, 450 aa, Bacillus sp.; identical to SEQ ID NO: 8

SEQ ID NO: 105 - protein, 451 aa, Bacillus sp.; identical to SEQ ID NO: 10

SEQ ID NO: 106 - protein, 450 aa, Bacillus sp.; identical to SEQ ID NO: 8

SEQ ID NO: 107 - protein, 450 aa, Bacillus sp.; identical to SEQ ID NO: 8

SEQ ID NO: 108 - protein, 450 aa, Bacillus sp.; identical to SEQ ID NO: 6

SEQ ID NO: 109 - protein, 450 aa, Bacillus sp.; identical to SEQ ID NO: 6

SEQ ID NO: 110 - RNA, 11 nt, Bacillus sp.
aggauugaaau

SEQ ID NO: 111 - RNA, 11 nt, Bacillus sp.
aggauugaaau

SEQ ID NO: 112 - RNA, 11 nt, Bacillus sp.
uggauugaaau

SEQ ID NO: 113 - RNA, 11 nt, Bacillus sp.
aggauugaaau

SEQ ID NO: 114 - RNA, 11 nt, Bacillus sp.
uggauugaaau

SEQ ID NO: 115 - RNA, 18 nt, Bacillus sp.
caauagauguauugaaau

SEQ ID NO: 116 - RNA, 37 nt, Gordonia sp.
gucgugaguacgacgcccucacgcuggcguugcgacc

SEQ ID NO: 117 - RNA, 13 nt, Micrococcus sp.
cagggaugagccc

SEQ ID NO: 118 - RNA, 13 nt, Micrococcus sp.
cagggaugagccc

SEQ ID NO: 119 - RNA, 13 nt, Micrococcus sp.
cagggaugagccc

SEQ ID NO: 120 - RNA, 201 nt, Bacillus sp.
aaacagcuagaauguaacuuaaaguaggucaauguuuaaauucgauguugcaauuuguuuggacaaguggauuaaaacguuccuugaaaaucauauaaagcagccaguuuacgggcuugggcgaauuugcguccaaagggugaggccagguguaaguaagaaccuacaaaagcacucaccaaagggucaacucgauacauu

SEQ ID NO: 121 - RNA, 201 nt, Bacillus sp.; identical to SEQ ID NO: 120

SEQ ID NO: 122 - RNA, 201 nt, Bacillus sp.
aaacagcuagaauguaacuuaaaguaggucaauguuuaaauucgauauugcaauuuguuuggacaaguggauuaaaacguuccuugaaaaucauauaaagcagccaguuuacgggcuugggcgaauuugcguccaaagggugaggccagguguaaguaagaaccuacaaaagcacucaccaaagggucaacucgauacauu

SEQ ID NO: 123 - RNA, 201 nt, Bacillus sp.; identical to SEQ ID NO: 120

SEQ ID NO: 124 - RNA, 201 nt, Bacillus sp.; identical to SEQ ID NO: 122

SEQ ID NO: 125 - RNA, 124 nt, Bacillus sp.
acggucuuuguguuacacccuucaaagcauccaggaugcuuauguaucaauguuuuuuuaacauucgauacuaccuuauggagcguuuacacuugguaaauucaccuuguauuacuucuccauu

SEQ ID NO: 126 - RNA, 97 nt, Micrococcus sp.
uucugggcgccaggcagagcgaagcccucccggcgcggaggggggauggccaggagggcuucuaagucgcaggcccugcgggaugggcucacguucg

SEQ ID NO: 127 - RNA, 96 nt, Micrococcus sp.
guuuacacaggucgaacgugagcccaucccguagggccugcgacuuagaagcccuccuggccauccccccuccgcgccgggagggcuucgcucugc

SEQ ID NO: 128 - RNA, 97 nt, Micrococcus sp.; identical to SEQ ID NO: 126

SEQ ID NO: 129 - RNA, 221 nt, Artificial Sequence
  source: /note="Description of Artificial Sequence: Synthetic polynucleotide"
aaacagcuagaauguaacuuaaaguaggucaauguuuaaauucgauguugcaauuuguuuggacaaguggauuaaaacguuccuugaaaaucauauaaagcagccaguuuacgggcuugggcgaauuugcguccaaagggugaggccagguguaaguaagaaccuacaaaagcacucaccaaagggucaaaaagauccuacauggaugugaggauugaaau

SEQ ID NO: 130 - RNA, 221 nt, Artificial Sequence; identical to SEQ ID NO: 129
  source: /note="Description of Artificial Sequence: Synthetic polynucleotide"

SEQ ID NO: 131 - RNA, 221 nt, Artificial Sequence; identical to SEQ ID NO: 129
  source: /note="Description of Artificial Sequence: Synthetic polynucleotide"

SEQ ID NO: 132 - RNA, 216 nt, Artificial Sequence
  source: /note="Description of Artificial Sequence: Synthetic polynucleotide"
aaacagcuagaauguaacuuaaaguaggucaauguuuaaauucgauauugcaauuuguuuggacaaguggauuaaaacguuccuugaaaaucauauaaagcagccaguuuacgggcuugggcgaauuugcguccaaagggugaggccagguguaaguaagaaccuacaaaagcacucaccaaagggucaacucgauacauuaaaguggauugaaau

SEQ ID NO: 133 - RNA, 114 nt, Artificial Sequence
  source: /note="Description of Artificial Sequence: Synthetic polynucleotide"
uucugggcgccaggcagagcgaagcccucccggcgcggaggggggauggccaggagggcuucuaagucgcaggcccugcgggaugggcucacguucgaaagcagggaugagccc

SEQ ID NO: 134 - RNA, 113 nt, Artificial Sequence
  source: /note="Description of Artificial Sequence: Synthetic polynucleotide"
guuuacacaggucgaacgugagcccaucccguagggccugcgacuuagaagcccuccuggccauccccccuccgcgccgggagggcuucgcucugcaaagcagggacgagccc

SEQ ID NO: 135 - RNA, 114 nt, Artificial Sequence; identical to SEQ ID NO: 133
  source: /note="Description of Artificial Sequence: Synthetic polynucleotide"

SEQ ID NO: 136 - protein, 4 aa, Artificial Sequence
  source: /note="Description of Artificial Sequence: Synthetic peptide"
AAAG

SEQ ID NO: 137 - protein, 82 aa, Artificial Sequence
  source: /note="Description of Artificial Sequence: Synthetic polypeptide"
TNLSDHEKETGKQLVIQESILMLPEEVEEVIGNKPESDILVHTAYDESTDENVMLLTSDAPEYKPWALVIQDSNGENKIKML

SEQ ID NO: 138 - protein, 1493 aa, Homo sapiens
MPNEGIPHSSQTQEQDCLQSQPVSNNEEMAIKQESGGDGEVEEYLSFRSVGDGLSTSAVGCASAAPRRGPALLHIDRHQIQAVEPSAQALELQGLGVDVYDQDVLEQGVLQQVDNAIHEASRASQLVDVEKEYRSVLDDLTSCTTSLRQINKIIEQLSPQAATSRDINRKLDSVKRQKYNKEQQLKKITAKQKHLQAILGGAEVKIELDHASLEEDAEPGPSSLGSMLMPVQETAWEELIRTGQMTPFGTQIPQKQEKKPRKIMLNEASGFEKYLADQAKLSFERKKQGCNKRAARKAPAPVTPPAPVQNKNKPNKKARVLSKKEERLKKHIKKLQKRALQFQGKVGLPKARRPWESDMRPEAEGDSEGEESEYFPTEEEEEEEDDEVEGAEADLSGDGTDYELKPLPKGGKRQKKVPVQEIDDDFFPSSGEEAEAASVGEGGGGGRKVGRYRDDGDEDYYKQRLRRWNKLRLQDKEKRLKLEDDSEESDAEFDEGFKVPGFLFKKLFKYQQTGVRWLWELHCQQAGGILGDEMGLGKTIQIIAFLAGLSYSKIRTRGSNYRFEGLGPTVIVCPTTVMHQWVKEFHTWWPPFRVAILHETGSYTHKKEKLIRDVAHCHGILITSYSYIRLMQDDISRYDWHYVILDEGHKIRNPNAAVTLACKQFRTPHRIILSGSPMQNNLRELWSLFDFIFPGKLGTLPVFMEQFSVPITMGGYSNASPVQVKTAYKCACVLRDTINPYLLRRMKSDVKMSLSLPDKNEQVLFCRLTDEQHKVYQNFVDSKEVYRILNGEMQIFSGLIALRKICNHPDLFSGGPKNLKGLPDDELEEDQFGYWKRSGKMIVVESLLKIWHKQGQRVLLFSQSRQMLDILEVFLRAQKYTYLKMDGTTTIASRQPLITRYNEDTSIFVFLLTTRVGGLGVNLTGANRVVIYDPDWNPSTDTQARERAWRIGQKKQVTVYRLLTAGTIEEKIYHRQIFKQFLTNRVLKDPKQRRFFKSNDLYELFTLTSPDASQSTETSAIFAGTGSDVQTPKCHLKRRIQPAFGADHDVPKRKKFPASNISVNDATSSEEKSEAKGAEVNAVTSNRSDPLKDDPHMSSNVTSNDRLGEETNAVSGPEELSVISGNGECSNSSGTGKTSMPSGDESIDEKLGLSYKRERPSQAQTEAFWENKQMENNFYKHKSKTKHHSVAEEETLEKHLRPKQKPKNSKHCRDAKFEGTRIPHLVKKRRYQKQDSENKSEAKEQSNDDYVLEKLFKKSVGVHSVMKHDAIMDGASPDYVLVEAEANRVAQDALKALRLSRQRCLGAVSGVPTWTGHRGISGAPAGKKSRFGKKRNSNFSVQHPSSTSPTEKCQDGIMKKEGKDNVPEHFSGRAEDADSSSGPLASSSLLAKMRARNHLILPERLESESGHLQEASALLPTTEHDDLLVEMRNFIAFQAHTDGQASTREILQEFESKLSASQSCVFRELLRNLCTFHRTSGGEGIWKLKPEYC

SEQ ID NO: 139 - DNA, 55 bp, Artificial Sequence
  source: /note="Description of Artificial Sequence: Synthetic oligonucleotide"
gtcacatcctacatggatgtgtggattgaaattggaatgggaactaaagtaatgg

SEQ ID NO: 140 - DNA, 201 bp, Artificial Sequence
  source: /note="Description of Artificial Sequence: Synthetic polynucleotide"
aaacagctagaatgtaacttaaagtaggtcaatgtttaaattcgatattgcaatttgtttggacaagtggattaaaacgttccttgaaaatcatataaagcagccagtttacgggcttgggcgaatttgcgtccaaagggtgaggccaggtgtaagtaagaacctacaaaagcactcaccaaagggtcaactcgatacatt

SEQ ID NO: 141 - DNA, 53 bp, Artificial Sequence
  source: /note="Description of Artificial Sequence: Synthetic oligonucleotide"
gtttaaacataacaatagatgtattgaaattggaatgggaactaaagtaatgg

SEQ ID NO: 142 - DNA, 124 bp, Artificial Sequence
  source: /note="Description of Artificial Sequence: Synthetic polynucleotide"
acggtctttgtgttacacccttcaaagcatccaggatgcttatgtatcaatgtttttttaacattcgatactaccttatggagcgtttacacttggtaaattcaccttgtattacttctccatt

SEQ ID NO: 143 - DNA, 55 bp, Artificial Sequence
  source: /note="Description of Artificial Sequence: Synthetic oligonucleotide"
gtcacatcctacatggatgtgaggattgaaattggaatgggaactaaagtaatgg

SEQ ID NO: 144 - DNA, 70 bp, Artificial Sequence
  source: /note="Description of Artificial Sequence: Synthetic oligonucleotide"
aaacagctagaatgtaacttaaagtaggtcaatgtttaaattcgatgttgcaatttgtttggacaagtgg

SEQ ID NO: 145 - DNA, 201 bp, Artificial Sequence
  source: /note="Description of Artificial Sequence: Synthetic polynucleotide"
aaacagctagaatgtaacttaaagtaggtcaatgtttaaattcgatgttgcaatttgtttggacaagtggattaaaacgttccttgaaaatcatataaagcagccagtttacgggcttgggcgaatttgcgtccaaagggtgaggccaggtgtaagtaagaacctacaaaagcactcaccaaagggtcaactcgatacatt

SEQ ID NO: 146 - DNA, 55 bp, Artificial Sequence; identical to SEQ ID NO: 139
  source: /note="Description of Artificial Sequence: Synthetic oligonucleotide"

SEQ ID NO: 147 - DNA, 201 bp, Artificial Sequence; identical to SEQ ID NO: 140
  source: /note="Description of Artificial Sequence: Synthetic polynucleotide"

SEQ ID NO: 148 - DNA, 101 bp, Artificial Sequence
  source: /note="Description of Artificial Sequence: Synthetic polynucleotide"
cagccagtttacgggcttgggcgaatttgcgtccaaagggtgaggccaggtgtaagtaagaacctacaaaagcactcaccaaagggtcaactcgatacatt

SEQ ID NO: 149 - DNA, 21 bp, Artificial Sequence
  source: /note="Description of Artificial Sequence: Synthetic oligonucleotide"
cctaagaagaaaagaaaggtg

SEQ ID NO: 150 - DNA, 48 bp, Artificial Sequence
  source: /note="Description of Artificial Sequence: Synthetic oligonucleotide"
aaaagacctgccgctacaaagaaggccggccaggccaagaaaaagaag

SEQ ID NO: 151 - DNA, 66 bp, Artificial Sequence
  source: /note="Description of Artificial Sequence: Synthetic oligonucleotide"
gactacaaggaccacgacggcgactacaaagatcacgatatcgactacaaggacgacgatgataag

SEQ ID NO: 152 - DNA, 203 bp, Artificial Sequence
  source: /note="Description of Artificial Sequence: Synthetic polynucleotide"
tgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 153 - DNA, 318 bp, Homo sapiens
tgtacaaaaaagcaggctttaaaggaaccaattcagtcgactggatccggtaccaaggtcgggcaggaagagggcctatttcccatgattccttcatatttgcatatacgatacaaggctgttagagagataattagaattaatttgactgtaaacacaaagatattagtacaaaatacgtgacgtagaaagtaataatttcttgggtagtttgcagttttaaaattatgttttaaaatggactatcatatgcttaccgtaacttgaaagtatttcgatttcttggctttatatatcttgtggaaaggacgaaacacc

SEQ ID NO: 154 - DNA, 1644 bp, Artificial Sequence
  source: /note="Description of Artificial Sequence: Synthetic polynucleotide"
gacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactagatccagcgcccgcagtgactcccgaggccagtcacctgttggaagatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttccggaggatctagcggaggctcctctggctctgagacacctggcacaagcgagagcgcaacacctgaaagcagcgggggcagcagcggggggtca

SEQ ID NO: 155 - DNA, 1518 bp, Artificial Sequence
  source: /note="Description of Artificial Sequence: Synthetic polynucleotide"
atgagcacccctctgcagcagcctcaccagaaatccaagaagaccagccagatgataacaacacgcaagttcaagctcgccatcgttagcgacaaccggaacgaggcctacagcttcatccggaacgagatcagaaaccagaacaaggccctgaacgccgcttataaccacctgtacttcgagcacatcgcaaccgagaaactgaagcacagcgatgaggaataccagaagcacctgacaaagtacagagaggtggccaccaacaagtaccaggactacctgaaggtgaaagaaaaagtgaacgacagcaaggacgacgagaagctgcagaagcgggtggataaggctagagaggcctacaacaaggcccaggagaaagtgtacaagatcgagaaggaattcaacaaaaagagcatggaaacctaccagaaggtggtcggcctgagcaaacagacaagaatcggaaagctgctcaagagccagttcaccctgcactacgacaccgaagatagaatcaccagcacagtgatcagccactttaacaatgatatgaagacaggagtgctgagaggcgatagaagcctgagaacatacaagaacagccatcctctgctggttagagccagatctatgaaggtgtacgaggaaaacggcgactacttcatcaagtgggtcaaggggatcgtgtttaagatcgtgatcagcgccggcagcaagcagaaagctaatatcggcgagctgaaatctgtgcttatcaacatcctgaatggccactataaggtgtgcgacagcagcatcagcctgaataaagacctgatcctgaacctgtctctgaacatccctgtgtctaaggagaacgtgttcgtgcccggcagagtggtgggcgtggacctgggcctgaagatcccagcctatgtgtccctgaacgacacaccctacatcaagaagggcatcggcaacatcgacgatttcctgaaggtgcggacccaactgcagagccagagaaagagactgcaaaagaccctggaatgtacctccggcggcaagggcagaaacaagaagctgaagggactggacagactgaaggccaaggaaaaaaacttcgtgaacacctacaaccacttcctgagcaagaaaatcatccagtttgccgtgaagaacaacgccggcgtgatccacctggaagagctgcagttcgacaagctgaaacataagtccctgctgcggaactggtcctactaccaactgcagacaatgatcgagtacaaggctgaacgggaaggcatcgaggtgaagtacgtggacgccagctacaccagccagacctgtagcaagtgcggccactacgaggaaggccagcgggtgctgcaagacaccttcacctgcaagaacaaagagtgcaagggctacgtgcacaaggttaacgccgacttcaacgcctctcagaatatcgccaagtctaccgacatcatccggtgcaccgagatggccaagaacaacgatatcgagaagaatgcc

SEQ ID NO: 156 - DNA, 1518 bp, Artificial Sequence
  source: /note="Description of Artificial Sequence: Synthetic polynucleotide"
atgagcacccctctgcagcagcctcaccagaaatccaagaagaccagccagatgataacaacacgcaagttcaagctcgccatcgttagcgacaaccggaacgaggcctacagcttcatccggaacgagatcagaaaccagaacaaggccctgaacgccgcttataaccacctgtacttcgagcacatcgcaaccgagaaactgaagcacagcgatgaggaataccagaagcacctgacaaagtacagagaggtggccaccaacaagtaccaggactacctgaaggtgaaagaaaaagtgaacgacagcaaggacgacgagaagctgcagaagcgggtggataaggctagagaggcctacaacaaggcccaggagaaagtgtacaagatcgagaaggaattcaacaaaaagagcatggaaacctaccagaaggtggtcggcctgagcaaacagacaagaatcggaaagctgctcaagagccagttcaccctgcactacgacaccgaagatagaatcaccagcacagtgatcagccactttaacaatgatatgaagacaggagtgctgagaggcgatagaagcctgagaacatacaagaacagccatcctctgctggttagagccagatctatgaaggtgtacgaggaaaacggcgactacttcatcaagtgggtcaaggggatcgtgtttaagatcgtgatcagcgccggcagcaagcagaaagctaatatcggcgagctgaaatctgtgcttatcaacatcctgaatggccactataaggtgtgcgacagcagcatcagcctgaataaagacctgatcctgaacctgtctctgaacatccctgtgtctaaggagaacgtgttcgtgcccggcagagtggtgggcgtggccctgggcctgaagatcccagcctatgtgtccctgaacgacacaccctacatcaagaagggcatcggcaacatcgacgatttcctgaaggtgcggacccaactgcagagccagagaaagagactgcaaaagaccctggaatgtacctccggcggcaagggcagaaacaagaagctgaagggactggacagactgaaggccaaggaaaaaaacttcgtgaacacctacaaccacttcctgagcaagaaaatcatccagtttgccgtgaagaacaacgccggcgtgatccacctggccgagctgcagttcgacaagctgaaacataagtccctgctgcggaactggtcctactaccaactgcagacaatgatcgagtacaaggctgaacgggaaggcatcgaggtgaagtacgtggacgccagctacaccagccagacctgtagcaagtgcggccactacgaggaaggccagcgggtgctgcaagacaccttcacctgcaagaacaaagagtgcaagggctacgtgcacaaggttaacgccgccttcaacgcctctcagaatatcgccaagtctaccgacatcatccggtgcaccgagatggccaagaacaacgatatcgagaagaatgcc

SEQ ID NO: 157 - DNA, 7494 bp, Artificial Sequence
  source: /note="Description of Artificial Sequence: Synthetic polynucleotide"
gcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgcgccaccatggcccctaagaagaaaagaaaggtggactacaaggaccacgacggcgactacaaagatcacgatatcgactacaaggacgacgatgataagatgagcacccctctgcagcagcctcaccagaaatccaagaagaccagccagatgataacaacacgcaagttcaagctcgccatcgttagcgacaaccggaacgaggcctacagcttcatccggaacgagatcagaaaccagaacaaggccctgaacgccgcttataaccacctgtacttcgagcacatcgcaaccgagaaactgaagcacagcgatgaggaataccagaagcacctgacaaagtacagagaggtggccaccaacaagtaccaggactacctgaaggtgaaagaaaaagtgaacgacagcaaggacgacgagaagctgcagaagcgggtggataaggctagagaggcctacaacaaggcccaggagaaagtgtacaagatcgagaaggaattcaacaaaaagagcatggaaacctaccagaaggtggtcggcctgagcaaacagacaagaatcggaaagctgctcaagagccagttcaccctgcactacgacaccgaagatagaatcaccagcacagtgatcagccactttaacaatgatatgaagacaggagtgctgagaggcgatagaagcctgagaacatacaagaacagccatcctctgctggttagagccagatctatgaaggtgtacgaggaaaacggcgactacttcatcaagtgggtcaaggggatcgtgtttaagatcgtgatcagcgccggcagcaagcagaaagctaatatcggcgagctgaaatctgtgcttatcaacatcctgaatggccactataaggtgtgcgacagcagcatcagcctgaataaagacctgatcctgaacctgtctctgaacatccctgtgtctaaggagaacgtgttcgtgcccggcagagtggtgggcgtggacctgggcctgaagatcccagcctatgtgtccctgaacgacacaccctacatcaagaagggcatcggcaacatcgacgatttcctgaaggtgcggacccaactgcagagccagagaaagagactgcaaaagaccctggaatgtacctccggcggcaagggcagaaacaagaagctgaagggactggacagactgaaggccaaggaaaaaaacttcgtgaacacctacaaccacttcctgagcaagaaaatcatccagtttgccgtgaagaacaacgccggcgtgatccacctggaagagctgcagttcgacaagctgaaacataagtccctgctgcggaactggtcctactaccaactgcagacaatgatcgagtacaaggctgaacgggaaggcatcgaggtgaagtacgtggacgccagctacaccagccagacctgtagcaagtgcggccactacgaggaaggccagcgggtgctgcaagacaccttcacctgcaagaacaaagagtgcaagggctacgtgcacaaggttaacgccgacttcaacgcctctcagaatatcgccaagtctaccgacatcatccggtgcaccgagatggccaagaacaacgatatcgagaagaatgccaaaagacctgccgctacaaagaaggccggccaggccaagaaaaagaagggtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactagatccagcgcccgcagtgactcccgaggccagtcacctgttggaagatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttaatgaggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggca

SEQ ID NO: 158 - DNA, 7494 bp, Artificial Sequence
  source: /note="Description of Artificial Sequence: Synthetic polynucleotide"
gcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgcgccaccatggcccctaagaagaaaagaaaggtggactacaaggaccacgacggcgactacaaagatcacgatatcgactacaaggacgacgatgataagatgagcacccctctgcagcagcctcaccagaaatccaagaagaccagccagatgataacaacacgcaagttcaagctcgccatcgttagcgacaaccggaacgaggcctacagcttcatccggaacgagatcagaaaccagaacaaggccctgaacgccgcttataaccacctgtacttcgagcacatcgcaaccgagaaactgaagcacagcgatgaggaataccagaagcacctgacaaagtacagagaggtggccaccaacaagtaccaggactacctgaaggtgaaagaaaaagtgaacgacagcaaggacgacgagaagctgcagaagcgggtggataaggctagagaggcctacaacaaggcccaggagaaagtgtacaagatcgagaaggaattcaacaaaaagagcatggaaacctaccagaaggtggtcggcctgagcaaacagacaagaatcggaaagctgctcaagagccagttcaccctgcactacgacaccgaagatagaatcaccagcacagtgatcagccactttaacaatgatatgaagacaggagtgctgagaggcgatagaagcctgagaacatacaagaacagccatcctctgctggttagagccagatctatgaaggtgtacgaggaaaacggcgactacttcatcaagtgggtcaaggggatcgtgtttaagatcgtgatcagcgccggcagcaagcagaaagctaatatcggcgagctgaaatctgtgcttatcaacatcctgaatggccactataaggtgtgcgacagcagcatcagcctgaataaagacctgatcctgaacctgtctctgaacatccctgtgtctaaggagaacgtgttcgtgcccggcagagtggtgggcgtggccctgggcctgaagatcccagcctatgtgtccctgaacgacacaccctacatcaagaagggcatcggcaacatcgacgatttcctgaaggtgcggacccaactgcagagccagagaaagagactgcaaaagaccctggaatgtacctccggcggcaagggcagaaacaagaagctgaagggactggacagactgaaggccaaggaaaaaaacttcgtgaacacctacaaccacttcctgagcaagaaaatcatccagtttgccgtgaagaacaacgccggcgtgatccacctggccgagctgcagttcgacaagctgaaacataagtccctgctgcggaactggtcctactaccaactgcagacaatgatcgagtacaaggctgaacgggaaggcatcgaggtgaagtacgtggacgccagctacaccagccagacctgtagcaagtgcggccactacgaggaaggccagcgggtgctgcaagacaccttcacctgcaagaacaaagagtgcaagggctacgtgcacaaggttaacgccgccttcaacgcctctcagaatatcgccaagtctaccgacatcatccggtgcaccgagatggccaagaacaacgatatcgagaagaatgccaaaagacctgccgctacaaagaaggccggccaggccaagaaaaagaagggtcgacttgacgcgttgatatcaacaagtttgtacaaaaaagcaggctacaaagaggccagcggttccggacgggctgacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactagatccagcgcccgcagtgactcccgaggccagtcacctgttggaagatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttaatgaggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggca

SEQ ID NO: 159 - DNA, 7512 bp, Artificial Sequence
  source: /note="Description of Artificial Sequence: Synthetic polynucleotide"
gcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgcgccaccatggcccctaagaagaaaagaaaggtggactacaaggaccacgacggcgactacaaagatcacgatatcgactacaaggacgacgatgataaggacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactagatccagcgcccgcagtgactcccgaggccagtcacctgttggaagatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttccggaggatctagcggaggctcctctggctctgagacacctggcacaagcgagagcgcaacacctgaaagcagcgggggcagcagcggggggtcaatgagcacccctctgcagcagcctcaccagaaatccaagaagaccagccagatgataacaacacgcaagttcaagctcgccatcgttagcgacaaccggaacgaggcctacagcttcatccggaacgagatcagaaaccagaacaaggccctgaacgccgcttataaccacctgtacttcgagcacatcgcaaccgagaaactgaagcacagcgatgaggaataccagaagcacctgacaaagtacagagaggtggccaccaacaagtaccaggactacctgaaggtgaaagaaaaagtgaacgacagcaaggacgacgagaagctgcagaagcgggtggataaggctagagaggcctacaacaaggcccaggagaaagtgtacaagatcgagaaggaattcaacaaaaagagcatggaaacctaccagaaggtggtcggcctgagcaaacagacaagaatcggaaagctgctcaagagccagttcaccctgcactacgacaccgaagatagaatcaccagcacagtgatcagccactttaacaatgatatgaagacaggagtgctgagaggcgatagaagcctgagaacatacaagaacagccatcctctgctggttagagccagatctatgaaggtgtacgaggaaaacggcgactacttcatcaagtgggtcaaggggatcgtgtttaagatcgtgatcagcgccggcagcaagcagaaagctaatatcggcgagctgaaatctgtgcttatcaacatcctgaatggccactataaggtgtgcgacagcagcatcagcctgaataaagacctgatcctgaacctgtctctgaacatccctgtgtctaaggagaacgtgttcgtgcccggcagagtggtgggcgtggacctgggcctgaagatcccagcctatgtgtccctgaacgacacaccctacatcaagaagggcatcggcaacatcgacgatttcctgaaggtgcggacccaactgcagagccagagaaagagactgcaaaagaccctggaatgtacctccggcggcaagggcagaaacaagaagctgaagggactggacagactgaaggccaaggaaaaaaacttcgtgaacacctacaaccacttcctgagcaagaaaatcatccagtttgccgtgaagaacaacgccggcgtgatccacctggaagagctgcagttcgacaagctgaaacataagtccctgctgcggaactggtcctactaccaactgcagacaatgatcgagtacaaggctgaacgggaaggcatcgaggtgaagtacgtggacgccagctacaccagccagacctgtagcaagtgcggccactacgaggaaggccagcgggtgctgcaagacaccttcacctgcaagaacaaagagtgcaagggctacgtgcacaaggttaacgccgacttcaacgcctctcagaatatcgccaagtctaccgacatcatccggtgcaccgagatggccaagaacaacgatatcgagaagaatgccaaaagacctgccgctacaaagaaggccggccaggccaagaaaaagaagtaatgaggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggca

SEQ ID NO: 160 - DNA, 7512 bp, Artificial Sequence
  source: /note="Description of Artificial Sequence: Synthetic polynucleotide"
gcgatcgcggctcccgacatcttggaccattagctccacaggtatcttcttccctctagtggtcataacagcagcttcagctacctctcaattcaaaaaacccctcaagacccgtttagaggccccaaggggttatgctatcaatcgttgcgttacacacacaaaaaaccaacacacatccatcttcgatggatagcgattttattatctaactgctgatcgagtgtagccagatctagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatgctgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctggtttagtgaaccgtcagatcagatctttgtcgatcctaccatccactcgacacacccgccagcggccgcgccaccatggcccctaagaagaaaagaaaggtggactacaaggaccacgacggcgactacaaagatcacgatatcgactacaaggacgacgatgataaggacgcattggacgattttgatctggatatgctgggaagtgacgccctcgatgattttgaccttgacatgcttggttcggatgcccttgatgactttgacctcgacatgctcggcagtgacgcccttgatgatttcgacctggacatgctgattaactctagaagttccggatctggtagccagtacctgcccgacaccgacgaccggcaccggatcgaggaaaagcggaagcggacctacgagacattcaagagcatcatgaagaagtcccccttcagcggccccaccgaccctagacctccacctagaagaatcgccgtgcccagcagatccagcgccagcgtgccaaaacctgccccccagccttaccccttcaccagcagcctgagcaccatcaactacgacgagttccctaccatggtgttccccagcggccagatctctcaggcctctgctctggctccagcccctcctcaggtgctgcctcaggctcctgctcctgcaccagctccagccatggtgtctgcactggctcaggcaccagcacccgtgcctgtgctggctcctggacctccacaggctgtggctccaccagcccctaaacctacacaggccggcgagggcacactgtctgaagctctgctgcagctgcagttcgacgacgaggatctgggagccctgctgggaaacagcaccgatcctgccgtgttcaccgacctggccagcgtggacaacagcgagttccagcagctgctgaaccagggcatccctgtggcccctcacaccaccgagcccatgctgatggaataccccgaggccatcacccggctcgtgacaggcgctcagaggcctcctgatccagctcctgcccctctgggagcaccaggcctgcctaatggactgctgtctggcgacgaggacttcagctctatcgccgatatggatttctcagccttgctgggctctggcagcggcagccgggattccagggaagggatgtttttgccgaagcctgaggccggctccgctattagtgacgtgtttgagggccgcgaggtgtgccagccaaaacgaatccggccatttcatcctccaggaagtccatgggccaaccgcccactccccgccagcctcgcaccaacaccaaccggtccagtacatgagccagtcgggtcactgaccccggcaccagtccctcagccactagatccagcgcccgcagtgactcccgaggccagtcacctgttggaagatcccgatgaagagacgagccaggctgtcaaagcccttcgggagatggccgatactgtgattccccagaaggaagaggctgcaatctgtggccaaatggacctttcccatccgcccccaaggggccatctggatgagctgacaaccacacttgagtccatgaccgaggatctgaacctggactcacccctgaccccggaattgaacgagattctggataccttcctgaacgacgagtgcctcttgcatgccatgcatatcagcacaggactgtccatcttcgacacatctctgttttccggaggatctagcggaggctcctctggctctgagacacctggcacaagcgagagcgcaacacctgaaagcagcgggggcagcagcggggggtcaatgagcacccctctgcagcagcctcaccagaaatccaagaagaccagccagatgataacaacacgcaagttcaagctcgccatcgttagcgacaaccggaacgaggcctacagcttcatccggaacgagatcagaaaccagaacaaggccctgaacgccgcttataaccacctgtacttcgagcacatcgcaaccgagaaactgaagcacagcgatgaggaataccagaagcacctgacaaagtacagagaggtggccaccaacaagtaccaggactacctgaaggtgaaagaaaaagtgaacgacagcaaggacgacgagaagctgcagaagcgggtggataaggctagagaggcctacaacaaggcccaggagaaagtgtacaagatcgagaaggaattcaacaaaaagagcatggaaacctaccagaaggtggtcggcctgagcaaacagacaagaatcggaaagctgctcaagagccagttcaccctgcactacgacaccgaagatagaatcaccagcacagtgatcagccactttaacaatgatatgaagacaggagtgctgagaggcgatagaagcctgagaacatacaagaacagccatcctctgctggttagagccagatctatgaaggtgtacgaggaaaacggcgactacttcatcaagtgggtcaaggggatcgtgtttaagatcgtgatcagcgccggcagcaagcagaaagctaatatcggcgagctgaaatctgtgcttatcaacatcctgaatggccactataaggtgtgcgacagcagcatcagcctgaataaagacctgatcctgaacctgtctctgaacatccctgtgtctaaggagaacgtgttcgtgcccggcagagtggtgggcgtggccctgggcctgaagatcccagcctatgtgtccctgaacgacacaccctacatcaagaagggcatcggcaacatcgacgatttcctgaaggtgcggacccaactgcagagccagagaaagagactgcaaaagaccctggaatgtacctccggcggcaagggcagaaacaagaagctgaagggactggacagactgaaggccaaggaaaaaaacttcgtgaacacctacaaccacttcctgagcaagaaaatcatccagtttgccgtgaagaacaacgccggcgtgatccacctggccgagctgcagttcgacaagctgaaacataagtccctgctgcggaactggtcctactaccaactgcagacaatgatcgagtacaaggctgaacgggaaggcatcgaggtgaagtacgtggacgccagctacaccagccagacctgtagcaagtgcggccactacgaggaaggccagcgggtgctgcaagacaccttcacctgcaagaacaaagagtgcaagggctacgtgcacaaggttaacgccgccttcaacgcctctcagaatatcgccaagtctaccgacatcatccggtgcaccgagatggccaagaacaacgatatcgagaagaatgccaaaagacctgccgctacaaagaaggccggccaggccaagaaaaagaagtaatgaggatccgcaggcctctgctagcttgactgactgagatacagcgtaccttcagctcacagacatgataagatacattgatgagtttggacaaaccacaactagaatgcagtgaaaaaaatgctttatttgtgaaatttgtgatgctattgctttatttgtaaccattataagctgcaataaacaagttaacaacaacaattgcattcattttatgtttcaggttcagggggaggtgtgggaggttttttaaagcaagtaaaacctctacaaatgtggtattggcccatctctatcggtatcgtagcataaccccttggggcctctaaacgggtcttgaggggttttttgtgcccctcgggccggattgctatctaccggcattggcgcagaaaaaaatgcctgatgcgacgctgcgcgtcttatactcccacatatgccagattcagcaacggatacggcttccccaacttgcccacttccatacgtgtcctccttaccagaaatttatccttaaggtcgtcagctatcctgcaggcgatctctcgatttcgatcaagacattcctttaatggtcttttctggacaccactaggggtcagaagtagttcatcaaactttcttccctccctaatctcattggttaccttgggctatcgaaacttaattaaccagtcaagtcagctacttggcgagatcgacttgtctgggtttcgactacgctcagaattgcgtcagtcaagttcgatctggtccttgctattgcacccgttctccgattacgagtttcatttaaatcatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgagattatcaaaaaggatcttcacctagatccttttaaattaaaaatgaagttttaaatcaatctaaagtatatatgagtaaacttggtctgacagttaccaatgcttaatcagtgaggcacctatctcagcgatctgtctatttcgttcatccatagttgcatttaaatttccgaactctccaaggccctcgtcggaaaatcttcaaacctttcgtccgatccatcttgcaggctacctctcgaacgaactatcgcaagtctcttggccggccttgcgccttggctattgcttggcagcgcctatcgccaggtattactccaatcccgaatatccgagatcgggatcacccgagagaagttcaacctacatcctcaatcccgatctatccgagatccgaggaatatcgaaatcggggcgcgcctggtgtaccgagaacgatcctctcagtgcgagtctcgacgatccatatcgttgcttggcagtcagccagtcggaatccagcttgggacccaggaagtccaatcgtcagatattgtactcaagcctggtcacggcagcgtaccgatctgtttaaacctagatattgatagtctgatcggtcaacgtataatcgagtcctagcttttgcaaacatctatcaagagacaggatcagcaggaggctttcgcatgagtattcaacatttccgtgtcgcccttattcccttttttgcggcattttgccttcctgtttttgctcacccagaaacgctggtgaaagtaaaagatgctgaagatcagttgggtgcgcgagtgggttacatcgaactggatctcaacagcggtaagatccttgagagttttcgccccgaagaacgctttccaatgatgagcacttttaaagttctgctatgtggcgcggtattatcccgtattgacgccgggcaagagcaactcggtcgccgcatacactattctcagaatgacttggttgagtattcaccagtcacagaaaagcatcttacggatggcatgacagtaagagaattatgcagtgctgccataaccatgagtgataacactgcggccaacttacttctgacaacgattggaggaccgaaggagctaaccgcttttttgcacaacatgggggatcatgtaactcgccttgatcgttgggaaccggagctgaatgaagccataccaaacgacgagcgtgacaccacgatgcctgtagcaatggcaacaaccttgcgtaaactattaactggcgaactacttactctagcttcccggcaacagttgatagactggatggaggcggataaagttgcaggaccacttctgcgctcggcccttccggctggctggtttattgctgataaatctggagccggtgagcgtgggtctcgcggtatcattgcagcactggggccagatggtaagccctcccgtatcgtagttatctacacgacggggagtcaggcaactatggatgaacgaaatagacagatcgctgagataggtgcctcactgattaagcattggtaaccgattctaggtgcattggcgcagaaaaaaatgcctgatgcgacgctgcgcgtcttatactcccacatatgccagattcagcaacggatacggcttccccaacttgcccacttccatacgtgtcctccttaccagaaatttatccttaagatcccgaatcgtttaaactcgactctggctctatcgaatctccgtcgtttcgagcttacgcgaacagccgtggcgctcatttgctcgtcgggcatcgaatctcgtcagctatcgtcagcttacctttttggca

SEQ ID NO: 161 - DNA, 239 bp, Artificial Sequence
  source: /note="Description of Artificial Sequence: Synthetic polynucleotide"
aaacagctagaatgtaacttaaagtaggtcaatgtttaaattcgatgttgcaatttgtttggacaagtggattaaaacgttccttgaaaatcatataaagcagccagtttacgggcttgggcgaatttgcgtccaaagggtgaggccaggtgtaagtaagaacctacaaaagcactcaccaaagggtcaaaaagatcctacatggatgtgaggattgaaataagctctcggggtgtgga

SEQ ID NO: 162 - DNA, 239 bp, Artificial Sequence
  source: /note="Description of Artificial Sequence: Synthetic polynucleotide"
aaacagctagaatgtaacttaaagtaggtcaatgtttaaattcgatgttgcaatttgtttggacaagtggattaaaacgttccttgaaaatcatataaagcagccagtttacgggcttgggcgaatttgcgtccaaagggtgaggccaggtgtaagtaagaacctacaaaagcactcaccaaagggtcaaaaagatcctacatggatgtgaggattgaaatactgtaaaagatgtaaag

SEQ ID NO: 163 - DNA, 239 bp, Artificial Sequence
  source: /note="Description of Artificial Sequence: Synthetic polynucleotide"
aaacagctagaatgtaacttaaagtaggtcaatgtttaaattcgatgttgcaatttgtttggacaagtggattaaaacgttccttgaaaatcatataaagcagccagtttacgggcttgggcgaatttgcgtccaaagggtgaggccaggtgtaagtaagaacctacaaaagcactcaccaaagggtcaaaaagatcctacatggatgtgaggattgaaatatgttttatgttactgta

SEQ ID NO: 164 - DNA, 239 bp, Artificial Sequence
  source: /note="Description of Artificial Sequence: Synthetic polynucleotide"
aaacagctagaatgtaacttaaagtaggtcaatgtttaaattcgatgttgcaatttgtttggacaagtggattaaaacgttccttgaaaatcatataaagcagccagtttacgggcttgggcgaatttgcgtccaaagggtgaggccaggtgtaagtaagaacctacaaaagcactcaccaaagggtcaaaaagatcctacatggatgtgaggattgaaatacagtaacataaaacata

SEQ ID NO: 165 - DNA, 239 bp, Artificial Sequence
  source: /note="Description of Artificial Sequence: Synthetic polynucleotide"
aaacagctagaatgtaacttaaagtaggtcaatgtttaaattcgatgttgcaatttgtttggacaagtggattaaaacgttccttgaaaatcatataaagcagccagtttacgggcttgggcgaatttgcgtccaaagggtgaggccaggtgtaagtaagaacctacaaaagcactcaccaaagggtcaaaaagatcctacatggatgtgaggattgaaatggcaaaggagcacatcag

SEQ ID NO: 166 - DNA, 239 bp, Artificial Sequence
  source: /note="Description of Artificial Sequence: Synthetic polynucleotide"
aaacagctagaatgtaacttaaagtaggtcaatgtttaaattcgatgttgcaatttgtttggacaagtggattaaaacgttccttgaaaatcatataaagcagccagtttacgggcttgggcgaatttgcgtccaaagggtgaggccaggtgtaagtaagaacctacaaaagcactcaccaaagggtcaaaaagatcctacatggatgtgaggattgaaatgccaaagcagatgtgttt

SEQ ID NO: 167 - DNA, 239 bp, Artificial Sequence
  source: /note="Description of Artificial Sequence: Synthetic polynucleotide"
aaacagctagaatgtaacttaaagtaggtcaatgtttaaattcgatgttgcaatttgtttggacaagtggattaaaacgttccttgaaaatcatataaagcagccagtttacgggcttgggcgaatttgcgtccaaagggtgaggccaggtgtaagtaagaacctacaaaagcactcaccaaagggtcaaaaagatcctacatggatgtgaggattgaaatgtcctgtagacaggcatg

SEQ ID NO: 168 - DNA, 239 bp, Artificial Sequence
  source: /note="Description of Artificial Sequence: Synthetic polynucleotide"
aaacagctagaatgtaacttaaagtaggtcaatgtttaaattcgatgttgcaatttgtttggacaagtggattaaaacgttccttgaaaatcatataaagcagccagtttacgggcttgggcgaatttgcgtccaaagggtgaggccaggtgtaagtaagaacctacaaaagcactcaccaaagggtcaaaaagatcctacatggatgtgaggattgaaatggtggtgtggggggggag

SEQ ID NO: 169 - DNA, 239 bp, Artificial Sequence
  source: /note="Description of Artificial Sequence: Synthetic polynucleotide"
aaacagctagaatgtaacttaaagtaggtcaatgtttaaattcgatgttgcaatttgtttggacaagtggattaaaacgttccttgaaaatcatataaagcagccagtttacgggcttgggcgaatttgcgtccaaagggtgaggccaggtgtaagtaagaacctacaaaagcactcaccaaagggtcaaaaagatcctacatggatgtgaggattgaaatccacactcacgcatgcct

SEQ ID NO: 170 - DNA, 239 bp, Artificial Sequence
  source: /note="Description of Artificial Sequence: Synthetic polynucleotide"
aaacagctagaatgtaacttaaagtaggtcaatgtttaaattcgatgttgcaatttgtttggacaagtggattaaaacgttccttgaaaatcatataaagcagccagtttacgggcttgggcgaatttgcgtccaaagggtgaggccaggtgtaagtaagaacctacaaaagcactcaccaaagggtcaaaaagatcctacatggatgtgaggattgaaatcagcacagcagcttcccc

SEQ ID NO: 171 - DNA, 239 bp, Artificial Sequence
  source: /note="Description of Artificial Sequence: Synthetic polynucleotide"
aaacagctagaatgtaacttaaagtaggtcaatgtttaaattcgatgttgcaatttgtttggacaagtggattaaaacgttccttgaaaatcatataaagcagccagtttacgggcttgggcgaatttgcgtccaaagggtgaggccaggtgtaagtaagaacctacaaaagcactcaccaaagggtcaaaaagatcctacatggatgtgaggattgaaatgcccggatgagggagagc

SEQ ID NO: 172 - DNA, 239 bp, Artificial Sequence
  source: /note="Description of Artificial Sequence: Synthetic polynucleotide"
aaacagctagaatgtaacttaaagtaggtcaatgtttaaattcgatgttgcaatttgtttggacaagtggattaaaacgttccttgaaaatcatataaagcagccagtttacgggcttgggcgaatttgcgtccaaagggtgaggccaggtgtaagtaagaacctacaaaagcactcaccaaagggtcaaaaagatcctacatggatgtgaggattgaaatacccctccccccccacac

SEQ ID NO: 173 - protein, 506 aa, Artificial Sequence
  source: /note="Description of Artificial Sequence: Synthetic polypeptide"
MSTPLQQPHQKSKKTSQMITTRKFKLAIVSDNRNEAYSFIRNEIRNQNKALNAAYNHLYFEHIATEKLKHSDEEYQKHLTKYREVATNKYQDYLKVKEKVNDSKDDEKLQKRVDKAREAYNKAQEKVYKIEKEFNKKSMETYQKVVGLSKQTRIGKLLKSQFTLHYDTEDRITSTVISHFNNDMKTGVLRGDRSLRTYKNSHPLLVRARSMKVYEENGDYFIKWVKGIVFKIVISAGSKQKANIGELKSVLINILNGHYKVCDSSISLNKDLILNLSLNIPVSKENVFVPGRVVGVALGLKIPAYVSLNDTPYIKKGIGNIDDFLKVRTQLQSQRKRLQKTLECTSGGKGRNKKLKGLDRLKAKEKNFVNTYNHFLSKKIIQFAVKNNAGVIHLAELQFDKLKHKSLLRNWSYYQLQTMIEYKAEREGIEVKYVDASYTSQTCSKCGHYEEGQRVLQDTFTCKNKECKGYVHKVNAAFNASQNIAKSTDIIRCTEMAKNNDIEKNA

SEQ ID NO: 174 - DNA, 56 bp, Artificial Sequence
  source: /note="Description of Artificial Sequence: Synthetic oligonucleotide"
ctacgaattcgtttgccattactttagttcccattccaaaaaccggatccatatgg

SEQ ID NO: 175 - RNA, 242 nt, Artificial Sequence
  source: /note="Description of Artificial Sequence: Synthetic polynucleotide"
gggaaacagcuagaauguaacuuaaaguaggucaauguuuaaauucgauguugcaauuuguuuggacaaguggauuaaaacguuccuugaaaaucauauaaagcagccaguuuacgggcuugggcgaauuugcguccaaagggugaggccagguguaaguaagaaccuacaaaagcacucaccaaagggucaaaaagauccuacauggaugugaggauugaaauuggaaugggaacuaaagu

SEQ ID NO: 176 - DNA, 8 bp, Artificial Sequence
  source: /note="Description of Artificial Sequence: Synthetic oligonucleotide"
  modified_base (1)..(8): a, c, t, g, unknown or other
nnnnnnnn

SEQ ID NO: 177 - protein, 199 aa, Homo sapiens
MEASPASGPRHLMDPHIFTSNFNNGIGRHKTYLCYEVERLDNGTSVKMDQHRGFLHNQAKNLLCGFYGRHAELRFLDLVPSLQLDPAQIYRVTWFISWSPCFSWGCAGEVRAFLQENTHVRLRIFAARIYDYDPLYKEALQMLRDAGAQVSIMTYDEFKHCWDTFVDHQGCPFQPWDGLDEHSQALSGRLRAILQNQGN

SEQ ID NO: 178 - protein, 173 aa, Gordonia sp.
MSGMKIRAYAPAGGPSLAEVALSNVTALCNRWLDQPASEVWREQAANEILAAMDTDPVSDLGIVGLNVKNGEATLDVASCGPASQATLLAVVAAIGDLLHTENPPNYVEFEVIQGTRSYIAHVRTGDRPSPHTLRRQAEQRITAALDVLDDHADDPTEVIARARAVLREQAEQ

SEQ ID NO: 179 - protein, 135 aa, Gordonia sp.
MTATPPAAETVWAVQFTGVGGEPVVHTRDLRGVPFADDAHAAAHATHLQGLGRRDARTVYQANGSTGWMDPTPQPPQGQPPDRAADVTEVASALHRHSGGRLAVTDAHSIAAVALRMAAAVTEARAPAARSRHSI

SEQ ID NO: 180 - protein, 191 aa, Gordonia sp.
MTDPTASAGTHPAALAAVAVANSTAATVQAICVDGVRRHMHVRTHFETALPHRTQHAGRRFRIVAEHDDRVAIEFTDRGDRMLAYPEEITADTYGYPEGAHLIRAMYAEAAESLSHRIFMLEFTREWWLELSEGRGGPLDTAPQSVVLLGSRMVRAADGGRAVRLYRRAADGAPDVGMLRSAARDFVGCLH

SEQ ID NO: 181 - protein, 234 aa, Gordonia sp.
MTAAADPVDMFVVVIEHRYGTNVGVHASRGDAVTEVAQFARNWWNDQPPATMSPDSRRPEIPADDGAVIDAYFHAMAGQESYTITSTPLARATVDGLMFSAAGAAAPPAASTIDAVRAEYTAQKARQDQARERARYLAVLLLVDVLARRLPDVKQLLVEGDSEEDWWNIRWPVASGTPDTEISQINADIGALPTDVGYKVWGHICTRASEDRNQLVVDVAELQKYAANGGDRRK

SEQ ID NO: 182 - protein, 99 aa, Micrococcus sp.
MTTFYYSVNEMARQAGFTSPSTWTRIKNDLRAPDAVMVSKIQGAGWTLDSWEDAANNELHTRREPLMKAITRLRLEEEALRQEYGVTDQDEPADQKAEG

SEQ ID NO: 183 - protein, 149 aa, Micrococcus sp.
MNAIELKCRREALGLSRDALADTLGVAEKSITRWEFGKNPPRDWSWIDSAMTRLEDYRERLVEELIAETLRVHEQAGAALMLTYASRGSFYRWLPEMEEPDMGDGIEVIPVELHRAATAEAARRLRVSHGLHAVIEAVPTPQEGEGAGS

SEQ ID NO: 184 - protein, 245 aa, Micrococcus sp.
MSVRRWVSSVVAVVAALGVVGAVGGGHVQGVQVPEVKLPKVALPSGVELPSLGDTSPTPAAGPVGGQLEQLALTSAAPASAYTRDAFGQRWADVDRNGCDTRNDVLRRDLTQVQIKPGTQGCKVLSGQLVDPYSGATIPFSSQDSQAVHIDHTVSLADAWASGAWAWDESQRTAFANDPANLLAVDGPANTSKSDATAADWLPDTVAGRCELVEHQVVVKAKWGLSVTERERAAMRRVLASCPSG

SEQ ID NO: 185 - protein, 99 aa, Micrococcus sp.; identical to SEQ ID NO: 182

SEQ ID NO: 186 - protein, 123 aa, Micrococcus sp.
  MOD_RES (107)..(107): Any amino acid
MNAIELKCRREALGLSRDALADTLGVAEKSITRWEFGKNPPRDWSWIDSAMTRLEDYRERLVEELIAETLRVHEQAGAALMLTYASRGSFYRWLPEMEEPDMGDGIXVIEAVPTPQEREGAGS

SEQ ID NO: 187 - protein, 245 aa, Micrococcus sp.
MSVRRWVSSVVAVVAALGVAGAVGGGHVQGVQVPEVKLPKVALPSGVELPSLGDTSPTPAAGPVGGQLEQLALTSAAPASAYTRDAFGQRWADVDRNGCDTRNDVLRRDLTQVQIKPGTQGCKVLSGQLVDPYSGATIPFSSQDSQAVQIDHTVSLADAWASGAWAWDESQRTAFANDPANLLAVDGPANNSKSDATAADWLPDTVAGRCELVEHQVVVKAKWGLSVTERERAAMRRVLASCPAG

SEQ ID NO: 188 - protein, 99 aa, Micrococcus sp.; identical to SEQ ID NO: 182

SEQ ID NO: 189 - protein, 149 aa, Micrococcus sp.
MNAIELKCRREALGLSRDALADTLGVAEKSITRWEFGKNPPRDWSWIDSAMTRLEDYRERLVEELIAETLRVHEQAGAALMLTYASRGSFYRWLPEMEEPDMGDGIEVIPVELHRAATAEAARRLRVSHGLHAVIEAVPTPQEREGAGS

SEQ ID NO: 190 - protein, 245 aa, Micrococcus sp.
MSVRRWVSGVVAVVAALGVAGAVGGGHVQGVQVPEVKLPKVALPSGVELPSLGDTSPTPAAGPVGGQLEQLALTSAAPASAYTRDAFGQRWADVDRNGCDTRNDVLRRDLTQVQIKPGTQGCKVLSGQLVDPYSGATIPFSSQDSQAVHIDHTVSLADAWASGAWAWDESQRTAFANDPANLLAVDGPANTSKSDATAADWLPGTVAGRCELVEHQVVVKAKWGLSVTERERAAMRRVLASCPAG

SEQ ID NO: 191 - protein, 206 aa, Paeniglutamicibacter sp.
MLGQGFDQGLEFGFVLGKGLVQQLLACPVQGHRVVVGLADIDADEHVDGFVVLDHCAPHSWGHRRVASDETVLACPAAPRLGIHVTTDNWTGAVPSRSGPYLRYPRCLSVPVTAPPGSWMTGGVNHAGTDRPAIPILAHGARLRDYKKGNGGGADAPLNAASCGGQWRMACDRTSVACGRQRPLSSGSRRLPRHWSLPHPGCTFIP

SEQ ID NO: 192 - protein, 106 aa, Streptomyces sp.
  MOD_RES (65)..(66): Any amino acid
MGSSAVRALRVLPPGAVDTTPPPAANRPASGPAAAPAGPVKTRETASAGYRLALDAHRAAVDPAXXSRIPADAQAAFADYRAQARETASAVQDAAIRRARVERTAA

SEQ ID NO: 193 - RNA, 244 nt, Artificial Sequence
  source: /note="Description of Artificial Sequence: Synthetic polynucleotide"
gggaaacagcuagaauguaacuuaaaguaggucaauguuuaaauucgauguugcaauuuguuuggacaaguggauuaaaacguuccuugaaaaucauauaaagcagccaguuuacgggcuugggcgaauuugcguccaaagggugaggccagguguaaguaagaaccuacaaaagcacucaccaaagggucaaaaagauccuacauggaugugaggauugaaauguacgagauauaggaagcug

SEQ ID NO: 194 - RNA, 242 nt, Artificial Sequence
  source: /note="Description of Artificial Sequence: Synthetic polynucleotide"
gggaaacagcuagaauguaacuuaaaguaggucaauguuuaaauucgauguugcaauuuguuuggacaaguggauuaaaacguuccuugaaaaucauauaaagcagccaguuuacgggcuugggcgaauuugcguccaaagggugaggccagguguaaguaagaaccuacaaaagcacucaccaaagggucaaaaagauccuacauggaugugaggauugaaauacuacaacagccacaacg

SEQ ID NO: 195 - DNA, 50 bp, Artificial Sequence
  source: /note="Description of Artificial Sequence: Synthetic oligonucleotide"
aggagcggacagcagcttcctatatctcgtacaggaccgtcaacatggtt

SEQ ID NO: 196 - DNA, 50 bp, Artificial Sequence
  source: /note="Description of Artificial Sequence: Synthetic oligonucleotide"
agccatgatatagacgttgtggctgttgtagtaggaccgtcaacatggtt

SEQ ID NO: 197 - DNA, 20 bp, Artificial Sequence
  source: /note="Description of Artificial Sequence: Synthetic oligonucleotide"
agcggtgggaaaggaatccc

SEQ ID NO: 198 - DNA, 22 bp, Artificial Sequence
  source: /note="Description of Artificial Sequence: Synthetic oligonucleotide"
ttcgattccggagagggagcct

SEQ ID NO: 199 - DNA, 50 bp, Artificial Sequence
  source: /note="Description of Artificial Sequence: Synthetic oligonucleotide"
aggagcggacagcagcttcctatatctcgtacagggaactcaacatggtt

SEQ ID NO: 200 - DNA, 50 bp, Artificial Sequence
  source: /note="Description of Artificial Sequence: Synthetic oligonucleotide"
agccatgatatagacgttgtggctgttgtagtagggaactcaacatggtt

SEQ ID NO: 201 - DNA, 32 bp, Bacillus sp.
gtcacatcctacatggatgtgaggattgaaat

SEQ ID NO: 202 - DNA, 32 bp, Bacillus sp.; identical to SEQ ID NO: 201

SEQ ID NO: 203 - DNA, 32 bp, Bacillus sp.
gtcacatcctacatgggtgtgtggattgaaat

SEQ ID NO: 204 - DNA, 32 bp, Bacillus sp.; identical to SEQ ID NO: 201

SEQ ID NO: 205 - DNA, 32 bp, Bacillus sp.
gtcacaccctacatgggtgtaaggattgaaat

SEQ ID NO: 206 - DNA, 30 bp, Bacillus sp.
gttwaaacataacaatagatgtwttgaaat

SEQ ID NO: 207 - DNA, 32 bp, Bacillus sp.; identical to SEQ ID NO: 203

SEQ ID NO: 208 - DNA, 30 bp, Bacillus sp.
gttaaaacaaaacaatagatgtattgaaat

SEQ ID NO: 209 - DNA, 30 bp, Bacillus sp.
gtttaaacttaacaatagatgtattgaaat

SEQ ID NO: 210 - DNA, 31 bp, Bacillus sp.
gtttactattaacattggatgtatttaaatt

SEQ ID NO: 211 - DNA, 37 bp, Gordonia sp.
gtcgtgagtacgacgccctcacgctggcgttgcgacc

SEQ ID NO: 212 - DNA, 29 bp, Micrococcus sp.
ggaagccccgcgcacgcagggatgagccc

SEQ ID NO: 213 - DNA, 29 bp, Micrococcus sp.
ggaagccccgcgcacgcagggatgagccc

SEQ ID NO: 214 - DNA, 29 bp, Paeniglutamicibacter sp.
gtccttcccgggcgcgcgcggaatggctc

SEQ ID NO: 215 - DNA, 29 bp, Streptomyces sp.
gtacggcccacacgcgcaggggacgaccg

SEQ ID NO: 216 - DNA, 29 bp, Micrococcus sp.
ggaagccccgcgcacgcagggatgagccc

SEQ ID NO: 217 - DNA, 29 bp, Bacillus sp.
gtcggccccgcgcacgcggagatgagccc

SEQ ID NO: 218 - DNA, 30 bp, Bacillus sp.
gtttgacactaacataagatgtatttaaat

SEQ ID NO: 219 - DNA, 30 bp, Bacillus sp.
gtttaacactaacataagatgtatttaaat

SEQ ID NO: 220 - DNA, 30 bp, Bacillus sp.
gtttaacattaacataagatgtatttaaat

SEQ ID NO: 221 - DNA, 32 bp, Bacillus sp.
gtcgcaccttatataggtgcgtggattgaaat

SEQ ID NO: 222 - DNA, 30 bp, Bacillus sp.
gtttastactaacataagatgtatttaaat

SEQ ID NO: 223 - DNA, 30 bp, Bacillus sp.
gtttaatactaacataagatgtatttaaat

SEQ ID NO: 224 - DNA, 32 bp, Bacillus sp.; identical to SEQ ID NO: 201

SEQ ID NO: 225 - DNA, 22 bp, Bacillus sp.
tactcatgaacacctcatgwgt

SEQ ID NO: 226 - DNA, 33 bp, Bacillus sp.
gtttactaytaacataagatgtatttaaatggt

SEQ ID NO: 227 - DNA, 30 bp, Bacillus sp.
gtttastaytaacataagatgtatttaaat

SEQ ID NO: 228 - DNA, 30 bp, Bacillus sp.
gtttagtactaacataagatgtatttaaat

SEQ ID NO: 229 - DNA, 29 bp, Bacillus sp.
gttttgaataaactatgtagaatgtgaat

SEQ ID NO: 230 - DNA, 30 bp, Bacillus sp.; identical to SEQ ID NO: 219

SEQ ID NO: 231 - DNA, 30 bp, Bacillus sp.; identical to SEQ ID NO: 219

SEQ ID NO: 232 - DNA, 30 bp, Bacillus sp.; identical to SEQ ID NO: 219

SEQ ID NO: 233 - DNA, 29 bp, Bacillus sp.
gttttgaataaactatgtagaatgtgaat

SEQ ID NO: 234 - DNA, 30 bp, Bacillus sp.; identical to SEQ ID NO: 219

SEQ ID NO: 235 - DNA, 19 bp, Artificial sequence
  modified_base (1)..(19): a, c, t, g, unknown or other
nnnnnnnnnnnnnnnnnnn

SEQ ID NO: 236 - DNA, 30 bp, Bacillus sp.; identical to SEQ ID NO: 228

SEQ ID NO: 237 - DNA, 30 bp, Bacillus sp.; identical to SEQ ID NO: 219

SEQ ID NO: 238 - DNA, 19 bp, Artificial sequence
  modified_base (1)..(19): a, c, t, g, unknown or other
nnnnnnnnnnnnnnnnnnn

SEQ ID NO: 239 - DNA, 30 bp, Bacillus sp.
gtttartactaacataagatgtatttaaat

SEQ ID NO: 240 - DNA, 30 bp, Bacillus sp.; identical to SEQ ID NO: 219

SEQ ID NO: 241 - DNA, 30 bp, Bacillus sp.
gttaaaacataacaatagatgtattgaaat

SEQ ID NO: 242 - DNA, 30 bp, Bacillus sp.; identical to SEQ ID NO: 241

SEQ ID NO: 243 - DNA, 30 bp, Bacillus sp.; identical to SEQ ID NO: 219

SEQ ID NO: 244 - DNA, 30 bp, Bacillus sp.
gtttracactaacataagatgtatttaaat

SEQ ID NO: 245 - DNA, 30 bp, Bacillus sp.; identical to SEQ ID NO: 220

SEQ ID NO: 246 - DNA, 32 bp, Bacillus sp.; identical to SEQ ID NO: 201

SEQ ID NO: 247 - DNA, 32 bp, Bacillus sp.
gtcacatcctacatggatgtgtggattgaaat

SEQ ID NO: 248 - DNA, 30 bp, Bacillus sp.; identical to SEQ ID NO: 228

SEQ ID NO: 249 - DNA, 30 bp, Bacillus sp.; identical to SEQ ID NO: 220

SEQ ID NO: 250 - DNA, 33 bp, Bacillus sp.
gtcacatcctacatggatgtgaggattgaaatt

SEQ ID NO: 251 - DNA, 19 bp, Artificial sequence
  modified_base (1)..(19): a, c, t, g, unknown or other
nnnnnnnnnnnnnnnnnnn

SEQ ID NO: 252 - DNA, 30 bp, Bacillus sp.; identical to SEQ ID NO: 220

SEQ ID NO: 253 - DNA, 30 bp, Bacillus sp.; identical to SEQ ID NO: 220

SEQ ID NO: 254 - DNA, 30 bp, Bacillus sp.; identical to SEQ ID NO: 227

SEQ ID NO: 255 - DNA, 30 bp, Bacillus sp.; identical to SEQ ID NO: 219

SEQ ID NO: 256 - DNA, 30 bp, Bacillus sp.
  modified_base (7)..(7): a, c, t, g, unknown or other
gkttganactaayataagatgtatttaaat

SEQ ID NO: 257 - DNA, 30 bp, Bacillus sp.; identical to SEQ ID NO: 219

SEQ ID NO: 258 - DNA, 30 bp, Bacillus sp.
gtttactaytaacataagatgtatttaaat

SEQ ID NO: 259 - DNA, 30 bp, Bacillus sp.; identical to SEQ ID NO: 227

SEQ ID NO: 260 - DNA, 30 bp, Bacillus sp.; identical to SEQ ID NO: 219

SEQ ID NO: 261 - DNA, 30 bp, Bacillus sp.; identical to SEQ ID NO: 223

SEQ ID NO: 262 - DNA, 30 bp, Bacillus sp.; identical to SEQ ID NO: 219

SEQ ID NO: 263 - DNA, 30 bp, Bacillus sp.; identical to SEQ ID NO: 239

SEQ ID NO: 264 - DNA, 30 bp, Bacillus sp.
gtttagtaytaacataagatgtatttaaat

SEQ ID NO: 265 - DNA, 30 bp, Bacillus sp.; identical to SEQ ID NO: 219

SEQ ID NO: 266 - DNA, 30 bp, Bacillus sp.; identical to SEQ ID NO: 223

SEQ ID NO: 267 - DNA, 30 bp, Bacillus sp.
gtttascattaacataagatgtatttaaat

SEQ ID NO: 268 - DNA, 30 bp, Bacillus sp.; identical to SEQ ID NO: 218

SEQ ID NO: 269 - DNA, 29 bp, Bacillus sp.
rtttgatatcaactatgtggaatgtaaat

SEQ ID NO: 270 - DNA, 29 bp, Bacillus sp.
rtttgatatcaactatgtggaatgtaaat

SEQ ID NO: 271 - DNA, 29 bp, Bacillus sp.
rtttgatatcaactatgtggaatgtaaat

SEQ ID NO: 272 - DNA, 30 bp, Bacillus sp.; identical to SEQ ID NO: 219

SEQ ID NO: 273 - DNA, 30 bp, Bacillus sp.; identical to SEQ ID NO: 239

SEQ ID NO: 274 - DNA, 30 bp, Bacillus sp.; identical to SEQ ID NO: 228

SEQ ID NO: 275 - DNA, 29 bp, Bacillus sp.
gttttgaataaactatgtagaatgtgaat

SEQ ID NO: 276 - DNA, 30 bp, Bacillus sp.; identical to SEQ ID NO: 219

SEQ ID NO: 277 - DNA, 30 bp, Bacillus sp.; identical to SEQ ID NO: 219

SEQ ID NO: 278 - DNA, 30 bp, Bacillus sp.; identical to SEQ ID NO: 219

SEQ ID NO: 279 - DNA, 34 bp, Bacillus sp.
agtaacaatacgtatagctacgttaattgtagga

SEQ ID NO: 280 - DNA, 30 bp, Bacillus sp.; identical to SEQ ID NO: 219

SEQ ID NO: 281 - DNA, 30 bp, Bacillus sp.; identical to SEQ ID NO: 219

SEQ ID NO: 282 - DNA, 30 bp, Bacillus sp.; identical to SEQ ID NO: 228

SEQ ID NO: 283 - DNA, 30 bp, Bacillus sp.; identical to SEQ ID NO: 222

SEQ ID NO: 284 - DNA, 30 bp, Bacillus sp.; identical to SEQ ID NO: 228

SEQ ID NO: 285 - DNA, 29 bp, Bacillus sp.
gttttaatcacaatatgtattgttagaat

SEQ ID NO: 286 - DNA, 30 bp, Bacillus sp.
gtttagyactaacataagatgtatttaaat

SEQ ID NO: 287 - DNA, 30 bp, Bacillus sp.; identical to SEQ ID NO: 222

SEQ ID NO: 288 - DNA, 30 bp, Bacillus sp.
gtttaaacataacaatagatgtattgaaat

SEQ ID NO: 289 - DNA, 29 bp, Bacillus sp.
rttttaaatacaatatgtattgttagaat

SEQ ID NO: 290 - DNA, 30 bp, Bacillus sp.; identical to SEQ ID NO: 227

SEQ ID NO: 291 - DNA, 30 bp, Bacillus sp.; identical to SEQ ID NO: 227

SEQ ID NO: 292 - DNA, 29 bp, Bacillus sp.
gtttgatatcaactatatggaatgtaaat

SEQ ID NO: 293 - DNA, 30 bp, Bacillus sp.; identical to SEQ ID NO: 227

SEQ ID NO: 294 - DNA, 32 bp, Bacillus sp.
gtcacatcctacatgggtgtgaggattgaaat

SEQ ID NO: 295 - DNA, 30 bp, Bacillus sp.
gtttaahattaacataagatgtatttaaat

SEQ ID NO: 296 - DNA, 30 bp, Bacillus sp.; identical to SEQ ID NO: 227

SEQ ID NO: 297 - DNA, 30 bp, Bacillus sp.; identical to SEQ ID NO: 227

SEQ ID NO: 298 - DNA, 21 bp, Bacillus sp.
ggtcatgttgtcattacattg

SEQ ID NO: 299 - DNA, 18 bp, Bacillus sp.
atcccaaaaacgttaggt

SEQ ID NO: 300 - DNA, 30 bp, Bacillus sp.; identical to SEQ ID NO: 219

SEQ ID NO: 301 - DNA, 29 bp, Bacillus sp.
gtcttaattacaatatgtattgttagaat

SEQ ID NO: 302 - DNA, 30 bp, Bacillus sp.; identical to SEQ ID NO: 219

SEQ ID NO: 303 - DNA, 19 bp, Bacillus sp.
  modified_base (5)..(5): a, c, t, g, unknown or other
gatanaaagtgtttcatat

SEQ ID NO: 304 - DNA, 19 bp, Artificial sequence
  modified_base (1)..(19): a, c, t, g, unknown or other
nnnnnnnnnnnnnnnnnnn

SEQ ID NO: 305 - DNA, 30 bp, Bacillus sp.
gtttactrttaacawtggatgtatttaaat

SEQ ID NO: 306 - DNA, 30 bp, Bacillus sp.; identical to SEQ ID NO: 208

SEQ ID NO: 307 - DNA, 30 bp, Bacillus sp.
gttaaaacawaacaatagatgtattgaaat

SEQ ID NO: 308 - DNA, 30 bp, Bacillus sp.; identical to SEQ ID NO: 206

SEQ ID NO: 309 - DNA, 30 bp, Bacillus sp.; identical to SEQ ID NO: 206

SEQ ID NO: 310 - RNA, 146 nt, Artificial Sequence
  source: /note="Description of Artificial Sequence: Synthetic polynucleotide"
acggucuuuguguuacacccuucaaagcauccaggaugcuuauguaucaauguuuuuuuaacauucgauacuaccuuauggagcguuuacacuugguaaauucaccuuguauuacuucuccauuaaagcaauagauguauugaaau

SEQ ID NO: 311 - DNA, 143 bp, Artificial Sequence
  source: /note="Description of Artificial Sequence: Synthetic polynucleotide"
  modified_base (52)..(59): a, c, t, g, unknown or other
tcgtcggcagcgtcagatgtgtataagagacagaacgacggccagtgaattnnnnnnnnactacaacagccacaacgtctatatcatgggatcctctagagtcgacctgctgtctcttatacacatctccgagcccacgagac

SEQ ID NO: 312 - DNA, 148 bp, Artificial Sequence
  source: /note="Description of Artificial Sequence: Synthetic polynucleotide"
  modified_base (52)..(56): a, c, t, g, unknown or other
tcgtcggcagcgtcagatgtgtataagagacagaacgacggccagtgaattnnnnntggaatgggaactaaagtaatggcaaacgaattcgtaggatcctctagagtcgacctgctgtctcttatacacatctccgagcccacgagac

SEQ ID NO: 313 - DNA, 30 bp, Artificial Sequence
  source: /note="Description of Artificial Sequence: Synthetic oligonucleotide"
actacaacagccacaacgtctatatcatgg

SEQ ID NO: 314 - DNA, 18 bp, Artificial Sequence
  source: /note="Description of Artificial Sequence: Synthetic oligonucleotide"
acacctgtaatcccagca

(19) United States
(12) Patent Application Publication
(10) Pub. No.: US 2023/0203463 A1
(43) Pub. Date: Jun. 29, 2023

(54) RNA-GUIDED NUCLEASES AND ACTIVE FRAGMENTS AND VARIANTS THEREOF AND METHODS OF USE

(71) Applicant: LifeEDIT Therapeutics, Inc., Morrisville, NC (US)

(72) Inventors: Alexandra Briner Crawley, Cary, NC (US); Philip Borden, Durham, NC (US); Tyson D. Bowen, Morrisville, NC (US); Michael Coyle, Chapel Hill, NC (US); Madison R. Rackear, New Haven, CT (US); Tedd D. Elich, Durham, NC (US)

(73) Assignee: LifeEDIT Therapeutics, Inc., Suite 101, NC (US)

(21) Appl. No.: 17/789,977

(22) PCT Filed: Dec. 28, 2020
(86) PCT No.: PCT/US2020/067138

§ 371 Date: Jun. 29, 2022

Related U.S. Application Data
(60) Provisional application No. 63/058,169, filed on Jul. 29, 2020.
(60) Provisional application No. 62/955,014, filed on Dec. 30, 2019.

(51) Int. Cl. C12N 9/22; C12N 15/10; C12N 15/63

(52) U.S. Cl. CPC C12N 9/22; C12N 15/63; C12N 15/102; C07K 2319/09; C07K 2319/21; C07K 2319/43; C12N 2310/20

(57) ABSTRACT

Compositions and methods for binding to a target sequence of interest are provided. The compositions find use in cleaving or modifying a target sequence of interest, detecting a target sequence of interest, and modifying the expression of a sequence of interest. Compositions comprise RNA-guided nuclease polypeptides, CRISPR RNAs, trans-activating CRISPR RNAs, guide RNAs, and nucleic acid molecules encoding the same. Vectors and host cells comprising the nucleic acid molecules are also provided. Further provided are CRISPR systems for binding a target sequence of interest, wherein the CRISPR system comprises an RNA-guided nuclease polypeptide and one or more guide RNAs.

### CROSS-REFERENCE TO RELATED APPLICATIONS

This application claims priority to U.S. Provisional Application No.62/955,014, filed Dec. 30, 2019, and U.S. Provisional Application No. 63/058,169, filed Jul. 29, 2020, each of which application is incorporated by reference herein in its entirety.

### STATEMENT REGARDING THE SEQUENCE LISTING

The Sequence Listing associated with this application is provided in ASCII format in lieu of a paper copy, and is hereby incorporated by reference into the specification. The ASCII copy named L103438_1180WO_(0077_8)_SL is 558,899 bytes in size, was created on Dec. 17, 2020, and is being submitted electronically via EFS-Web.

### FIELD OF THE INVENTION

The present invention relates to the field of molecular biology and gene editing.

### BACKGROUND OF THE INVENTION

Targeted genome editing or modification is rapidly becoming an important tool for basic and applied research. Initial methods involved engineering nucleases such as meganucleases, zinc finger fusion proteins or TALENs, requiring the generation of chimeric nucleases with engineered, programmable, sequence-specific DNA-binding domains specific for each particular target sequence. RNA-guided nucleases, such as the Clustered Regularly Interspaced Short Palindromic Repeats (CRISPR)-associated (Cas) proteins of the CRISPR-Cas bacterial system, allow for the targeting of specific sequences by complexing the nucleases with guide RNA that specifically hybridizes with a particular target sequence. Producing target-specific guide RNAs is less costly and more efficient than generating chimeric nucleases for each target sequence. Such RNA-guided nucleases can be used to edit genomes through the introduction of a sequence-specific break that is repaired via error-prone non-homologous end-joining (NHEJ) to introduce a mutation at a specific genomic location. Alternatively, heterologous DNA may be introduced into the genomic site via homology-directed repair. RNA-guided nucleases (RGNs) can also be used for base editing when fused with a deaminase or for detecting specific nucleotide sequences.

### BRIEF SUMMARY OF THE INVENTION

Compositions and methods for binding a target sequence of interest are provided. The compositions find use in cleaving or modifying a target sequence of interest, detection of a target sequence of interest, and modifying the expression of a sequence of interest. Compositions comprise RNA-guided nuclease (RGN) polypeptides, CRISPR RNAs (crRNAs), trans-activating CRISPR RNAs (tracrRNAs), guide RNAs (gRNAs), nucleic acid molecules encoding the same, vectors and host cells comprising the nucleic acid molecules, and kits comprising an RGN, gRNA, and detector single-stranded DNA. Also provided are CRISPR systems for binding a target sequence of interest, wherein the CRISPR system comprises an RNA-guided nuclease polypeptide and one or more guide RNAs. Thus, methods disclosed herein are drawn to binding a target sequence of interest, and in some embodiments, cleaving or modifying the target sequence of interest. The target sequence of interest can be modified, for example, as a result of non-homologous end joining, homology-directed repair with an introduced donor sequence, or base editing. Further provided are methods and kits for detecting a target DNA sequence of a DNA molecule using detector single-stranded DNA.

### BRIEF DESCRIPTION OF THE FIGURES

FIG. 1 shows the bacterial genomic loci of representative RGNs of the invention.

### DETAILED DESCRIPTION

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended embodiments. Although specific terms are employed herein, they are used in a generic and descriptive sense only and not for purposes of limitation.

### I. Overview

RNA-guided nucleases (RGNs) allow for the targeted manipulation of specific site(s) within a genome and are useful in the context of gene targeting for therapeutic and research applications. In a variety of organisms, including mammals, RNA-guided nucleases have been used for genome engineering by stimulating non-homologous end joining and homologous recombination, for example. The compositions and methods described herein are useful for creating single- or double-stranded breaks in polynucleotides, modifying polynucleotides, detecting a particular site within a polynucleotide, or modifying the expression of a particular gene.

The RNA-guided nucleases disclosed herein can alter gene expression by modifying a target sequence. In specific embodiments, the RNA-guided nucleases are directed to the target sequence by a guide RNA (gRNA) as part of a Clustered Regularly Interspaced Short Palindromic Repeats (CRISPR) RNA-guided nuclease system. The RGNs are considered “RNA-guided” because guide RNAs form a complex with the RNA-guided nucleases to direct the RNA-guided nuclease to bind to a target sequence and in some embodiments, introduce a single-stranded or double-stranded break at the target sequence. After the target sequence has been cleaved, the break can be repaired such that the DNA sequence of the target sequence is modified during the repair process. Thus, provided herein are methods for using the RNA-guided nucleases to modify a target sequence in the DNA of host cells. For example, RNA-guided nucleases can be used to modify a target sequence at a genomic locus of eukaryotic cells or prokaryotic cells.

### II. RNA-Guided Nucleases

Provided herein are RNA-guided nucleases. The term RNA-guided nuclease (RGN) refers to a polypeptide that binds to a particular target nucleotide sequence in a sequence-specific manner and is directed to the target nucleotide sequence by a guide RNA molecule that is complexed with the polypeptide and hybridizes with the target sequence. Although an RNA-guided nuclease can be capable of cleaving the target sequence upon binding, the term “RNA-guided nuclease” also encompasses nuclease-dead RNA-guided nucleases that are capable of binding to, but not cleaving, a target sequence. Cleavage of a target sequence by an RNA-guided nuclease can result in a single- or double-stranded break. RNA-guided nucleases only capable of cleaving a single strand of a double-stranded nucleic acid molecule are referred to herein as nickases.

The RGNs of the invention are members of the Class 2 CRISPR-Cas systems. More specifically, they are members of the Type V CRISPR-Cas systems. Type V CRISPR-Cas systems are broadly defined as systems that contain a single effector nuclease that is responsible for using the guide RNA to target dsDNA (double-stranded DNA); additionally, the single effector nuclease contains a split RuvC nuclease domain that is responsible for the catalytic activity (Jinek et al 2014, Science doi:10.1126/science.1247997; Zetsche et al 2015, Cell doi: 10.1016/j.cell.2015.09.038; Shmakov et al 2017, Nat Rev Microbiol doi:10.1038/nrmicro.2016.184; Yan et al 2018, Science doi:10.1126/science.aav7271; Harrington et al 2018, Science doi: 10.1126/science.aav4294; each of which is incorporated herein by reference in its entirety). Most Type V effectors can also target ssDNA (single-stranded DNA), often without a PAM requirement (Zetsche et al 2015; Yan et al 2018; Harrington et al 2018).

The Type V-A signature protein is Cas12a. It is 1,000-1,400 amino acids in length and has several domains in addition to the RuvC domain, including a wedge domain with recognition lobes (Yamano et al (2016) Cell 165:949-962). In contrast, the Type V-U systems are smaller in size (500-700 amino acids in length) compared to most other Type V systems. The V-U’s also possess a split RuvC domain and a positively charged bridge helix (Shmakov et al 2017). The V-U proteins often do not have accessory Cas proteins encoded with the effector protein, while Cas12a co-localizes with cas1, cas2 and occasionally cas4 (Shmakov et al 2017). Based on these differences between the Type V-U systems and other Type V members, it was suggested by Shmakov et al (2017) that upon determination of functionality, the Type V-U systems should receive a new type/subtype designation.

For example, Cas14 enzymes are 400-700 amino acids in length (Harrington et al 2018). Upon first publication, these systems were touted as separate Cas enzymes from the canonical Cas12 effector protein for Type Vs. Later publications from Yan et al., have dubbed Cas14a, -b, and -c as subtype V-F within the Type V nomenclature. Cas14a and b are most closely related to c2c10, which is Type V-U3. Cas14c is most closely related to c2c8 and c2c9, which are Types V-U2 and V-U4, respectively (Harrington et al 2018; Yan et al 2018). The genomic loci of the Cas14 RGNs are associated with accessory Cas proteins, and the tracrRNAs are encoded between the Cas14 and the repeat-spacer arrays. These systems cannot process their own guide RNAs, unlike Cas12a which is capable of processing individual guides from a single transcript containing multiple guide RNAs (Harrington et al 2018).

All RGNs of the invention contain a split RuvC domain, with the exception of APG06369. However, many of the RGNs of the invention have unique locus arrangements, which suggests that these RGNs are novel to the Class 2 CRISPR-Cas system of classification. None of the loci from which the RGNs of the invention are derived (see Table 1 in Example 1) contain Cas1 or Cas2.

As disclosed herein, APG07339, APG09624, APG03003, APG05405, APG09777, APG05680, APG02119, APG03285, APG04998, and APG07078 are standalone Cas effectors that are not encoded with accessory genes and may require a tracrRNA in addition to the crRNA. Based on the disclosures herein, these CRISPR-Cas systems need to receive a new classification. Additionally, phylogenetic analysis reveals that these RGNs can be grouped into three different subtypes. One subtype contains APG07078. The second subtype contains APG05680 and APG03285. The third sub-type contains APG07339, APG09624, APG03003, APG05405, APG09777, APG02119, and APG04998.

APG06369 is a unique effector nuclease that lacks a distinguishable RuvC domain and sits in a never-before seen CRISPR locus with non-canonical accessory genes. APG06369 has four accessory genes (the four accessory proteins are set forth as SEQ ID NOs: 178-181), none of which possess an annotated domain or function. APG06369 is a unique Cas protein.

Phylogenetically, APG03847, APG05625, APG03759, APG05123, and APG03524 form a clade of unique RuvC containing effector nucleases. These RGNs possess up to three accessory genes: one is an HNH endonuclease, one is an HTH transcriptional regulator, and the third has no known function or domains. The accessory proteins for APG03847 are set forth as SEQ ID NOs: 182, 183, and 184. The accessory proteins for APG05625 are set forth as SEQ ID NOs: 185, 186, and 187. The accessory proteins for APG03524 are set forth as SEQ ID NOs: 188, 189, and 190. The accessory proteins for APG03759 and APG05123 are set forth as SEQ ID NOs: 191 and 192, respectively. They have a unique CRISPR repeat arrangement at their loci, where the repeats associated with APG03847, APG05625, APG03759, APG05123, and APG03524 are flush with coding sequences for the numerous proteins. This is an extremely unusual feature for CRISPR-Cas systems, and suggests a form of CRISPR expression that does not require the leader sequence. Such a form of CRISPR expression is unlike any system known to date.

The RNA-guided nucleases disclosed herein include the RNA-guided nucleases shown in Table 1, the amino acid sequences of which are set forth as SEQ ID NOs: 1 to 109, and active fragments or variants thereof that retain the ability to bind to a target nucleotide sequence in an RNA-guided sequence-specific manner. In some embodiments, such an active fragment or variant of the RGN is capable of cleaving a single- or double-stranded target sequence. In some embodiments, an active variant of the RGN of the invention comprises an amino acid sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to any one of the amino acid sequences set forth as SEQ ID NOs: 1 to 109. In certain embodiments, an active fragment of the RGN of the invention comprises at least 50, 100, 150, 200, 250, 300, 350, 400, 450, 500, 550, 600, 650, 700, 750, 800, 850, 900, 950, 1000, 1050 or more contiguous amino acid residues of any one of the amino acid sequences set forth as SEQ ID NOs: 1 to 109. RNA-guided nucleases provided herein can comprise at least one nuclease domain (e.g., DNase, RNase domain) and at least one RNA recognition and/or RNA binding domain to interact with guide RNAs. Further domains that can be found in RNA-guided nucleases provided herein include, but are not limited to DNA binding domains, helicase domains, protein-protein interaction domains, and dimerization domains. In specific embodiments, the RNA-guided nucleases provided herein can comprise at least 70%, 75%, 80%, 85%, 90%, 91%, 92%, 93%, 94%, 95%, 96%, 97%, 98%, 99% or more sequence identity to one or more of a DNA binding domain, helicase domain, protein-protein interaction domain, and dimerization domain.

In various embodiments, a target nucleotide sequence is bound by an RNA-guided nuclease provided herein and hybridizes with the guide RNA associated with the RNA-guided nuclease. The target sequence can then be subsequently cleaved by the RNA-guided nuclease if the polypeptide possesses nuclease activity. The terms “cleave” or “cleavage” refer to the hydrolysis of at least one phosphodiester bond within the backbone of a target nucleotide sequence that can result in either single-stranded or double-stranded breaks within the target sequence. In various embodiments, the presently disclosed RGNs can cleave nucleotides within a polynucleotide, functioning as an endonuclease or can be an exonuclease, removing successive nucleotides from the end (the 5′ and/or the 3′ end) of a polynucleotide. In some embodiments, the disclosed RGNs can cleave nucleotides of a target sequence within any position of a polynucleotide and thus function as both an endonuclease and exonuclease. The cleavage of a target polynucleotide by the presently disclosed RGNs can result in staggered breaks or blunt ends.

In some embodiments, the RGN requires the expression or presence of at least one RGN accessory protein in order to bind to and/or cleave a target polynucleotide. In some of these embodiments, the RGN requires at least one RGN accessory protein set forth as SEQ ID NOs: 178-192 or an active variant or fragment thereof. In particular embodiments wherein the RGN is APG06369 (SEQ ID NO: 11) or a variant or fragment thereof, at least one RGN accessory protein set forth as SEQ ID NOs: 178-181 or an active variant or fragment thereof is required for activity. In some of those embodiments wherein the RGN is APG03847 (SEQ ID NO: 12) or a variant or fragment thereof, at least one RGN accessory protein set forth as SEQ ID NOs: 182-184 or an active variant or fragment thereof is required for activity. In certain embodiments wherein the RGN is APG05625 (SEQ ID NO: 13) or a variant or fragment thereof, at least one RGN accessory protein set forth as SEQ ID NOs: 185-187 or an active variant or fragment thereof is required for activity. In some embodiments wherein the RGN is APG03524 (SEQ ID NO: 16) or a variant or fragment thereof, at least one RGN accessory protein set forth as SEQ ID NOs: 188-190 or an active variant or fragment thereof is required for activity. In particular embodiments wherein the RGN is APG03759 (SEQ ID NO: 14) or a variant or fragment thereof, the RGN accessory protein set forth as SEQ ID NO: 191 or an active variant or fragment thereof is required for activity. In certain embodiments wherein the RGN is APG05123 (SEQ ID NO: 15) or a variant or fragment thereof, the RGN accessory protein set forth as SEQ ID NO: 192 or an active variant or fragment thereof is required for activity.

In some embodiments, the presently disclosed RNA-guided nucleases can be wild-type sequences derived from bacterial or archaeal species. In some embodiments, the RNA-guided nucleases can be variants or fragments of wild-type polypeptides. The wild-type RGN can be modified to alter nuclease activity or alter PAM specificity, for example. In some embodiments, the RNA-guided nuclease is not naturally-occurring.

In certain embodiments, the RNA-guided nuclease functions as a nickase, only cleaving a single strand of the target nucleotide sequence. Such RNA-guided nucleases have a single functioning nuclease domain. In particular embodiments, the nickase is capable of cleaving the positive strand or negative strand. In some of these embodiments, additional nuclease domains have been mutated such that the nuclease activity is reduced or eliminated.

In some embodiments, the RNA-guided nuclease lacks nuclease activity altogether and is referred to herein as nuclease-dead or nuclease inactive. Any method known in the art for introducing mutations into an amino acid sequence, such as PCR-mediated mutagenesis and site-directed mutagenesis, can be used for generating nickases or nuclease-dead RGNs. See, e.g., U.S. Publ. No. 2014/0068797 and U.S. Pat. No. 9,790,490; each of which is incorporated herein by reference in its entirety.

RNA-guided nucleases that lack nuclease activity can be used to deliver a fused polypeptide, polynucleotide, or small molecule payload to a particular genomic location. In some of these embodiments, the RGN polypeptide or guide RNA can be fused to a detectable label to allow for detection of a particular sequence. As a non-limiting example, a nuclease-dead RGN can be fused to a detectable label (e.g., fluorescent protein) and targeted to a particular sequence associated with a disease to allow for detection of the disease-associated sequence.

In some embodiments, nuclease-dead RGNs can be targeted to particular genomic locations to alter the expression of a desired sequence. In some embodiments, the binding of a nuclease-dead RNA-guided nuclease to a target sequence results in the reduction in expression of the target sequence or a gene under transcriptional control by the target sequence by interfering with the binding of RNA polymerase or transcription factors within the targeted genomic region. In other embodiments, the RGN (e.g., a nuclease-dead RGN) or its complexed guide RNA further comprises an expression modulator that, upon binding to a target sequence, serves to either repress or activate the expression of the target sequence or a gene under transcriptional control by the target sequence. In some of these embodiments, the expression modulator modulates the expression of the target sequence or regulated gene through epigenetic mechanisms.

In some embodiments, the nuclease-dead RGNs or an RGN with only nickase activity can be targeted to particular genomic locations to modify the sequence of a target polynucleotide through fusion to a base-editing polypeptide, for example a deaminase polypeptide or active variant or fragment thereof, that directly chemically modifies (e.g., deaminates) a nucleobase, resulting in conversion from one nucleobase to another. The base-editing polypeptide can be fused to the RGN at its N-terminal or C-terminal end. Additionally, the base-editing polypeptide may be fused to the RGN via a peptide linker. A non-limiting example of a deaminase polypeptide that is useful for such compositions and methods include a cytidine deaminase or an adenine deaminase (such as the adenosine base editor described in Gaudelli et al. (2017) Nature 551:464-471, U.S. Publ. Nos. 2017/0121693 and 2018/0073012, International Publ. No. WO 2018/027078, or any of the deaminases disclosed in International Publ. No. WO 2020/139873, and U.S. Provisional Appl. Nos. 62/785,391 (filed Dec. 27, 2018), 62/932,169 (filed Nov. 7, 2019), and 63/077,089 (filed Sep. 11, 2020), each of which is herein incorporated by reference in its entirety. Further, it is known in the art that certain fusion proteins between an RGN and a base-editing enzyme may also comprise at least one uracil stabilizing polypeptide that increases the mutation rate of a cytidine, deoxycytidine, or cytosine to a thymidine, deoxythymidine, or thymine in a nucleic acid molecule by a deaminase. Non-limiting examples of uracil stabilizing polypeptides include those disclosed in U.S. Provisional Appl. No. 63/052,175, filed Jul. 15, 2020, and a uracil glycosylase inhibitor (UGI) domain (SEQ ID NO: 137), which may increase base editing efficiency. In particular embodiments, the present disclosure provides a fusion protein comprising an RGN described herein or a variant thereof, a deaminase, and optionally at least one uracil stabilizing polypeptides, such as UGI. In certain embodiments, the RGN that is fused to the base-editing polypeptide is a nickase that cleaves the DNA strand that is not acted upon by the base-editing polypeptide (e.g., deaminase). RNA-guided nucleases that are fused to a polypeptide or domain can be separated or joined by a linker. The term “linker,” as used herein, refers to a chemical group or a molecule linking two molecules or moieties, e.g., a binding domain and a cleavage domain of a nuclease. In some embodiments, a linker joins a gRNA binding domain of an RNA guided nuclease and a base-editing polypeptide, such as a deaminase. In some embodiments, a linker joins a nuclease-dead RGN and a deaminase. Typically, the linker is positioned between, or flanked by, two groups, molecules, or other moieties and connected to each one via a covalent bond, thus connecting the two. In some embodiments, the linker is an amino acid or a plurality of amino acids (e.g., a peptide or protein). In some embodiments, the linker is an organic molecule, group, polymer, or chemical moiety. In some embodiments, the linker is 5-100 amino acids in length, for example, 5, 6, 7, 8, 9, 10, 11, 12, 13, 14, 15, 16, 17, 18, 19, 20, 21, 22, 23, 24, 25, 26, 27, 28, 29, 30, 30-35, 35-40, 40-45, 45-50, 50-60, 60-70, 70-80, 80-90, 90-100, 100-150, or 150-200 amino acids in length. Longer or shorter linkers are also contemplated.

In various embodiments, the present disclosure provides the presently disclosed RNA-guided nucleases comprising at least one nuclear localization signal (NLS) to enhance transport of the RGN to the nucleus of a cell. Nuclear localization signals are known in the art and generally comprise a stretch of basic amino acids (see, e.g., Lange et al., J. Biol. Chem. (2007) 282:5101-5105). In particular embodiments, the RGN comprises 2, 3, 4, 5, 6 or more nuclear localization signals. The nuclear localization signal(s) can be a heterologous NLS. Non-limiting examples of nuclear localization signals useful for the presently disclosed RGNs are the nuclear localization signals of SV40 Large T-antigen, nucleopasmin, and c-Myc (see, e.g., Ray et al. (2015) Bioconjug Chem 26(6):1004-7). In particular embodiments, the RGN comprises the NLS sequence set forth as SEQ ID NO: 149 or 150. The RGN can comprise one or more NLS sequences at its N-terminus, C- terminus, or both the N-terminus and C-terminus. For example, the RGN can comprise two NLS sequences at the N-terminal region and four NLS sequences at the C-terminal region.

Other localization signal sequences known in the art that localize polypeptides to particular subcellular location(s) can also be used to target the RGNs, including, but not limited to, plastid localization sequences, mitochondrial localization sequences, and dual-targeting signal sequences that target to both the plastid and mitochondria (see, e.g., Nassoury and Morse (2005) Biochim Biophys Acta 1743:5-19; Kunze and Berger (2015) Front Physiol dx.doi.org/10.3389/fphys.2015.00259; Herrmann and Neupert (2003) IUBMB Life 55:219-225; Soll (2002) Curr Opin Plant Biol 5:529-535; Carrie and Small (2013) Biochim Biophys Acta 1833:253-259; Carrie et al. (2009) FEBS J 276:1187-1195; Silva-Filho (2003) Curr Opin Plant Biol 6:589-595; Peeters and Small (2001) Biochim Biophys Acta 1541:54-63; Murcha et al. (2014) J Exp Bot 65:6301-6335; Mackenzie (2005) Trends Cell Biol 15:548-554; Glaser et al. (1998) Plant Mol Biol 38:311-338).

In certain embodiments, the presently disclosed RNA-guided nucleases comprise at least one cell-penetrating domain that facilitates cellular uptake of the RGN. Cell-penetrating domains are known in the art and generally comprise stretches of positively charged amino acid residues (i.e., polycationic cell-penetrating domains), alternating polar amino acid residues and non-polar amino acid residues (i.e., amphipathic cell-penetrating domains), or hydrophobic amino acid residues (i.e., hydrophobic cell-penetrating domains) (see, e.g., Milletti F. (2012) Drug Discov Today 17:850-860). A non-limiting example of a cell-penetrating domain is the trans-activating transcriptional activator (TAT) from the human immunodeficiency virus 1.

The nuclear localization signal, plastid localization signal, mitochondrial localization signal, dual-targeting localization signal, and/or cell-penetrating domain can be located at the amino-terminus (N-terminus), the carboxyl-terminus (C-terminus), or in an internal location of the RNA-guided nuclease.

In certain embodiments, the presently disclosed RGNs are fused to an effector domain, such as a cleavage domain, a deaminase domain, or an expression modulator domain, either directly or indirectly via a linker peptide. Such a domain can be located at the N-terminus, the C-terminus, or an internal location of the RNA-guided nuclease. In some of these embodiments, the RGN component of the fusion protein is a nuclease-dead RGN.

In some embodiments, the RGN fusion protein comprises a cleavage domain, which is any domain that is capable of cleaving a polynucleotide (i.e., RNA, DNA, or RNA/DNA hybrid) and includes, but is not limited to, restriction endonucleases and homing endonucleases, such as Type IIS endonucleases (e.g., FokI) (see, e.g., Belfort et al. (1997) Nucleic Acids Res. 25:3379-3388; Linn et al. (eds.) Nucleases, Cold Spring Harbor Laboratory Press, 1993).

In some embodiments, the RGN fusion protein comprises a deaminase domain that deaminates a nucleobase, resulting in conversion from one nucleobase to another, and includes, but is not limited to, a cytidine deaminase or an adenine deaminase base editor (see, e.g., Gaudelli et al. (2017) Nature 551:464-471, U.S. Publ. Nos. 2017/0121693 and 2018/0073012, U.S. Pat. No. 9,840,699, International Publ. No. WO/2018/027078, International Appl. No. PCT/US2019/068079, and U.S. Provisional Appl. Nos. 62/785,391 (filed Dec. 27, 2018) and 62/932,169 (filed Nov. 7, 2019)).

In some embodiments, the effector domain of the RGN fusion protein can be an expression modulator domain, which is a domain that either serves to upregulate or downregulate transcription. The expression modulator domain can be an epigenetic modification domain, a transcriptional repressor domain or a transcriptional activation domain.

In some of these embodiments, the expression modulator of the RGN fusion protein comprises an epigenetic modification domain that covalently modifies DNA or histone proteins to alter histone structure and/or chromosomal structure without altering the DNA sequence, leading to changes in gene expression (i.e., upregulation or downregulation). Non-limiting examples of epigenetic modifications include acetylation or methylation of lysine residues, arginine methylation, serine and threonine phosphorylation, and lysine ubiquitination and sumoylation of histone proteins, and methylation and hydroxymethylation of cytosine residues in DNA. Non-limiting examples of epigenetic modification domains include histone acetyltransferase domains, histone deacetylase domains, histone methyltransferase domains, histone demethylase domains, DNA methyltransferase domains, and DNA demethylase domains.

In some embodiments, the expression modulator of the fusion protein comprises a transcriptional repressor domain, which interacts with transcriptional control elements and/or transcriptional regulatory proteins, such as RNA polymerases and transcription factors, to reduce or terminate transcription of at least one gene. Transcriptional repressor domains are known in the art and include, but are not limited to, Sp1-like repressors, IκB, and Krüppel associated box (KRAB) domains.

In some embodiments, the expression modulator of the fusion protein comprises a transcriptional activation domain, which interacts with transcriptional control elements and/or transcriptional regulatory proteins, such as RNA polymerases and transcription factors, to increase or activate transcription of at least one gene. Transcriptional activation domains are known in the art and include, but are not limited to, a herpes simplex virus VP16 activation domain and an NFAT activation domain.

In some embodiments, the presently disclosed RGN polypeptides comprise a detectable label or a purification tag. The detectable label or purification tag can be located at the N-terminus, the C-terminus, or an internal location of the RNA-guided nuclease, either directly or indirectly via a linker peptide. In some of these embodiments, the RGN component of the fusion protein is a nuclease-dead RGN. In other embodiments, the RGN component of the fusion protein is an RGN with nickase activity.

A detectable label is a molecule that can be visualized or otherwise observed. The detectable label may be fused to the RGN as a fusion protein (e.g., fluorescent protein) or may be a small molecule conjugated to the RGN polypeptide that can be detected visually or by other means. Detectable labels that can be fused to the presently disclosed RGNs as a fusion protein include any detectable protein domain, including but not limited to, a fluorescent protein or a protein domain that can be detected with a specific antibody. Non-limiting examples of fluorescent proteins include green fluorescent proteins (e.g., GFP, EGFP, ZsGreen1) and yellow fluorescent proteins (e.g., YFP, EYFP, ZsYellow1). Non-limiting examples of small molecule detectable labels include radioactive labels, such as ³H and ³⁵S.

In some embodiments the presently disclosed RGN polypeptides comprise a purification tag, which is any molecule that can be utilized to isolate a protein or fused protein from a mixture (e.g., biological sample, culture medium). Non-limiting examples of purification tags include biotin, myc, maltose binding protein (MBP), and glutathione-S-transferase (GST).

### III. Guide RNA

The present disclosure provides guide RNAs and polynucleotides encoding the same. The term “guide RNA” refers to a nucleotide sequence having sufficient complementarity with a target nucleotide sequence to hybridize with the target sequence and direct sequence-specific binding of an associated RNA-guided nuclease to the target nucleotide sequence. Thus, an RGN’s respective guide RNA is one or more RNA molecules (generally, one or two), that can bind to the RGN and guide the RGN to bind to a particular target nucleotide sequence, and in those embodiments wherein the RGN has nickase or nuclease activity, also cleave the target nucleotide sequence. In some embodiments, a guide RNA comprises a CRISPR RNA (crRNA) and in some embodiments, a trans-activating CRISPR RNA (tracrRNA). Native guide RNAs that comprise both a crRNA and a tracrRNA generally comprise two separate RNA molecules that hybridize to each other through the repeat sequence of the crRNA and the anti-repeat sequence of the tracrRNA.

In some embodiments, native direct repeat sequences within a CRISPR array range in length from 28 to 37 base pairs. In some embodiments, native direct repeat sequences within a CRISPR array range in length from about 23 bp to about 55 bp (e.g., from 23 bp to 55 bp). In some embodiments, spacer sequences within a CRISPR array range from about 32 to about 38 bp in length. In some embodiments, spacer sequences within a CRISPR array range from about 21 bp to about 72 bp (e.g., from 21 bp to 72 bp). In some embodiments a CRISPR array disclosed herein comprises less than 50 units of the CRISPR repeat-spacer sequence. The CRISPRs are transcribed as part of a long transcript termed the primary CRISPR transcript, which comprises much of the CRISPR array. The primary CRISPR transcript is cleaved by Cas proteins to produce crRNAs or in some cases, to produce pre-crRNAs that are further processed by additional Cas proteins into mature crRNAs. Mature crRNAs comprise a spacer sequence and a CRISPR repeat sequence. In some embodiments in which pre-crRNAs are processed into mature (or processed) crRNAs, maturation involves the removal of about one to about six or more 5′, 3′, or 5′ and 3′ nucleotides. For the purposes of genome editing or targeting a particular target nucleotide sequence of interest, these nucleotides that are removed during maturation of the pre-crRNA molecule are not necessary for generating or designing a guide RNA. The consensus repeat sequence for each of the presently disclosed RGN proteins (SEQ ID NOs: 1-109) is disclosed in SEQ ID NOs: 201-309, respectively. The processed crRNA repeat sequence for each of APG07339 (SEQ ID NO: 1), APG09624 (SEQ ID NO: 2), APG03003 (SEQ ID NO: 3), APG05405 (SEQ ID NO: 4), APG09777 (SEQ ID NO: 5), APG05680 (SEQ ID NO: 6), APG06369 (SEQ ID NO: 11), APG03847 (SEQ ID NO: 12), APG05625 (SEQ ID NO: 13), and APG03524 (SEQ ID NO: 16) is disclosed in SEQ ID NOs: 110-119, respectively.

A CRISPR RNA (crRNA) comprises a spacer sequence and a CRISPR repeat sequence. The “spacer sequence” is the nucleotide sequence that directly hybridizes with the target nucleotide sequence of interest. The spacer sequence is engineered to be fully or partially complementary with the target sequence of interest. In various embodiments, the spacer sequence can comprise from about 8 nucleotides to about 30 nucleotides, or more. For example, the spacer sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the spacer sequence is about 10 to about 26 nucleotides in length, or about 12 to about 30 nucleotides in length. In particular embodiments, the spacer sequence is about 30 nucleotides in length. In some embodiments, the degree of complementarity between a spacer sequence and its corresponding target sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In particular embodiments, the spacer sequence is free of secondary structure, which can be predicted using any suitable polynucleotide folding algorithm known in the art, including but not limited to mFold (see, e.g., Zuker and Stiegler (1981) Nucleic Acids Res. 9:133-148) and RNAfold (see, e.g., Gruber et al. (2008) Cell 106(1):23-24).

The CRISPR RNA repeat sequence comprises a nucleotide sequence that forms a structure, either on its own or in concert with a hybridized tracrRNA, that is recognized by the RGN molecule. In various embodiments, the CRISPR RNA repeat sequence can comprise from about 8 nucleotides to about 30 nucleotides, or more. For example, the CRISPR repeat sequence can be about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In some embodiments, the CRISPR repeat sequence is about 21 nucleotides in length. In some embodiments, the degree of complementarity between a CRISPR repeat sequence and its corresponding tracrRNA sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more. In particular embodiments, the CRISPR repeat sequence comprises any one of the nucleotide sequences of SEQ ID NOs: 110 to 119, 139, 141, 143, 146, and 201 to 309, or an active variant or fragment thereof that when comprised within a guide RNA, is capable of directing the sequence-specific binding of an associated RNA-guided nuclease provided herein to a target sequence of interest. In certain embodiments, an active CRISPR repeat sequence variant of a wild-type sequence comprises a nucleotide sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to any one of the nucleotide sequences set forth as SEQ ID NOs: 110 to 119, 139, 141, 143, 146, and 201 to 309. In certain embodiments, an active CRISPR repeat sequence fragment of a wild-type sequence comprises at least 5, 6, 7, 8, 9, 10, 11, 12, 13, 14, 15, 16, 17, 18, 19, or 20 contiguous nucleotides of any one of the nucleotide sequences set forth as SEQ ID NOs: 110 to 119, 139, 141, 143, 146, and 201 to 309.

In certain embodiments, the crRNA is not naturally-occurring. In some of these embodiments, the specific CRISPR repeat sequence is not linked to the engineered spacer sequence in nature and the CRISPR repeat sequence is considered heterologous to the spacer sequence. In certain embodiments, the spacer sequence is an engineered sequence that is not naturally occurring.

In some embodiments, the guideRNA further comprises a tracrRNA molecule. A trans-activating CRISPR RNA or tracrRNA molecule comprises a nucleotide sequence comprising a region that has sufficient complementarity to hybridize to a CRISPR repeat sequence of a crRNA, which is referred to herein as the anti-repeat region. In some embodiments, the tracrRNA molecule further comprises a region with secondary structure (e.g., stem-loop) or forms secondary structure upon hybridizing with its corresponding crRNA. In particular embodiments, the region of the tracrRNA that is fully or partially complementary to a CRISPR repeat sequence is at the 5′ end of the molecule and the 3′ end of the tracrRNA comprises secondary structure. This region of secondary structure generally comprises several hairpin structures, including the nexus hairpin, which is found adjacent to the anti-repeat sequence. There are often terminal hairpins at the 3′ end of the tracrRNA that can vary in structure and number, but often comprise a GC-rich Rho-independent transcriptional terminator hairpin followed by a string of Us at the 3′ end. See, for example, Briner et al. (2014) Molecular Cell 56:333-339, Briner and Barrangou (2016) Cold SpringHarb Protoc; doi: 10.1101/pdb.top090902, and U.S. Publication No. 2017/0275648, each of which is herein incorporated by reference in its entirety.

In various embodiments, the anti-repeat region of the tracrRNA that is fully or partially complementary to the CRISPR repeat sequence comprises from about 6 nucleotides to about 30 nucleotides, or more. For example, the region of base pairing between the tracrRNA anti-repeat sequence and the CRISPR repeat sequence can be about 6, about 7, about 8, about 9, about 10, about 11, about 12, about 13, about 14, about 15, about 16, about 17, about 18, about 19, about 20, about 21, about 22, about 23, about 24, about 25, about 26, about 27, about 28, about 29, about 30, or more nucleotides in length. In particular embodiments, the anti-repeat region of the tracrRNA that is fully or partially complementary to a CRISPR repeat sequence is about 10 nucleotides in length. In some embodiments, the degree of complementarity between a CRISPR repeat sequence and its corresponding tracrRNA anti-repeat sequence, when optimally aligned using a suitable alignment algorithm, is about or more than about 50%, about 60%, about 70%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, or more.

In various embodiments, the entire tracrRNA can comprise from about 60 nucleotides to more than about 210 nucleotides. For example, the tracrRNA can be about 60, about 65, about 70, about 75, about 80, about 85, about 90, about 95, about 100, about 105, about 110, about 115, about 120, about 125, about 130, about 135, about 140, about 150, about 160, about 170, about 180, about 190, about 200, about 210 or more nucleotides in length. In particular embodiments, the tracrRNA is about 100 to about 201 nucleotides in length, including about 95, about 96, about 97, about 98, about 99, about 100, about 105, about 106, about 107, about 108, about 109, and about 100 nucleotides in length. In certain embodiments, the tracrRNA is about 96 nucleotides in length.

In particular embodiments, the tracrRNA comprises any one of the nucleotide sequences of SEQ ID NOs: 120 to 128, 140, 142, 145, 147, and 148, or an active variant or fragment thereof that when comprised within a guide RNA is capable of directing the sequence-specific binding of an associated RNA-guided nuclease provided herein to a target sequence of interest. In certain embodiments, an active tracrRNA sequence variant of a wild-type sequence comprises a nucleotide sequence having at least 40%, 45%, 50%, 55%, 60%, 65%, 70%, 75%, 80%, 85%, 90%, 91%, 92%, 93%, 94%, 95%, 96%, 97%, 98%, 99% or more sequence identity to any one of the nucleotide sequences set forth as SEQ ID NOs: 120 to 128, 140, 142, 145, 147, and 148. In certain embodiments, an active tracrRNA sequence fragment of a wild-type sequence comprises at least 5, 10, 15, 20, 25, 30, 35, 40, 45, 50, 55, 60, 65, 70, 75, 80, or more contiguous nucleotides of any one of the nucleotide sequences set forth as SEQ ID NOs: 120 to 128, 140, 142, 145, 147, and 148.

Two polynucleotide sequences can be considered to be substantially complementary when the two sequences hybridize to each other under stringent conditions. Likewise, an RGN is considered to bind to a particular target sequence within a sequence-specific manner if the guide RNA bound to the RGN binds to the target sequence under stringent conditions. By “stringent conditions” or “stringent hybridization conditions” is intended conditions under which the two polynucleotide sequences will hybridize to each other to a detectably greater degree than to other sequences (e.g., at least 2-fold over background). Stringent conditions are sequence-dependent and will be different in different circumstances. Typically, stringent conditions will be those in which the salt concentration is less than about 1.5 M Na ion, typically about 0.01 to 1.0 M Na ion concentration (or other salts) at pH 7.0 to 8.3, and the temperature is at least about 30° C. for short sequences (e.g., 10 to 50 nucleotides) and at least about 60° C. for long sequences (e.g., greater than 50 nucleotides). Stringent conditions may also be achieved with the addition of destabilizing agents such as formamide. Exemplary low stringency conditions include hybridization with a buffer solution of 30 to 35% formamide, 1 M NaCl, 1% SDS (sodium dodecyl sulfate) at 37° C., and a wash in 1X to 2X SSC (20X SSC = 3.0 M NaCl/0.3 M trisodium citrate) at 50 to 55° C. Exemplary moderate stringency conditions include hybridization in 40 to 45% formamide, 1.0 M NaCl, 1% SDS at 37° C., and a wash in 0.5X to 1X SSC at 55 to 60° C. Exemplary high stringency conditions include hybridization in 50% formamide, 1 M NaCl, 1% SDS at 37° C., and a wash in 0.1X SSC at 60 to 65° C. Optionally, wash buffers may comprise about 0.1% to about 1% SDS. Duration of hybridization is generally less than about 24 hours, usually about 4 to about 12 hours. The duration of the wash time will be at least a length of time sufficient to reach equilibrium.

The Tm is the temperature (under defined ionic strength and pH) at which 50% of a complementary target sequence hybridizes to a perfectly matched sequence. For DNA-DNA hybrids, the Tm can be approximated from the equation of Meinkoth and Wahl (1984) Anal. Biochem. 138:267-284: Tm = 81.5° C. + 16.6 (log M) + 0.41 (%GC) - 0.61 (% form) - 500/L; where M is the molarity of monovalent cations, %GC is the percentage of guanosine and cytosine nucleotides in the DNA, % form is the percentage of formamide in the hybridization solution, and L is the length of the hybrid in base pairs. Generally, stringent conditions are selected to be about 5° C. lower than the thermal melting point (Tm) for the specific sequence and its complement at a defined ionic strength and pH. However, severely stringent conditions can utilize a hybridization and/or wash at 1, 2, 3, or 4° C. lower than the thermal melting point (Tm); moderately stringent conditions can utilize a hybridization and/or wash at 6, 7, 8, 9, or 10° C. lower than the thermal melting point (Tm); low stringency conditions can utilize a hybridization and/or wash at 11, 12, 13, 14, 15, or 20° C. lower than the thermal melting point (Tm). Using the equation, hybridization and wash compositions, and desired Tm, those of ordinary skill will understand that variations in the stringency of hybridization and/or wash solutions are inherently described. An extensive guide to the hybridization of nucleic acids is found in Tijssen (1993) Laboratory Techniques in Biochemistry and Molecular Biology—Hybridization with Nucleic Acid Probes, Part I, Chapter 2 (Elsevier, New York); and Ausubel et al., eds. (1995) Current Protocols in Molecular Biology, Chapter 2 (Greene Publishing and Wiley-Interscience, New York). See Sambrook et al. (1989) Molecular Cloning: A Laboratory Manual (2d ed., Cold Spring Harbor Laboratory Press, Plainview, New York).

The term “sequence specific” can also refer to the binding of a target sequence at a greater frequency than binding to a randomized background sequence.

In some embodiments, e.g., wherein the guide RNA comprises both a crRNA and a tracrRNA, the guide RNA can be a single guide RNA or a dual-guide RNA system. A single guide RNA comprises the crRNA and tracrRNA on a single molecule of RNA, whereas a dual-guide RNA system comprises a crRNA and a tracrRNA present on two distinct RNA molecules, hybridized to one another through at least a portion of the CRISPR repeat sequence of the crRNA and at least a portion of the tracrRNA, which may be fully or partially complementary to the CRISPR repeat sequence of the crRNA. In some of those embodiments wherein the guide RNA is a single guide RNA, the crRNA and tracrRNA are separated by a linker nucleotide sequence. In general, the linker nucleotide sequence is one that does not include complementary bases in order to avoid the formation of secondary structure within or comprising nucleotides of the linker nucleotide sequence. In some embodiments, the linker nucleotide sequence between the crRNA and tracrRNA is at least 3, at least 4, at least 5, at least 6, at least 7, at least 8, at least 9, at least 10, at least 11, at least 12, or more nucleotides in length. In particular embodiments, the linker nucleotide sequence of a single guide RNA is at least 4 nucleotides in length. In certain embodiments, the linker nucleotide sequence is the nucleotide sequence set forth as SEQ ID NO: 136. In other embodiments, the linker nucleotide sequence is at least 6 nucleotides in length.

The single guide RNA or dual-guide RNA can be synthesized chemically or via in vitro transcription. Assays for determining sequence-specific binding between an RGN and a guide RNA are known in the art and include, but are not limited to, in vitro binding assays between an expressed RGN and the guide RNA, which can be tagged with a detectable label (e.g., biotin) and used in a pull-down detection assay in which the guide RNA:RGN complex is captured via the detectable label (e.g., with streptavidin beads). A control guide RNA with an unrelated sequence or structure to the guide RNA can be used as a negative control for non-specific binding of the RGN to RNA. In certain embodiments, the guide RNA is any one of SEQ ID NOs: 129 to 135 and 310, wherein the spacer sequence can be any sequence and is indicated as a poly-N sequence.

In certain embodiments, the guide RNA can be introduced into a target cell, organelle, or embryo as an RNA molecule. The guide RNA can be transcribed in vitro or chemically synthesized. In other embodiments, a nucleotide sequence encoding the guide RNA is introduced into the cell, organelle, or embryo. In some of these embodiments, the nucleotide sequence encoding the guide RNA is operably linked to a promoter (e.g., an RNA polymerase III promoter). The promoter can be a native promoter or heterologous to the guide RNA-encoding nucleotide sequence.

In various embodiments, the guide RNA can be introduced into a target cell, organelle, or embryo as a ribonucleoprotein complex, as described herein, wherein the guide RNA is bound to an RNA-guided nuclease polypeptide.

The guide RNA directs an associated RNA-guided nuclease to a particular target nucleotide sequence of interest through hybridization of the guide RNA to the target nucleotide sequence. A target nucleotide sequence can comprise DNA, RNA, or a combination of both and can be single-stranded or double-stranded. A target nucleotide sequence can be genomic DNA (i.e., chromosomal DNA), plasmid DNA, or an RNA molecule (e.g., messenger RNA, ribosomal RNA, transfer RNA, micro RNA, small interfering RNA). The target nucleotide sequence can be bound (and in some embodiments, cleaved) by an RNA-guided nuclease in vitro or in a cell. The chromosomal sequence targeted by the RGN can be a nuclear, plastid or mitochondrial chromosomal sequence. In some embodiments, the target nucleotide sequence is unique in the target genome.

In some embodiments, the target nucleotide sequence is adjacent to a protospacer adjacent motif (PAM). In certain embodiments, cleavage of a double-stranded target sequence is dependent upon the presence of a PAM, whereas cleavage of a single-stranded target sequence is PAM-independent. A protospacer adjacent motif is generally within about 1 to about 10 nucleotides from the target nucleotide sequence, including about 1, about 2, about 3, about 4, about 5, about 6, about 7, about 8, about 9, or about 10 nucleotides from the target nucleotide sequence. The PAM can be 5′ or 3′ of the target sequence. In some embodiments, the PAM is 5′ of the target sequence for the presently disclosed RGNs. Generally, the PAM is a consensus sequence of about 3-4 nucleotides, but in particular embodiments, can be 1, 2, 3, 4, 5, 6, 7, 8, 9, or more nucleotides in length. In some embodiments, the PAM is 5′ of the target sequence and is T-rich.

In some embodiments, the RGN binds to a guide sequence comprising a CRISPR repeat sequence set forth in any one of SEQ ID NOs: 110 to 119, 139, 141, 143, 146, and 201 to 309, or an active variant or fragment thereof, and a tracrRNA sequence set forth in any one of SEQ ID NOs: 120 to 128, 140, 142, 145, 147, and 148, respectively, or an active variant or fragment thereof. The RGN systems are described further in Example 1 and Table 1 of the present specification.

It is well-known in the art that PAM sequence specificity for a given nuclease enzyme is affected by enzyme concentration (see, e.g., Karvelis et al. (2015) Genome Biol 16:253), which may be modified by altering the promoter used to express the RGN, or the amount of ribonucleoprotein complex delivered to the cell, organelle, or embryo.

In those embodiments wherein binding and cleavage by the RGN is dependent upon a PAM sequence, upon recognizing its corresponding PAM sequence, the RGN can cleave the target nucleotide sequence at a specific cleavage site. As used herein, a cleavage site is made up of the two particular nucleotides within a target nucleotide sequence between which the nucleotide sequence is cleaved by an RGN. The cleavage site can comprise the 1^{st} and 2^{nd}, 2ⁿ^{d} and 3^{rd}, 3^{rd} and 4^{th}, 4^{th} and 5^{th}, 5^{th} and 6^{th}, 7^{th} and 8^{th}, or 8^{th} and 9^{th} nucleotides from the PAM in either the 5′ or 3′ direction. In some embodiments, the cleavage site may be over 10, 11, 12, 13, 14, 15, 16, 17, 18, 19, or 20 nucleotides from the PAM in either the 5′ or 3′ direction. In some embodiments, the cleavage site is 4 nucleotides away from the PAM. In other embodiments, the cleavage site is at least 15 nucleotides away from the PAM. As RGNs can cleave a target nucleotide sequence resulting in staggered ends, in some embodiments, the cleavage site is defined based on the distance of the two nucleotides from the PAM on the positive (+) strand of the polynucleotide and the distance of the two nucleotides from the PAM on the negative (-) strand of the polynucleotide.

### IV. Nucleotides Encoding RNA-Guided Nucleases, CRISPR RNA, and/or TracrRNA

The present disclosure provides polynucleotides comprising the presently disclosed CRISPR RNAs, tracrRNAs, and/or sgRNAs and polynucleotides comprising a nucleotide sequence encoding the presently disclosed RNA-guided nucleases, CRISPR RNAs, tracrRNAs, and/or sgRNAs. Presently disclosed polynucleotides include those comprising or encoding a CRISPR repeat sequence comprising any one of the nucleotide sequences of SEQ ID NOs: 110 to 119, 139, 141, 143, 146, and 201 to 309, or an active variant or fragment thereof that when comprised within a guide RNA is capable of directing the sequence-specific binding of an associated RNA-guided nuclease to a target sequence of interest. Also disclosed are polynucleotides comprising or encoding a tracrRNA comprising any one of the nucleotide sequences of SEQ ID NOs: 120 to 128, 140, 142, 145, 147, and 148, or an active variant or fragment thereof that when comprised within a guide RNA is capable of directing the sequence-specific binding of an associated RNA-guided nuclease to a target sequence of interest. Polynucleotides are also provided that encode an RNA-guided nuclease comprising any one of the amino acid sequences set forth as SEQ ID NOs: 1 to 109, and active fragments or variants thereof that retain the ability to bind to a target nucleotide sequence in an RNA-guided sequence-specific manner.

The use of the term “polynucleotide” is not intended to limit the present disclosure to polynucleotides comprising DNA, though such DNA polynucleotides are contemplated. Those of ordinary skill in the art will recognize that polynucleotides can comprise ribonucleotides (RNA) and combinations of ribonucleotides and deoxyribonucleotides. Such deoxyribonucleotides and ribonucleotides include both naturally occurring molecules and synthetic analogues. These include, e.g., peptide nucleic acids (PNAs), PNA-DNA chimers, locked nucleic acids (LNAs), and phosphothiorate linked sequences. The polynucleotides disclosed herein also encompass all forms of sequences including, but not limited to, single-stranded forms, double-stranded forms, DNA-RNA hybrids, triplex structures, stem-and-loop structures, circular forms (e.g., including circular RNA), and the like.

In some embodiments, the nucleic acid molecules encoding RGNs are codon optimized for expression in an organism of interest. A “codon-optimized” coding sequence is a polynucleotide coding sequence having its frequency of codon usage designed to mimic the frequency of preferred codon usage or transcription conditions of a particular host cell. Expression in the particular host cell or organism is enhanced as a result of the alteration of one or more codons at the nucleic acid level such that the translated amino acid sequence is not changed. Nucleic acid molecules can be codon optimized, either wholly or in part. Codon tables and other references providing preference information for a wide range of organisms are available in the art (see, e.g., Campbell and Gowri (1990) Plant Physiol. 92:1-11 for a discussion of plant-preferred codon usage). Methods are available in the art for synthesizing plant-preferred genes. See, for example, U.S. Pat. Nos. 5,380,831, and 5,436,391, and Murray et al. (1989) Nucleic Acids Res. 17:477-498, herein incorporated by reference.

Polynucleotides encoding the RGNs, crRNAs, tracrRNAs, and/or sgRNAs provided herein are in some embodiments provided in expression cassettes for in vitro expression or expression in a cell, organelle, embryo, or organism of interest. The cassette may include 5′ and 3′ regulatory sequences operably linked to a polynucleotide encoding an RGN, crRNA, tracrRNAs, and/or sgRNAs provided herein that allows for expression of the polynucleotide. The cassette may additionally contain at least one additional gene or genetic element to be cotransformed into the organism. Where additional genes or elements are included, the components are operably linked. The term “operably linked” is intended to mean a functional linkage between two or more elements. For example, an operable linkage between a promoter and a coding region of interest (e.g., region coding for an RGN, crRNA, tracrRNAs, and/or sgRNAs) is a functional link that allows for expression of the coding region of interest. Operably linked elements may be contiguous or non-contiguous. When used to refer to the joining of two protein coding regions, by operably linked is intended that the coding regions are in the same reading frame. Alternatively, the additional gene(s) or element(s) can be provided on multiple expression cassettes. For example, the nucleotide sequence encoding a presently disclosed RGN can be present on one expression cassette, whereas the nucleotide sequence encoding a crRNA, tracrRNA, or complete guide RNA can be on a separate expression cassette. Such an expression cassette is provided with a plurality of restriction sites and/or recombination sites for insertion of the polynucleotides to be under the transcriptional regulation of the regulatory regions. The expression cassette may additionally contain a selectable marker gene.

The expression cassette may include in the 5′-3′ direction of transcription, a transcriptional (and, in some embodiments, translational) initiation region (i.e., a promoter), an RGN-, crRNA-, tracrRNA-and/or sgRNA- encoding polynucleotide of the invention, and a transcriptional (and in some embodiments, translational) termination region (i.e., termination region) functional in the organism of interest. The promoters of the invention are capable of directing or driving expression of a coding sequence in a host cell. The regulatory regions (e.g., promoters, transcriptional regulatory regions, and translational termination regions) may be endogenous or heterologous to the host cell or to each other. As used herein, “heterologous” in reference to a sequence is a sequence that originates from a foreign species, or, if from the same species, is substantially modified from its native form in composition and/or genomic locus by deliberate human intervention. As used herein, a chimeric gene comprises a coding sequence operably linked to a transcription initiation region that is heterologous to the coding sequence.

Convenient termination regions are available from the Ti-plasmid of A.tumefaciens, such as the octopine synthase and nopaline synthase termination regions. See also Guerineau et al. (1991) Mol. Gen. Genet. 262: 141-144; Proudfoot (1991) Cell 64:671-674; Sanfacon et al. (1991) Genes Dev. 5: 141-149; Mogen et al. (1990) Plant Cell 2: 1261-1272; Munroe et al. (1990) Gene 91: 151-158; Ballas et al. (1989) Nucleic Acids Res. 17:7891-7903; and Joshi et al. (1987) Nucleic Acids Res. 15:9627-9639.

Additional regulatory signals include, but are not limited to, transcriptional initiation start sites, operators, activators, enhancers, other regulatory elements, ribosomal binding sites, an initiation codon, termination signals, and the like. See, for example, U.S. Pat. Nos. 5,039,523 and 4,853,331; EPO0480762A2; Sambrook et al. (1992) Molecular Cloning: A Laboratory Manual, ed. Maniatis et al. (Cold Spring Harbor Laboratory Press, Cold Spring Harbor, N.Y.), hereinafter “Sambrook 11”; Davis et al., eds.

Advanced Bacterial Genetics (Cold Spring Harbor Laboratory Press), Cold Spring Harbor, N.Y., and the references cited therein.

In preparing the expression cassette, the various DNA fragments may be manipulated, so as to provide for the DNA sequences in the proper orientation and, as appropriate, in the proper reading frame. Toward this end, adapters or linkers may be employed to join the DNA fragments or other manipulations may be involved to provide for convenient restriction sites, removal of superfluous DNA, removal of restriction sites, or the like. For this purpose, in vitro mutagenesis, primer repair, restriction, annealing, resubstitutions, e.g., transitions and transversions, may be involved.

A number of promoters can be used in the practice of the invention. The promoters can be selected based on the desired outcome. The nucleic acids can be combined with constitutive, inducible, growth stage-specific, cell type-specific, tissue-preferred, tissue-specific, or other promoters for expression in the organism of interest. See, for example, promoters set forth in WO 99/43838 and in U.S. Pat. Nos: 8,575,425; 7,790,846; 8,147,856; 8,586832; 7,772,369; 7,534,939; 6,072,050; 5,659,026; 5,608,149; 5,608,144; 5,604,121; 5,569,597; 5,466,785; 5,399,680; 5,268,463; 5,608,142; and 6,177,611; herein incorporated by reference.

For expression in plants, constitutive promoters also include CaMV 35S promoter (Odell et al. (1985) Nature 313:810-812); rice actin (McElroy et al. (1990) Plant Cell 2:163-171); ubiquitin (Christensen et al. (1989) Plant Mol. Biol. 12:619-632 and Christensen et al. (1992) Plant Mol. Biol. 18:675-689); pEMU (Last et al. (1991) Theor. Appl. Genet. 81:581-588); and MAS (Velten et al. (1984) EMBO J. 3:2723-2730).

Examples of inducible promoters are the Adh1 promoter which is inducible by hypoxia or cold stress, the Hsp70 promoter which is inducible by heat stress, the PPDK promoter and the pepcarboxylase promoter which are both inducible by light. Also useful are promoters which are chemically inducible, such as the In2-2 promoter which is safener induced (U.S. Pat. No. 5,364,780), the Axig1 promoter which is auxin induced and tapetum specific but also active in callus (PCT US01/22169), the steroid-responsive promoters (see, for example, the ERE promoter which is estrogen induced, and the glucocorticoid-inducible promoter in Schena et al. (1991) Proc. Natl. Acad. Sci. USA 88:10421-10425 and McNellis et al. (1998) Plant J. 14(2):247-257) and tetracycline-inducible and tetracycline-repressible promoters (see, for example, Gatz et al. (1991) Mol. Gen. Genet. 227:229-237, and U.S. Pat. Nos. 5,814,618 and 5,789,156), herein incorporated by reference.

Tissue-specific or tissue-preferred promoters can be utilized to target expression of an expression construct within a particular tissue. In certain embodiments, the tissue-specific or tissue-preferred promoters are active in plant tissue. Examples of promoters under developmental control in plants include promoters that initiate transcription preferentially in certain tissues, such as leaves, roots, fruit, seeds, or flowers. A “tissue specific” promoter is a promoter that initiates transcription only in certain tissues. Unlike constitutive expression of genes, tissue-specific expression is the result of several interacting levels of gene regulation. As such, promoters from homologous or closely related plant species can be preferable to use to achieve efficient and reliable expression of transgenes in particular tissues. In some embodiments, the expression comprises a tissue-preferred promoter. A “tissue preferred” promoter is a promoter that initiates transcription preferentially, but not necessarily entirely or solely in certain tissues.

In some embodiments, the nucleic acid molecules encoding an RGN, crRNA, and/or tracrRNA comprise a cell type-specific promoter. A “cell type specific” promoter is a promoter that primarily drives expression in certain cell types in one or more organs. Some examples of plant cells in which cell type specific promoters functional in plants may be primarily active include, for example, BETL cells, vascular cells in roots, leaves, stalk cells, and stem cells. The nucleic acid molecules can also include cell type preferred promoters. A “cell type preferred” promoter is a promoter that primarily drives expression mostly, but not necessarily entirely or solely in certain cell types in one or more organs. Some examples of plant cells in which cell type preferred promoters functional in plants may be preferentially active include, for example, BETL cells, vascular cells in roots, leaves, stalk cells, and stem cells.

The nucleic acid sequences encoding the RGNs, crRNAs, tracrRNAs, and/or sgRNAs can be operably linked to a promoter sequence that is recognized by a phage RNA polymerase for example, for in vitro mRNA synthesis. In such embodiments, the in vitro-transcribed RNA can be purified for use in the methods described herein. For example, the promoter sequence can be a T7, T3, or SP6 promoter sequence or a variation of a T7, T3, or SP6 promoter sequence. In such embodiments, the expressed protein and/or RNAs can be purified for use in the methods of genome modification described herein.

In certain embodiments, the polynucleotide encoding the RGN, crRNA, tracrRNA, and/or sgRNA also can be linked to a polyadenylation signal (e.g., SV40 polyA signal and other signals functional in plants) and/or at least one transcriptional termination sequence. Additionally, the sequence encoding the RGN also can be linked to sequence(s) encoding at least one nuclear localization signal, at least one cell-penetrating domain, and/or at least one signal peptide capable of trafficking proteins to particular subcellular locations, as described elsewhere herein.

The polynucleotide encoding the RGN, crRNA, tracrRNA, and/or sgRNA can be present in a vector or multiple vectors. A “vector” refers to a polynucleotide composition for transferring, delivering, or introducing a nucleic acid into a host cell. Suitable vectors include plasmid vectors, phagemids, cosmids, artificial/mini-chromosomes, transposons, and viral vectors (e.g., lentiviral vectors, adeno-associated viral vectors, baculoviral vector). The vector can comprise additional expression control sequences (e.g., enhancer sequences, Kozak sequences, polyadenylation sequences, transcriptional termination sequences), selectable marker sequences (e.g., antibiotic resistance genes), origins of replication, and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3rd edition, 2001.

The vector can also comprise a selectable marker gene for the selection of transformed cells. Selectable marker genes are utilized for the selection of transformed cells or tissues. Marker genes include genes encoding antibiotic resistance, such as those encoding neomycin phosphotransferase II (NEO) and hygromycin phosphotransferase (HPT), as well as genes conferring resistance to herbicidal compounds, such as glufosinate ammonium, bromoxynil, imidazolinones, and 2,4-dichlorophenoxyacetate (2,4-D).

In some embodiments, the expression cassette or vector comprising the sequence encoding the RGN polypeptide can further comprise a sequence encoding a crRNA and/or a tracrRNA, or the crRNA and tracrRNA combined to create a guide RNA. The sequence(s) encoding the crRNA and/or tracrRNA can be operably linked to at least one transcriptional control sequence for expression of the crRNA and/or tracrRNA in the organism or host cell of interest. For example, the polynucleotide encoding the crRNA and/or tracrRNA can be operably linked to a promoter sequence that is recognized by RNA polymerase III (Pol III). Examples of suitable Pol III promoters include, but are not limited to, mammalian U6, U3, H1, and 7SL RNA promoters and rice U6 and U3 promoters.

As indicated, expression constructs comprising nucleotide sequences encoding the RGNs, crRNA, tracrRNA, and/or sgRNA can be used to transform organisms of interest. Methods for transformation involve introducing a nucleotide construct into an organism of interest. By “introducing” is intended to introduce the nucleotide construct to the host cell in such a manner that the construct gains access to the interior of the host cell. The methods of the invention do not require a particular method for introducing a nucleotide construct to a host organism, only that the nucleotide construct gains access to the interior of at least one cell of the host organism. The host cell can be a eukaryotic or prokaryotic cell. In particular embodiments, the eukaryotic host cell is a plant cell, a mammalian cell, or an insect cell. Methods for introducing nucleotide constructs into plants and other host cells are known in the art including, but not limited to, stable transformation methods, transient transformation methods, and virus-mediated methods.

The methods result in a transformed organism, such as a plant, including whole plants, as well as plant organs (e.g., leaves, stems, roots, etc.), seeds, plant cells, propagules, embryos and progeny of the same. Plant cells can be differentiated or undifferentiated (e.g. callus, suspension culture cells, protoplasts, leaf cells, root cells, phloem cells, pollen).

“Transgenic organisms” or “transformed organisms” or “stably transformed” organisms or cells or tissues refers to organisms that have incorporated or integrated a polynucleotide encoding an RGN, crRNA, and/or tracrRNA of the invention. It is recognized that other exogenous or endogenous nucleic acid sequences or DNA fragments may also be incorporated into the host cell. Agrobacterium-and biolistic-mediated transformation remain the two predominantly employed approaches for transformation of plant cells. However, transformation of a host cell may be performed by infection, transfection, microinjection, electroporation, microprojection, biolistics or particle bombardment, electroporation, silica/carbon fibers, ultrasound mediated, PEG mediated, calcium phosphate co-precipitation, polycation DMSO technique, DEAE dextran procedure, and viral mediated, liposome mediated and the like. Viral-mediated introduction of a polynucleotide encoding an RGN, crRNA, and/or tracrRNA includes retroviral, lentiviral, adenoviral, and adeno-associated viral mediated introduction and expression, as well as the use of Caulimoviruses, Geminiviruses, and RNA plant viruses.

Transformation protocols as well as protocols for introducing polypeptides or polynucleotide sequences into plants may vary depending on the type of host cell (e.g., monocot or dicot plant cell) targeted for transformation. Methods for transformation are known in the art and include those set forth in U.S. Pat. Nos.: 8,575,425; 7,692,068; 8,802,934; 7,541,517; each of which is herein incorporated by reference. See, also, Rakoczy-Trojanowska, M. (2002) Cell Mol Biol Lett. 7:849-858; Jones et al. (2005) Plant Methods 1:5; Rivera et al. (2012) Physics of Life Reviews 9:308-345; Bartlett et al. (2008) Plant Methods 4:1-12; Bates, G.W. (1999) Methods in Molecular Biology 111:359-366; Binns and Thomashow (1988) Annual Reviews in Microbiology 42:575-606; Christou, P. (1992) The Plant Journal 2:275-281; Christou, P. (1995) Euphytica 85:13-27; Tzfira et al. (2004) TRENDS in Genetics 20:375-383; Yao et al. (2006) Journal of Experimental Botany 57:3737-3746; Zupan and Zambryski (1995) Plant Physiology 107:1041-1047; Jones et al. (2005) Plant Methods 1:5;

Transformation may result in stable or transient incorporation of the nucleic acid into the cell. “Stable transformation” is intended to mean that the nucleotide construct introduced into a host cell integrates into the genome of the host cell and is capable of being inherited by the progeny thereof. “Transient transformation” is intended to mean that a polynucleotide is introduced into the host cell and does not integrate into the genome of the host cell.

Methods for transformation of chloroplasts are known in the art. See, for example, Svab et al. (1990) Proc. Nail. Acad. Sci. USA 87:8526-8530; Svab and Maliga (1993) Proc. Natl. Acad. Sci. USA 90:913-917; Svab and Maliga (1993) EMBO J. 12:601-606. The method relies on particle gun delivery of DNA containing a selectable marker and targeting of the DNA to the plastid genome through homologous recombination. Additionally, plastid transformation can be accomplished by transactivation of a silent plastid-borne transgene by tissue-preferred expression of a nuclear-encoded and plastid-directed RNA polymerase. Such a system has been reported in McBride et al. (1994) Proc. Natl. Acad. Sci. USA 91:7301-7305.

The cells that have been transformed may be grown into a transgenic organism, such as a plant, in accordance with conventional ways. See, for example, McCormick et al. (1986) Plant Cell Reports 5:81-84. These plants may then be grown, and either pollinated with the same transformed strain or different strains, and the resulting hybrid having constitutive expression of the desired phenotypic characteristic identified. Two or more generations may be grown to ensure that expression of the desired phenotypic characteristic is stably maintained and inherited and then seeds harvested to ensure expression of the desired phenotypic characteristic has been achieved. In this manner, the present invention provides transformed seed (also referred to as “transgenic seed”) having a nucleotide construct of the invention, for example, an expression cassette of the invention, stably incorporated into their genome.

Alternatively, cells that have been transformed may be introduced into an organism. These cells could have originated from the organism, wherein the cells are transformed in an ex vivo approach.

The sequences provided herein may be used for transformation of any plant species, including, but not limited to, monocots and dicots. Examples of plants of interest include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers.

Vegetables include, but are not limited to, tomatoes, lettuce, green beans, lima beans, peas, and members of the genus Curcumis such as cucumber, cantaloupe, and musk melon. Ornamentals include, but are not limited to, azalea, hydrangea, hibiscus, roses, tulips, daffodils, petunias, carnation, poinsettia, and chrysanthemum. Preferably, plants of the present invention are crop plants (for example, maize, sorghum, wheat, sunflower, tomato, crucifers, peppers, potato, cotton, rice, soybean, sugarbeet, sugarcane, tobacco, barley, oilseed rape, etc.).

As used herein, the term plant includes plant cells, plant protoplasts, plant cell tissue cultures from which plants can be regenerated, plant calli, plant clumps, and plant cells that are intact in plants or parts of plants such as embryos, pollen, ovules, seeds, leaves, flowers, branches, fruit, kernels, ears, cobs, husks, stalks, roots, root tips, anthers, and the like. Grain is intended to mean the mature seed produced by commercial growers for purposes other than growing or reproducing the species. Progeny, variants, and mutants of the regenerated plants are also included within the scope of the invention, provided that these parts comprise the introduced polynucleotides. Further provided is a processed plant product or byproduct that retains the sequences disclosed herein, including for example, soymeal.

The polynucleotides encoding the RGNs, crRNAs, and/or tracrRNAs can also be used to transform any prokaryotic species, including but not limited to, archaea and bacteria (e.g., Bacillus sp., Klebsiella sp. Streptomyces sp., Rhizobium sp., Escherichia sp., Pseudomonas sp., Salmonella sp., Shigella sp., Vibrio sp., Yersinia sp., Mycoplasma sp., Agrobacterium, Lactobacillus sp.).

The polynucleotides encoding the RGNs, crRNAs, and/or tracrRNAs can be used to transform any eukaryotic species, including but not limited to animals (e.g., mammals, insects, fish, birds, and reptiles), fungi, amoeba, algae, and yeast.

Conventional viral and non-viral based gene transfer methods can be used to introduce nucleic acids in mammalian cells or target tissues. Such methods can be used to administer nucleic acids encoding components of a CRISPR system to cells in culture, or in a host organism. Non-viral vector delivery systems include DNA plasmids, RNA (e.g. a transcript of a vector described herein), naked nucleic acid, and nucleic acid complexed with a delivery vehicle, such as a liposome. Viral vector delivery systems include DNA and RNA viruses, which have either episomal or integrated genomes after delivery to the cell. For a review of gene therapy procedures, see Anderson, Science 256: 808- 813 (1992); Nabel & Feigner, TIBTECH 11:211-217 (1993); Mitani & Caskey, TIBTECH 11:162-166 (1993); Dillon, TIBTECH 11:167-175 (1993); Miller, Nature 357:455-460 (1992); Van Brunt, Biotechnology 6(10): 1149-1154 (1988); Vigne, Restorative Neurology and Neuroscience 8:35-36 (1995); Kremer & Perricaudet, British Medical Bulletin 51(1):31-44 (1995); Haddada et al., in Current Topics in Microbiology and Immunology, Doerfler and Bohm (eds) (1995); and Yu et al., Gene Therapy 1:13-26 (1994).

Methods of non-viral delivery of nucleic acids include lipofection, nucleofection, microinjection, biolistics, virosomes, liposomes, immunoliposomes, polycation or lipid: nucleic acid conjugates, naked DNA, artificial virions, and agent-enhanced uptake of DNA. Lipofection is described in e.g., U.S. Pat. Nos. 5,049,386, 4,946,787; and 4,897,355) and lipofection reagents are sold commercially (e.g., Transfectam™ and Lipofectin™). Cationic and neutral lipids that are suitable for efficient receptor-recognition lipofection of polynucleotides include those of Feigner, WO 91/17424; WO 91/16024. Delivery can be to cells (e.g. in vitro or ex vivo administration) or target tissues (e.g. in vivo administration). The preparation of lipid:nucleic acid complexes, including targeted liposomes such as immunolipid complexes, is well known to one of skill in the art (see, e.g., Crystal, Science 270:404-410 (1995); Blaese et al., Cancer Gene Ther. 2:291- 297 (1995); Behr et al., Bioconjugate Chem. 5:382-389 (1994); Remy et al., Bioconjugate Chem. 5:647-654 (1994); Gao et al., Gene Therapy 2:710-722 (1995); Ahmad et al., Cancer Res. 52:4817-4820 (1992); U.S. Pat. Nos. 4,186,183, 4,217,344, 4,235,871, 4,261,975, 4,485,054, 4,501,728, 4,774,085, 4,837,028, and 4,946,787).

The use of RNA or DNA viral based systems for the delivery of nucleic acids takes advantage of highly evolved processes for targeting a virus to specific cells in the body and trafficking the viral payload to the nucleus. Viral vectors can be administered directly to patients ( in vivo) or they can be used to treat cells in vitro, and the modified cells may optionally be administered to patients ( ex vivo). Conventional viral based systems could include retroviral, lentivirus, adenoviral, adeno-associated and herpes simplex virus vectors for gene transfer. Integration in the host genome is possible with the retrovirus, lentivirus, and adeno-associated virus gene transfer methods, often resulting in long term expression of the inserted transgene. Additionally, high transduction efficiencies have been observed in many different cell types and target tissues.

The tropism of a retrovirus can be altered by incorporating foreign envelope proteins, expanding the potential target population of target cells. Lentiviral vectors are retroviral vectors that are able to transduce or infect non-dividing cells and typically produce high viral titers. Selection of a retroviral gene transfer system would therefore depend on the target tissue. Retroviral vectors are comprised of cis-acting long terminal repeats with packaging capacity for up to 6-10 kb of foreign sequence. The minimum cis-acting LTRs are sufficient for replication and packaging of the vectors, which are then used to integrate the therapeutic gene into the target cell to provide permanent transgene expression. Widely used retroviral vectors include those based upon murine leukemia virus (MuLV), gibbon ape leukemia virus (GaLV), Simian Immuno deficiency virus (SIV), human immuno deficiency virus (HIV), and combinations thereof (see, e.g., Buchscher et al., J. Viral. 66:2731-2739 (1992); Johann et al., J. Viral. 66:1635-1640 (1992); Sommnerfelt et al., Viral. 176:58-59 (1990); Wilson et al., J. Viral. 63:2374-2378 (1989); Miller et al., l. Viral. 65:2220-2224 (1991); PCT/US94/05700).

In applications where transient expression is preferred, adenoviral based systems may be used. Adenoviral based vectors are capable of very high transduction efficiency in many cell types and do not require cell division. With such vectors, high titer and levels of expression have been obtained. This vector can be produced in large quantities in a relatively simple system. Adeno-associated virus (“AAV”) vectors may also be used to transduce cells with target nucleic acids, e.g., in the in vitro production of nucleic acids and peptides, and for in vivo and ex vivo gene therapy procedures (see, e.g., West et al., Virology 160:38-47 (1987); U.S. Pat. No. 4,797,368; WO 93/24641; Katin, Human Gene Therapy 5:793-801 (1994); Muzyczka, 1. Clin. Invest. 94:1351 (1994). Construction of recombinant AAV vectors are described in a number of publications, including U.S. Pat. No. 5,173,414; Tratschin et al., Mol. Cell. Biol. 5:3251-3260 (1985); Tratschin, et al., Mol. Cell. Biol. 4:2072-2081 (1984); Hermonat & Muzyczka, PNAS 81:6466-6470 (1984); and Samulski et al., l. Viral. 63:03822-3828 (1989). Packaging cells are typically used to form virus particles that are capable of infecting a host cell. Such cells include 293 cells, which package adenovirus, and ψJ2 cells or PA317 cells, which package retrovirus.

Viral vectors used in gene therapy are usually generated by producing a cell line that packages a nucleic acid vector into a viral particle. The vectors typically contain the minimal viral sequences required for packaging and subsequent integration into a host, other viral sequences being replaced by an expression cassette for the polynucleotide(s) to be expressed. The missing viral functions are typically supplied in trans by the packaging cell line. For example, AAV vectors used in gene therapy typically only possess ITR sequences from the AAV genome which are required for packaging and integration into the host genome. Viral DNA is packaged in a cell line, which contains a helper plasmid encoding the other AAV genes, namely rep and cap, but lacking ITR sequences.

The cell line may also be infected with adenovirus as a helper. The helper virus promotes replication of the AAV vector and expression of AAV genes from the helper plasmid. The helper plasmid is not packaged in significant amounts due to a lack of ITR sequences. Contamination with adenovirus can be reduced by, e.g., heat treatment to which adenovirus is more sensitive than AAV. Additional methods for the delivery of nucleic acids to cells are known to those skilled in the art. See, for example, US20030087817, incorporated herein by reference.

In some embodiments, a host cell is transiently or non-transiently transfected with one or more vectors described herein. In some embodiments, a cell is transfected as it naturally occurs in a subject. In some embodiments, a cell that is transfected is taken from a subject. In some embodiments, the cell is derived from cells taken from a subject, such as a cell line. A wide variety of cell lines for tissue culture are known in the art. Examples of cell lines include, but are not limited to, C8161, CCRF-CEM, MOLT, mIMCD-3, NHDF, HeLaS3, Huh1, Huh4, Huh7, HUVEC, HASMC, HEKn, HEKa, MiaPaCell, Panel, PC-3, TFl, CTLL-2, CIR, Rat6, CVI, RPTE, AlO, T24, 182, A375, ARH-77, Calul, SW480, SW620, SKOV3, SK-UT, CaCo2, P388D1, SEM-K2, WEHI- 231, HB56, TIB55, lurkat, 145.01, LRMB, Bcl-1, BC-3, IC21, DLD2, Raw264.7, NRK, NRK-52E, MRC5, MEF, Hep G2, HeLa B, HeLa T4. COS, COS-1, COS-6, COS-M6A, BS-C-1 monkey kidney epithelial, BALB/3T3 mouse embryo fibroblast, 3T3 Swiss, 3T3-L1, 132-d5 human fetal fibroblasts; 10.1 mouse fibroblasts, 293-T, 3T3, 721, 9L, A2780, A2780ADR, A2780cis, A172, A20, A253, A431, A-549, ALC, B16, B35, BCP-I cells, BEAS-2B, bEnd.3, BHK-21, BR 293, BxPC3, C3H-10T1/2, C6/36, Cal-27, CHO, CHO-7, CHO-IR, CHO-Kl, CHO-K2, CHO-T, CHO Dhfr-/-, COR-L23, COR-L23/CPR, COR-L235010, CORL23/ R23, COS-7, COV-434, CML T1, CMT, CT26, D17, DH82, DU145, DuCaP, EL4, EM2, EM3, EMT6/AR1, EMT6/AR10.0, FM3, H1299, H69, HB54, HB55, HCA2, HEK-293, HeLa, Hepalclc7, HL-60, HMEC, HT-29, lurkat, lY cells, K562 cells, Ku812, KCL22, KGl, KYO1, LNCap, Ma-Mel 1-48, MC-38, MCF-7, MCF-10A, MDA-MB-231, MDA-MB-468, MDA-MB-435, MDCKII, MDCKII, MOR/ 0.2R, MONO-MAC 6, MTD-1A, MyEnd, NCI-H69/CPR, NCI-H69/LX10, NCI-H69/LX20, NCI-H69/LX4, NIH-3T3, NALM-1, NW-145, OPCN/OPCT cell lines, Peer, PNT-1A/ PNT 2, RenCa, RIN-5F, RMA/RMAS, Saos-2 cells, Sf-9, SkBr3, T2, T-47D, T84, THP1 cell line, U373, U87, U937, VCaP, Vero cells, WM39, WT-49, X63, YAC-1, YAR, and transgenic varieties thereof. Cell lines are available from a variety of sources known to those with skill in the art (see, e.g., the American Type Culture Collection (ATCC) (Manassas, Va.)).

In some embodiments, a cell transfected with one or more vectors described herein is used to establish a new cell line comprising one or more vector-derived sequences. In some embodiments, a cell transiently transfected with the components of a CRISPR system as described herein (such as by transient transfection of one or more vectors, or transfection with RNA), and modified through the activity of a CRISPR complex, is used to establish a new cell line comprising cells containing the modification but lacking any other exogenous sequence. In some embodiments, cells transiently or non-transiently transfected with one or more vectors described herein, or cell lines derived from such cells are used in assessing one or more test compounds.

In some embodiments, one or more vectors described herein are used to produce a non-human transgenic animal or transgenic plant. In some embodiments, the transgenic animal is a mammal, such as a mouse, rat, or rabbit.

### V. Variants and Fragments of Polypeptides and Polynucleotides

The present disclosure provides active variants and fragments of a naturally-occurring (i.e., wild-type) RNA-guided nuclease, the amino acid sequences of which are set forth as SEQ ID NOs: 1 to 109, as well as active variants and fragments of naturally-occurring CRISPR repeats, such as the sequences set forth as SEQ ID NOs: 110 to 119, 139, 141, 143, 146, and 201 to 309, and active variant and fragments of naturally-occurring tracrRNAs, such as the sequences set forth as SEQ ID NOs: 120 to 128, 140, 142, 145, 147, and 148, and polynucleotides encoding the same. Also provided are active variants and fragments of naturally-occurring RGN accessory proteins, such as the sequences set forth as SEQ ID NOs: 178-192.

While the activity of a variant or fragment may be altered compared to the polynucleotide or polypeptide of interest, the variant and fragment should retain the functionality of the polynucleotide or polypeptide of interest. For example, a variant or fragment may have increased activity, decreased activity, different spectrum of activity or any other alteration in activity when compared to the polynucleotide or polypeptide of interest.

Fragments and variants of naturally-occurring RGN polypeptides, such as those disclosed herein, will retain sequence-specific, RNA-guided DNA-binding activity. In particular embodiments, fragments and variants of naturally-occurring RGN polypeptides, such as those disclosed herein, will retain nuclease activity (single-stranded or double-stranded).

Fragments and variants of naturally-occurring CRISPR repeats, such as those disclosed herein, will retain the ability, when part of a guide RNA (comprising a tracrRNA), to bind to and guide an RNA-guided nuclease (complexed with the guide RNA) to a target nucleotide sequence in a sequence-specific manner.

Fragments and variants of naturally-occurring tracrRNAs, such as those disclosed herein, will retain the ability, when part of a guide RNA (comprising a CRISPR RNA), to guide an RNA-guided nuclease (complexed with the guide RNA) to a target nucleotide sequence in a sequence-specific manner.

Fragments and variants of naturally-occurring RGN accessory proteins, such as those disclosed herein, will retain the ability, when part of an RGN system (i.e., RGN protein and guide RNA), to allow for the RGN system to bind to a target nucleotide sequence in a sequence-specific manner.

The term “fragment” refers to a portion of a polynucleotide or polypeptide sequence of the invention. “Fragments” or “biologically active portions” include polynucleotides comprising a sufficient number of contiguous nucleotides to retain the biological activity (i.e., binding to and directing an RGN in a sequence-specific manner to a target nucleotide sequence when comprised within a guideRNA). “Fragments” or “biologically active portions” include polypeptides comprising a sufficient number of contiguous amino acid residues to retain the biological activity (i.e., binding to a target nucleotide sequence in a sequence-specific manner when complexed with a guide RNA). Fragments of the RGN proteins include those that are shorter than the full-length sequences due to the use of an alternate downstream start site. A biologically active portion of an RGN protein can be a polypeptide that comprises, for example, 10, 25, 50, 100, 150, 200, 250, 300, 350, 400, 450, 500, 550, 600 or more contiguous amino acid residues of SEQ ID NOs: 1 to 109. Such biologically active portions can be prepared by recombinant techniques and evaluated for sequence-specific, RNA-guided DNA-binding activity. A biologically active fragment of a CRISPR repeat sequence can comprise at least 8 contiguous amino acids of any one of SEQ ID NOs: 110 to 119, 139, 141, 143, 146, and 201 to 309. A biologically active portion of a CRISPR repeat sequence can be a polynucleotide that comprises, for example, 8, 9, 10, 11, 12, 13, 14, 15, 16, 17, 18, 19, or 20 contiguous nucleotides of any one of SEQ ID NOs: 110 to 119, 139, 141, 143, 146, and 201 to 309. A biologically active portion of a tracrRNA can be a polynucleotide that comprises, for example, 8, 9, 10, 11, 12, 13, 14, 15, 16, 17, 18, 19, 20, 21, 22, 23, 24, 25, 26, 27, 28, 29, 30, 35, 40, 45, 50, 55, 60, 65, 70, 75, 80 or more contiguous nucleotides of any one of SEQ ID NOs: 120 to 128, 140, 142, 145, 147, and 148. Fragments of the RGN accessory proteins include those that are shorter than the full-length sequences due to the use of an alternate downstream start site. A biologically active portion of an RGN accessory protein can be a polypeptide that comprises, for example, 10, 25, 50, 100, 150, 200, or more contiguous amino acid residues of SEQ ID NOs: 178 to 192. Such biologically active portions can be prepared by recombinant techniques and evaluated for biological activity.

In general, “variants” is intended to mean substantially similar sequences. For polynucleotides, a variant comprises a deletion and/or addition of one or more nucleotides at one or more internal sites within the native polynucleotide and/or a substitution of one or more nucleotides at one or more sites in the native polynucleotide. As used herein, a “native” or “wild type” polynucleotide or polypeptide comprises a naturally occurring nucleotide sequence or amino acid sequence, respectively. For polynucleotides, conservative variants include those sequences that, because of the degeneracy of the genetic code, encode the native amino acid sequence of the gene of interest. Naturally occurring allelic variants such as these can be identified with the use of well-known molecular biology techniques, as, for example, with polymerase chain reaction (PCR) and hybridization techniques as outlined below. Variant polynucleotides also include synthetically derived polynucleotides, such as those generated, for example, by using site-directed mutagenesis but which still encode the polypeptide or the polynucleotide of interest. Generally, variants of a particular polynucleotide disclosed herein will have at least about 40%, 45%, 50%, 55%, 60%, 65%, 70%, 75%, 80%, 85%, 90%, 91%, 92%, 93%, 94%, 95%, 96%, 97%, 98%, 99% or more sequence identity to that particular polynucleotide as determined by sequence alignment programs and parameters described elsewhere herein.

Variants of a particular polynucleotide disclosed herein (i.e., the reference polynucleotide) can also be evaluated by comparison of the percent sequence identity between the polypeptide encoded by a variant polynucleotide and the polypeptide encoded by the reference polynucleotide. Percent sequence identity between any two polypeptides can be calculated using sequence alignment programs and parameters described elsewhere herein. Where any given pair of polynucleotides disclosed herein is evaluated by comparison of the percent sequence identity shared by the two polypeptides they encode, the percent sequence identity between the two encoded polypeptides is at least about 40%, 45%, 50%, 55%, 60%, 65%, 70%, 75%, 80%, 85%, 90%, 91%, 92%, 93%, 94%, 95%, 96%, 97%, 98%, 99% or more sequence identity.

In particular embodiments, the presently disclosed polynucleotides encode an RNA-guided nuclease polypeptide comprising an amino acid sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to an amino acid sequence of any one of SEQ ID NOs: 1 to 109.

A biologically active variant of an RGN polypeptide of the invention may differ by as few as about 1-15 amino acid residues, as few as about 1-10, such as about 6-10, as few as 5, as few as 4, as few as 3, as few as 2, or as few as 1 amino acid residue. In specific embodiments, the polypeptides can comprise an N-terminal or a C-terminal truncation, which can comprise at least a deletion of 10, 15, 20, 25, 30, 35, 40, 45, 50, 55, 60, 65, 70, 75, 80, 85, 90, 95, 100, 150, 200, 250, 300, 350, 400, 450, 500, 550, 600 amino acids or more from either the N or C terminus of the polypeptide.

In certain embodiments, the presently disclosed polynucleotides encode an RNA-guided nuclease accessory polypeptide comprising an amino acid sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to an amino acid sequence of any one of SEQ ID NOs: 178 to 192.

A biologically active variant of an RGN accessory polypeptide of the invention may differ by as few as about 1-15 amino acid residues, as few as about 1-10, such as about 6-10, as few as 5, as few as 4, as few as 3, as few as 2, or as few as 1 amino acid residue. In specific embodiments, the polypeptides can comprise an N-terminal or a C-terminal truncation, which can comprise at least a deletion of 10, 15, 20, 25, 30, 35, 40, 45, 50, 55, 60, 65, 70, 75, 80, 85, 90, 95, 100, 150, 200 amino acids or more from either the N or C terminus of the polypeptide.

In certain embodiments, the presently disclosed polynucleotides comprise or encode a CRISPR repeat comprising a nucleotide sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to any one of the nucleotide sequences set forth as SEQ ID NOs: 110 to 119, 139, 141, 143, 146, and 201 to 309.

The presently disclosed polynucleotides can comprise or encode a tracrRNA comprising a nucleotide sequence having at least 40%, 45%, 50%, 55%, 60%, 65%, 70%, 75%, 80%, 81%, 82%, 83%, 84%, 85%, 86%, 87%, 88%, 89%, 90%, 91%, 92%, 93%, 94%, 95%, 96%, 97%, 98%, 99%, or greater identity to any one of the nucleotide sequences set forth as SEQ ID NOs: 120 to 128, 140, 142, 145, 147, and 148.

Biologically active variants of a CRISPR repeat or tracrRNA of the invention may differ by as few as about 1-15 nucleotides, as few as about 1-10, such as about 6-10, as few as 5, as few as 4, as few as 3, as few as 2, or as few as 1 nucleotide. In specific embodiments, the polynucleotides can comprise a 5′ or 3′ truncation, which can comprise at least a deletion of 10, 15, 20, 25, 30, 35, 40, 45, 50, 55, 60, 65, 70, 75, 80 nucleotides or more from either the 5′ or 3′ end of the polynucleotide.

It is recognized that modifications may be made to the RGN polypeptides, CRISPR repeats, and tracrRNAs provided herein creating variant proteins and polynucleotides. Changes designed by man may be introduced through the application of site-directed mutagenesis techniques. Alternatively, native, as yet-unknown or as yet unidentified polynucleotides and/or polypeptides structurally and/or functionally-related to the sequences disclosed herein may also be identified that fall within the scope of the present invention. Conservative amino acid substitutions may be made in nonconserved regions that do not alter the function of the RGN proteins. Alternatively, modifications may be made that improve the activity of the RGN.

Variant polynucleotides and proteins also encompass sequences and proteins derived from a mutagenic and recombinogenic procedure such as DNA shuffling. With such a procedure, one or more different RGN proteins disclosed herein (e.g., SEQ ID NOs: 1 to 109) is manipulated to create a new RGN protein possessing the desired properties. In this manner, libraries of recombinant polynucleotides are generated from a population of related sequence polynucleotides comprising sequence regions that have substantial sequence identity and can be homologously recombined in vitro or in vivo. For example, using this approach, sequence motifs encoding a domain of interest may be shuffled between the RGN sequences provided herein and other known RGN genes to obtain a new gene coding for a protein with an improved property of interest, such as an increased Kₘ in the case of an enzyme. Strategies for such DNA shuffling are known in the art. See, for example, Stemmer (1994) Proc. Natl. Acad. Sci. USA 91:10747-10751; Stemmer (1994) Nature 370:389-391; Crameri et al. (1997) Nature Biotech. 15:436-438; Moore et al. (1997) J. Mol. Biol. 272:336-347; Zhang et al. (1997) Proc. Natl. Acad. Sci. USA 94:4504-4509; Crameri et al. (1998) Nature 391:288-291; and U.S. Pat. Nos. 5,605,793 and 5,837,458. A “shuffled” nucleic acid is a nucleic acid produced by a shuffling procedure such as any shuffling procedure set forth herein. Shuffled nucleic acids are produced by recombining (physically or virtually) two or more nucleic acids (or character strings), for example in an artificial, and optionally recursive, fashion. Generally, one or more screening steps are used in shuffling processes to identify nucleic acids of interest; this screening step can be performed before or after any recombination step. In some (but not all) shuffling embodiments, it is desirable to perform multiple rounds of recombination prior to selection to increase the diversity of the pool to be screened. The overall process of recombination and selection are optionally repeated recursively. Depending on context, shuffling can refer to an overall process of recombination and selection, or, alternately, can simply refer to the recombinational portions of the overall process.

As used herein, “sequence identity” or “identity” in the context of two polynucleotides or polypeptide sequences makes reference to the residues in the two sequences that are the same when aligned for maximum correspondence over a specified comparison window. When percentage of sequence identity is used in reference to proteins it is recognized that residue positions which are not identical often differ by conservative amino acid substitutions, where amino acid residues are substituted for other amino acid residues with similar chemical properties (e.g., charge or hydrophobicity) and therefore do not change the functional properties of the molecule. When sequences differ in conservative substitutions, the percent sequence identity may be adjusted upwards to correct for the conservative nature of the substitution. Sequences that differ by such conservative substitutions are said to have “sequence similarity” or “similarity”. Means for making this adjustment are well known to those of skill in the art. Typically, this involves scoring a conservative substitution as a partial rather than a full mismatch, thereby increasing the percentage sequence identity. Thus, for example, where an identical amino acid is given a score of 1 and a non-conservative substitution is given a score of zero, a conservative substitution is given a score between zero and 1. The scoring of conservative substitutions is calculated, e.g., as implemented in the program PC/GENE (Intelligenetics, Mountain View, California).

As used herein, “percentage of sequence identity” means the value determined by comparing two optimally aligned sequences over a comparison window, wherein the portion of the polynucleotide sequence in the comparison window may comprise additions or deletions (i.e., gaps) as compared to the reference sequence (which does not comprise additions or deletions) for optimal alignment of the two sequences. The percentage is calculated by determining the number of positions at which the identical nucleic acid base or amino acid residue occurs in both sequences to yield the number of matched positions, dividing the number of matched positions by the total number of positions in the window of comparison, and multiplying the result by 100 to yield the percentage of sequence identity.

Unless otherwise stated, sequence identity/similarity values provided herein refer to the value obtained using GAP Version 10 using the following parameters: % identity and % similarity for a nucleotide sequence using GAP Weight of 50 and Length Weight of 3, and the nwsgapdna.cmp scoring matrix; % identity and % similarity for an amino acid sequence using GAP Weight of 8 and Length Weight of 2, and the BLOSUM62 scoring matrix; or any equivalent program thereof. By “equivalent program” is intended any sequence comparison program that, for any two sequences in question, generates an alignment having identical nucleotide or amino acid residue matches and an identical percent sequence identity when compared to the corresponding alignment generated by GAP Version 10.

Two sequences are “optimally aligned” when they are aligned for similarity scoring using a defined amino acid substitution matrix (e.g., BLOSUM62), gap existence penalty and gap extension penalty so as to arrive at the highest score possible for that pair of sequences. Amino acid substitution matrices and their use in quantifying the similarity between two sequences are well-known in the art and described, e.g., in Dayhoff et al. (1978) “A model of evolutionary change in proteins.” In “Atlas of Protein Sequence and Structure,” Vol. 5, Suppl. 3 (ed. M. O. Dayhoff), pp. 345-352. Natl. Biomed. Res. Found., Washington, D.C. and Henikoff et al. (1992) Proc. Natl. Acad. Sci. USA 89:10915-10919. The BLOSUM62 matrix is often used as a default scoring substitution matrix in sequence alignment protocols. The gap existence penalty is imposed for the introduction of a single amino acid gap in one of the aligned sequences, and the gap extension penalty is imposed for each additional empty amino acid position inserted into an already opened gap. The alignment is defined by the amino acid positions of each sequence at which the alignment begins and ends, and optionally by the insertion of a gap or multiple gaps in one or both sequences, so as to arrive at the highest possible score. While optimal alignment and scoring can be accomplished manually, the process is facilitated by the use of a computer-implemented alignment algorithm, e.g., gapped BLAST 2.0, described in Altschul et al. (1997) Nucleic Acids Res. 25:3389-3402, and made available to the public at the National Center for Biotechnology Information Website (on the world wide web at ncbi.nlm.nih.gov). Optimal alignments, including multiple alignments, can be prepared using, e.g., PSI-BLAST, available through www.ncbi.nlm.nih.gov and described by Altschul et al. (1997) Nucleic Acids Res. 25:3389-3402.

With respect to an amino acid sequence that is optimally aligned with a reference sequence, an amino acid residue “corresponds to” the position in the reference sequence with which the residue is paired in the alignment. The “position” is denoted by a number that sequentially identifies each amino acid in the reference sequence based on its position relative to the N-terminus. Owing to deletions, insertion, truncations, fusions, etc., that must be taken into account when determining an optimal alignment, in general the amino acid residue number in a test sequence as determined by simply counting from the N-terminal will not necessarily be the same as the number of its corresponding position in the reference sequence. For example, in a case where there is a deletion in an aligned test sequence, there will be no amino acid that corresponds to a position in the reference sequence at the site of deletion. Where there is an insertion in an aligned reference sequence, that insertion will not correspond to any amino acid position in the reference sequence. In the case of truncations or fusions there can be stretches of amino acids in either the reference or aligned sequence that do not correspond to any amino acid in the corresponding sequence.

### VI. Antibodies

Antibodies to the RGN polypeptides or ribonucleoproteins comprising the RGN polypeptides of the present invention, including those having any one of the amino acid sequences set forth as SEQ ID NOs: 1 to 109 or active variants or fragments thereof, or the RGN accessory proteins of the present invention, including those having any one of the amino acid sequences set forth as SEQ ID NOs: 178 to 192 or active variants or fragments thereof, are also encompassed. Methods for producing antibodies are well known in the art (see, for example, Harlow and Lane (1988) Antibodies: A Laboratory Manual, Cold Spring Harbor Laboratory, Cold Spring Harbor, N.Y.; and U.S. Pat. No. 4,196,265). These antibodies can be used in kits for the detection and isolation of RGN polypeptides or ribonucleoproteins. Thus, this disclosure provides kits comprising antibodies that specifically bind to the polypeptides or ribonucleoproteins described herein, including, for example, polypeptides having the sequence of any one of SEQ ID NOs: 1 to 109 or 178 to 192.

### VII. Systems and Ribonucleoprotein Complexes for Binding a Target Sequence of Interest and Methods of Making the Same

The present disclosure provides a system for binding a target sequence of interest, wherein the system comprises at least one guide RNA or a nucleotide sequence encoding the same, and at least one RNA-guided nuclease or a nucleotide sequence encoding the same. The guide RNA hybridizes to the target sequence of interest and also forms a complex with the RGN polypeptide, thereby directing the RGN polypeptide to bind to the target sequence. In some of these embodiments, the RGN comprises an amino acid sequence of any one of SEQ ID NOs: 1 to 109, or an active variant or fragment thereof. In various embodiments, the guide RNA comprises a CRISPR repeat sequence comprising the nucleotide sequence of any one of SEQ ID NOs: 110 to 119, 139, 141, 143, 146, and 201 to 309, or an active variant or fragment thereof. In particular embodiments, the guide RNA comprises a tracrRNA comprising a nucleotide sequence of any one of SEQ ID NOs: 120 to 128, 140, 142, 145, 147, and 148, or an active variant or fragment thereof. The guide RNA of the system can be a single guide RNA or a dual-guide RNA. In particular embodiments, the system comprises an RNA-guided nuclease that is heterologous to the guideRNA, wherein the RGN and guideRNA are not found complexed to one another (i.e., bound to one another) in nature.

In some embodiments, the system further comprises at least one RGN accessory protein in order to bind to and/or cleave a target polynucleotide. In some of these embodiments, the system further comprises at least one RGN accessory protein set forth as SEQ ID NOs: 178-192 or an active variant or fragment thereof. In particular embodiments wherein the RGN is APG06369 (SEQ ID NO: 11) or a variant or fragment thereof, the system can further comprise at least one RGN accessory protein set forth as SEQ ID NOs: 178-181 or an active variant or fragment thereof. In some of those embodiments wherein the RGN is APG03847 (SEQ ID NO: 12) or a variant or fragment thereof, the system can further comprise at least one RGN accessory protein set forth as SEQ ID NOs: 182-184 or an active variant or fragment thereof. In certain embodiments wherein the RGN is APG05625 (SEQ ID NO: 13) or a variant or fragment thereof, the system can further comprise at least one RGN accessory protein set forth as SEQ ID NOs: 185-187 or an active variant or fragment thereof. In some embodiments wherein the RGN is APG03524 (SEQ ID NO: 16) or a variant or fragment thereof, the system can further comprise at least one RGN accessory protein set forth as SEQ ID NOs: 188-190 or an active variant or fragment thereof. In particular embodiments wherein the RGN is APG03759 (SEQ ID NO: 14) or a variant or fragment thereof, the system can further comprise the RGN accessory protein set forth as SEQ ID NO: 191 or an active variant or fragment thereof. In certain embodiments wherein the RGN is APG05123 (SEQ ID NO: 15) or a variant or fragment thereof, the system can further comprise the RGN accessory protein set forth as SEQ ID NO: 192 or an active variant or fragment thereof.

The system for binding a target sequence of interest provided herein can be a ribonucleoprotein complex, which is at least one molecule of an RNA bound to at least one protein. The ribonucleoprotein complexes provided herein comprise at least one guide RNA as the RNA component and an RNA-guided nuclease as the protein component. Such ribonucleoprotein complexes can be purified from a cell or organism that naturally expresses an RGN polypeptide and has been engineered to express a particular guide RNA that is specific for a target sequence of interest. Alternatively, the ribonucleoprotein complex can be purified from a cell or organism that has been transformed with polynucleotides that encode an RGN polypeptide and a guide RNA and cultured under conditions to allow for the expression of the RGN polypeptide and guide RNA. Thus, methods are provided for making an RGN polypeptide or an RGN ribonucleoprotein complex. Such methods comprise culturing a cell comprising a nucleotide sequence encoding an RGN polypeptide, and in some embodiments a nucleotide sequence encoding a guide RNA, under conditions in which the RGN polypeptide (and in some embodiments, the guide RNA) is expressed. The RGN polypeptide or RGN ribonucleoprotein can then be purified from a lysate of the cultured cells.

Methods for purifying an RGN polypeptide or RGN ribonucleoprotein complex from a lysate of a biological sample are known in the art (e.g., size exclusion and/or affinity chromatography, 2D-PAGE, HPLC, reversed-phase chromatography, immunoprecipitation). In particular methods, the RGN polypeptide is recombinantly produced and comprises a purification tag to aid in its purification, including but not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6xHis, 10xHis, biotin carboxyl carrier protein (BCCP), and calmodulin. Generally, the tagged RGN polypeptide or RGN ribonucleoprotein complex is purified using immobilized metal affinity chromatography. It will be appreciated that other similar methods known in the art may be used, including other forms of chromatography or for example immunoprecipitation, either alone or in combination.

An “isolated” or “purified” polypeptide, or biologically active portion thereof, is substantially or essentially free from components that normally accompany or interact with the polypeptide as found in its naturally occurring environment. Thus, an isolated or purified polypeptide is substantially free of other cellular material, or culture medium when produced by recombinant techniques, or substantially free of chemical precursors or other chemicals when chemically synthesized. A protein that is substantially free of cellular material includes preparations of protein having less than about 30%, 20%, 10%, 5%, or 1% (by dry weight) of contaminating protein. When the protein of the invention or biologically active portion thereof is recombinantly produced, optimally culture medium represents less than about 30%, 20%, 10%, 5%, or 1% (by dry weight) of chemical precursors or non-protein-of-interest chemicals.

Particular methods provided herein for binding and/or cleaving a target sequence of interest involve the use of an in vitro assembled RGN ribonucleoprotein complex. In vitro assembly of an RGN ribonucleoprotein complex can be performed using any method known in the art in which an RGN polypeptide is contacted with a guide RNA under conditions to allow for binding of the RGN polypeptide to the guide RNA. As used herein, “contact”, “contacting”, “contacted,” refer to placing the components of a desired reaction together under conditions suitable for carrying out the desired reaction. The RGN polypeptide can be purified from a biological sample, cell lysate, or culture medium, produced via in vitro translation, or chemically synthesized. The guide RNA can be purified from a biological sample, cell lysate, or culture medium, transcribed in vitro, or chemically synthesized. The RGN polypeptide and guide RNA can be brought into contact in solution (e.g., buffered saline solution) to allow for in vitro assembly of the RGN ribonucleoprotein complex.

### VIII. Methods of Binding, Cleaving, or Modifying a Target Sequence

The present disclosure provides methods for binding, cleaving, and/or modifying a target nucleotide sequence of interest. The methods include delivering a system comprising at least one guide RNA or a polynucleotide encoding the same, and at least one RGN polypeptide or a polynucleotide encoding the same to the target sequence or a cell, organelle, or embryo comprising the target sequence. In some of these embodiments, the RGN comprises any one of the amino acid sequences of SEQ ID NOs: 1 to 109, or an active variant or fragment thereof. In various embodiments, the guide RNA comprises a CRISPR repeat sequence comprising any one of the nucleotide sequences of SEQ ID NOs: 110 to 119, 139, 141, 143, 146, and 201 to 309, or an active variant or fragment thereof. In particular embodiments, the guide RNA comprises a tracrRNA comprising any one of the nucleotide sequences of SEQ ID NOs: 120 to 128, 140, 142, 145, 147, and 148, or an active variant or fragment thereof. The guide RNA of the system can be a single guide RNA or a dual-guide RNA. The RGN of the system may be nuclease dead RGN, have nickase activity, or may be a fusion polypeptide. In some embodiments, the fusion polypeptide comprises a base-editing polypeptide, for example a cytidine deaminase or an adenosine deaminase. In other embodiments, the RGN fusion protein comprises a reverse transcriptase. In other embodiments, the RGN fusion protein comprises a polypeptide that recruits members of a functional nucleic acid repair complex, such as a member of the nucleotide excision repair (NER) or transcription coupled-nucleotide excision repair (TC-NER) pathway (Wei et al., 2015, PNAS USA 112(27):E3495-504 ; Troelstra et al., 1992, Cell 71:939-953; Marnef et al., 2017, J Mol Biol 429(9):1277-1288), as described in U.S. Provisional Application No. 62/966,203, which was filed on Jan. 27, 2020, and is incorporated herein by reference in its entirety. In some embodiments, the RGN fusion protein comprises CSB (van den Boom et al., 2004, J Cell Biol 166(1):27-36; van Gool et al., 1997, EMBO J 16(19):5955-65; an example of which is set forth as SEQ ID NO: 138), which is a member of the TC-NER (nucleotide excision repair) pathway and functions in the recruitment of other members. In further embodiments, the RGN fusion protein comprises an active domain of CSB, such as the acidic domain of CSB which comprises amino acid residues 356-394 of SEQ ID NO: 138 (Teng et al., 2018, Nat Commun 9(1):4115).

In particular embodiments, the RGN and/or guide RNA is heterologous to the cell, organelle, or embryo to which the RGN and/or guide RNA (or polynucleotide(s) encoding at least one of the RGN and guide RNA) are introduced.

In some embodiments, the method further requires delivering at least one RGN accessory protein or polynucleotide(s) encoding the same in order for the RGN to bind to and/or cleave a target polynucleotide. In some of these embodiments, the method further requires delivering at least one RGN accessory protein set forth as SEQ ID NOs: 178-192 or an active variant or fragment thereof, or polynucleotide(s) encoding the same. In particular embodiments wherein the RGN is APG06369 (SEQ ID NO: 11) or a variant or fragment thereof, the method further comprises delivering at least one RGN accessory protein set forth as SEQ ID NOs: 178-181 or an active variant or fragment thereof, or polynucleotide(s) encoding the same. In some of those embodiments wherein the RGN is APG03847 (SEQ ID NO: 12) or a variant or fragment thereof, the method further comprises delivering at least one RGN accessory protein set forth as SEQ ID NOs: 315-317 or an active variant or fragment thereof, or polynucleotide(s) encoding the same. In certain embodiments wherein the RGN is APG05625 (SEQ ID NO: 13) or a variant or fragment thereof, the method further comprises delivering at least one RGN accessory protein set forth as SEQ ID NOs: 185-187 or an active variant or fragment thereof, or polynucleotide(s) encoding the same. In some embodiments wherein the RGN is APG03524 (SEQ ID NO: 16) or a variant or fragment thereof, the method further comprises delivering at least one RGN accessory protein set forth as SEQ ID NOs: 188-190 or an active variant or fragment thereof, or polynucleotide(s) encoding the same. In particular embodiments wherein the RGN is APG03759 (SEQ ID NO: 14) or a variant or fragment thereof, the method further comprises delivering the RGN accessory protein set forth as SEQ ID NO: 191 or an active variant or fragment thereof, or polynucleotide(s) encoding the same. In certain embodiments wherein the RGN is APG05123 (SEQ ID NO: 15) or a variant or fragment thereof, the method further comprises delivering the RGN accessory protein set forth as SEQ ID NO: 192 or an active variant or fragment thereof, or polynucleotide(s) encoding the same.

In those embodiments wherein the method comprises delivering a polynucleotide encoding a guide RNA and/or an RGN polypeptide, the cell or embryo can then be cultured under conditions in which the guide RNA and/or RGN polypeptide are expressed. In various embodiments, the method comprises contacting a target sequence with an RGN ribonucleoprotein complex. The RGN ribonucleoprotein complex may comprise an RGN that is nuclease dead or has nickase activity. In some embodiments, the RGN of the ribonucleoprotein complex is a fusion polypeptide comprising a base-editing polypeptide. In certain embodiments, the method comprises introducing into a cell, organelle, or embryo comprising a target sequence an RGN ribonucleoprotein complex. The RGN ribonucleoprotein complex can be one that has been purified from a biological sample, recombinantly produced and subsequently purified, or in vitro-assembled as described herein. In those embodiments wherein the RGN ribonucleoprotein complex that is contacted with the target sequence or a cell organelle, or embryo has been assembled in vitro, the method can further comprise the in vitro assembly of the complex prior to contact with the target sequence, cell, organelle, or embryo.

A purified or in vitro assembled RGN ribonucleoprotein complex can be introduced into a cell, organelle, or embryo using any method known in the art, including, but not limited to electroporation. Alternatively, an RGN polypeptide and/or polynucleotide encoding or comprising the guide RNA can be introduced into a cell, organelle, or embryo using any method known in the art (e.g., electroporation).

Upon delivery to or contact with the target sequence or cell, organelle, or embryo comprising the target sequence, the guide RNA directs the RGN to bind to the target sequence in a sequence-specific manner. In those embodiments wherein the RGN has nuclease activity, the RGN polypeptide cleaves the target sequence of interest upon binding. The target sequence can subsequently be modified via endogenous repair mechanisms, such as non-homologous end joining, or homology-directed repair with a provided donor polynucleotide.

Methods to measure binding of an RGN polypeptide to a target sequence are known in the art and include chromatin immunoprecipitation assays, gel mobility shift assays, DNA pull-down assays, reporter assays, microplate capture and detection assays. Likewise, methods to measure cleavage or modification of a target sequence are known in the art and include in vitro or in vivo cleavage assays wherein cleavage is confirmed using PCR, sequencing, or gel electrophoresis, with or without the attachment of an appropriate label (e.g., radioisotope, fluorescent substance) to the target sequence to facilitate detection of degradation products. Alternatively, the nicking triggered exponential amplification reaction (NTEXPAR) assay can be used (see, e.g., Zhang et al. (2016) Chem. Sci. 7:4951-4957). In vivo cleavage can be evaluated using the Surveyor assay (Guschin et al. (2010) Methods Mol Biol 649:247-256).

In some embodiments, the methods involve the use of a single type of RGN complexed with more than one guide RNA. The more than one guide RNA can target different regions of a single gene or can target multiple genes.

In those embodiments wherein a donor polynucleotide is not provided, a double-stranded break introduced by an RGN polypeptide can be repaired by a non-homologous end-joining (NHEJ) repair process. Due to the error-prone nature of NHEJ, repair of the double-stranded break can result in a modification to the target sequence. As used herein, a “modification” in reference to a nucleic acid molecule refers to a change in the nucleotide sequence of the nucleic acid molecule, which can be a deletion, insertion, or substitution of one or more nucleotides, or a combination thereof. Modification of the target sequence can result in the expression of an altered protein product or inactivation of a coding sequence.

In those embodiments wherein a donor polynucleotide is present, the donor sequence in the donor polynucleotide can be integrated into or exchanged with the target nucleotide sequence during the course of repair of the introduced double-stranded break, resulting in the introduction of the exogenous donor sequence. A donor polynucleotide thus comprises a donor sequence that is desired to be introduced into a target sequence of interest. In some embodiments, the donor sequence alters the original target nucleotide sequence such that the newly integrated donor sequence will not be recognized and cleaved by the RGN. Integration of the donor sequence can be enhanced by the inclusion within the donor polynucleotide of flanking sequences, referred to herein as “homology arms” that have substantial sequence identity with the sequences flanking the target nucleotide sequence, allowing for a homology-directed repair process. In some embodiments, homology arms have a length of at least 50 base pairs, at least 100 base pairs, and up to 2000 base pairs or more, and have at least 90%, at least 95%, or more, sequence homology to their corresponding sequence within the target nucleotide sequence.

In those embodiments wherein the RGN polypeptide introduces double-stranded staggered breaks, the donor polynucleotide can comprise a donor sequence flanked by compatible overhangs, allowing for direct ligation of the donor sequence to the cleaved target nucleotide sequence comprising overhangs by a non-homologous repair process during repair of the double-stranded break.

In those embodiments wherein the method involves the use of an RGN that is a nickase (i.e., is only able to cleave a single strand of a double-stranded polynucleotide), the method can comprise introducing two RGN nickases that target identical or overlapping target sequences and cleave different strands of the polynucleotide. For example, an RGN nickase that only cleaves the positive (+) strand of a double-stranded polynucleotide can be introduced along with a second RGN nickase that only cleaves the negative (-) strand of a double-stranded polynucleotide.

In various embodiments, a method is provided for binding a target nucleotide sequence and detecting the target sequence, wherein the method comprises introducing into a cell, organelle, or embryo at least one guide RNA or a polynucleotide encoding the same, and at least one RGN polypeptide or a polynucleotide encoding the same, expressing the guide RNA and/or RGN polypeptide (if coding sequences are introduced), wherein the RGN polypeptide is a nuclease-dead RGN and further comprises a detectable label, and the method further comprises detecting the detectable label. The detectable label may be fused to the RGN as a fusion protein (e.g., fluorescent protein) or may be a small molecule conjugated to or incorporated within the RGN polypeptide that can be detected visually or by other means.

Also provided herein are methods for modulating the expression of a target sequence or a gene of interest under the regulation of a target sequence. The methods comprise introducing into a cell, organelle, or embryo at least one guide RNA or a polynucleotide encoding the same, and at least one RGN polypeptide or a polynucleotide encoding the same, expressing the guide RNA and/or RGN polypeptide (if coding sequences are introduced), wherein the RGN polypeptide is a nuclease-dead RGN. In some of these embodiments, the nuclease-dead RGN is a fusion protein comprising an expression modulator domain (i.e., epigenetic modification domain, transcriptional activation domain or a transcriptional repressor domain) as described herein.

The present disclosure also provides methods for binding and/or modifying a target nucleotide sequence of interest. The methods include delivering a system comprising at least one guide RNA or a polynucleotide encoding the same, and at least one fusion polypeptide comprises an RGN of the invention and a base-editing polypeptide, for example a cytidine deaminase or an adenosine deaminase, or a polynucleotide encoding the fusion polypeptide, to the target sequence or a cell, organelle, or embryo comprising the target sequence.

One of ordinary skill in the art will appreciate that any of the presently disclosed methods can be used to target a single target sequence or multiple target sequences. Thus, methods comprise the use of a single RGN polypeptide in combination with multiple, distinct guide RNAs, which can target multiple, distinct sequences within a single gene and/or multiple genes. Also encompassed herein are methods wherein multiple, distinct guide RNAs are introduced in combination with multiple, distinct RGN polypeptides. These guide RNAs and guide RNA/RGN polypeptide systems can target multiple, distinct sequences within a single gene and/or multiple genes.

In one aspect, the invention provides kits containing any one or more of the elements disclosed in the above methods and compositions. In some embodiments, the kit comprises a vector system and instructions for using the kit. In some embodiments, the vector system comprises (a) a first regulatory element operably linked to a DNA sequence encoding the crRNA sequence and one or more insertion sites for inserting a guide sequence upstream of the encoded crRNA sequence, wherein when expressed, the guide sequence directs sequence-specific binding of a CRISPR complex to a target sequence in a eukaryotic cell, wherein the CRISPR complex comprises a CRISPR enzyme complexed with (a) the guide RNA polynucleotide; and/or (b) a second regulatory element operably linked to an enzyme coding sequence encoding said CRISPR enzyme comprising a nuclear localization sequence.

In some embodiments, the kit comprises one or more oligonucleotides corresponding to a guide sequence for insertion into a vector so as to operably link the guide sequence and a regulatory element. In some embodiments, the kit comprises a homologous recombination template polynucleotide. In one aspect, the invention provides methods for using one or more elements of a CRISPR system. The CRISPR complex of the invention provides an effective means for modifying a target polynucleotide. The CRISPR complex of the invention has a wide variety of utility including modifying (e.g., deleting, inserting, translocating, inactivating, activating, base editing) a target polynucleotide in a multiplicity of cell types. As such the CRISPR complex of the invention has a broad spectrum of applications in, e.g., gene therapy, drug screening, disease diagnosis, and prognosis. An exemplary CRISPR complex comprises a CRISPR enzyme complexed with a guide sequence hybridized to a target sequence within the target polynucleotide.

### IX. Target Polynucleotides

In one aspect, the invention provides for methods of modifying a target polynucleotide in a eukaryotic cell, which may be in vivo, ex vivo or in vitro. In some embodiments, the method comprises sampling a cell or population of cells from a human or non-human animal or plant (including microalgae) and modifying the cell or cells. Culturing may occur at any stage ex vivo. The cell or cells may even be reintroduced into the non-human animal or plant (including micro-algae).

Using natural variability, plant breeders combine most useful genes for desirable qualities, such as yield, quality, uniformity, hardiness, and resistance against pests. These desirable qualities also include growth, day length preferences, temperature requirements, initiation date of floral or reproductive development, fatty acid content, insect resistance, disease resistance, nematode resistance, fungal resistance, herbicide resistance, tolerance to various environmental factors including drought, heat, wet, cold, wind, and adverse soil conditions including high salinity The sources of these useful genes include native or foreign varieties, heirloom varieties, wild plant relatives, and induced mutations, e.g., treating plant material with mutagenic agents. Using the present invention, plant breeders are provided with a new tool to induce mutations. Accordingly, one skilled in the art can analyze the genome for sources of useful genes, and in varieties having desired characteristics or traits employ the present invention to induce the rise of useful genes, with more precision than previous mutagenic agents and hence accelerate and improve plant breeding programs.

The target polynucleotide of an RGN system can be any polynucleotide endogenous or exogenous to the eukaryotic cell. For example, the target polynucleotide can be a polynucleotide residing in the nucleus of the eukaryotic cell. The target polynucleotide can be a sequence coding a gene product (e.g., a protein) or a non-coding sequence (e.g., a regulatory polynucleotide or a junk DNA). In some embodiments, the target sequence is associated with a PAM (protospacer adjacent motif); that is, a short sequence recognized by the CRISPR complex. The precise sequence and length requirements for the PAM differ depending on the CRISPR enzyme used (and in some embodiments, the RGN does not require a PAM sequence), but PAMs are typically 2-5 base pair sequences adjacent the protospacer (that is, the target sequence).

The target polynucleotide of a CRISPR complex may include a number of disease-associated genes and polynucleotides as well as signaling biochemical pathway-associated genes and polynucleotides. Examples of target polynucleotides include a sequence associated with a signaling biochemical pathway, e.g., a signaling biochemical pathway-associated gene or polynucleotide. Examples of target polynucleotides include a disease associated gene or polynucleotide. A “disease-associated” gene or polynucleotide refers to any gene or polynucleotide which is yielding transcription or translation products at an abnormal level or in an abnormal form in cells derived from a disease-affected tissues compared with tissues or cells of a non-disease control. It may be a gene that becomes expressed at an abnormally high level; it may be a gene that becomes expressed at an abnormally low level, where the altered expression correlates with the occurrence and/or progression of the disease. A disease-associated gene also refers to a gene possessing mutation(s) or genetic variation that is directly responsible or is in linkage disequilibrium with a gene(s) that is responsible for the etiology of a disease (e.g., a causal mutation). The transcribed or translated products may be known or unknown, and further may be at a normal or abnormal level. Examples of disease-associated genes and polynucleotides are available from McKusick-Nathans Institute of Genetic Medicine, Johns Hopkins University (Baltimore, Md.) and National Center for Biotechnology Information, National Library of Medicine (Bethesda, Md.), available on the World Wide Web.

Although CRISPR systems are particularly useful for their relative ease in targeting to genomic sequences of interest, there still remains an issue of what the RGN can do to address a causal mutation. One approach is to produce a fusion protein between an RGN (preferably an inactive or nickase variant of the RGN) and a base-editing enzyme or the active domain of a base editing enzyme, such as a cytidine deaminase or an adenosine deaminase base editor (U.S. Pat. No. 9,840, 699, herein incorporated by reference). In some embodiments, the methods comprise contacting a DNA molecule with (a) a fusion protein comprising an RGN of the invention and a base-editing polypeptide such as a deaminase; and (b) a gRNA targeting the fusion protein of (a) to a target nucleotide sequence of the DNA strand; wherein the DNA molecule is contacted with the fusion protein and the gRNA in an amount effective and under conditions suitable for the deamination of a nucleobase. In some embodiments, the target DNA sequence comprises a sequence associated with a disease or disorder, and wherein the deamination of the nucleobase results in a sequence that is not associated with a disease or disorder. In some embodiments, the target DNA sequence resides in an allele of a crop plant, wherein the particular allele of the trait of interest results in a plant of lesser agronomic value. The deamination of the nucleobase results in an allele that improves the trait and increases the agronomic value of the plant.

In some embodiments, the DNA sequence comprises a T→C or A→G point mutation associated with a disease or disorder, and wherein the deamination of the mutant C or G base results in a sequence that is not associated with a disease or disorder. In some embodiments, the deamination corrects a point mutation in the sequence associated with the disease or disorder.

In some embodiments, the sequence associated with the disease or disorder encodes a protein, and wherein the deamination introduces a stop codon into the sequence associated with the disease or disorder, resulting in a truncation of the encoded protein. In some embodiments, the contacting is performed in vivo in a subject susceptible to having, having, or diagnosed with the disease or disorder. In some embodiments, the disease or disorder is a disease associated with a point mutation, or a single-base mutation, in the genome. In some embodiments, the disease is a genetic disease, a cancer, a metabolic disease, or a lysosomal storage disease.

### X. Pharmaceutical Compositions and Methods of Treatment

Pharmaceutical compositions comprising the presently disclosed RGN polypeptides and active variants and fragments thereof, as well as polynucleotides encoding the same, the presently disclosed gRNAs or polynucleotides encoding the same, the presently disclosed systems, or cells comprising any of the RGN polypeptides or RGN-encoding polynucleotides, gRNA or gRNA-encoding polynucleotides, or the RGN systems, and a pharmaceutically acceptable carrier are provided.

A pharmaceutical composition is a composition that is employed to prevent, reduce in intensity, cure or otherwise treat a target condition or disease that comprises an active ingredient (i.e., RGN polypeptides, RGN-encoding polynucleotides, gRNA, gRNA-encoding polynucleotides, RGN systems, or cells comprising any one of these) and a pharmaceutically acceptable carrier.

As used herein, a “pharmaceutically acceptable carrier” refers to a material that does not cause significant irritation to an organism and does not abrogate the activity and properties of the active ingredient (i.e., RGN polypeptides, RGN-encoding polynucleotides, gRNA, gRNA-encoding polynucleotides, RGN systems, or cells comprising any one of these). Carriers must be of sufficiently high purity and of sufficiently low toxicity to render them suitable for administration to a subject being treated. The carrier can be inert, or it can possess pharmaceutical benefits. In some embodiments, a pharmaceutically acceptable carrier comprises one or more compatible solid or liquid filler, diluents or encapsulating substances which are suitable for administration to a human or other vertebrate animal. In some embodiments, the pharmaceutically acceptable carrier is not naturally-occurring. In some embodiments, the pharmaceutically acceptable carrier and the active ingredient are not found together in nature.

Pharmaceutical compositions used in the presently disclosed methods can be formulated with suitable carriers, excipients, and other agents that provide suitable transfer, delivery, tolerance, and the like. A multitude of appropriate formulations are known to those skilled in the art. See, e.g., Remington, The Science and Practice of Pharmacy (21st ed. 2005). Suitable formulations include, for example, powders, pastes, ointments, jellies, waxes, oils, lipids, lipid (cationic or anionic) containing vesicles (such as LIPOFECTIN vesicles), lipid nanoparticles, DNA conjugates, anhydrous absorption pastes, oil-in-water and water-in-oil emulsions, emulsions carbowax (polyethylene glycols of various molecular weights), semi-solid gels, and semi-solid mixtures containing carbowax. Pharmaceutical compositions for oral or parenteral use may be prepared into dosage forms in a unit dose suited to fit a dose of the active ingredients. Such dosage forms in a unit dose include, for example, tablets, pills, capsules, injections (ampoules), suppositories, etc.

In some embodiments wherein cells comprising or modified with the presently disclosed RGN, gRNAs, RGN systems or polynucleotides encoding the same are administered to a subject, the cells are administered as a suspension with a pharmaceutically acceptable carrier. One of skill in the art will recognize that a pharmaceutically acceptable carrier to be used in a cell composition will not include buffers, compounds, cryopreservation agents, preservatives, or other agents in amounts that substantially interfere with the viability of the cells to be delivered to the subject. A formulation comprising cells can include e.g., osmotic buffers that permit cell membrane integrity to be maintained, and optionally, nutrients to maintain cell viability or enhance engraftment upon administration. Such formulations and suspensions are known to those of skill in the art and/or can be adapted for use with the cells described herein using routine experimentation.

A cell composition can also be emulsified or presented as a liposome composition, provided that the emulsification procedure does not adversely affect cell viability. The cells and any other active ingredient can be mixed with excipients that are pharmaceutically acceptable and compatible with the active ingredient, and in amounts suitable for use in the therapeutic methods described herein.

Additional agents included in a cell composition can include pharmaceutically acceptable salts of the components therein. Pharmaceutically acceptable salts include the acid addition salts (formed with the free amino groups of the polypeptide) that are formed with inorganic acids, such as, for example, hydrochloric or phosphoric acids, or such organic acids as acetic, tartaric, mandelic and the like. Salts formed with the free carboxyl groups can also be derived from inorganic bases, such as, for example, sodium, potassium, ammonium, calcium or ferric hydroxides, and such organic bases as isopropylamine, trimethylamine, 2-ethylamino ethanol, histidine, procaine and the like.

Physiologically tolerable and pharmaceutically acceptable carriers are well known in the art. Exemplary liquid carriers are sterile aqueous solutions that contain no materials in addition to the active ingredients and water, or contain a buffer such as sodium phosphate at physiological pH value, physiological saline or both, such as phosphate-buffered saline. Still further, aqueous carriers can contain more than one buffer salt, as well as salts such as sodium and potassium chlorides, dextrose, polyethylene glycol and other solutes. Liquid compositions can also contain liquid phases in addition to and to the exclusion of water. Exemplary of such additional liquid phases are glycerin, vegetable oils such as cottonseed oil, and water-oil emulsions. The amount of an active compound used in the cell compositions that is effective in the treatment of a particular disorder or condition can depend on the nature of the disorder or condition, and can be determined by standard clinical techniques.

The presently disclosed RGN polypeptides, guide RNAs, RGN systems or polynucleotides encoding the same can be formulated with pharmaceutically acceptable excipients such as carriers, solvents, stabilizers, adjuvants, diluents, etc., depending upon the particular mode of administration and dosage form. In some embodiments, these pharmaceutical compositions are formulated to achieve a physiologically compatible pH, and range from a pH of about 3 to a pH of about 11, about pH 3 to about pH 7, depending on the formulation and route of administration. In some embodiments, the pH can be adjusted to a range from about pH 5.0 to about pH 8. In some embodiments, the compositions can comprise a therapeutically effective amount of at least one compound as described herein, together with one or more pharmaceutically acceptable excipients. In some embodiments, the compositions comprise a combination of the compounds described herein, or include a second active ingredient useful in the treatment or prevention of bacterial growth (for example and without limitation, anti-bacterial or anti-microbial agents), or include a combination of reagents of the present disclosure.

Suitable excipients include, for example, carrier molecules that include large, slowly metabolized macromolecules such as proteins, polysaccharides, polylactic acids, polyglycolic acids, polymeric amino acids, amino acid copolymers, and inactive virus particles. Other exemplary excipients can include antioxidants (for example and without limitation, ascorbic acid), chelating agents (for example and without limitation, EDTA), carbohydrates (for example and without limitation, dextrin, hydroxyalkylcellulose, and hydroxyalkylmethylcellulose), stearic acid, liquids (for example and without limitation, oils, water, saline, glycerol and ethanol), wetting or emulsifying agents, pH buffering substances, and the like.

In some embodiments, the formulations are provided in unit-dose or multi-dose containers, for example sealed ampules and vials, and may be stored in a freeze-dried (lyophilized) condition requiring the addition of the sterile liquid carrier, for example, saline, water-for-injection, a semi-liquid foam, or gel, immediately prior to use. Extemporaneous injection solutions and suspensions may be prepared from sterile powders, granules and tablets of the kind previously described. In some embodiments, the active ingredient is dissolved in a buffered liquid solution that is frozen in a unit-dose or multi-dose container and later thawed for injection or kept/stabilized under refrigeration until use.

The therapeutic agent(s) may be contained in controlled release systems. In order to prolong the effect of a drug, it often is desirable to slow the absorption of the drug from subcutaneous, intrathecal, or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle. In some embodiments, the use of a long-term sustained release implant may be particularly suitable for treatment of chronic conditions. Long-term sustained release implants are well-known to those of ordinary skill in the art.

Methods of treating a disease in a subject in need thereof are provided herein. The methods comprise administering to a subject in need thereof an effective amount of a presently disclosed RGN polypeptide or active variant or fragment thereof or a polynucleotide encoding the same, a presently disclosed gRNA or a polynucleotide encoding the same, a presently disclosed RGN system, or a cell modified by or comprising any one of these compositions.

In some embodiments, the treatment comprises in vivo gene editing by administering a presently disclosed RGN polypeptide, gRNA, or RGN system or polynucleotide(s) encoding the same. In some embodiments, the treatment comprises ex vivo gene editing wherein cells are genetically modified ex vivo with a presently disclosed RGN polypeptide, gRNA, or RGN system or polynucleotide(s) encoding the same and then the modified cells are administered to a subject. In some embodiments, the genetically modified cells originate from the subject that is then administered the modified cells, and the transplanted cells are referred to herein as autologous. In some embodiments, the genetically modified cells originate from a different subject (i.e., donor) within the same species as the subject that is administered the modified cells (i.e., recipient), and the transplanted cells are referred to herein as allogeneic. In some examples described herein, the cells can be expanded in culture prior to administration to a subject in need thereof.

In some embodiments, the disease to be treated with the presently disclosed compositions is one that can be treated with immunotherapy, such as with a chimeric antigen receptor (CAR) T cell. Such diseases include but are not limited to cancer.

In some embodiments, the disease to be treated with the presently disclosed compositions is associated with a sequence (i.e., the sequence is causal for the disease or disorder or causal for symptoms associated with the disease or disorder) that is mutated in order to treat the disease or disorder or the reduction of symptoms associated with the disease or disorder. In some embodiments, the disease to be treated with the presently disclosed compositions is associated with a causal mutation. As used herein, a “causal mutation” refers to a particular nucleotide, nucleotides, or nucleotide sequence in the genome that contributes to the severity or presence of a disease or disorder in a subject. The correction of the causal mutation leads to the improvement of at least one symptom resulting from a disease or disorder. In some embodiments, the causal mutation is adjacent to a PAM site recognized by an RGN disclosed herein. The causal mutation can be corrected with a presently disclosed RGN or a fusion polypeptide comprising a presently disclosed RGN and a base-editing polypeptide (i.e., a base editor). Non-limiting examples of diseases associated with a causal mutation include cystic fibrosis, Hurler syndrome, Friedreich’s Ataxia, Huntington’s Disease, and sickle cell disease. Additional non-limiting examples of disease-associated genes and mutations are available from McKusick-Nathans Institute of Genetic Medicine, Johns Hopkins University (Baltimore, Md.) and National Center for Biotechnology Information, National Library of Medicine (Bethesda, Md.), available on the World Wide Web.

In some embodiments, the methods provided herein are used to introduce a deactivating point mutation into a gene or allele that encodes a gene product that is associated with a disease or disorder. For example, in some embodiments, methods are provided herein that employ a presently disclosed composition to introduce a deactivating point mutation into an oncogene (e.g., in the treatment of a proliferative disease). A deactivating mutation may, in some embodiments, generate a premature stop codon in a coding sequence, which results in the expression of a truncated gene product, e.g., a truncated protein lacking the function of the full-length protein. In some embodiments, the purpose of the methods provided herein is to restore the function of a dysfunctional gene via genome editing. The presently disclosed RGN polypeptides and systems comprising the same can be validated for gene editing-based human therapeutics in vitro, e.g., by correcting a disease associated mutation in human cell culture.

As used herein, “treatment” or “treating,” or “palliating” or “ameliorating” are used interchangeably. These terms refer to an approach for obtaining beneficial or desired results including but not limited to a therapeutic benefit and/or a prophylactic benefit. By therapeutic benefit is meant any therapeutically relevant improvement in or effect on one or more diseases, conditions, or symptoms under treatment. For prophylactic benefit, the compositions may be administered to a subject at risk of developing a particular disease, condition, or symptom, or to a subject reporting one or more of the physiological symptoms of a disease, even though the disease, condition, or symptom may not have yet been manifested.

The term “effective amount” or “therapeutically effective amount” refers to the amount of an agent that is sufficient to effect beneficial or desired results. The therapeutically effective amount may vary depending upon one or more of: the subject and disease condition being treated, the weight and age of the subject, the severity of the disease condition, the manner of administration and the like, which can readily be determined by one of ordinary skill in the art. The specific dose may vary depending on one or more of: the particular agent chosen, the dosing regimen to be followed, whether it is administered in combination with other compounds, timing of administration, and the delivery system in which it is carried.

The term “administering” refers to the placement of an active ingredient into a subject, by a method or route that results in at least partial localization of the introduced active ingredient at a desired site, such as a site of injury or repair, such that a desired effect(s) is produced. In those embodiments wherein cells are administered, the cells can be administered by any appropriate route that results in delivery to a desired location in the subject where at least a portion of the implanted cells or components of the cells remain viable. The period of viability of the cells after administration to a subject can be as short as a few hours, e.g., twenty-four hours, to a few days, to as long as several years, or even the life time of the patient, i.e., long-term engraftment. For example, in some aspects described herein, an effective amount of photoreceptor cells or retinal progenitor cells is administered via a systemic route of administration, such as an intraperitoneal or intravenous route.

In some embodiments, the administering comprises administering by viral delivery. In some embodiments, the administering comprises administering by electroporation. In some embodiments, the administering comprises administering by nanoparticle delivery. In some embodiments, the administering comprises administering by liposome delivery. Any effective route of administration can be used to administer an effective amount of a pharmaceutical composition described herein. In some embodiments, the administering comprises administering by a method selected from the group consisting of: intravenously, subcutaneously, intramuscularly, orally, rectally, by aerosol, parenterally, ophthalmicly, pulmonarily, transdermally, vaginally, otically, nasally, and by topical administration, or any combination thereof. In some embodiments, for the delivery of cells, administration by injection or infusion is used.

As used herein, the term “subject” refers to any individual for whom diagnosis, treatment or therapy is desired. In some embodiments, the subject is an animal. In some embodiments, the subject is a mammal. In some embodiments, the subject is a human being.

The efficacy of a treatment can be determined by the skilled clinician. However, a treatment is considered an “effective treatment,” if any one or all of the signs or symptoms of a disease or disorder are altered in a beneficial manner (e.g., decreased by at least 10%), or other clinically accepted symptoms or markers of disease are improved or ameliorated. Efficacy can also be measured by failure of an individual to worsen as assessed by hospitalization or need for medical interventions (e.g., progression of the disease is halted or at least slowed). Methods of measuring these indicators are known to those of skill in the art. Treatment includes: (1) inhibiting the disease, e.g., arresting, or slowing the progression of symptoms; or (2) relieving the disease, e.g., causing regression of symptoms; and (3) preventing or reducing the likelihood of the development of symptoms.

### A. Modifying Causal Mutations Using Base-Editing

In some embodiments, RGNs of the invention are used to modify causal mutations using base-editing. An example of a genetically inherited disease which could be corrected using an approach that relies on an RGN-base editor fusion protein of the invention is Hurler Syndrome. Hurler Syndrome, also known as MPS-1, is the result of a deficiency of α-L-iduronidase (IDUA) resulting in a lysosomal storage disease characterized at the molecular level by the accumulation of dermatan sulfate and heparan sulfate in lysosomes. This disease is generally an inherited genetic disorder caused by mutations in the IDUA gene encoding α-L-iduronidase. Common IDUA mutations are W402X and Q70X, both nonsense mutations resulting in premature termination of translation. Such mutations are well addressed by precise genome editing (PGE) approaches, since reversion of a single nucleotide, for example by a base-editing approach, would restore the wild-type coding sequence and result in protein expression controlled by the endogenous regulatory mechanisms of the genetic locus. Additionally, since heterozygotes are known to be asymptomatic, a PGE therapy that targets one of these mutations would be useful to a large proportion of patients with this disease, as only one of the mutated alleles needs to be corrected (Bunge et al. (1994) Hum. Mol. Genet. 3(6): 861-866, herein incorporated by reference).

Current treatments for Hurler Syndrome include enzyme replacement therapy and bone marrow transplants (Vellodi et al. (1997) Arch. Dis. Child. 76(2): 92-99; Peters et al. (1998) Blood 91(7): 2601-2608, herein incorporated by reference). While enzyme replacement therapy has had a dramatic effect on the survival and quality of life of Hurler Syndrome patients, this approach requires costly and time-consuming weekly infusions. Additional approaches include the delivery of the IDUA gene on an expression vector or the insertion of the gene into a highly expressed locus such as that of serum albumin (U.S. Pat. No. 9,956,247, herein incorporated by reference). However, these approaches do not restore the original IDUA locus to the correct coding sequence. A genome-editing strategy would have a number of advantages, most notably that regulation of gene expression would be controlled by the natural mechanisms present in healthy individuals. Additionally, using base editing does not necessitate causing a double stranded DNA breaks, which could lead to large chromosomal rearrangements, cell death, or oncogenecity by the disruption of tumor suppression mechanisms. A general strategy may be directed toward using RGN-base editor fusion proteins of the invention to target and correct certain disease-causing mutations in the human genome. It will be appreciated that similar approaches to target diseases that can be corrected by base-editing may also be pursued. It will be further appreciated that similar approaches to target disease-causing mutations in other species, particularly common household pets or livestock, can also be deployed using the RGNs of the invention. Common household pets and livestock include dogs, cats, horses, pigs, cows, sheep, chickens, donkeys, snakes, ferrets, and fish including salmon and shrimp.

### B. Modifying Causal Mutations by Targeted Deletion

RGNs of the invention could also be useful in human therapeutic approaches where the causal mutation is more complicated. For example, some diseases such as Friedreich’s Ataxia and Huntington’s Disease are the result of a significant increase in repeats of a three nucleotide motif at a particular region of a gene, which affects the ability of the expressed protein to function or to be expressed. Friedreich’s Ataxia (FRDA) is an autosomal recessive disease resulting in progressive degeneration of nervous tissue in the spinal cord. Reduced levels of the frataxin (FXN) protein in the mitochondria cause oxidative damages and iron deficiencies at the cellular level. The reduced FXN expression has been linked to a GAA triplet expansion within the intron 1 of the somatic and germline FXN gene. In FRDA patients, the GAA repeat frequently consists of more than 70, sometimes even more than 1000 (most commonly 600-900) triplets, whereas unaffected individuals have about 40 repeats or less (Pandolfo et al. (2012) Handbook of Clinical Neurology 103: 275-294; Campuzano et al. (1996) Science 271: 1423-1427; Pandolfo (2002) Adv. Exp. Med. Biol. 516: 99-118; all herein incorporated by reference).

The expansion of the trinucleotide repeat sequence causing Friedreich’s Ataxia (FRDA) occurs in a defined genetic locus within the FXN gene, referred to as the FRDA instability region. RNA guided nucleases (RGNs) may be used for excising the instability region in FRDA patient cells. This approach requires 1) an RGN and guide RNA sequence that can be programmed to target the allele in the human genome; and 2) a delivery approach for the RGN and guide sequence. Many nucleases used for genome editing, such as the commonly used Cas9 nuclease from S. pyogenes (SpCas9), are too large to be packaged into adeno-associated viral (AAV) vectors, especially when considering the length of the SpCas9 gene and the guide RNA in addition to other genetic elements required for functional expression cassettes. This makes an approach using SpCas9 more difficult.

Certain RNA guided nucleases of the invention are well suited for packaging into an AAV vector along with a guide RNA. Packing two guide RNAs would likely require a second vector, but this approach still compares favorably to what would be required of a larger nuclease such as SpCas9, which may require splitting the protein sequence between two vectors. The present invention encompasses a strategy using RGNs of the invention in which a region of genomic instability is removed. Such a strategy is applicable to other diseases and disorders which have a similar genetic basis, such as Huntington’s Disease. Similar strategies using RGNs of the invention may also be applicable to similar diseases and disorders in non-human animals of agronomic or economic importance, including dogs, cats, horses, pigs, cows, sheep, chickens, donkeys, snakes, ferrets, and fish including salmon and shrimp.

### C. Modifying Causal Mutations by Targeted Mutagenesis

RGNs of the invention could also be to introduce disruptive mutations that may result in a beneficial effect. Genetic defects in the genes encoding hemoglobin, particularly the beta globin chain (the HBB gene), can be responsible for a number of diseases known as hemoglobinopathies, including sickle cell anemia and thalassemias.

In adult humans, hemoglobin is a heterotetramer comprising two alpha (α)-like globin chains and two beta (β)-like globin chains and 4 heme groups. In adults the α2β2 tetramer is referred to as Hemoglobin A (HbA) or adult hemoglobin. Typically, the alpha and beta globin chains are synthesized in an approximate 1:1 ratio and this ratio seems to be critical in terms of hemoglobin and red blood cell (RBC) stabilization. In a developing fetus, a different form of hemoglobin, fetal hemoglobin (HbF), is produced which has a higher binding affinity for oxygen than Hemoglobin A such that oxygen can be delivered to the baby’s system via the mother’s blood stream. Fetal hemoglobin also contains two α globin chains, but in place of the adult β-globin chains, it has two fetal gamma (γ)-globin chains (i.e., fetal hemoglobin is α2γ2). The regulation of the switch from production of gamma- to beta-globin is quite complex, and primarily involves a down-regulation of gamma globin transcription with a simultaneous up-regulation of beta globin transcription. At approximately 30 weeks of gestation, the synthesis of gamma globin in the fetus starts to drop while the production of beta globin increases. By approximately 10 months of age, the newborn’s hemoglobin is nearly all α2β2 although some HbF persists into adulthood (approximately 1-3% of total hemoglobin). In the majority of patients with hemoglobinopathies, the genes encoding gamma globin remain present, but expression is relatively low due to normal gene repression occurring around parturition as described above.

Sickle cell disease is caused by a V6E mutation in the β globin gene (HBB) (a GAG to GTG at the DNA level), where the resultant hemoglobin is referred to as “hemoglobinS” or “HbS.” Under lower oxygen conditions, HbS molecules aggregate and form fibrous precipitates. These aggregates cause the abnormality or ‘sickling’ of the RBCs, resulting in a loss of flexibility of the cells. The sickling RBCs are no longer able to squeeze into the capillary beds and can result in vaso-occlusive crisis in sickle cell patients. In addition, sickled RBCs are more fragile than normal RBCs, and tend towards hemolysis, eventually leading to anemia in the patient.

Treatment and management of sickle cell patients is a life-long proposition involving antibiotic treatment, pain management and transfusions during acute episodes. One approach is the use of hydroxyurea, which exerts its effects in part by increasing the production of gamma globin. Long term side effects of chronic hydroxyurea therapy are still unknown, however, and treatment gives unwanted side effects and can have variable efficacy from patient to patient. Despite an increase in the efficacy of sickle cell treatments, the life expectancy of patients is still only in the mid to late 50’s and the associated morbidities of the disease have a profound impact on a patient’s quality of life.

Thalassemias (alpha thalassemias and beta thalassemia) are also diseases relating to hemoglobin and typically involve a reduced expression of globin chains. This can occur through mutations in the regulatory regions of the genes or from a mutation in a globin coding sequence that results in reduced expression or reduced levels or functional globin protein. Treatment of thalassemias usually involves blood transfusions and iron chelation therapy. Bone marrow transplants are also being used for treatment of people with severe thalassemias if an appropriate donor can be identified, but this procedure can have significant risks.

One approach that has been proposed for the treatment of both sickle cell disease (SCD) and beta thalassemias is to increase the expression of gamma globin so that HbF functionally replaces the aberrant adult hemoglobin. As mentioned above, treatment of SCD patients with hydroxyurea is thought to be successful in part due to its effect on increasing gamma globin expression (DeSimone (1982) Proc Nat’l Acad Sci USA 79(14):4428-31; Ley, et al., (1982) N. Engl. J. Medicine, 307: 1469-1475; Ley, et al., (1983) Blood 62: 370-380; Constantoulakis et al., (1988) Blood 72(6):1961-1967, all herein incorporated by reference). Increasing the expression of HbF involves identification of genes whose products play a role in the regulation of gamma globin expression. One such gene is BCL11A. BCL11A encodes a zinc finger protein that expressed in adult erythroid precursor cells, and down-regulation of its expression leads to an increase in gamma globin expression (Sankaran et at (2008) Science 322: 1839, herein incorporated by reference). Use of an inhibitory RNA targeted to the BCL11A gene has been proposed (e.g., U.S. Pat. Publication 2011/0182867, herein incorporated by reference) but this technology has several potential drawbacks, including that complete knock down may not be achieved, delivery of such RNAs may be problematic, and the RNAs must be present continuously, requiring multiple treatments for life.

RGNs of the invention may be used to target the BCL11A enhancer region to disrupt expression of BCL11A, thereby increasing gamma globin expression. This targeted disruption can be achieved by non-homologous end joining (NHEJ), whereby an RGN of the invention targets to a particular sequence within the BCL11A enhancer region, makes a double-stranded break, and the cell’s machinery repairs the break, typically simultaneously introducing deleterious mutations. Similar to what is described for other disease targets, RGNs of the invention may have advantages over other known RGNs due to their relatively small size, which enables packaging expression cassettes for the RGN and its guide RNA into a single AAV vector for in vivo delivery. Similar strategies using RGNs of the invention may also be applicable to similar diseases and disorders in both humans and in non-human animals of agronomic or economic importance.

### XI. Cells Comprising a Polynucleotide Genetic Modification

Provided herein are cells and organisms comprising a target sequence of interest that has been modified using a process mediated by an RGN, crRNA, and/or tracrRNA as described herein. In some of these embodiments, the RGN comprises any one of the amino acid sequences of SEQ ID NOs: 1 to 109, or an active variant or fragment thereof. In various embodiments, the guide RNA comprises a CRISPR repeat sequence comprising any one of the nucleotide sequences of SEQ ID NOs: 110 to 119, 139, 141, 143, 146, and 201 to 309, or an active variant or fragment thereof. In particular embodiments, the guide RNA comprises a tracrRNA comprising any one of the nucleotide sequences of SEQ ID NOs: 120 to 128, 140, 142, 145, 147, and 148, or an active variant or fragment thereof. The guide RNA of the system can be a single guide RNA or a dual-guide RNA.

The modified cells can be eukaryotic (e.g., mammalian, plant, insect cell) or prokaryotic. Also provided are organelles and embryos comprising at least one nucleotide sequence that has been modified by a process utilizing an RGN, crRNA, and/or tracrRNA as described herein. The genetically modified cells, organisms, organelles, and embryos can be heterozygous or homozygous for the modified nucleotide sequence.

The chromosomal modification of the cell, organism, organelle, or embryo can result in altered expression (up-regulation or down-regulation), inactivation, or the expression of an altered protein product or an integrated sequence. In those embodiments wherein the chromosomal modification results in either the inactivation of a gene or the expression of a non-functional protein product, the genetically modified cell, organism, organelle, or embryo is referred to as a “knock out”. The knock out phenotype can be the result of a deletion mutation (i.e., deletion of at least one nucleotide), an insertion mutation (i.e., insertion of at least one nucleotide), or a nonsense mutation (i.e., substitution of at least one nucleotide such that a stop codon is introduced).

In some embodiments, the chromosomal modification of a cell, organism, organelle, or embryo can produce a “knock in”, which results from the chromosomal integration of a nucleotide sequence that encodes a protein. In some of these embodiments, the coding sequence is integrated into the chromosome such that the chromosomal sequence encoding the wild-type protein is inactivated, but the exogenously introduced protein is expressed.

In some embodiments, the chromosomal modification results in the production of a variant protein product. The expressed variant protein product can have at least one amino acid substitution and/or the addition or deletion of at least one amino acid. The variant protein product encoded by the altered chromosomal sequence can exhibit modified characteristics or activities when compared to the wild-type protein, including but not limited to altered enzymatic activity or substrate specificity.

In some embodiments, the chromosomal modification can result in an altered expression pattern of a protein. As a non-limiting example, chromosomal alterations in the regulatory regions controlling the expression of a protein product can result in the overexpression or downregulation of the protein product or an altered tissue or temporal expression pattern.

The cells that have been modified can be grown into an organism, such as a plant, in accordance with conventional ways. See, for example, McCormick et al. (1986) Plant Cell Reports 5:81-84. These plants may then be grown, and either pollinated with the same modified strain or different strains, and the resulting hybrid having the genetic modification. The present invention provides genetically modified seed. Progeny, variants, and mutants of the regenerated plants are also included within the scope of the invention, provided that these parts comprise the genetic modification. Further provided is a processed plant product or byproduct that retains the genetic modification, including for example, soymeal.

The methods provided herein may be used for modification of any plant species, including, but not limited to, monocots and dicots. Examples of plants of interest include, but are not limited to, corn (maize), sorghum, wheat, sunflower, tomato, crucifers, peppers, potato, cotton, rice, soybean, sugarbeet, sugarcane, tobacco, barley, and oilseed rape, Brassica sp., alfalfa, rye, millet, safflower, peanuts, sweet potato, cassava, coffee, coconut, pineapple, citrus trees, cocoa, tea, banana, avocado, fig, guava, mango, olive, papaya, cashew, macadamia, almond, oats, vegetables, ornamentals, and conifers.

Vegetables include, but are not limited to, tomatoes, lettuce, green beans, lima beans, peas, and members of the genus Curcumis such as cucumber, cantaloupe, and musk melon. Ornamentals include, but are not limited to, azalea, hydrangea, hibiscus, roses, tulips, daffodils, petunias, carnation, poinsettia, and chrysanthemum. Preferably, plants of the present invention are crop plants (for example, maize, sorghum, wheat, sunflower, tomato, crucifers, peppers, potato, cotton, rice, soybean, sugarbeet, sugarcane, tobacco, barley, oilseed rape, etc.).

The methods provided herein can also be used to genetically modify any prokaryotic species, including but not limited to, archaea and bacteria (e.g., Bacillus sp., Klebsiella sp. Streptomyces sp., Rhizobium sp., Escherichia sp., Pseudomonas sp., Salmonella sp., Shigella sp., Vibrio sp., Yersinia sp., Mycoplasma sp., Agrobacterium, Lactobacillus sp.).

The methods provided herein can be used to genetically modify any eukaryotic species or cells therefrom, including but not limited to animals (e.g., mammals, insects, fish, birds, and reptiles), fungi, amoeba, algae, and yeast. In some embodiments, the cell that is modified by the presently disclosed methods include cells of hematopoietic origin, such as cells of the immune system (i.e., immune cells) including but not limited to B cells, T cells, natural killer (NK) cells, stem cells including pluripotent stem cells and induced pluripotent stem cells, chimeric antigen receptor T (CAR-T) cells, monocytes, macrophages, and dendritic cells.

Cells that have been modified may be introduced into an organism. These cells could have originated from the same organism (e.g., person) in the case of autologous cellular transplants, wherein the cells are modified in an ex vivo approach. Alternatively, the cells originated from another organism within the same species (e.g., another person) in the case of allogeneic cellular transplants.

### XII. Kits and Methods for Detecting Target DNA or Cleaving a Population of Single-Stranded DNA

The presently disclosed RGNs, particularly, APG09624 and APG05405 (set forth as SEQ ID NOs: 2 and 4), can promiscuously cleave non-targeted single-stranded DNA (ssDNA) once activated by detection of a target DNA. Thus, provided herein are compositions and methods for detecting a target DNA (double-stranded or single-stranded) in a sample.

Methods of detecting a target DNA of a DNA molecule comprise contacting a sample with an RGN (or a polynucleotide encoding the same), a guide RNA (or a polynucleotide encoding the same) capable of hybridizing with the RGN and a target DNA sequence in a DNA molecule, and a detector single-stranded DNA (detector ssDNA) that does not hybridize with the guide RNA, followed by measuring a detectable signal produced by cleavage of the ssDNA by the RGN, thereby detecting the target DNA sequence of the DNA molecule. In some embodiments, the method can comprise a step of amplification of the nucleic acid molecules within a sample, either before or simultaneously with contact with the RGN and guideRNA. In some of these embodiments, specific sequences to which the guide RNA will hybridize can be amplified in order to increase sensitivity of a detection method.

In those embodiments wherein a sample is contacted with a polynucleotide encoding an RGN polypeptide and/or a polynucleotide encoding the guide RNA, the sample comprises intact cells and the polynucleotides are introduced into the cells in which they are then expressed. In some of these embodiments, at least one of the polynucleotides further comprises a promoter that is operably linked to the nucleotide sequence encoding the RGN polypeptide and/or guide RNA.

In some embodiments, the desired target may exist as RNA, such as the genome or part of a genome of an RNA virus, such as for example a coronavirus. In some embodiments, the coronavirus may be a SARS-like coronavirus. In further embodiments, the coronavirus may be SARS-CoV-2, SARS-CoV, or a bat SARS-like coronavirus such as bat-SL-CoVZC45 (accession MG772933). In embodiments where the target exists as RNA, the target may be reverse-transcribed into a DNA molecule which can be effectively targeted by the RGN. Reverse-transcription may be followed by an amplification step, such as RT-PCR methods known in the art, which involve thermocycling, or may be by isothermal methods such as RT-LAMP (reverse transcription loop-mediated isothermal amplification) (Notomi et al., Nucleic Acids Res 28: E63, (2000)).

The nucleic acid amplification can occur before the sample is contacted with the RGN, guide RNA, and detector ssDNA or amplification can occur simultaneously with the contacting step.

In certain embodiments, the method involves contacting a sample with an RGN and more than one guide RNA. The guide RNAs, each capable of hybridizing with the RGN, can bind to unique target sequences of a single DNA molecule in order to amplify the detectable signal and lead to the detection of that DNA molecule.

These compositions and methods involve the use of a detector ssDNA that does not hybridize with the guideRNA and is a non-target ssDNA. In some embodiments, the detector ssDNA comprises a detectable label that provides a detectable signal after cleavage of the detector ssDNA. A non-limiting example is a detector ssDNA that comprises a fluorophore/quencher pair wherein the fluorophore does not fluoresce when the detector ssDNA is whole (i.e., uncleaved) as its signal is suppressed by the presence of the quencher in close proximity. Cleavage of the detector ssDNA results in removal of the quencher and the fluorescent label can then be detected. Non-limiting examples of fluorescent labels or dyes include Cy5, fluorescein (e.g., FAM, 6 FAM, 5(6) FAM, FITC), Cy3, Alexa Fluor® dyes, and Texas Red. Non-limiting examples of quenchers include Iowa Black®FQ, Iowa Black® RQ, a Qx1 quencher, an ATT0 quencher, and a QSY dye. In some embodiments, the detector ssDNA comprises a second quencher, such as an internal quencher like ZEN™, TAO™, and Black Hole Quencher®, which can lower background and increase signal detection.

In other embodiments, the detector ssDNA comprises a detectable label that provides a detectable signal before cleavage of the detector ssDNA and cleavage of the ssDNA inhibits or prevents detection of the signal. A non-limiting example of such a scenario is a detector ssDNA that comprises a fluorescence resonance energy transfer (FRET) pair. FRET is a process by which radiationless transfer of energy occurs from an excited state of a first (donor) fluorophore to a second (acceptor) fluorophore in close proximity. The emission spectrum of the donor fluorophore overlaps with the excitation spectrum of the acceptor fluorophore. Thus, the acceptor fluorophore will fluoresce when the detector ssDNA is whole (i.e., uncleaved) and the acceptor fluorophore will no longer fluoresce when the detector ssDNA is cleaved because the donor and acceptor fluorophore will no longer be in close proximity to one another. FRET donor and acceptor fluorophores are known in the art and include, but are not limited to cyan fluorescent protein (CFP)/green fluorescent protein (GFP), Cy3/Cy5, and GFP/yellow fluorescent protein (YFP).

In some embodiments, the detector ssDNA has a length of from about 2 nucleotides to about 30 nucleotides, including but not limited to about 2, about 3, about 4, about 5, about 6, about 7, about 8, about 9, about 10, about 11, about 12, about 13, about 14, about 15, about 16, about 17, about 18, about 19, about 20, about 21, about 22, about 23, about 24, about 25 nucleotides, about 26 nucleotides, about 27 nucleotides, about 28 nucleotides, about 29 nucleotides, and about 30 nucleotides.

The sample in which a target DNA can be detected using these compositions and methods comprising a detector ssDNA include any sample comprising or believed to comprise a nucleic acid (e.g., DNA or RNA molecule). The sample can be derived from any source including a synthetic combination of purified nucleic acids or a biological sample such as respiratory swab (e.g., nasopharyngeal swab) extracts, a cell lysate, a patient sample, cells, tissues, saliva, blood, serum, plasma, urine, aspirate, biopsy samples, cerebral spinal fluid, or organism (e.g., bacteria, virus).

The contacting of the sample with the RGN, guide RNA, and detector ssDNA can include contacting in vitro, ex vivo, or in vivo. In some embodiments, the detector ssDNA and/or the RGN and/or guide RNA is immobilized on for example, a lateral flow device, wherein the sample contacts the immobilized detector ssDNA and/or RGN and/or guide RNA. In some embodiments, antibodies against antigen moieties on the detector ssDNA are immobilized on, for example, a lateral flow device in a manner that allows differentiation of cleaved detector ssDNA from intact detector ssDNA. Also provided are devices (e.g., lateral flow, microfluidic), such as those described in International Publ. No. WO 2020/028729, which is herein incorporated by reference in its entirety, that comprise an immobilized detector ssDNA. The RGN and guide RNA can be added to a sample prior to, simultaneous with, or after the addition of the sample to the device and when the target DNA is present within the sample, the RGN will cleave the target DNA as well as the detector ssDNA, leading to the increase or reduction of a detectable signal that can be measured to detect the presence of the target DNA sequence. Alternatively, the RGN and/or guide RNA is immobilized on the device (e.g., lateral flow, microfluidic) and the sample and the detector ssDNA are added to the device. The detector ssDNA can be added to the sample before, during or after addition of the sample to the device. Another alternative device (e.g., lateral flow, microfluidic) comprises an immobilized antibody (or antibodies) against antigen moieties on the detector ssDNA and the sample, detector ssDNA, RGN and guide RNA are added to the device. In some embodiments, the methods can further comprise determining the amount of the target DNA present in the sample. The measurement of the detectable signal in the test sample can be compared to a reference measurement (e.g., a measurement of a reference sample or series thereof comprising a known amount of target DNA).

Non-limiting examples of applications of the compositions and methods include single-nucleotide polymorphism (SNP) detection, cancer screening, detection of a bacterial infection, detection of antibiotic resistance, and detection of a viral infection.

The detectable signal produced by cleavage of the ssDNA by the RGN can be measured using any suitable method known in the art including but not limited to measuring fluorescent signal, a visual analysis of bands on a gel, a colorimetric change, and the presence or absence of an electrical signal.

The present invention provides kits for detecting a target DNA of a DNA molecule in a sample, wherein the kit comprises an RGN polypeptide of the invention (or a polynucleotide comprising a nucleotide sequence encoding the RGN polypeptide), a guide RNA (or a polynucleotide comprising a nucleotide sequence encoding the guide RNA) capable of hybridizing with the RGN and a target DNA sequence in a DNA molecule, and a detector ssDNA that does not hybridize with the guide RNA. In those embodiments wherein the target to be detected is an RNA, the kit can further comprise a reverse transcriptase. In those embodiments wherein nucleic acid amplification is used, the kit comprising the RGN and guide RNA (or polynucleotides encoding the same), and detector ssDNA can further comprise nucleic acid amplification reagents (e.g., DNA polymerase, nucleotides, buffer). In those embodiments wherein the kit comprises a polynucleotide encoding an RGN polypeptide and/or a polynucleotide encoding the guide RNA, the polynucleotides are introduced into a cell in which they are then expressed. In some of these embodiments at least one of the polynucleotides further comprises a promoter that is operably linked to the nucleotide sequence encoding the RGN polypeptide and/or guide RNA. In certain embodiments, the kit comprises more than one guide RNA (or polynucleotide(s) encoding more than one guide RNA) each capable of hybridizing with the RGN. The guide RNAs can bind to unique target sequences of a single DNA molecule in order to amplify the detectable signal and lead to the detection of that DNA molecule.

Elements of the kit may be provided individually or in combinations, and may be provided in any suitable container, such as a vial, a bottle, or a tube. In some embodiments, the kit includes instructions in one or more languages. In some embodiments, a kit comprises one or more reagents for use in a process utilizing one or more of the elements described herein. Reagents may be provided in any suitable container. For example, a kit may provide one or more reaction or storage buffers. Reagents may be provided in a form that is usable in a particular assay, or in a form that requires addition of one or more other components before use (e.g. in concentrate or lyophilized form). A buffer can be any buffer, including but not limited to a sodium carbonate buffer, a sodium bicarbonate buffer, a borate buffer, a Tris buffer, a MOPS buffer, a HEPES buffer, and combinations thereof. In some embodiments, the buffer is alkaline. In some embodiments, the buffer has a pH from about 7 to about 10.

Also provided herein are methods of cleaving single-stranded DNAs by contacting a population of nucleic acids, wherein the population comprises a target DNA sequence of a DNA molecule and a plurality of non-target ssDNAs with an RGN and a guide RNA capable of hybridizing with the RGN and the target DNA sequence. In some of these embodiments, the population of nucleic acids are within a cell lysate. In some of these embodiments, the non-target ssDNAs are foreign to the cell and in some of these embodiments, the non-target ssDNAs are viral DNAs. In particular embodiments, the target DNA sequence is a viral sequence. The method can be performed in vitro, in vivo, or ex vivo. For example, the method could be performed in vivo wherein a subject is administered an RGN polypeptide and a guide RNA or one or more polynucleotides comprising a nucleotide sequence that encodes the RGN polypeptide and/or the guide RNA and the binding and cleavage of a viral target DNA sequence by the RGN can result in the cleavage of non-target viral ssDNAs within the infected cell.

The article “a” and “an” are used herein to refer to one or more than one (i. e., to at least one) of the grammatical object of the article. By way of example, “a polypeptide” means one or more polypeptides.

All publications and patent applications mentioned in the specification are indicative of the level of those skilled in the art to which this disclosure pertains. All publications and patent applications are herein incorporated by reference to the same extent as if each individual publication or patent application was specifically and individually indicated to be incorporated herein by reference.

Although the foregoing invention has been described in some detail by way of illustration and example for purposes of clarity of understanding, it will be obvious that certain changes and modifications may be practiced within the scope of the appended embodiments.

Non-limiting embodiments include:
1. A nucleic acid molecule comprising a polynucleotide encoding an RNA-guided nuclease (RGN) polypeptide, wherein said polynucleotide comprises a nucleotide sequence encoding an RGN polypeptide comprising an amino acid sequence having at least 90% sequence identity to any one of SEQ ID NOs: 1 to 109;
   wherein said RGN polypeptide is capable of binding a target DNA sequence of a DNA molecule in an RNA-guided sequence specific manner when bound to a guide RNA (gRNA) capable of hybridizing to said target DNA sequence, and
   wherein said polynucleotide encoding an RGN polypeptide is operably linked to a promoter heterologous to said polynucleotide.
2. The nucleic acid molecule of embodiment 1, wherein said RGN polypeptide comprises an amino acid sequence having at least 95% sequence identity to any one of SEQ ID NOs: 1 to 109.
3. The nucleic acid molecule of embodiment 1, wherein said RGN polypeptide comprises an amino acid sequence having 100% sequence identity to any one of SEQ ID NOs: 1 to 109.
4. The nucleic acid molecule of any one of embodiments 1-3, wherein said target DNA sequence is within a region of said DNA molecule that is single-stranded.
5. The nucleic acid molecule of embodiment 4, wherein said RGN polypeptide is capable of cleaving said target DNA sequence upon binding.
6. The nucleic acid molecule of any one of embodiments 1-3, wherein said target DNA sequence is within a region of said DNA molecule that is double-stranded.
7. The nucleic acid molecule of embodiment 6, wherein said RGN polypeptide is capable of cleaving said target DNA sequence upon binding.
8. The nucleic acid molecule of embodiment 7, wherein cleavage by said RGN polypeptide generates a double-stranded break.
9. The nucleic acid molecule of embodiment 7, wherein cleavage by said RGN polypeptide generates a single-stranded break.
10. The nucleic acid molecule of any one of embodiments 1-9, wherein the RGN polypeptide is operably fused to a base-editing polypeptide.
11. The nucleic acid molecule of embodiment 10, wherein the base-editing polypeptide is a deaminase.
12. The nucleic acid molecule of any one of embodiments 1-11, wherein said target DNA sequence is located adjacent to a protospacer adjacent motif (PAM).
13. The nucleic acid molecule of any one of embodiments 1-12, wherein the RGN polypeptide comprises one or more nuclear localization signals.
14. The nucleic acid molecule of any one of embodiments 1-13, wherein the RGN polypeptide is codon optimized for expression in a eukaryotic cell.
15. A vector comprising the nucleic acid molecule of any one of embodiments 1-14.
16. The vector of embodiment 15, further comprising at least one nucleotide sequence encoding an RGN accessory protein selected from the group consisting of:
   a) at least one RGN accessory protein having at least 90% sequence identity to any one of SEQ ID NOs: 178-181, wherein said RGN polypeptide comprises an amino acid sequence having at least 90% sequence identity to SEQ ID NO: 11;
   b) at least one RGN accessory protein having at least 90% sequence identity to any one of SEQ ID NOs: 182-184, wherein said RGN polypeptide comprises an amino acid sequence having at least 90% sequence identity to SEQ ID NO: 12;
   c) at least one RGN accessory protein having at least 90% sequence identity to any one of SEQ ID NOs: 185-187, wherein said RGN polypeptide comprises an amino acid sequence having at least 90% sequence identity to SEQ ID NO: 13;
   d) an RGN accessory protein having at least 90% sequence identity to SEQ ID NO: 191, wherein said RGN polypeptide comprises an amino acid sequence having at least 90% sequence identity to SEQ ID NO: 14;
   e) an RGN accessory protein having at least 90% sequence identity to SEQ ID NO: 192, wherein said RGN polypeptide comprises an amino acid sequence having at least 90% sequence identity to SEQ ID NO: 15; and
   f) at least one RGN accessory protein having at least 90% sequence identity to any one of SEQ ID NOs: 188-190, wherein said RGN polypeptide comprises an amino acid sequence having at least 90% sequence identity to SEQ ID NO: 16.
17. The vector of embodiment 16, wherein said RGN accessory protein is selected from the group consisting of:
   a) at least one RGN accessory protein having at least 95% sequence identity to any one of SEQ ID NOs: 178-181, wherein said RGN polypeptide comprises an amino acid sequence having at least 95% sequence identity to SEQ ID NO: 11;
   b) at least one RGN accessory protein having at least 95% sequence identity to any one of SEQ ID NOs: 182-184, wherein said RGN polypeptide comprises an amino acid sequence having at least 95% sequence identity to SEQ ID NO: 12;
   c) at least one RGN accessory protein having at least 95% sequence identity to any one of SEQ ID NOs: 185-187, wherein said RGN polypeptide comprises an amino acid sequence having at least 95% sequence identity to SEQ ID NO: 13;
   d) an RGN accessory protein having at least 95% sequence identity to SEQ ID NO: 191, wherein said RGN polypeptide comprises an amino acid sequence having at least 95% sequence identity to SEQ ID NO: 14;
   e) an RGN accessory protein having at least 95% sequence identity to SEQ ID NO: 192, wherein said RGN polypeptide comprises an amino acid sequence having at least 95% sequence identity to SEQ ID NO: 15; and f) at least one RGN accessory protein having at least 95% sequence identity to any one of SEQ ID NOs: 188-190, wherein said RGN polypeptide comprises an amino acid sequence having at least 95% sequence identity to SEQ ID NO: 16.
18. The vector of embodiment 16, wherein said RGN accessory protein is selected from the group consisting of:
   a) at least one RGN accessory protein having 100% sequence identity to any one of SEQ ID NOs: 178-181, wherein said RGN polypeptide comprises an amino acid sequence having 100% sequence identity to SEQ ID NO: 11;
   b) at least one RGN accessory protein having 100% sequence identity to any one of SEQ ID NOs: 182-184, wherein said RGN polypeptide comprises an amino acid sequence having 100% sequence identity to SEQ ID NO: 12;
   c) at least one RGN accessory protein having 100% sequence identity to any one of SEQ ID NOs: 185-187, wherein said RGN polypeptide comprises an amino acid sequence having 100% sequence identity to SEQ ID NO: 13;
   d) an RGN accessory protein having 100% sequence identity to SEQ ID NO: 191, wherein said RGN polypeptide comprises an amino acid sequence having 100% sequence identity to SEQ ID NO: 14;
   e) an RGN accessory protein having 100% sequence identity to SEQ ID NO: 192, wherein said RGN polypeptide comprises an amino acid sequence having 100% sequence identity to SEQ ID NO: 15; and
   f) at least one RGN accessory protein having 100% sequence identity to any one of SEQ ID NOs: 188-190, wherein said RGN polypeptide comprises an amino acid sequence having 100% sequence identity to SEQ ID NO: 16.
19. The vector of any one of embodiments 15-18, further comprising at least one nucleotide sequence encoding said gRNA capable of hybridizing to said target DNA sequence.
20. The vector of embodiment 19, wherein said RGN polypeptide comprises an amino acid sequence having at least 90% sequence identity to SEQ ID NO: 11 and said gRNA comprises a CRISPR RNA comprising a CRISPR repeat sequence having at least 90% sequence identity to SEQ ID NO: 116.
21. The vector of embodiment 19, wherein said RGN polypeptide comprises an amino acid sequence having at least 95% sequence identity to SEQ ID NO: 11 and said gRNA comprises a CRISPR RNA comprising a CRISPR repeat sequence having at least 95% sequence identity to SEQ ID NO: 116.
22. The vector of embodiment 19, wherein said RGN polypeptide comprises an amino acid sequence having 100% sequence identity to SEQ ID NO: 11 and said gRNA comprises a CRISPR RNA comprising a CRISPR repeat sequence having 100% sequence identity to SEQ ID NO: 116.
23. The vector of embodiment 19, wherein said gRNA comprises a tracrRNA.
24. The vector of embodiment 23, wherein said tracrRNA is selected from the group consisting of:
   a) a tracrRNA having at least 90% sequence identity to SEQ ID NO: 120, wherein said gRNA further comprises a CRISPR RNA comprising a CRISPR repeat sequence having at least 90% sequence identity to SEQ ID NO: 110, and wherein said RGN polypeptide comprises an amino acid sequence having at least 90% sequence identity to SEQ ID NO: 1;
   b) a tracrRNA having at least 90% sequence identity to SEQ ID NO: 121, wherein said gRNA further comprises a CRISPR RNA comprising a CRISPR repeat sequence having at least 90% sequence identity to SEQ ID NO: 111, and wherein said RGN polypeptide comprises an amino acid sequence having at least 90% sequence identity to SEQ ID NO: 2;
   c) a tracrRNA having at least 90% sequence identity to SEQ ID NO: 122, wherein said gRNA further comprises a CRISPR RNA comprising a CRISPR repeat sequence having at least 90% sequence identity to SEQ ID NO: 112, and wherein said RGN polypeptide comprises an amino acid sequence having at least 90% sequence identity to SEQ ID NO: 3;
   d) a tracrRNA having at least 90% sequence identity to SEQ ID NO: 123, wherein said gRNA further comprises a CRISPR RNA comprising a CRISPR repeat sequence having at least 90% sequence identity to SEQ ID NO: 113, and wherein said RGN polypeptide comprises an amino acid sequence having at least 90% sequence identity to SEQ ID NO: 4;
   e) a tracrRNA having at least 90% sequence identity to SEQ ID NO: 124, wherein said gRNA further comprises a CRISPR RNA comprising a CRISPR repeat sequence having at least 90% sequence identity to SEQ ID NO: 114, and wherein said RGN polypeptide comprises an amino acid sequence having at least 90% sequence identity to SEQ ID NO: 5;
   f) a tracrRNA having at least 90% sequence identity to SEQ ID NO: 125, wherein said gRNA further comprises a CRISPR RNA comprising a CRISPR repeat sequence having at least 90% sequence identity to SEQ ID NO: 115, and wherein said RGN polypeptide comprises an amino acid sequence having at least 90% sequence identity to SEQ ID NO: 6;
   g) a tracrRNA having at least 90% sequence identity to SEQ ID NO: 126, wherein said gRNA further comprises a CRISPR RNA comprising a CRISPR repeat sequence having at least 90% sequence identity to SEQ ID NO: 117, and wherein said RGN polypeptide comprises an amino acid sequence having at least 90% sequence identity to SEQ ID NO: 12;
   h) a tracrRNA having at least 90% sequence identity to SEQ ID NO: 127, wherein said gRNA further comprises a CRISPR RNA comprising a CRISPR repeat sequence having at least 90% sequence identity to SEQ ID NO: 118, and wherein said RGN polypeptide comprises an amino acid sequence having at least 90% sequence identity to SEQ ID NO: 13; and
   i) a tracrRNA having at least 90% sequence identity to SEQ ID NO: 128, wherein said gRNA further comprises a CRISPR RNA comprising a CRISPR repeat sequence having at least 90% sequence identity to SEQ ID NO: 119, and wherein said RGN polypeptide comprises an amino acid sequence having at least 90% sequence identity to SEQ ID NO: 16.
25. The vector of embodiment 23, wherein said tracrRNA is selected from the group consisting of:
   a) a tracrRNA having at least 95% sequence identity to SEQ ID NO: 121, wherein said gRNA further comprises a CRISPR RNA comprising a CRISPR repeat sequence having at least 95% sequence identity to SEQ ID NO: 111, and wherein said RGN polypeptide comprises an amino acid sequence having at least 95% sequence identity to SEQ ID NO: 2;
   b) a tracrRNA having at least 95% sequence identity to SEQ ID NO: 123, wherein said gRNA further comprises a CRISPR RNA comprising a CRISPR repeat sequence having at least 95% sequence identity to SEQ ID NO: 113, and wherein said RGN polypeptide comprises an amino acid sequence having at least 95% sequence identity to SEQ ID NO: 4;
   c) a tracrRNA having at least 95% sequence identity to SEQ ID NO: 120, wherein said gRNA further comprises a CRISPR RNA comprising a CRISPR repeat sequence having at least 95% sequence identity to SEQ ID NO: 110, and wherein said RGN polypeptide comprises an amino acid sequence having at least 95% sequence identity to SEQ ID NO: 1;
   d) a tracrRNA having at least 95% sequence identity to SEQ ID NO: 122, wherein said gRNA further comprises a CRISPR RNA comprising a CRISPR repeat sequence having at least 95% sequence identity to SEQ ID NO: 112, and wherein said RGN polypeptide comprises an amino acid sequence having at least 95% sequence identity to SEQ ID NO: 3;
   e) a tracrRNA having at least 95% sequence identity to SEQ ID NO: 124, wherein said gRNA further comprises a CRISPR RNA comprising a CRISPR repeat sequence having at least 95% sequence identity to SEQ ID NO: 114, and wherein said RGN polypeptide comprises an amino acid sequence having at least 95% sequence identity to SEQ ID NO: 5;
   f) a tracrRNA having at least 95% sequence identity to SEQ ID NO: 125, wherein said gRNA further comprises a CRISPR RNA comprising a CRISPR repeat sequence having at least 95% sequence identity to SEQ ID NO: 115, and wherein said RGN polypeptide comprises an amino acid sequence having at least 95% sequence identity to SEQ ID NO: 6;
   g) a tracrRNA having at least 95% sequence identity to SEQ ID NO: 126, wherein said gRNA further comprises a CRISPR RNA comprising a CRISPR repeat sequence having at least 95% sequence identity to SEQ ID NO: 117, and wherein said RGN polypeptide comprises an amino acid sequence having at least 95% sequence identity to SEQ ID NO: 12;
   h) a tracrRNA having at least 95% sequence identity to SEQ ID NO: 127, wherein said gRNA further comprises a CRISPR RNA comprising a CRISPR repeat sequence having at least 95% sequence identity to SEQ ID NO: 118, and wherein said RGN polypeptide comprises an amino acid sequence having at least 95% sequence identity to SEQ ID NO: 13; and
   i) a tracrRNA having at least 95% sequence identity to SEQ ID NO: 128, wherein said gRNA further comprises a CRISPR RNA comprising a CRISPR repeat sequence having at least 95% sequence identity to SEQ ID NO: 119, and wherein said RGN polypeptide comprises an amino acid sequence having at least 95% sequence identity to SEQ ID NO: 16.
26. The vector of embodiment 23, wherein said tracrRNA is selected from the group consisting of:
   a) a tracrRNA having 100% sequence identity to SEQ ID NO: 121, wherein said gRNA further comprises a CRISPR RNA comprising a CRISPR repeat sequence having 100% sequence identity to SEQ ID NO: 111, and wherein said RGN polypeptide comprises an amino acid sequence having 100% sequence identity to SEQ ID NO: 2;
   b) a tracrRNA having 100% sequence identity to SEQ ID NO: 123, wherein said gRNA further comprises a CRISPR RNA comprising a CRISPR repeat sequence having 100% sequence identity to SEQ ID NO: 113, and wherein said RGN polypeptide comprises an amino acid sequence having 100% sequence identity to SEQ ID NO: 4;
   c) a tracrRNA having 100% sequence identity to SEQ ID NO: 120, wherein said gRNA further comprises a CRISPR RNA comprising a CRISPR repeat sequence having 100% sequence identity to SEQ ID NO: 110, and wherein said RGN polypeptide comprises an amino acid sequence having 100% sequence identity to SEQ ID NO: 1;
   d) a tracrRNA having 100% sequence identity to SEQ ID NO: 122, wherein said gRNA further comprises a CRISPR RNA comprising a CRISPR repeat sequence having 100% sequence identity to SEQ ID NO: 112, and wherein said RGN polypeptide comprises an amino acid sequence having 100% sequence identity to SEQ ID NO: 3;
   e) a tracrRNA having 100% sequence identity to SEQ ID NO: 124, wherein said gRNA further comprises a CRISPR RNA comprising a CRISPR repeat sequence having 100% sequence identity to SEQ ID NO: 114, and wherein said RGN polypeptide comprises an amino acid sequence having 100% sequence identity to SEQ ID NO: 5;
   f) a tracrRNA having 100% sequence identity to SEQ ID NO: 125, wherein said gRNA further comprises a CRISPR RNA comprising a CRISPR repeat sequence having 100% sequence identity to SEQ ID NO: 115, and wherein said RGN polypeptide comprises an amino acid sequence having 100% sequence identity to SEQ ID NO: 6;
   g) a tracrRNA having 100% sequence identity to SEQ ID NO: 126, wherein said gRNA further comprises a CRISPR RNA comprising a CRISPR repeat sequence having 100% sequence identity to SEQ ID NO: 117, and wherein said RGN polypeptide comprises an amino acid sequence having 100% sequence identity to SEQ ID NO: 12;
   h) a tracrRNA having 100% sequence identity to SEQ ID NO: 127, wherein said gRNA further comprises a CRISPR RNA comprising a CRISPR repeat sequence having 100% sequence identity to SEQ ID NO: 118, and wherein said RGN polypeptide comprises an amino acid sequence having 100% sequence identity to SEQ ID NO: 13; and
   i) a tracrRNA having 100% sequence identity to SEQ ID NO: 128, wherein said gRNA further comprises a CRISPR RNA comprising a CRISPR repeat sequence having 100% sequence identity to SEQ ID NO: 119, and wherein said RGN polypeptide comprises an amino acid sequence having 100% sequence identity to SEQ ID NO: 16.
27. The vector of any one of embodiments 23-26, where said gRNA is a single guide RNA.
28. The vector of any one of embodiments 23-26, wherein said gRNA is a dual-guide RNA.
29. A cell comprising the nucleic acid molecule of any one of embodiments 1-14 or the vector of any one of embodiments 15-28.
30. A method for making an RGN polypeptide comprising culturing the cell of embodiment 29 under conditions in which the RGN polypeptide is expressed.
31. A method for making an RGN polypeptide comprising introducing into a cell a heterologous nucleic acid molecule comprising a nucleotide sequence encoding an RNA-guided nuclease (RGN) polypeptide comprising an amino acid sequence having at least 90% sequence identity to any one of SEQ ID NOs: 1 to 109;
   wherein said RGN polypeptide binds a target DNA sequence of a DNA molecule in an RNA-guided sequence specific manner when bound to a guide RNA (gRNA) capable of hybridizing to said target DNA sequence;
   and culturing said cell under conditions in which the RGN polypeptide is expressed.
32. The method of embodiment 31, wherein said RGN polypeptide comprises an amino acid sequence having at least 95% sequence identity to any one of SEQ ID NOs: 1 to 109.
33. The method of embodiment 31, wherein said RGN polypeptide comprises an amino acid sequence having 100% sequence identity to any one of SEQ ID NOs: 1 to 109.
34. The method of any one of embodiments 30-33, further comprising purifying said RGN polypeptide.
35. The method of any one of embodiments 30-33, wherein said cell further expresses one or more guide RNAs that binds to said RGN polypeptide to form an RGN ribonucleoprotein complex.
36. The method of embodiment 35, further comprising purifying said RGN ribonucleoprotein complex.
37. An isolated RNA-guided nuclease (RGN) polypeptide, wherein said RGN polypeptide comprises an amino acid sequence having at least 90% sequence identity to any one of SEQ ID NOs: 1 to 109; and wherein said RGN polypeptide is capable of binding a target DNA sequence of a DNA molecule in an RNA-guided sequence specific manner when bound to a guide RNA (gRNA) capable of hybridizing to said target DNA sequence.
38. The isolated RGN polypeptide of embodiment 37, wherein said RGN polypeptide comprises an amino acid sequence having at least 95% sequence identity to any one of SEQ ID NOs: 1 to 109.
39. The isolated RGN polypeptide of embodiment 37, wherein said RGN polypeptide comprises an amino acid sequence having 100% sequence identity to any one of SEQ ID NOs: 1 to 109.
40. The isolated RGN polypeptide of any one of embodiments 37-39, wherein said target DNA sequence is within a region of said DNA molecule that is single-stranded.
41. The isolated RGN polypeptide of embodiment 40, wherein said RGN polypeptide is capable of cleaving said target DNA sequence upon binding.
42. The isolated RGN polypeptide of any one of embodiments 37-39, wherein said target DNA sequence is within a region of said DNA molecule that is double-stranded.
43. The isolated RGN polypeptide of embodiment 42, wherein said RGN polypeptide is capable of cleaving said target DNA sequence upon binding.
44. The isolated RGN polypeptide of embodiment 43, wherein cleavage by said RGN polypeptide generates a double-stranded break.
45. The isolated RGN polypeptide of embodiment 43, wherein cleavage by said RGN polypeptide generates a single-stranded break.
46. The isolated RGN polypeptide of any one of embodiments 37-45, wherein the RGN polypeptide is operably fused to a base-editing polypeptide.
47. The isolated RGN polypeptide of embodiment 46, wherein the base-editing polypeptide is a deaminase.
48. The isolated RGN polypeptide of any one of embodiments 37-47, wherein said target DNA sequence is located adjacent to a protospacer adjacent motif (PAM).
49. The isolated RGN polypeptide of any one of embodiments 37-48, wherein the RGN polypeptide comprises one or more nuclear localization signals.
50. A nucleic acid molecule comprising a polynucleotide encoding a CRISPR RNA (crRNA), wherein said crRNA comprises a spacer sequence and a CRISPR repeat sequence, wherein said CRISPR repeat sequence comprises a nucleotide sequence having at least 90% sequence identity to any one of SEQ ID NOs: 110 to 119;
   wherein a guide RNA comprising:
      a) said crRNA; or
      b) said crRNA and a trans-activating CRISPR RNA (tracrRNA) capable of hybridizing to said CRISPR repeat sequence of said crRNA;
   is capable of hybridizing to a target DNA sequence of a DNA molecule in a sequence specific manner through the spacer sequence of said crRNA when said guide RNA is bound to an RNA-guided nuclease (RGN) polypeptide, and
   wherein said polynucleotide encoding a crRNA is operably linked to a promoter heterologous to said polynucleotide.
51. The nucleic acid molecule of embodiment 50, wherein said CRISPR repeat sequence comprises a nucleotide sequence having at least 95% sequence identity to any one of SEQ ID NOs: 110 to 119.
52. The nucleic acid molecule of embodiment 50, wherein said CRISPR repeat sequence comprises a nucleotide sequence having 100% sequence identity to any one of SEQ ID NOs: 110 to 119.
53. A vector comprising the nucleic acid molecule of any one of embodiments 50-52.
54. The vector of embodiment 53, wherein said vector further comprises a polynucleotide encoding said tracrRNA.
55. The vector of embodiment 54, wherein said CRISPR repeat sequence has at least 90% sequence identity to SEQ ID NO: 110, and said tracrRNA comprises a nucleotide sequence having at least 90% sequence identity to SEQ ID NO: 120.
56. The vector of embodiment 54, wherein said CRISPR repeat sequence has at least 95% sequence identity to SEQ ID NO: 110, and said tracrRNA comprises a nucleotide sequence having at least 95% sequence identity to SEQ ID NO: 120.
57. The vector of embodiment 54, wherein said CRISPR repeat sequence has 100% sequence identity to SEQ ID NO: 110, and said tracrRNA comprises a nucleotide sequence having 100% sequence identity to SEQ ID NO: 120.
58. The vector of any one of embodiments 55-57, wherein said vector further comprises a polynucleotide encoding said RGN polypeptide comprising an amino acid sequence having at least 90% sequence identity to SEQ ID NO: 1.
59. The vector of embodiment 58, wherein said RGN polypeptide comprises an amino acid sequence having at least 95% sequence identity to SEQ ID NO: 1.
60. The vector of embodiment 58, wherein said RGN polypeptide comprises an amino acid sequence having 100% sequence identity to SEQ ID NO: 1.
61. The vector of embodiment 54, wherein said CRISPR repeat sequence has at least 90% sequence identity to SEQ ID NO: 111, and said tracrRNA comprises a nucleotide sequence having at least 90% sequence identity to SEQ ID NO: 121.
62. The vector of embodiment 54, wherein said CRISPR repeat sequence has at least 95% sequence identity to SEQ ID NO: 111, and said tracrRNA comprises a nucleotide sequence having at least 95% sequence identity to SEQ ID NO: 121.
63. The vector of embodiment 54, wherein said CRISPR repeat sequence has 100% sequence identity to SEQ ID NO: 111, and said tracrRNA comprises a nucleotide sequence having 100% sequence identity to SEQ ID NO: 121.
64. The vector of any one of embodiments 61-63, wherein said vector further comprises a polynucleotide encoding said RGN polypeptide comprising an amino acid sequence having at least 90% sequence identity to SEQ ID NO: 2.
65. The vector of embodiment 64, wherein RGN polypeptide comprises an amino acid sequence having at least 95% sequence identity to SEQ ID NO: 2.
66. The vector of embodiment 64, wherein RGN polypeptide comprises an amino acid sequence having 100% sequence identity to SEQ ID NO: 2.
67. The vector of embodiment 54, wherein said CRISPR repeat sequence has at least 90% sequence identity to SEQ ID NO: 112, and said tracrRNA comprises a nucleotide sequence having at least 90% sequence identity to SEQ ID NO: 122.
68. The vector of embodiment 54, wherein said CRISPR repeat sequence has at least 95% sequence identity to SEQ ID NO: 112, and said tracrRNA comprises a nucleotide sequence having at least 95% sequence identity to SEQ ID NO: 122.
69. The vector of embodiment 54, wherein said CRISPR repeat sequence has 100% sequence identity to SEQ ID NO: 112, and said tracrRNA comprises a nucleotide sequence having 100% sequence identity to SEQ ID NO: 122.
70. The vector of any one of embodiments 67-69, wherein said vector further comprises a polynucleotide encoding said RGN polypeptide comprising an amino acid sequence having at least 90% sequence identity to SEQ ID NO: 3.
71. The vector of embodiment 70, wherein said RGN polypeptide comprises an amino acid sequence having at least 95% sequence identity to SEQ ID NO: 3.
72. The vector of embodiment 70, wherein said RGN polypeptide comprises an amino acid sequence having 100% sequence identity to SEQ ID NO: 3.
73. The vector of embodiment 54, wherein said CRISPR repeat sequence has at least 90% sequence identity to SEQ ID NO: 113, and said tracrRNA comprises a nucleotide sequence having at least 90% sequence identity to SEQ ID NO: 123.
74. The vector of embodiment 54, wherein said CRISPR repeat sequence has at least 95% sequence identity to SEQ ID NO: 113, and said tracrRNA comprises a nucleotide sequence having at least 95% sequence identity to SEQ ID NO: 123.
75. The vector of embodiment 54, wherein said CRISPR repeat sequence has 100% sequence identity to SEQ ID NO: 113, and said tracrRNA comprises a nucleotide sequence having 100% sequence identity to SEQ ID NO: 123.
76. The vector of any one of embodiments 73-75, wherein said vector further comprises a polynucleotide encoding said RGN polypeptide comprising an amino acid sequence having at least 90% sequence identity to SEQ ID NO: 4.
77. The vector of embodiment 76, wherein said RGN polypeptide comprises an amino acid sequence having at least 95% sequence identity to SEQ ID NO: 4.
78. The vector of embodiment 76, wherein said RGN polypeptide comprises an amino acid sequence having 100% sequence identity to SEQ ID NO: 4.
79. The vector of embodiment 54, wherein said CRISPR repeat sequence comprises a nucleotide sequence having at least 90% sequence identity to SEQ ID NO: 114, and said tracrRNA comprises a nucleotide sequence having at least 90% sequence identity to SEQ ID NO: 124.
80. The vector of embodiment 54, wherein said CRISPR repeat sequence comprises a nucleotide sequence having at least 95% sequence identity to SEQ ID NO: 114, and said tracrRNA comprises a nucleotide sequence having at least 95% sequence identity to SEQ ID NO: 124.
81. The vector of embodiment 54, wherein said CRISPR repeat sequence comprises a nucleotide sequence having 100% sequence identity to SEQ ID NO: 114, and said tracrRNA comprises a nucleotide sequence having 100% sequence identity to SEQ ID NO: 124.
82. The vector of any one of embodiments 79-81, wherein said vector further comprises a polynucleotide encoding said RGN polypeptide comprising an amino acid sequence having at least 90% sequence identity to SEQ ID NO: 5.
83. The vector of embodiment 82, wherein said RGN polypeptide comprises an amino acid sequence having at least 95% sequence identity to SEQ ID NO: 5.
84. The vector of embodiment 82, wherein said RGN polypeptide comprises an amino acid sequence having 100% sequence identity to SEQ ID NO: 5.
85. The vector of embodiment 54, wherein said CRISPR repeat sequence comprises a nucleotide sequence having at least 90% sequence identity to SEQ ID NO: 115, and said tracrRNA comprises a nucleotide sequence having at least 90% sequence identity to SEQ ID NO: 125.
86. The vector of embodiment 54, wherein said CRISPR repeat sequence comprises a nucleotide sequence having at least 95% sequence identity to SEQ ID NO: 115, and said tracrRNA comprises a nucleotide sequence having at least 95% sequence identity to SEQ ID NO: 125.
87. The vector of embodiment 54, wherein said CRISPR repeat sequence comprises a nucleotide sequence having 100% sequence identity to SEQ ID NO: 115, and said tracrRNA comprises a nucleotide sequence having 100% sequence identity to SEQ ID NO: 125.
88. The vector of any one of embodiments 85-87, wherein said vector further comprises a polynucleotide encoding said RGN polypeptide comprising an amino acid sequence having at least 90% sequence identity to SEQ ID NO: 6.
89. The vector of embodiment 88, wherein said RGN polypeptide comprises an amino acid sequence having at least 95% sequence identity to SEQ ID NO: 6.
90. The vector of embodiment 88, wherein said RGN polypeptide comprises an amino acid sequence having 100% sequence identity to SEQ ID NO: 6.
91. The vector of embodiment 54, wherein said CRISPR repeat sequence comprises a nucleotide sequence having at least 90% sequence identity to SEQ ID NO: 117, and said tracrRNA comprises a nucleotide sequence having at least 90% sequence identity to SEQ ID NO: 126.
92. The vector of embodiment 54, wherein said CRISPR repeat sequence comprises a nucleotide sequence having at least 95% sequence identity to SEQ ID NO: 117, and said tracrRNA comprises a nucleotide sequence having at least 95% sequence identity to SEQ ID NO: 126.
93. The vector of embodiment 54, wherein said CRISPR repeat sequence comprises a nucleotide sequence having 100% sequence identity to SEQ ID NO: 117, and said tracrRNA comprises a nucleotide sequence having 100% sequence identity to SEQ ID NO: 126.
94. The vector of any one of embodiments 91-93, wherein said vector further comprises a polynucleotide encoding said RGN polypeptide comprising an amino acid sequence having at least 90% sequence identity to SEQ ID NO: 12.
95. The vector of embodiment 94, wherein said RGN polypeptide comprises an amino acid sequence having at least 95% sequence identity to SEQ ID NO: 12.
96. The vector of embodiment 94, wherein said RGN polypeptide comprises an amino acid sequence having 100% sequence identity to SEQ ID NO: 12.
97. The vector of embodiment 54, wherein said CRISPR repeat sequence comprises a nucleotide sequence having at least 90% sequence identity to SEQ ID NO: 118, and said tracrRNA comprises a nucleotide sequence having at least 90% sequence identity to SEQ ID NO: 127.
98. The vector of embodiment 54, wherein said CRISPR repeat sequence comprises a nucleotide sequence having at least 95% sequence identity to SEQ ID NO: 118, and said tracrRNA comprises a nucleotide sequence having at least 95% sequence identity to SEQ ID NO: 127.
99. The vector of embodiment 54, wherein said CRISPR repeat sequence comprises a nucleotide sequence having 100% sequence identity to SEQ ID NO: 118, and said tracrRNA comprises a nucleotide sequence having 100% sequence identity to SEQ ID NO: 127.
100. The vector of any one of embodiments 97-99, wherein said vector further comprises a polynucleotide encoding said RGN polypeptide comprising an amino acid sequence having at least 90% sequence identity to SEQ ID NO: 13.
101. The vector of embodiment 100, wherein said RGN polypeptide comprises an amino acid sequence having at least 95% sequence identity to SEQ ID NO: 13.
102. The vector of embodiment 100, wherein said RGN polypeptide comprises an amino acid sequence having 100% sequence identity to SEQ ID NO: 13.
103. The vector of embodiment 54, wherein said CRISPR repeat sequence comprises a nucleotide sequence having at least 90% sequence identity to SEQ ID NO: 119, and said tracrRNA comprises a nucleotide sequence having at least 90% sequence identity to SEQ ID NO: 128.
104. The vector of embodiment 54, wherein said CRISPR repeat sequence comprises a nucleotide sequence having at least 95% sequence identity to SEQ ID NO: 119, and said tracrRNA comprises a nucleotide sequence having at least 95% sequence identity to SEQ ID NO: 128.
105. The vector of embodiment 54, wherein said CRISPR repeat sequence comprises a nucleotide sequence having 100% sequence identity to SEQ ID NO: 119, and said tracrRNA comprises a nucleotide sequence having 100% sequence identity to SEQ ID NO: 128.
106. The vector of any one of embodiments 103-105, wherein said vector further comprises a polynucleotide encoding said RGN polypeptide comprising an amino acid sequence having at least 90% sequence identity to SEQ ID NO: 16.
107. The vector of embodiment 106, wherein said RGN polypeptide comprises an amino acid sequence having at least 95% sequence identity to SEQ ID NO: 16.
108. The vector of embodiment 106, wherein said RGN polypeptide comprises an amino acid sequence having 100% sequence identity to SEQ ID NO: 16.
109. The vector of any one of embodiments 54-109, wherein said polynucleotide encoding said crRNA and said polynucleotide encoding said tracrRNA are operably linked to the same promoter and are encoded as a single guide RNA.
110. The vector of any one of embodiments 54-109, wherein said polynucleotide encoding said crRNA and said polynucleotide encoding said tracrRNA are operably linked to separate promoters.
111. A nucleic acid molecule comprising a polynucleotide encoding a trans-activating CRISPR RNA (tracrRNA) comprising a nucleotide sequence having at least 90% sequence identity to any one of SEQ ID NOs: 120 to 128;
   wherein a guide RNA comprising:
      a) said tracrRNA; and
      b) a crRNA comprising a spacer sequence and a CRISPR repeat sequence, wherein said tracrRNA is capable of hybridizing with said CRISPR repeat sequence of said crRNA;
   is capable of hybridizing to a target DNA sequence in a sequence specific manner through the spacer sequence of said crRNA when said guide RNA is bound to an RNA-guided nuclease (RGN) polypeptide, and
   wherein said polynucleotide encoding a tracrRNA is operably linked to a promoter heterologous to said polynucleotide.
112. The nucleic acid molecule of embodiment 111, wherein said tracrRNA comprises a nucleotide sequence having at least 95% sequence identity to any one of SEQ ID NOs: 120 to 128.
113. The nucleic acid molecule of embodiment 111, wherein said tracrRNA comprises a nucleotide sequence having 1000% sequence identity to any one of SEQ ID NOs: 120 to 128.
114. A vector comprising the nucleic acid molecule of any one of embodiments 111-113.
115. The vector of embodiment 114, wherein said vector further comprises a polynucleotide encoding said crRNA.
116. The vector of embodiment 115, wherein said crRNA comprises a CRISPR repeat sequence having at least 90% sequence identity to SEQ ID NO: 110 and said tracrRNA comprises a nucleotide sequence having at least 90% sequence identity to SEQ ID NO: 120.
117. The vector of embodiment 116, wherein said CRISPR repeat sequence has at least 95% sequence identity to SEQ ID NO: 110 and said tracrRNA comprises a nucleotide sequence having at least 95% sequence identity to SEQ ID NO: 120. 118. The vector of embodiment 116, wherein said CRISPR repeat sequence has 100% sequence identity to SEQ ID NO: 110 and said tracrRNA comprises a nucleotide sequence having 100% sequence identity to SEQ ID NO: 120.
119. The vector of any one of embodiments 116-118, wherein said vector further comprises a polynucleotide encoding said RGN polypeptide comprising an amino acid sequence having at least 90% sequence identity to SEQ ID NO: 1.
120. The vector of embodiment 119, wherein said RGN polypeptide comprises an amino acid sequence having at least 95% sequence identity to SEQ ID NO: 1.
121. The vector of embodiment 119, wherein said RGN polypeptide comprises an amino acid sequence having 100% sequence identity to SEQ ID NO: 1.
122. The vector of embodiment 115, wherein said crRNA comprises a CRISPR repeat sequence having at least 90% sequence identity to SEQ ID NO: 111 and said tracrRNA comprises a nucleotide sequence having at least 90% sequence identity to SEQ ID NO: 121.
123. The vector of embodiment 122, wherein said CRISPR repeat sequence has at least 95% sequence identity to SEQ ID NO: 111 and said tracrRNA comprises a nucleotide sequence having at least 95% sequence identity to SEQ ID NO: 121.
124. The vector of embodiment 122, wherein said CRISPR repeat sequence has 100% sequence identity to SEQ ID NO: 111 and said tracrRNA comprises a nucleotide sequence having 100% sequence identity to SEQ ID NO: 121.
125. The vector of any one of embodiments 122-124, wherein said vector further comprises a polynucleotide encoding said RGN polypeptide comprising an amino acid sequence having at least 90% sequence identity to SEQ ID NO: 2.
126. The vector of embodiment 125, wherein said RGN polypeptide comprises an amino acid sequence having at least 95% sequence identity to SEQ ID NO: 2.
127. The vector of embodiment 125, wherein said RGN polypeptide comprises an amino acid sequence having 100% sequence identity to SEQ ID NO: 2.
128. The vector of embodiment 115, wherein said crRNA comprises a CRISPR repeat sequence having at least 90% sequence identity to SEQ ID NO: 112 and said tracrRNA comprises a nucleotide sequence having at least 90% sequence identity to SEQ ID NO: 122.
129. The vector of embodiment 128, wherein said CRISPR repeat sequence has at least 95% sequence identity to SEQ ID NO: 112 and said tracrRNA comprises a nucleotide sequence having at least 95% sequence identity to SEQ ID NO: 122.
130. The vector of embodiment 128, wherein said CRISPR repeat sequence has 100% sequence identity to SEQ ID NO: 112 and said tracrRNA comprises a nucleotide sequence having 100% sequence identity to SEQ ID NO: 122.
131. The vector of any one of embodiments 128-130, wherein said vector further comprises a polynucleotide encoding said RGN polypeptide comprising an amino acid sequence having at least 90% sequence identity to SEQ ID NO: 3.
132. The vector of embodiment 131, wherein said RGN polypeptide comprises an amino acid sequence having at least 95% sequence identity to SEQ ID NO: 3.
133. The vector of embodiment 131, wherein said RGN polypeptide comprises an amino acid sequence having 100% sequence identity to SEQ ID NO: 3.
134. The vector of embodiment 115, wherein said crRNA comprises a CRISPR repeat sequence having at least 90% sequence identity to SEQ ID NO: 113 and said tracrRNA comprises a nucleotide sequence having at least 90% sequence identity to SEQ ID NO: 123.
135. The vector of embodiment 134, wherein said CRISPR repeat sequence has at least 95% sequence identity to SEQ ID NO: 113 and said tracrRNA comprises a nucleotide sequence having at least 95% sequence identity to SEQ ID NO: 123.
136. The vector of embodiment 134, wherein said CRISPR repeat sequence has 100% sequence identity to SEQ ID NO: 113 and said tracrRNA comprises a nucleotide sequence having 100% sequence identity to SEQ ID NO: 123.
137. The vector of any one of embodiments 134-136, wherein said vector further comprises a polynucleotide encoding said RGN polypeptide comprising an amino acid sequence having at least 90% sequence identity to SEQ ID NO: 4.
138. The vector of embodiment 137, wherein said RGN polypeptide comprises an amino acid sequence having at least 95% sequence identity to SEQ ID NO: 4.
139. The vector of embodiment 137, wherein said RGN polypeptide comprises an amino acid sequence having 100% sequence identity to SEQ ID NO: 4.
140. The vector of embodiment 115, wherein said crRNA comprises a CRISPR repeat sequence having at least 90% sequence identity to SEQ ID NO: 114 and said tracrRNA comprises a nucleotide sequence having at least 90% sequence identity to SEQ ID NO: 124.
141. The vector of embodiment 140, wherein said CRISPR repeat sequence has at least 95% sequence identity to SEQ ID NO: 114 and said tracrRNA comprises a nucleotide sequence having at least 95% sequence identity to SEQ ID NO: 124.
142. The vector of embodiment 140, wherein said CRISPR repeat sequence has 100% sequence identity to SEQ ID NO: 114 and said tracrRNA comprises a nucleotide sequence having 100% sequence identity to SEQ ID NO: 124.
143. The vector of any one of embodiments 140-142, wherein said vector further comprises a polynucleotide encoding said RGN polypeptide comprising an amino acid sequence having at least 90% sequence identity to SEQ ID NO: 5.
144. The vector of embodiment 143, wherein said RGN polypeptide comprises an amino acid sequence having at least 95% sequence identity to SEQ ID NO: 5.
145. The vector of embodiment 143, wherein said RGN polypeptide comprises an amino acid sequence having 100% sequence identity to SEQ ID NO: 5.
146. The vector of embodiment 115, wherein said crRNA comprises a CRISPR repeat sequence having at least 90% sequence identity to SEQ ID NO: 115 and said tracrRNA comprises a nucleotide sequence having at least 90% sequence identity to SEQ ID NO: 125.
147. The vector of embodiment 146, wherein said CRISPR repeat sequence has at least 95% sequence identity to SEQ ID NO: 115 and said tracrRNA comprises a nucleotide sequence having at least 95% sequence identity to SEQ ID NO: 125.
148. The vector of embodiment 146, wherein said CRISPR repeat sequence has 100% sequence identity to SEQ ID NO: 115 and said tracrRNA comprises a nucleotide sequence having 100% sequence identity to SEQ ID NO: 125.
149. The vector of any one of embodiments 146-148, wherein said vector further comprises a polynucleotide encoding said RGN polypeptide comprising an amino acid sequence having at least 90% sequence identity to SEQ ID NO: 6.
150. The vector of embodiment 149, wherein said RGN polypeptide comprises an amino acid sequence having at least 95% sequence identity to SEQ ID NO: 6.
151. The vector of embodiment 149, wherein said RGN polypeptide comprises an amino acid sequence having 100% sequence identity to SEQ ID NO: 6.
152. The vector of embodiment 115, wherein said crRNA comprises a CRISPR repeat sequence having at least 90% sequence identity to SEQ ID NO: 117 and said tracrRNA comprises a nucleotide sequence having at least 90% sequence identity to SEQ ID NO: 126.
153. The vector of embodiment 152, wherein said CRISPR repeat sequence has at least 95% sequence identity to SEQ ID NO: 117 and said tracrRNA comprises a nucleotide sequence having at least 95% sequence identity to SEQ ID NO: 126.
154. The vector of embodiment 152, wherein said CRISPR repeat sequence has 100% sequence identity to SEQ ID NO: 117 and said tracrRNA comprises a nucleotide sequence having 100% sequence identity to SEQ ID NO: 126.
155. The vector of any one of embodiments embodiment 152-154, wherein said vector further comprises a polynucleotide encoding said RGN polypeptide comprising an amino acid sequence having at least 90% sequence identity to SEQ ID NO: 16.
156. The vector of embodiment 155, wherein said RGN polypeptide comprises an amino acid sequence having at least 95% sequence identity to SEQ ID NO: 16.
157. The vector of embodiment 155, wherein said RGN polypeptide comprises an amino acid sequence having 100% sequence identity to SEQ ID NO: 16.
158. The vector of embodiment 115, wherein said crRNA comprises a CRISPR repeat sequence having at least 90% sequence identity to SEQ ID NO: 118 and said tracrRNA comprises a nucleotide sequence having at least 90% sequence identity to SEQ ID NO: 127.
159. The vector of embodiment 158, wherein said CRISPR repeat sequence having at least 95% sequence identity to SEQ ID NO: 118 and said tracrRNA comprises a nucleotide sequence having at least 95% sequence identity to SEQ ID NO: 127.
160. The vector of embodiment 158, wherein said CRISPR repeat sequence having 100% sequence identity to SEQ ID NO: 118 and said tracrRNA comprises a nucleotide sequence having 100% sequence identity to SEQ ID NO: 127.
161. The vector of any one of embodiments 158-160, wherein said vector further comprises a polynucleotide encoding said RGN polypeptide comprising an amino acid sequence having at least 90% sequence identity to SEQ ID NO: 13.
162. The vector of embodiment 161, wherein said RGN polypeptide comprises an amino acid sequence having at least 95% sequence identity to SEQ ID NO: 13.
163. The vector of embodiment 161, wherein said RGN polypeptide comprises an amino acid sequence having 100% sequence identity to SEQ ID NO: 13.
164. The vector of embodiment 115, wherein said crRNA comprises a CRISPR repeat sequence having at least 90% sequence identity to SEQ ID NO: 119 and said tracrRNA comprises a nucleotide sequence having at least 90% sequence identity to SEQ ID NO: 128.
165. The vector of embodiment 164, wherein said CRISPR repeat sequence has at least 95% sequence identity to SEQ ID NO: 119 and said tracrRNA comprises a nucleotide sequence having at least 95% sequence identity to SEQ ID NO: 128.
166. The vector of embodiment 164, wherein said CRISPR repeat sequence has 100% sequence identity to SEQ ID NO: 119 and said tracrRNA comprises a nucleotide sequence having 100% sequence identity to SEQ ID NO: 128.
167. The vector of any one of embodiments 164-166, wherein said vector further comprises a polynucleotide encoding said RGN polypeptide comprising an amino acid sequence having at least 90% sequence identity to SEQ ID NO: 16.
168. The vector of embodiment 167, wherein said RGN polypeptide comprises an amino acid sequence having at least 95% sequence identity to SEQ ID NO: 16.
169. The vector of embodiment 167, wherein said RGN polypeptide comprises an amino acid sequence having 100% sequence identity to SEQ ID NO: 16.
170. The vector of any one of embodiments 115-169, wherein said polynucleotide encoding said crRNA and said polynucleotide encoding said tracrRNA are operably linked to the same promoter and are encoded as a single guide RNA.
171. The vector of any one of embodiments 115-169, wherein said polynucleotide encoding said crRNA and said polynucleotide encoding said tracrRNA are operably linked to separate promoters.
172. A system for binding a target DNA sequence of a DNA molecule, said system comprising:
   a) one or more guide RNAs capable of hybridizing to said target DNA sequence or one or more polynucleotides comprising one or more nucleotide sequences encoding the one or more guide RNAs (gRNAs); and
   b) an RNA-guided nuclease (RGN) polypeptide comprising an amino acid sequence having at least 90% sequence identity to any one of SEQ ID NOs: 1 to 109 or a polynucleotide comprising a nucleotide sequence encoding the RGN polypeptide;
   wherein at least one of said nucleotide sequence encoding the one or more guide RNAs and said nucleotide sequence encoding the RGN polypeptide is operably linked to a promoter heterologous to said nucleotide sequence; and
   wherein the one or more guide RNAs are capable of forming a complex with the RGN polypeptide in order to direct said RGN polypeptide to bind to said target DNA sequence of the DNA molecule.
173. A system for binding a target DNA sequence of a DNA molecule, said system comprising:
   a) one or more guide RNAs capable of hybridizing to said target DNA sequence or one or more polynucleotides comprising one or more nucleotide sequences encoding the one or more guide RNAs (gRNAs); and
   b) an RNA-guided nuclease (RGN) polypeptide comprising an amino acid sequence having at least 90% sequence identity to any one of SEQ ID NOs: 1 to 109;
   wherein the one or more guide RNAs are capable of hybridizing to the target DNA sequence, and
   wherein the one or more guide RNAs are capable of forming a complex with the RGN polypeptide in order to direct said RGN polypeptide to bind to said target DNA sequence of the DNA molecule.
174. The system of embodiment 173, wherein at least one of said nucleotides sequences encoding the one or more guide RNAs is operably linked to a promoter heterologous to said nucleotide sequence.
175. The system of any one of embodiments 172-174, wherein said RGN polypeptide comprises an amino acid sequence having at least 95% sequence identity to any one of SEQ ID NOs: 1 to 109.
176. The system of any one of embodiments 172-174, wherein said RGN polypeptide comprises an amino acid sequence having 100% sequence identity to any one of SEQ ID NOs: 1 to 109.
177. The system of any one of embodiments 172-176, wherein said RGN polypeptide and said one or more guide RNAs are not found complexed to one another in nature.
178. The system of any one of embodiments 172-177, wherein said target DNA sequence is a eukaryotic target DNA sequence.
179. The system of any one of embodiments 172-178, wherein said RGN polypeptide comprises an amino acid sequence having at least 90% sequence identity to SEQ ID NO: 11 and said one or more guide RNAs comprise a CRISPR RNA comprising a CRISPR repeat sequence having at least 90% sequence identity to SEQ ID NO: 116.
180. The system of any one of embodiments 172-178, wherein said RGN polypeptide comprises an amino acid sequence having at least 95% sequence identity to SEQ ID NO: 11 and said one or more guide RNAs comprise a CRISPR RNA comprising a CRISPR repeat sequence having at least 95% sequence identity to SEQ ID NO: 116.
181. The system of any one of embodiments 172-178, wherein said RGN polypeptide comprises an amino acid sequence having 100% sequence identity to SEQ ID NO: 11 and said one or more guide RNAs comprise a CRISPR RNA comprising a CRISPR repeat sequence having 100% sequence identity to SEQ ID NO: 116.
182. The system of any one of embodiments 172-181, wherein said one or more guide RNAscomprise a tracrRNA.
183. The system of embodiment 182, wherein said tracrRNA is selected from the group consisting of:
   a) a tracrRNA having at least 90% sequence identity to SEQ ID NO: 120, wherein said one or more guide RNAs further comprise a CRISPR RNA comprising a CRISPR repeat sequence having at least 90% sequence identity to SEQ ID NO: 110, and wherein said RGN polypeptide comprises an amino acid sequence having at least 90% sequence identity to SEQ ID NO: 1;
   b) a tracrRNA having at least 90% sequence identity to SEQ ID NO: 121, wherein said one or more guide RNAs further comprise a CRISPR RNA comprising a CRISPR repeat sequence having at least 90% sequence identity to SEQ ID NO: 111, and wherein said RGN polypeptide comprises an amino acid sequence having at least 90% sequence identity to SEQ ID NO: 2;
   c) a tracrRNA having at least 90% sequence identity to SEQ ID NO: 122, wherein said one or more guide RNAs further comprise a CRISPR RNA comprising a CRISPR repeat sequence having at least 90% sequence identity to SEQ ID NO: 112, and wherein said RGN polypeptide comprises an amino acid sequence having at least 90% sequence identity to SEQ ID NO: 3;
   d) a tracrRNA having at least 90% sequence identity to SEQ ID NO: 123, wherein said one or more guide RNAs further comprise a CRISPR RNA comprising a CRISPR repeat sequence having at least 90% sequence identity to SEQ ID NO: 113, and wherein said RGN polypeptide comprises an amino acid sequence having at least 90% sequence identity to SEQ ID NO: 4;
   e) a tracrRNA having at least 90% sequence identity to SEQ ID NO: 124, wherein said one or more guide RNAs further comprise a CRISPR RNA comprising a CRISPR repeat sequence having at least 90% sequence identity to SEQ ID NO: 114, and wherein said RGN polypeptide comprises an amino acid sequence having at least 90% sequence identity to SEQ ID NO: 5;
   f) a tracrRNA having at least 90% sequence identity to SEQ ID NO: 125, wherein said one or more guide RNAs further comprise a CRISPR RNA comprising a CRISPR repeat sequence having at least 90% sequence identity to SEQ ID NO: 115, and wherein said RGN polypeptide comprises an amino acid sequence having at least 90% sequence identity to SEQ ID NO: 6;
   g) a tracrRNA having at least 90% sequence identity to SEQ ID NO: 126, wherein said one or more guide RNAs further comprise a CRISPR RNA comprising a CRISPR repeat sequence having at least 90% sequence identity to SEQ ID NO: 117, and wherein said RGN polypeptide comprises an amino acid sequence having at least 90% sequence identity to SEQ ID NO: 12;
   h) a tracrRNA having at least 90% sequence identity to SEQ ID NO: 127, wherein said one or more guide RNAs further comprise a CRISPR RNA comprising a CRISPR repeat sequence having at least 90% sequence identity to SEQ ID NO: 118, and wherein said RGN polypeptide comprises an amino acid sequence having at least 90% sequence identity to SEQ ID NO: 13; and
   i) a tracrRNA having at least 90% sequence identity to SEQ ID NO: 128, wherein said one or more guide RNAs further comprise a CRISPR RNA comprising a CRISPR repeat sequence having at least 90% sequence identity to SEQ ID NO: 119, and wherein said RGN polypeptide comprises an amino acid sequence having at least 90% sequence identity to SEQ ID NO: 16.
184. The system of embodiment 182, wherein said tracrRNA is selected from the group consisting of:
   a) a tracrRNA having at least 95% sequence identity to SEQ ID NO: 120, wherein said one or more guide RNAs further comprise a CRISPR RNA comprising a CRISPR repeat sequence having at least 95% sequence identity to SEQ ID NO: 110, and wherein said RGN polypeptide comprises an amino acid sequence having at least 95% sequence identity to SEQ ID NO: 1;
   b) a tracrRNA having at least 95% sequence identity to SEQ ID NO: 121, wherein said one or more guide RNAs further comprise a CRISPR RNA comprising a CRISPR repeat sequence having at least 95% sequence identity to SEQ ID NO: 111, and wherein said RGN polypeptide comprises an amino acid sequence having at least 95% sequence identity to SEQ ID NO: 2;
   c) a tracrRNA having at least 95% sequence identity to SEQ ID NO: 122, wherein said one or more guide RNAs further comprise a CRISPR RNA comprising a CRISPR repeat sequence having at least 95% sequence identity to SEQ ID NO: 112, and wherein said RGN polypeptide comprises an amino acid sequence having at least 95% sequence identity to SEQ ID NO: 3;
   d) a tracrRNA having at least 95% sequence identity to SEQ ID NO: 123, wherein said one or more guide RNAs further comprise a CRISPR RNA comprising a CRISPR repeat sequence having at least 95% sequence identity to SEQ ID NO: 113, and wherein said RGN polypeptide comprises an amino acid sequence having at least 95% sequence identity to SEQ ID NO: 4;
   e) a tracrRNA having at least 95% sequence identity to SEQ ID NO: 124, wherein said one or more guide RNAs further comprise a CRISPR RNA comprising a CRISPR repeat sequence having at least 95% sequence identity to SEQ ID NO: 114, and wherein said RGN polypeptide comprises an amino acid sequence having at least 95% sequence identity to SEQ ID NO: 5;
   f) a tracrRNA having at least 95% sequence identity to SEQ ID NO: 125, wherein said one or more guide RNAs further comprise a CRISPR RNA comprising a CRISPR repeat sequence having at least 95% sequence identity to SEQ ID NO: 115, and wherein said RGN polypeptide comprises an amino acid sequence having at least 95% sequence identity to SEQ ID NO: 6;
   g) a tracrRNA having at least 95% sequence identity to SEQ ID NO: 126, wherein said one or more guide RNAs further comprise a CRISPR RNA comprising a CRISPR repeat sequence having at least 95% sequence identity to SEQ ID NO: 117, and wherein said RGN polypeptide comprises an amino acid sequence having at least 95% sequence identity to SEQ ID NO: 12;
   h) a tracrRNA having at least 95% sequence identity to SEQ ID NO: 127, wherein said one or more guide RNAs further comprise a CRISPR RNA comprising a CRISPR repeat sequence having at least 95% sequence identity to SEQ ID NO: 118, and wherein said RGN polypeptide comprises an amino acid sequence having at least 95% sequence identity to SEQ ID NO: 13; and
   i) a tracrRNA having at least 95% sequence identity to SEQ ID NO: 128, wherein said one or more guide RNAs further comprise a CRISPR RNA comprising a CRISPR repeat sequence having at least 95% sequence identity to SEQ ID NO: 119, and wherein said RGN polypeptide comprises an amino acid sequence having at least 95% sequence identity to SEQ ID NO: 16.
185. The system of embodiment 182, wherein said tracrRNA is selected from the group consisting of:
   a) a tracrRNA having 100% sequence identity to SEQ ID NO: 120, wherein said one or more guide RNAs further comprise a CRISPR RNA comprising a CRISPR repeat sequence having 100% sequence identity to SEQ ID NO: 110, and wherein said RGN polypeptide comprises an amino acid sequence having 100% sequence identity to SEQ ID NO: 1;
   b) a tracrRNA having 100% sequence identity to SEQ ID NO: 121, wherein said one or more guide RNAs further comprise a CRISPR RNA comprising a CRISPR repeat sequence having 100% sequence identity to SEQ ID NO: 111, and wherein said RGN polypeptide comprises an amino acid sequence having 100% sequence identity to SEQ ID NO: 2;
   c) a tracrRNA having 100% sequence identity to SEQ ID NO: 122, wherein said one or more guide RNAs further comprise a CRISPR RNA comprising a CRISPR repeat sequence having 100% sequence identity to SEQ ID NO: 112, and wherein said RGN polypeptide comprises an amino acid sequence having 100% sequence identity to SEQ ID NO: 3;
   d) a tracrRNA having 100% sequence identity to SEQ ID NO: 123, wherein said one or more guide RNAs further comprise a CRISPR RNA comprising a CRISPR repeat sequence having 100% sequence identity to SEQ ID NO: 113, and wherein said RGN polypeptide comprises an amino acid sequence having 100% sequence identity to SEQ ID NO: 4;
   e) a tracrRNA having 100% sequence identity to SEQ ID NO: 124, wherein said one or more guide RNAs further comprise a CRISPR RNA comprising a CRISPR repeat sequence having 100% sequence identity to SEQ ID NO: 114, and wherein said RGN polypeptide comprises an amino acid sequence having 100% sequence identity to SEQ ID NO: 5;
   f) a tracrRNA having 100% sequence identity to SEQ ID NO: 125, wherein said one or more guide RNAs further comprise a CRISPR RNA comprising a CRISPR repeat sequence having 100% sequence identity to SEQ ID NO: 115, and wherein said RGN polypeptide comprises an amino acid sequence having 100% sequence identity to SEQ ID NO: 6;
   g) a tracrRNA having 100% sequence identity to SEQ ID NO: 126, wherein said one or more guide RNAs further comprise a CRISPR RNA comprising a CRISPR repeat sequence having 100% sequence identity to SEQ ID NO: 117, and wherein said RGN polypeptide comprises an amino acid sequence having 100% sequence identity to SEQ ID NO: 12;
   h) a tracrRNA having 100% sequence identity to SEQ ID NO: 127, wherein said one or more guide RNAs further comprise a CRISPR RNA comprising a CRISPR repeat sequence having 100% sequence identity to SEQ ID NO: 118, and wherein said RGN polypeptide comprises an amino acid sequence having 100% sequence identity to SEQ ID NO: 13; and
   i) a tracrRNA having 100% sequence identity to SEQ ID NO: 128, wherein said one or more guide RNAs further comprise a CRISPR RNA comprising a CRISPR repeat sequence having 100% sequence identity to SEQ ID NO: 119, and wherein said RGN polypeptide comprises an amino acid sequence having 100% sequence identity to SEQ ID NO: 16.
186. The system of any one of embodiments 182-185, wherein said one or more guide RNAs are a single guide RNA (sgRNA).
187. The system of any one of embodiments 182-185, wherein said one or more guide RNAs are a dual-guide RNA.
188. The system of any one of embodiments 172-187, wherein said system further comprises at least one RGN accessory protein or a polynucleotide comprising a nucleotide sequence encoding the same selected from the group consisting of:
   a) at least one RGN accessory protein having at least 90% sequence identity to any one of SEQ ID NOs: 178-181, wherein said RGN polypeptide comprises an amino acid sequence having at least 90% sequence identity to SEQ ID NO: 11;
   b) at least one RGN accessory protein having at least 90% sequence identity to any one of SEQ ID NOs: 182-184, wherein said RGN polypeptide comprises an amino acid sequence having at least 90% sequence identity to SEQ ID NO: 12;
   c) at least one RGN accessory protein having at least 90% sequence identity to any one of SEQ ID NOs: 185-187, wherein said RGN polypeptide comprises an amino acid sequence having at least 90% sequence identity to SEQ ID NO: 13;
   d) an RGN accessory protein having at least 90% sequence identity to SEQ ID NO: 191, wherein said RGN polypeptide comprises an amino acid sequence having at least 90% sequence identity to SEQ ID NO: 14;
   e) an RGN accessory protein having at least 90% sequence identity to SEQ ID NO: 192, wherein said RGN polypeptide comprises an amino acid sequence having at least 90% sequence identity to SEQ ID NO: 15; and
   f) at least one RGN accessory protein having at least 90% sequence identity to any one of SEQ ID NOs: 188-190, wherein said RGN polypeptide comprises an amino acid sequence having at least 90% sequence identity to SEQ ID NO: 16.
189. The system of embodiment 188, wherein said at least one RGN accessory protein is selected from the group consisting of:
   a) at least one RGN accessory protein having at least 95% sequence identity to any one of SEQ ID NOs: 178-181, wherein said RGN polypeptide comprises an amino acid sequence having at least 95% sequence identity to SEQ ID NO: 11;
   b) at least one RGN accessory protein having at least 95% sequence identity to any one of SEQ ID NOs: 182-184, wherein said RGN polypeptide comprises an amino acid sequence having at least 95% sequence identity to SEQ ID NO: 12;
   c) at least one RGN accessory protein having at least 95% sequence identity to any one of SEQ ID NOs: 185-187, wherein said RGN polypeptide comprises an amino acid sequence having at least 95% sequence identity to SEQ ID NO: 13;
   d) an RGN accessory protein having at least 95% sequence identity to SEQ ID NO: 191, wherein said RGN polypeptide comprises an amino acid sequence having at least 95% sequence identity to SEQ ID NO: 14;
   e) an RGN accessory protein having at least 95% sequence identity to SEQ ID NO: 192, wherein said RGN polypeptide comprises an amino acid sequence having at least 95% sequence identity to SEQ ID NO: 15; and
   f) at least one RGN accessory protein having at least 95% sequence identity to any one of SEQ ID NOs: 188-190, wherein said RGN polypeptide comprises an amino acid sequence having at least 95% sequence identity to SEQ ID NO: 16.
190. The system of embodiment 188, wherein said at least one RGN accessory protein is selected from the group consisting of:
   a) at least one RGN accessory protein having 100% sequence identity to any one of SEQ ID NOs: 178-181, wherein said RGN polypeptide comprises an amino acid sequence having 100% sequence identity to SEQ ID NO: 11;
   b) at least one RGN accessory protein having 100% sequence identity to any one of SEQ ID NOs: 182-184, wherein said RGN polypeptide comprises an amino acid sequence having 100% sequence identity to SEQ ID NO: 12;
   c) at least one RGN accessory protein having 100% sequence identity to any one of SEQ ID NOs: 185-187, wherein said RGN polypeptide comprises an amino acid sequence having 100% sequence identity to SEQ ID NO: 13;
   d) an RGN accessory protein having 100% sequence identity to SEQ ID NO: 191, wherein said RGN polypeptide comprises an amino acid sequence having 100% sequence identity to SEQ ID NO: 14;
   e) an RGN accessory protein having 100% sequence identity to SEQ ID NO: 192, wherein said RGN polypeptide comprises an amino acid sequence having 100% sequence identity to SEQ ID NO: 15; and
   f) at least one RGN accessory protein having 100% sequence identity to any one of SEQ ID NOs: 188-190, wherein said RGN polypeptide comprises an amino acid sequence having 100% sequence identity to SEQ ID NO: 16.
191. The system of any one of embodiments 172-190, wherein the target DNA sequence is within a cell.
192. The system of embodiment 191, wherein the cell is a eukaryotic cell.
193. The system of embodiment 192, wherein the eukaryotic cell is a plant cell.
194. The system of embodiment 192, wherein the eukaryotic cell is a mammalian cell.
195. The system of embodiment 194, wherein the mammalian cell is a human cell.
196. The system of embodiment 195, wherein the human cell is an immune cell.
197. The system of embodiment 196, wherein the human cell is a stem cell.
198. The system of embodiment 197, wherein the stem cell is an induced pluripotent stem cell. 199. The system of embodiment 192, wherein the eukaryotic cell is an insect cell.
200. The system of embodiment 191, wherein the cell is a prokaryotic cell.
201. The system of any one of embodiments 172-200, wherein said target DNA sequence is within a region of said DNA molecule that is single-stranded.
202. The system of embodiment 201, wherein when transcribed the one or more guide RNAs is capable of hybridizing to the target DNA sequence and the guide RNA is capable of forming a complex with the RGN polypeptide to direct cleavage of the target DNA sequence.
203. The system of any one of embodiments 172-200, wherein said target DNA sequence is within a region of said DNA molecule that is double-stranded.
204. The system of embodiment 203, wherein when transcribed the one or more guide RNAs is capable of hybridizing to the target DNA sequence and the guide RNA is capable of forming a complex with the RGN polypeptide to direct cleavage of the target DNA sequence.
205. The system of embodiment 204, wherein said RGN polypeptide is capable of generating a double-stranded break.
206. The system of embodiment 204, wherein said RGN polypeptide is capable of generating a single-stranded break.
207. The system of any one of embodiments 172-206, wherein the RGN polypeptide is operably linked to a base-editing polypeptide.
208. The system of embodiment 207, wherein said base-editing polypeptide is a deaminase.
209. The system of any one of embodiments 172-208, wherein said target DNA sequence is located adjacent to a protospacer adjacent motif (PAM).
210. The system of any one of embodiments 172-209, wherein the RGN polypeptide comprises one or more nuclear localization signals.
211. The system of any one of embodiments 172-210, wherein the RGN polypeptide is codon optimized for expression in a eukaryotic cell.
212. The system of any one of embodiments 172-211, wherein polynucleotides comprising the nucleotide sequences encoding the one or more guide RNAs and the polynucleotide comprising the nucleotide sequence encoding an RGN polypeptide are located on one vector.
213. The system of any one of embodiments 172-212, wherein said system further comprises one or more donor polynucleotides or one or more polynucleotides comprising one or more nucleotide sequences encoding the one or more donor polynucleotides.
214. A pharmaceutical composition comprising the nucleic acid molecule of any one of embodiments 1-14, 50-52, and 111-113, the vector of any one of embodiments 15-28, 53-110, and 114-171, the cell of embodiment 29, the isolated RGN polypeptide of any one of embodiments 37-49, or the system of any one of embodiments 172-213, and a pharmaceutically acceptable carrier.
215. A method for binding a target DNA sequence of a DNA molecule comprising delivering a system according to any one of embodiments 172-213, to said target DNA sequence or a cell comprising the target DNA sequence.
216. The method of embodiment 215, wherein said RGN polypeptide or said guide RNA further comprises a detectable label, thereby allowing for detection of said target DNA sequence.
217. The method of embodiment 215, wherein said guide RNA or said RGN polypeptide further comprises an expression modulator, thereby modulating expression of said target DNA sequence or a gene under transcriptional control by said target DNA sequence.
218. A method for cleaving a target DNA sequence of a DNA molecule comprising delivering a system according to any one of embodiments 172-213, to said target DNA sequence or a cell comprising the target DNA sequence.
219. The method of embodiment 218, wherein said modified target DNA sequence comprises insertion of heterologous DNA into the target DNA sequence.
220. The method of embodiment 218, wherein said modified target DNA sequence comprises deletion of at least one nucleotide from the target DNA sequence.
221. The method of embodiment 218, wherein said modified target DNA sequence comprises mutation of at least one nucleotide in the target DNA sequence.
222. A method for binding a target DNA sequence of a DNA molecule, said method comprising:
   a) assembling an RNA-guided nuclease (RGN) ribonucleotide complex in vitro by combining:
      i) one or more guide RNAs capable of hybridizing to the target DNA sequence; and
      ii) an RGN polypeptide comprising an amino acid sequence having at least 90% sequence identity to any one of SEQ ID NOs: 1 to 109; under conditions suitable for formation of the RGN ribonucleotide complex; and
   b) contacting said target DNA sequence or a cell comprising said target DNA sequence with the in vitro-assembled RGN ribonucleotide complex;
   wherein the one or more guide RNAs hybridize to the target DNA sequence, thereby directing said RGN polypeptide to bind to said target DNA sequence.
223. The method of embodiment 222, wherein said target DNA sequence is within a region of said DNA molecule that is single-stranded.
224. The method of embodiment 222, wherein said target DNA sequence is within a region of said DNA molecule that is double-stranded.
225. The method of any one of embodiments 222-224, wherein said target DNA sequence is located adjacent to a protospacer adjacent motif (PAM).
226. The method of any one of embodiments 222-225, wherein said RGN polypeptide or said guide RNA further comprises a detectable label, thereby allowing for detection of said target DNA sequence.
227. The method of any one of embodiments 222-225, wherein said guide RNA or said RGN polypeptide further comprises an expression modulator, thereby allowing for the modulation of expression of said target DNA sequence.
228. A method for cleaving and/or modifying a target DNA sequence of a DNA molecule, comprising contacting the DNA molecule with:
   a) an RNA-guided nuclease (RGN) polypeptide, wherein said RGN comprises an amino acid sequence having at least 90% sequence identity to any one of SEQ ID NOs: 1 to 109; and
   b) one or more guide RNAs capable of targeting the RGN of (a) to the target DNA sequence;
   wherein the one or more guide RNAs hybridize to the target DNA sequence, thereby directing said RGN polypeptide to bind to said target DNA sequence and cleavage and/or modification of said target DNA sequence occurs.
229. The method of embodiment 228, wherein said target DNA sequence is within a region of said DNA molecule that is single-stranded.
230. The method of embodiment 228, wherein said target DNA sequence is within a region ofsaid DNA molecule that is double-stranded.
231. The method of embodiment 230, wherein cleavage by said RGN polypeptide generates a double-stranded break.
232. The method of embodiment 230, wherein cleavage by said RGN polypeptide generates a single-stranded break.
233. The method of any one of embodiments 228-232, wherein said RGN polypeptide isoperably linked to a base-editing polypeptide.
234. The method of embodiment 233, wherein said base-editing polypeptide comprises a deaminase.
235. The method of any one of embodiments 228-234, wherein said target DNA sequence is located adjacent to a protospacer adjacent motif (PAM).
236. The method of any one of embodiments 228-235, wherein said modified target DNAsequence comprises insertion of heterologous DNA into the target DNA sequence.
237. The method of any one of embodiments 228-235, wherein said modified target DNA sequence comprises deletion of at least one nucleotide from the target DNA sequence.
238. The method of any one of embodiments 228-235, wherein said modified target DNA sequence comprises mutation of at least one nucleotide in the target DNA sequence.
239. The method of any one of embodiments 222-238, wherein said RGN comprises an amino acid sequence having at least 95% sequence identity to any one of SEQ ID NOs: 1 to 109.
240. The method of any one of embodiments 222-238, wherein said RGN comprises an amino acid sequence having 100% sequence identity to any one of SEQ ID NOs: 1 to 109.
241. The method of any one of embodiments 222-240, wherein said RGN polypeptide and said one or more guide RNAs are not found complexed to one another in nature.
242. The method of any one of embodiments 222-241, wherein said target DNA sequence is a eukaryotic target DNA sequence.
243. The method of any one of embodiments 222-242, wherein said RGN polypeptide comprises an amino acid sequence having at least 90% sequence identity to SEQ ID NO: 11 and said one or more guide RNAs comprise a CRISPR RNA comprising a CRISPR repeat sequence having at least 90% sequence identity to SEQ ID NO: 116.
244. The method of any one of embodiments 222-242, wherein said RGN polypeptide comprises an amino acid sequence having at least 95% sequence identity to SEQ ID NO: 11 and said one or more guide RNAs comprise a CRISPR RNA comprising a CRISPR repeat sequence having at least 95% sequence identity to SEQ ID NO: 116.
245. The method of any one of embodiments 222-242, wherein said RGN polypeptide comprises an amino acid sequence having 100% sequence identity to SEQ ID NO: 11 and said one or more guide RNAs comprise a CRISPR RNA comprising a CRISPR repeat sequence having 100% sequence identity to SEQ ID NO: 116.
246. The method of any one of embodiments 222-242, wherein said one or more guide RNAs comprise a tracrRNA.
247. The method of embodiment 246, wherein said tracrRNA is selected from the group consisting of:
   a) a tracrRNA having at least 90% sequence identity to SEQ ID NO: 120, wherein said one or more guide RNAs further comprise a CRISPR RNA comprising a CRISPR repeat sequence having at least 90% sequence identity to SEQ ID NO: 110, and wherein said RGN polypeptide comprises an amino acid sequence having at least 90% sequence identity to SEQ ID NO: 1;
   b) a tracrRNA having at least 90% sequence identity to SEQ ID NO: 121, wherein said one or more guide RNAs further comprise a CRISPR RNA comprising a CRISPR repeat sequence having at least 90% sequence identity to SEQ ID NO: 111, and wherein said RGN polypeptide comprises an amino acid sequence having at least 90% sequence identity to SEQ ID NO: 2;
   c) a tracrRNA having at least 90% sequence identity to SEQ ID NO: 122, wherein said one or more guide RNAs further comprise a CRISPR RNA comprising a CRISPR repeat sequence having at least 90% sequence identity to SEQ ID NO: 112, and wherein said RGN polypeptide comprises an amino acid sequence having at least 90% sequence identity to SEQ ID NO: 3;
   d) a tracrRNA having at least 90% sequence identity to SEQ ID NO: 123, wherein said one or more guide RNAs further comprise a CRISPR RNA comprising a CRISPR repeat sequence having at least 90% sequence identity to SEQ ID NO: 113, and wherein said RGN polypeptide comprises an amino acid sequence having at least 90% sequence identity to SEQ ID NO: 4;
   e) a tracrRNA having at least 90% sequence identity to SEQ ID NO: 124, wherein said one or more guide RNAs further comprise a CRISPR RNA comprising a CRISPR repeat sequence having at least 90% sequence identity to SEQ ID NO: 114, and wherein said RGN polypeptide comprises an amino acid sequence having at least 90% sequence identity to SEQ ID NO: 5;
   f) a tracrRNA having at least 90% sequence identity to SEQ ID NO: 125, wherein said one or more guide RNAs further comprise a CRISPR RNA comprising a CRISPR repeat sequence having at least 90% sequence identity to SEQ ID NO: 115, and wherein said RGN polypeptide comprises an amino acid sequence having at least 90% sequence identity to SEQ ID NO: 6;
   g) a tracrRNA having at least 90% sequence identity to SEQ ID NO: 126, wherein said one or more guide RNAs further comprise a CRISPR RNA comprising a CRISPR repeat sequence having at least 90% sequence identity to SEQ ID NO: 117, and wherein said RGN polypeptide comprises an amino acid sequence having at least 90% sequence identity to SEQ ID NO: 12;
   h) a tracrRNA having at least 90% sequence identity to SEQ ID NO: 127, wherein said one or more guide RNAs further comprise a CRISPR RNA comprising a CRISPR repeat sequence having at least 90% sequence identity to SEQ ID NO: 118, and wherein said RGN polypeptide comprises an amino acid sequence having at least 90% sequence identity to SEQ ID NO: 13; and
   i) a tracrRNA having at least 90% sequence identity to SEQ ID NO: 128, wherein said one or more guide RNAs further comprise a CRISPR RNA comprising a CRISPR repeat sequence having at least 90% sequence identity to SEQ ID NO: 119, and wherein said RGN polypeptide comprises an amino acid sequence having at least 90% sequence identity to SEQ ID NO: 16.
248. The method of embodiment 246, wherein said tracrRNA is selected from the group consisting of:
   a) a tracrRNA having at least 95% sequence identity to SEQ ID NO: 120, wherein said one or more guide RNAs further comprise a CRISPR RNA comprising a CRISPR repeat sequence having at least 95% sequence identity to SEQ ID NO: 110, and wherein said RGN polypeptide comprises an amino acid sequence having at least 95% sequence identity to SEQ ID NO: 1; b) a tracrRNA having at least 95% sequence identity to SEQ ID NO: 121, wherein said one or more guide RNAs further comprise a CRISPR RNA comprising a CRISPR repeat sequence having at least 95% sequence identity to SEQ ID NO: 111, and wherein said RGN polypeptide comprises an amino acid sequence having at least 95% sequence identity to SEQ ID NO: 2;
   c) a tracrRNA having at least 95% sequence identity to SEQ ID NO: 122, wherein said one or more guide RNAs further comprise a CRISPR RNA comprising a CRISPR repeat sequence having at least 95% sequence identity to SEQ ID NO: 112, and wherein said RGN polypeptide comprises an amino acid sequence having at least 95% sequence identity to SEQ ID NO: 3;
   d) a tracrRNA having at least 95% sequence identity to SEQ ID NO: 123, wherein said one or more guide RNAs further comprise a CRISPR RNA comprising a CRISPR repeat sequence having at least 95% sequence identity to SEQ ID NO: 113, and wherein said RGN polypeptide comprises an amino acid sequence having at least 95% sequence identity to SEQ ID NO: 4;
   e) a tracrRNA having at least 95% sequence identity to SEQ ID NO: 124, wherein said one or more guide RNAs further comprise a CRISPR RNA comprising a CRISPR repeat sequence having at least 95% sequence identity to SEQ ID NO: 114, and wherein said RGN polypeptide comprises an amino acid sequence having at least 95% sequence identity to SEQ ID NO: 5;
   f) a tracrRNA having at least 95% sequence identity to SEQ ID NO: 125, wherein said one or more guide RNAs further comprise a CRISPR RNA comprising a CRISPR repeat sequence having at least 95% sequence identity to SEQ ID NO: 115, and wherein said RGN polypeptide comprises an amino acid sequence having at least 95% sequence identity to SEQ ID NO: 6;
   g) a tracrRNA having at least 95% sequence identity to SEQ ID NO: 126, wherein said one or more guide RNAs further comprise a CRISPR RNA comprising a CRISPR repeat sequence having at least 95% sequence identity to SEQ ID NO: 117, and wherein said RGN polypeptide comprises an amino acid sequence having at least 95% sequence identity to SEQ ID NO: 12;
   h) a tracrRNA having at least 95% sequence identity to SEQ ID NO: 127, wherein said one or more guide RNAs further comprise a CRISPR RNA comprising a CRISPR repeat sequence having at least 95% sequence identity to SEQ ID NO: 118, and wherein said RGN polypeptide comprises an amino acid sequence having at least 95% sequence identity to SEQ ID NO: 13; and
   i) a tracrRNA having at least 95% sequence identity to SEQ ID NO: 128, wherein said one or more guide RNAs further comprise a CRISPR RNA comprising a CRISPR repeat sequence having at least 95% sequence identity to SEQ ID NO: 119, and wherein said RGN polypeptide comprises an amino acid sequence having at least 95% sequence identity to SEQ ID NO: 16.
249. The method of embodiment 246, wherein said tracrRNA is selected from the group consisting of:
   a) a tracrRNA having 100% sequence identity to SEQ ID NO: 120, wherein said one or more guide RNAs further comprise a CRISPR RNA comprising a CRISPR repeat sequence having 100% sequence identity to SEQ ID NO: 110, and wherein said RGN polypeptide comprises an amino acid sequence having 100% sequence identity to SEQ ID NO: 1;
   b) a tracrRNA having 100% sequence identity to SEQ ID NO: 121, wherein said one or more guide RNAs further comprise a CRISPR RNA comprising a CRISPR repeat sequence having 100% sequence identity to SEQ ID NO: 111, and wherein said RGN polypeptide comprises an amino acid sequence having 100% sequence identity to SEQ ID NO: 2;
   c) a tracrRNA having 100% sequence identity to SEQ ID NO: 122, wherein said one or more guide RNAs further comprise a CRISPR RNA comprising a CRISPR repeat sequence having 100% sequence identity to SEQ ID NO: 112, and wherein said RGN polypeptide comprises an amino acid sequence having 100% sequence identity to SEQ ID NO: 3;
   d) a tracrRNA having 100% sequence identity to SEQ ID NO: 123, wherein said one or more guide RNAs further comprise a CRISPR RNA comprising a CRISPR repeat sequence having 100% sequence identity to SEQ ID NO: 113, and wherein said RGN polypeptide comprises an amino acid sequence having 100% sequence identity to SEQ ID NO: 4;
   e) a tracrRNA having 100% sequence identity to SEQ ID NO: 124, wherein said one or more guide RNAs further comprise a CRISPR RNA comprising a CRISPR repeat sequence having 100% sequence identity to SEQ ID NO: 114, and wherein said RGN polypeptide comprises an amino acid sequence having 100% sequence identity to SEQ ID NO: 5;
   f) a tracrRNA having 100% sequence identity to SEQ ID NO: 125, wherein said one or more guide RNAs further comprise a CRISPR RNA comprising a CRISPR repeat sequence having 100% sequence identity to SEQ ID NO: 115, and wherein said RGN polypeptide comprises an amino acid sequence having 100% sequence identity to SEQ ID NO: 6;
   g) a tracrRNA having 100% sequence identity to SEQ ID NO: 126, wherein said one or more guide RNAs further comprise a CRISPR RNA comprising a CRISPR repeat sequence having 100% sequence identity to SEQ ID NO: 117, and wherein said RGN polypeptide comprises an amino acid sequence having 100% sequence identity to SEQ ID NO: 12;
   h) a tracrRNA having 100% sequence identity to SEQ ID NO: 127, wherein said one or more guide RNAs further comprise a CRISPR RNA comprising a CRISPR repeat sequence having 100% sequence identity to SEQ ID NO: 118, and wherein said RGN polypeptide comprises an amino acid sequence having 100% sequence identity to SEQ ID NO: 13; and
   i) a tracrRNA having 100% sequence identity to SEQ ID NO: 128, wherein said one or more guide RNAs further comprise a CRISPR RNA comprising a CRISPR repeat sequence having 100% sequence identity to SEQ ID NO: 119, and wherein said RGN polypeptide comprises an amino acid sequence having 100% sequence identity to SEQ ID NO: 16.
250. The method of any one of embodiments 246-249, wherein said one or more guide RNAs are a single guide RNA (sgRNA).
251. The method of any one of embodiments 246-249, wherein said one or more guide RNAs are a dual-guide RNA.
252. The method of any one of embodiments 222-251, wherein said method further comprises contacting the DNA molecule with one or more RGN accessory proteins selected from the group consisting of:
   a) at least one RGN accessory protein having at least 90% sequence identity to any one of SEQ ID NOs: 178-181, wherein said RGN polypeptide comprises an amino acid sequence having at least 90% sequence identity to SEQ ID NO: 11;
   b) at least one RGN accessory protein having at least 90% sequence identity to any one of SEQ ID NOs: 182-184, wherein said RGN polypeptide comprises an amino acid sequence having at least 90% sequence identity to SEQ ID NO: 12;
   c) at least one RGN accessory protein having at least 90% sequence identity to any one of SEQ ID NOs: 185-187, wherein said RGN polypeptide comprises an amino acid sequence having at least 90% sequence identity to SEQ ID NO: 13;
   d) an RGN accessory protein having at least 90% sequence identity to SEQ ID NO: 191, wherein said RGN polypeptide comprises an amino acid sequence having at least 90% sequence identity to SEQ ID NO: 14;
   e) an RGN accessory protein having at least 90% sequence identity to SEQ ID NO: 192, wherein said RGN polypeptide comprises an amino acid sequence having at least 90% sequence identity to SEQ ID NO: 15; and
   f) at least one RGN accessory protein having at least 90% sequence identity to any one of SEQ ID NOs: 188-190, wherein said RGN polypeptide comprises an amino acid sequence having at least 90% sequence identity to SEQ ID NO: 16.
253. The method of embodiment 252, wherein said one or more RGN accessory proteins are selected from the group consisting of:
   a) at least one RGN accessory protein having at least 95% sequence identity to any one of SEQ ID NOs: 178-181, wherein said RGN polypeptide comprises an amino acid sequence having at least 95% sequence identity to SEQ ID NO: 11;
   b) at least one RGN accessory protein having at least 95% sequence identity to any one of SEQ ID NOs: 182-184, wherein said RGN polypeptide comprises an amino acid sequence having at least 95% sequence identity to SEQ ID NO: 12;
   c) at least one RGN accessory protein having at least 95% sequence identity to any one of SEQ ID NOs: 185-187, wherein said RGN polypeptide comprises an amino acid sequence having at least 95% sequence identity to SEQ ID NO: 13;
   d) an RGN accessory protein having at least 95% sequence identity to SEQ ID NO: 191, wherein said RGN polypeptide comprises an amino acid sequence having at least 95% sequence identity to SEQ ID NO: 14;
   e) an RGN accessory protein having at least 95% sequence identity to SEQ ID NO: 192, wherein said RGN polypeptide comprises an amino acid sequence having at least 95% sequence identity to SEQ ID NO: 15; and
   f) at least one RGN accessory protein having at least 95% sequence identity to any one of SEQ ID NOs: 188-190, wherein said RGN polypeptide comprises an amino acid sequence having at least 95% sequence identity to SEQ ID NO: 16.
254. The method of embodiment 252, wherein said one or more RGN accessory proteins are selected from the group consisting of:
   a) at least one RGN accessory protein having 100% sequence identity to any one of SEQ ID NOs: 178-181, wherein said RGN polypeptide comprises an amino acid sequence having 100% sequence identity to SEQ ID NO: 11;
   b) at least one RGN accessory protein having 100% sequence identity to any one of SEQ ID NOs: 182-184, wherein said RGN polypeptide comprises an amino acid sequence having 100% sequence identity to SEQ ID NO: 12;
   c) at least one RGN accessory protein having 100% sequence identity to any one of SEQ ID NOs: 185-187, wherein said RGN polypeptide comprises an amino acid sequence having 100% sequence identity to SEQ ID NO: 13;
   d) an RGN accessory protein having 100% sequence identity to SEQ ID NO: 191, wherein said RGN polypeptide comprises an amino acid sequence having 100% sequence identity to SEQ ID NO: 14;
   e) an RGN accessory protein having 100% sequence identity to SEQ ID NO: 192, wherein said RGN polypeptide comprises an amino acid sequence having 100% sequence identity to SEQ ID NO: 15; and
   f) at least one RGN accessory protein having 100% sequence identity to any one of SEQ ID NOs: 188-190, wherein said RGN polypeptide comprises an amino acid sequence having 100% sequence identity to SEQ ID NO: 16.
255. The method of any one of embodiments 215-254, wherein the target DNA sequence is within a cell.
256. The method of embodiment 255, wherein the cell is a eukaryotic cell.
257. The method of embodiment 256, wherein the eukaryotic cell is a plant cell.
258. The method of embodiment 256, wherein the eukaryotic cell is a mammalian cell.
259. The method of embodiment 258, wherein the mammalian cell is a human cell.
260. The method of embodiment 259, wherein the human cell is an immune cell.
261. The method of embodiment 260, wherein the human cell is a stem cell.
262. The method of embodiment 261, wherein the stem cell is an induced pluripotent stem cell. 263. The method of embodiment 256, wherein the eukaryotic cell is an insect cell.
264. The method of embodiment 255, wherein the cell is a prokaryotic cell.
265. A cell comprising a modified target DNA sequence according to the method of any one of embodiments 228-254.
266. The cell of embodiment 265, wherein the cell is a eukaryotic cell.
267. The cell of embodiment 266, wherein the eukaryotic cell is a plant cell.
268. A plant comprising the cell of embodiment 267.
269. A seed comprising the cell of embodiment 267.
270. The cell of embodiment 266, wherein the eukaryotic cell is a mammalian cell.
271. The cell of embodiment 270, wherein the mammalian cell is a human cell.
272. The cell of embodiment 271, wherein the human cell is an immune cell.
273. The cell of embodiment 272, wherein the human cell is a stem cell.
274. The cell of embodiment 273, wherein the stem cell is an induced pluripotent stem cell.
275. The cell of embodiment 266, wherein the eukaryotic cell is an insect cell.
276. The cell of embodiment 265, wherein the cell is a prokaryotic cell.
277. A pharmaceutical composition comprising the cell of any one of embodiments 266 and 270-274, and a pharmaceutically acceptable carrier.
278. A kit for detecting a target DNA sequence of a DNA molecule in a sample, the kit comprising:
   a) an RNA-guided nuclease (RGN) polypeptide comprising an amino acid sequence having at least 90% sequence identity to any one of SEQ ID NOs: 1 to 109 or a polynucleotide comprising a nucleotide sequence encoding the RGN polypeptide, wherein said RGN polypeptide is capable of binding and cleaving said target DNA sequence of a DNA molecule in an RNA-guided sequence specific manner when bound to a guide RNA capable of hybridizing to said target DNA sequence;
   b) said guide RNA or a polynucleotide comprising a nucleotide sequence encoding said guide RNA; and
   c) a detector single-stranded DNA (ssDNA) that does not hybridize with the guide RNA.
279. The kit of embodiment 278, wherein said RGN polypeptide comprises an amino acid sequence having at least 95% sequence identity to any one of SEQ ID NOs: 1 to 109.
280. The kit of embodiment 278, wherein said RGN polypeptide comprises an amino acid sequence having 100% sequence identity to any one of SEQ ID NOs: 1 to 109.
281. The kit of any one of embodiments 278-280, wherein at least one of said nucleotide sequence encoding the guide RNA and said nucleotide sequence encoding the RGN polypeptide is operably linked to a promoter heterologous to said nucleotide sequence.
282. The kit of any one of embodiments 278-281, wherein said RGN polypeptide and said one or more guide RNAs are not found complexed to one another in nature.
283. The kit of any one of embodiments 278-282, wherein said target DNA sequence is a eukaryotic target DNA sequence.
284. The kit of any one of embodiments 278-283, wherein said detector ssDNA comprises a fluorophore/quencher pair.
285. The kit of any one of embodiments 278-283, wherein said detector ssDNA comprises a fluorescence resonance energy transfer (FRET) pair.
286. The kit of any one of embodiments 278-285, wherein said kit further comprises at least one RGN accessory protein selected from the group consisting of:
   a) at least one RGN accessory protein having at least 90% sequence identity to any one of SEQ ID NOs: 178-181, wherein said RGN polypeptide comprises an amino acid sequence having at least 90% sequence identity to SEQ ID NO: 11;
   b) at least one RGN accessory protein having at least 90% sequence identity to any one of SEQ ID NOs: 182-184, wherein said RGN polypeptide comprises an amino acid sequence having at least 90% sequence identity to SEQ ID NO: 12;
   c) at least one RGN accessory protein having at least 90% sequence identity to any one of SEQ ID NOs: 185-187, wherein said RGN polypeptide comprises an amino acid sequence having at least 90% sequence identity to SEQ ID NO: 13;
   d) an RGN accessory protein having at least 90% sequence identity to SEQ ID NO: 191, wherein said RGN polypeptide comprises an amino acid sequence having at least 90% sequence identity to SEQ ID NO: 14;
   e) an RGN accessory protein having at least 90% sequence identity to SEQ ID NO: 192, wherein said RGN polypeptide comprises an amino acid sequence having at least 90% sequence identity to SEQ ID NO: 15; and
   f) at least one RGN accessory protein having at least 90% sequence identity to any one of SEQ ID NOs: 188-190, wherein said RGN polypeptide comprises an amino acid sequence having at least 90% sequence identity to SEQ ID NO: 16.
287. The kit of embodiment 286, wherein said at least one RGN accessory protein is selected from the group consisting of:
   a) at least one RGN accessory protein having at least 95% sequence identity to any one of SEQ ID NOs: 178-181, wherein said RGN polypeptide comprises an amino acid sequence having at least 95% sequence identity to SEQ ID NO: 11;
   b) at least one RGN accessory protein having at least 95% sequence identity to any one of SEQ ID NOs: 182-184, wherein said RGN polypeptide comprises an amino acid sequence having at least 95% sequence identity to SEQ ID NO: 12;
   c) at least one RGN accessory protein having at least 95% sequence identity to any one of SEQ ID NOs: 185-187, wherein said RGN polypeptide comprises an amino acid sequence having at least 95% sequence identity to SEQ ID NO: 13;
   d) an RGN accessory protein having at least 95% sequence identity to SEQ ID NO: 191, wherein said RGN polypeptide comprises an amino acid sequence having at least 95% sequence identity to SEQ ID NO: 14;
   e) an RGN accessory protein having at least 95% sequence identity to SEQ ID NO: 192, wherein said RGN polypeptide comprises an amino acid sequence having at least 95% sequence identity to SEQ ID NO: 15; and
   f) at least one RGN accessory protein having at least 95% sequence identity to any one of SEQ ID NOs: 188-190, wherein said RGN polypeptide comprises an amino acid sequence having at least 95% sequence identity to SEQ ID NO: 16.
288. The kit of embodiment 286, wherein said at least one RGN accessory protein is selected from the group consisting of:
   a) at least one RGN accessory protein having 100% sequence identity to any one of SEQ ID NOs: 178-181, wherein said RGN polypeptide comprises an amino acid sequence having 100% sequence identity to SEQ ID NO: 11;
   b) at least one RGN accessory protein having 100% sequence identity to any one of SEQ ID NOs: 182-184, wherein said RGN polypeptide comprises an amino acid sequence having 100% sequence identity to SEQ ID NO: 12;
   c) at least one RGN accessory protein having 100% sequence identity to any one of SEQ ID NOs: 185-187, wherein said RGN polypeptide comprises an amino acid sequence having 100% sequence identity to SEQ ID NO: 13;
   d) an RGN accessory protein having 100% sequence identity to SEQ ID NO: 191, wherein said RGN polypeptide comprises an amino acid sequence having 100% sequence identity to SEQ ID NO: 14;
   e) an RGN accessory protein having 100% sequence identity to SEQ ID NO: 192, wherein said RGN polypeptide comprises an amino acid sequence having 100% sequence identity to SEQ ID NO: 15; and
   f) at least one RGN accessory protein having 100% sequence identity to any one of SEQ ID NOs: 188-190, wherein said RGN polypeptide comprises an amino acid sequence having 100% sequence identity to SEQ ID NO: 16.
289. The kit of any one of embodiments 278-288, wherein said RGN polypeptide comprises an amino acid sequence having at least 90% sequence identity to SEQ ID NO: 11 and said one or more guide RNAs comprise a CRISPR RNA comprising a CRISPR repeat sequence having at least 90% sequence identity to SEQ ID NO: 116.
290. The kit of any one of embodiments 278-288, wherein said RGN polypeptide comprises an amino acid sequence having at least 95% sequence identity to SEQ ID NO: 11 and said one or more guide RNAs comprise a CRISPR RNA comprising a CRISPR repeat sequence having at least 95% sequence identity to SEQ ID NO: 116.
291. The kit of any one of embodiments 278-288, wherein said RGN polypeptide comprises an amino acid sequence having 100% sequence identity to SEQ ID NO: 11 and said one or more guide RNAs comprise a CRISPR RNA comprising a CRISPR repeat sequence having 100% sequence identity to SEQ ID NO: 116.
292. The kit of any one of embodiments 278-288, wherein said one or more guide RNAs comprise a tracrRNA.
293. The kit of embodiment 292, wherein said tracrRNA is selected from the group consisting of:
   a) a tracrRNA having at least 90% sequence identity to SEQ ID NO: 120, wherein said one or more guide RNAs further comprise a CRISPR RNA comprising a CRISPR repeat sequence having at least 90% sequence identity to SEQ ID NO: 110, and wherein said RGN polypeptide comprises an amino acid sequence having at least 90% sequence identity to SEQ ID NO: 1;
   b) a tracrRNA having at least 90% sequence identity to SEQ ID NO: 121, wherein said one or more guide RNAs further comprise a CRISPR RNA comprising a CRISPR repeat sequence having at least 90% sequence identity to SEQ ID NO: 111, and wherein said RGN polypeptide comprises an amino acid sequence having at least 90% sequence identity to SEQ ID NO: 2;
   c) a tracrRNA having at least 90% sequence identity to SEQ ID NO: 122, wherein said one or more guide RNAs further comprise a CRISPR RNA comprising a CRISPR repeat sequence having at least 90% sequence identity to SEQ ID NO: 112, and wherein said RGN polypeptide comprises an amino acid sequence having at least 90% sequence identity to SEQ ID NO: 3;
   d) a tracrRNA having at least 90% sequence identity to SEQ ID NO: 123, wherein said one or more guide RNAs further comprise a CRISPR RNA comprising a CRISPR repeat sequence having at least 90% sequence identity to SEQ ID NO: 113, and wherein said RGN polypeptide comprises an amino acid sequence having at least 90% sequence identity to SEQ ID NO: 4;
   e) a tracrRNA having at least 90% sequence identity to SEQ ID NO: 124, wherein said one or more guide RNAs further comprise a CRISPR RNA comprising a CRISPR repeat sequence having at least 90% sequence identity to SEQ ID NO: 114, and wherein said RGN polypeptide comprises an amino acid sequence having at least 90% sequence identity to SEQ ID NO: 5;
   f) a tracrRNA having at least 90% sequence identity to SEQ ID NO: 125, wherein said one or more guide RNAs further comprise a CRISPR RNA comprising a CRISPR repeat sequence having at least 90% sequence identity to SEQ ID NO: 115, and wherein said RGN polypeptide comprises an amino acid sequence having at least 90% sequence identity to SEQ ID NO: 6;
   g) a tracrRNA having at least 90% sequence identity to SEQ ID NO: 126, wherein said one or more guide RNAs further comprise a CRISPR RNA comprising a CRISPR repeat sequence having at least 90% sequence identity to SEQ ID NO: 117, and wherein said RGN polypeptide comprises an amino acid sequence having at least 90% sequence identity to SEQ ID NO: 12;
   h) a tracrRNA having at least 90% sequence identity to SEQ ID NO: 127, wherein said one or more guide RNAs further comprise a CRISPR RNA comprising a CRISPR repeat sequence having at least 90% sequence identity to SEQ ID NO: 118, and wherein said RGN polypeptide comprises an amino acid sequence having at least 90% sequence identity to SEQ ID NO: 13; and
   i) a tracrRNA having at least 90% sequence identity to SEQ ID NO: 128, wherein said one or more guide RNAs further comprise a CRISPR RNA comprising a CRISPR repeat sequence having at least 90% sequence identity to SEQ ID NO: 119, and wherein said RGN polypeptide comprises an amino acid sequence having at least 90% sequence identity to SEQ ID NO: 16.
294. The kit of embodiment 292, wherein said tracrRNA is selected from the group consisting of:
   a) a tracrRNA having at least 95% sequence identity to SEQ ID NO: 120, wherein said one or more guide RNAs further comprise a CRISPR RNA comprising a CRISPR repeat sequence having at least 95% sequence identity to SEQ ID NO: 110, and wherein said RGN polypeptide comprises an amino acid sequence having at least 95% sequence identity to SEQ ID NO: 1;
   b) a tracrRNA having at least 95% sequence identity to SEQ ID NO: 121, wherein said one or more guide RNAs further comprise a CRISPR RNA comprising a CRISPR repeat sequence having at least 95% sequence identity to SEQ ID NO: 111, and wherein said RGN polypeptide comprises an amino acid sequence having at least 95% sequence identity to SEQ ID NO: 2;
   c) a tracrRNA having at least 95% sequence identity to SEQ ID NO: 122, wherein said one or more guide RNAs further comprise a CRISPR RNA comprising a CRISPR repeat sequence having at least 95% sequence identity to SEQ ID NO: 112, and wherein said RGN polypeptide comprises an amino acid sequence having at least 95% sequence identity to SEQ ID NO: 3;
   d) a tracrRNA having at least 95% sequence identity to SEQ ID NO: 123, wherein said one or more guide RNAs further comprise a CRISPR RNA comprising a CRISPR repeat sequence having at least 95% sequence identity to SEQ ID NO: 113, and wherein said RGN polypeptide comprises an amino acid sequence having at least 95% sequence identity to SEQ ID NO: 4;
   e) a tracrRNA having at least 95% sequence identity to SEQ ID NO: 124, wherein said one or more guide RNAs further comprise a CRISPR RNA comprising a CRISPR repeat sequence having at least 95% sequence identity to SEQ ID NO: 114, and wherein said RGN polypeptide comprises an amino acid sequence having at least 95% sequence identity to SEQ ID NO: 5;
   f) a tracrRNA having at least 95% sequence identity to SEQ ID NO: 125, wherein said one or more guide RNAs further comprise a CRISPR RNA comprising a CRISPR repeat sequence having at least 95% sequence identity to SEQ ID NO: 115, and wherein said RGN polypeptide comprises an amino acid sequence having at least 95% sequence identity to SEQ ID NO: 6;
   g) a tracrRNA having at least 95% sequence identity to SEQ ID NO: 126, wherein said one or more guide RNAs further comprise a CRISPR RNA comprising a CRISPR repeat sequence having at least 95% sequence identity to SEQ ID NO: 117, and wherein said RGN polypeptide comprises an amino acid sequence having at least 95% sequence identity to SEQ ID NO: 12;
   h) a tracrRNA having at least 95% sequence identity to SEQ ID NO: 127, wherein said one or more guide RNAs further comprise a CRISPR RNA comprising a CRISPR repeat sequence having at least 95% sequence identity to SEQ ID NO: 118, and wherein said RGN polypeptide comprises an amino acid sequence having at least 95% sequence identity to SEQ ID NO: 13; and
   i) a tracrRNA having at least 95% sequence identity to SEQ ID NO: 128, wherein said one or more guide RNAs further comprise a CRISPR RNA comprising a CRISPR repeat sequence having at least 95% sequence identity to SEQ ID NO: 119, and wherein said RGN polypeptide comprises an amino acid sequence having at least 95% sequence identity to SEQ ID NO: 16.
295. The kit of embodiment 292, wherein said tracrRNA is selected from the group consisting of:
   a) a tracrRNA having 100% sequence identity to SEQ ID NO: 120, wherein said one or more guide RNAs further comprise a CRISPR RNA comprising a CRISPR repeat sequence having 100% sequence identity to SEQ ID NO: 110, and wherein said RGN polypeptide comprises an amino acid sequence having 100% sequence identity to SEQ ID NO: 1;
   b) a tracrRNA having 100% sequence identity to SEQ ID NO: 121, wherein said one or more guide RNAs further comprise a CRISPR RNA comprising a CRISPR repeat sequence having 100% sequence identity to SEQ ID NO: 111, and wherein said RGN polypeptide comprises an amino acid sequence having 100% sequence identity to SEQ ID NO: 2;
   c) a tracrRNA having 100% sequence identity to SEQ ID NO: 122, wherein said one or more guide RNAs further comprise a CRISPR RNA comprising a CRISPR repeat sequence having 100% sequence identity to SEQ ID NO: 112, and wherein said RGN polypeptide comprises an amino acid sequence having 100% sequence identity to SEQ ID NO: 3;
   d) a tracrRNA having 100% sequence identity to SEQ ID NO: 123, wherein said one or more guide RNAs further comprise a CRISPR RNA comprising a CRISPR repeat sequence having 100% sequence identity to SEQ ID NO: 113, and wherein said RGN polypeptide comprises an amino acid sequence having 100% sequence identity to SEQ ID NO: 4;
   e) a tracrRNA having 100% sequence identity to SEQ ID NO: 124, wherein said one or more guide RNAs further comprise a CRISPR RNA comprising a CRISPR repeat sequence having 100% sequence identity to SEQ ID NO: 114, and wherein said RGN polypeptide comprises an amino acid sequence having 100% sequence identity to SEQ ID NO: 5;
   f) a tracrRNA having 100% sequence identity to SEQ ID NO: 125, wherein said one or more guide RNAs further comprise a CRISPR RNA comprising a CRISPR repeat sequence having 100% sequence identity to SEQ ID NO: 115, and wherein said RGN polypeptide comprises an amino acid sequence having 100% sequence identity to SEQ ID NO: 6;
   g) a tracrRNA having 100% sequence identity to SEQ ID NO: 126, wherein said one or more guide RNAs further comprise a CRISPR RNA comprising a CRISPR repeat sequence having 100% sequence identity to SEQ ID NO: 117, and wherein said RGN polypeptide comprises an amino acid sequence having 100% sequence identity to SEQ ID NO: 12;
   h) a tracrRNA having 100% sequence identity to SEQ ID NO: 127, wherein said one or more guide RNAs further comprise a CRISPR RNA comprising a CRISPR repeat sequence having 100% sequence identity to SEQ ID NO: 118, and wherein said RGN polypeptide comprises an amino acid sequence having 100% sequence identity to SEQ ID NO: 13; and
   i) a tracrRNA having 100% sequence identity to SEQ ID NO: 128, wherein said one or more guide RNAs further comprise a CRISPR RNA comprising a CRISPR repeat sequence having 100% sequence identity to SEQ ID NO: 119, and wherein said RGN polypeptide comprises an amino acid sequence having 100% sequence identity to SEQ ID NO: 16.
296. The kit of any one of embodiments 292-295, wherein said one or more guide RNAs are a single guide RNA (sgRNA).
297. The kit of any one of embodiments 292-295, wherein said one or more guide RNAs are a dual-guide RNA.
298. The kit of any one of embodiments 278-297, wherein said target DNA sequence is within a region of said DNA molecule that is single-stranded.
299. The kit of any one of embodiments 278-297, wherein said target DNA sequence is within a region of said DNA molecule that is double-stranded.
300. The kit of embodiment 299, wherein cleavage by said RGN polypeptide generates a double-stranded break.
301. The kit of embodiment 299, wherein cleavage by said RGN polypeptide generates a single-stranded break.
302. The kit of any one of embodiments 278-301, wherein said target DNA sequence is located adjacent to a protospacer adjacent motif (PAM).
303. A method of detecting a target DNA sequence of a DNA molecule in a sample, the method comprising:
   a) contacting the sample with:
      i) an RNA-guided nuclease (RGN) polypeptide comprising an amino acid sequence having at least 90% sequence identity to any one of SEQ ID NOs: 1 to 109, wherein said RGN polypeptide is capable of binding and cleaving said target DNA sequence of a DNA molecule in an RNA-guided sequence specific manner when bound to a guide RNA capable of hybridizing to said target DNA sequence;
      ii) said guide RNA; and
      iii) a detector single-stranded DNA (ssDNA) that does not hybridize with the guide RNA; and
   b) measuring a detectable signal produced by cleavage of the detector ssDNA by the RGN, thereby detecting the target DNA sequence.
304. The method of embodiment 303, wherein said RGN polypeptide comprises an amino acid sequence having at least 95% sequence identity to any one of SEQ ID NOs: 1 to 109.
305. The method of embodiment 303, wherein said RGN polypeptide comprises an amino acid sequence having 100% sequence identity to any one of SEQ ID NOs: 1 to 109.
306. The method of any one of embodiments 303-305, wherein said sample comprises DNA molecules from a cell lysate.
307. The method of any one of embodiments 303-305, wherein said sample comprises cells. 308. The method of embodiment 307, wherein said cells are eukaryotic cells.
309. The method of any one of embodiments 303-305, wherein the DNA molecule comprising the target DNA sequence is produced by reverse-transcription of an RNA template molecule present in a sample comprising RNA.
310. The method of embodiment 309, wherein the RNA template molecule is an RNA virus.
311. The method of embodiment 310, wherein the RNA virus is a coronavirus.
312. The method of embodiment 311, wherein the coronavirus is a bat SARS-like coronavirus, SARS-CoV, or SARS-CoV-2.
313. The method of any one of embodiments 309-312, wherein the samples comprising RNA isderived from a sample comprising cells.
314. The method of any one of embodiments 303-313, wherein said detector ssDNA comprises afluorophore/quencher pair.
315. The method of any one of embodiments 303-313, wherein said detector ssDNA comprises a fluorescence resonance energy transfer (FRET) pair.
316. The method of any one of embodiments 303-315, wherein said method further comprisesamplifying nucleic acids in the sample prior to or together with the contacting of step a).
317. The method of any one of embodiments 303-316, wherein said method further comprisescontacting the sample with one or more RGN accessory proteins selected from the group consisting of:
   a) at least one RGN accessory protein having at least 90% sequence identity to any one of SEQ ID NOs: 178-181, wherein said RGN polypeptide comprises an amino acid sequence having at least 90% sequence identity to SEQ ID NO: 11;
   b) at least one RGN accessory protein having at least 90% sequence identity to any one of SEQ ID NOs: 182-184, wherein said RGN polypeptide comprises an amino acid sequence having at least 90% sequence identity to SEQ ID NO: 12;
   c) at least one RGN accessory protein having at least 90% sequence identity to any one of SEQ ID NOs: 185-187, wherein said RGN polypeptide comprises an amino acid sequence having at least 90% sequence identity to SEQ ID NO: 13;
   d) an RGN accessory protein having at least 90% sequence identity to SEQ ID NO: 191, wherein said RGN polypeptide comprises an amino acid sequence having at least 90% sequence identity to SEQ ID NO: 14;
   e) an RGN accessory protein having at least 90% sequence identity to SEQ ID NO: 192, wherein said RGN polypeptide comprises an amino acid sequence having at least 90% sequence identity to SEQ ID NO: 15; and
   f) at least one RGN accessory protein having at least 90% sequence identity to any one of SEQ ID NOs: 188-190, wherein said RGN polypeptide comprises an amino acid sequence having at least 90% sequence identity to SEQ ID NO: 16.
318. The method of embodiment 317, wherein said one or more RGN accessory proteins is selected from the group consisting of:
   a) at least one RGN accessory protein having at least 95% sequence identity to any one of SEQ ID NOs: 178-181, wherein said RGN polypeptide comprises an amino acid sequence having at least 95% sequence identity to SEQ ID NO: 11;
   b) at least one RGN accessory protein having at least 95% sequence identity to any one of SEQ ID NOs: 182-184, wherein said RGN polypeptide comprises an amino acid sequence having at least 95% sequence identity to SEQ ID NO: 12;
   c) at least one RGN accessory protein having at least 95% sequence identity to any one of SEQ ID NOs: 185-187, wherein said RGN polypeptide comprises an amino acid sequence having at least 95% sequence identity to SEQ ID NO: 13;
   d) an RGN accessory protein having at least 95% sequence identity to SEQ ID NO: 191, wherein said RGN polypeptide comprises an amino acid sequence having at least 95% sequence identity to SEQ ID NO: 14;
   e) an RGN accessory protein having at least 95% sequence identity to SEQ ID NO: 192, wherein said RGN polypeptide comprises an amino acid sequence having at least 95% sequence identity to SEQ ID NO: 15; and
   f) at least one RGN accessory protein having at least 95% sequence identity to any one of SEQ ID NOs: 188-190, wherein said RGN polypeptide comprises an amino acid sequence having at least 95% sequence identity to SEQ ID NO: 16.
319. The method of embodiment 317, wherein said one or more RGN accessory proteins isselected from the group consisting of:
   a) at least one RGN accessory protein having 100% sequence identity to any one of SEQ ID NOs: 178-181, wherein said RGN polypeptide comprises an amino acid sequence having 100% sequence identity to SEQ ID NO: 11;
   b) at least one RGN accessory protein having 100% sequence identity to any one of SEQ ID NOs: 182-184, wherein said RGN polypeptide comprises an amino acid sequence having 100% sequence identity to SEQ ID NO: 12;
   c) at least one RGN accessory protein having 100% sequence identity to any one of SEQ ID NOs: 185-187, wherein said RGN polypeptide comprises an amino acid sequence having 100% sequence identity to SEQ ID NO: 13;
   d) an RGN accessory protein having 100% sequence identity to SEQ ID NO: 191, wherein said RGN polypeptide comprises an amino acid sequence having 100% sequence identity to SEQ ID NO: 14;
   e) an RGN accessory protein having 100% sequence identity to SEQ ID NO: 192, wherein said RGN polypeptide comprises an amino acid sequence having 100% sequence identity to SEQ ID NO: 15; and
   f) at least one RGN accessory protein having 100% sequence identity to any one of SEQ ID NOs: 188-190, wherein said RGN polypeptide comprises an amino acid sequence having 100% sequence identity to SEQ ID NO: 16.
320. A method of cleaving single-stranded DNAs (ssDNAs), the method comprising contacting a population of nucleic acids, wherein said population comprises a DNA molecule comprising a target DNA sequence and a plurality of non-target ssDNAs with:
   a) an RNA-guided nuclease (RGN) polypeptide comprising an amino acid sequence having at least 90% sequence identity to any one of SEQ ID NOs: 1 to 109, wherein said RGN polypeptide is capable of binding and cleaving said target DNA sequence in an RNA-guided sequence specific manner when bound to a guide RNA capable of hybridizing to said target DNA sequence; and
   b) said guide RNA; wherein the RGN polypeptide cleaves non-target ssDNAs of said plurality.
321. The method of embodiment 320, wherein said RGN polypeptide comprises an amino acid sequence having at least 95% sequence identity to any one of SEQ ID NOs: 1 to 109.
322. The method of embodiment 320, wherein said RGN polypeptide comprises an amino acid sequence having 100% sequence identity to any one of SEQ ID NOs: 1 to 109.
323. The method of any one of embodiments 320-322, wherein said population of nucleic acids are within a cell lysate.
324. The method of any one of embodiments 320-323, wherein the DNA molecule comprising the target DNA sequence is produced by reverse-transcription of an RNA template molecule.
325. The method of any one of embodiments 320-324, wherein said method further comprises contacting the population with one or more RGN accessory proteins selected from the group consisting of:
   a) at least one RGN accessory protein having at least 90% sequence identity to any one of SEQ ID NOs: 178-181, wherein said RGN polypeptide comprises an amino acid sequence having at least 90% sequence identity to SEQ ID NO: 11;
   b) at least one RGN accessory protein having at least 90% sequence identity to any one of SEQ ID NOs: 182-184, wherein said RGN polypeptide comprises an amino acid sequence having at least 90% sequence identity to SEQ ID NO: 12;
   c) at least one RGN accessory protein having at least 90% sequence identity to any one of SEQ ID NOs: 185-187, wherein said RGN polypeptide comprises an amino acid sequence having at least 90% sequence identity to SEQ ID NO: 13;
   d) an RGN accessory protein having at least 90% sequence identity to SEQ ID NO: 191, wherein said RGN polypeptide comprises an amino acid sequence having at least 90% sequence identity to SEQ ID NO: 14;
   e) an RGN accessory protein having at least 90% sequence identity to SEQ ID NO: 192, wherein said RGN polypeptide comprises an amino acid sequence having at least 90% sequence identity to SEQ ID NO: 15; and
   f) at least one RGN accessory protein having at least 90% sequence identity to any one of SEQ ID NOs: 188-190, wherein said RGN polypeptide comprises an amino acid sequence having at least 90% sequence identity to SEQ ID NO: 16.
326. The method of embodiment 325, wherein said one or more RGN accessory proteins is selected from the group consisting of:
   a) at least one RGN accessory protein having at least 95% sequence identity to any one of SEQ ID NOs: 178-181, wherein said RGN polypeptide comprises an amino acid sequence having at least 95% sequence identity to SEQ ID NO: 11;
   b) at least one RGN accessory protein having at least 95% sequence identity to any one of SEQ ID NOs: 182-184, wherein said RGN polypeptide comprises an amino acid sequence having at least 95% sequence identity to SEQ ID NO: 12;
   c) at least one RGN accessory protein having at least 95% sequence identity to any one of SEQ ID NOs: 185-187, wherein said RGN polypeptide comprises an amino acid sequence having at least 95% sequence identity to SEQ ID NO: 13;
   d) an RGN accessory protein having at least 95% sequence identity to SEQ ID NO: 191, wherein said RGN polypeptide comprises an amino acid sequence having at least 95% sequence identity to SEQ ID NO: 14;
   e) an RGN accessory protein having at least 95% sequence identity to SEQ ID NO: 192, wherein said RGN polypeptide comprises an amino acid sequence having at least 95% sequence identity to SEQ ID NO: 15; and
   f) at least one RGN accessory protein having at least 95% sequence identity to any one of SEQ ID NOs: 188-190, wherein said RGN polypeptide comprises an amino acid sequence having at least 95% sequence identity to SEQ ID NO: 16.
327. The method of embodiment 325, wherein said one or more RGN accessory proteins is selected from the group consisting of:
   a) at least one RGN accessory protein having 100% sequence identity to any one of SEQ ID NOs: 178-181, wherein said RGN polypeptide comprises an amino acid sequence having 100% sequence identity to SEQ ID NO: 11;
   b) at least one RGN accessory protein having 100% sequence identity to any one of SEQ ID NOs: 182-184, wherein said RGN polypeptide comprises an amino acid sequence having 100% sequence identity to SEQ ID NO: 12;
   c) at least one RGN accessory protein having 100% sequence identity to any one of SEQ ID NOs: 185-187, wherein said RGN polypeptide comprises an amino acid sequence having 100% sequence identity to SEQ ID NO: 13;
   d) an RGN accessory protein having 100% sequence identity to SEQ ID NO: 191, wherein said RGN polypeptide comprises an amino acid sequence having 100% sequence identity to SEQ ID NO: 14;
   e) an RGN accessory protein having 100% sequence identity to SEQ ID NO: 192, wherein said RGN polypeptide comprises an amino acid sequence having 100% sequence identity to SEQ ID NO: 15; and f) at least one RGN accessory protein having 100% sequence identity to any one of SEQ ID NOs: 188-190, wherein said RGN polypeptide comprises an amino acid sequence having 100% sequence identity to SEQ ID NO: 16.
328. The method of any one of embodiments 303-327, wherein said RGN polypeptide and said guide RNA are not found complexed to one another in nature.
329. The method of any one of embodiments 303-328, wherein said target DNA sequence is a eukaryotic target DNA sequence.
330. The method of any one of embodiments 303-329, wherein said RGN polypeptide comprises an amino acid sequence having at least 90% sequence identity to SEQ ID NO: 11 and said guide RNA comprises a CRISPR RNA comprising a CRISPR repeat sequence having at least 90% sequence identity to SEQ ID NO: 116.
331. The method of any one of embodiments 303-329, wherein said RGN polypeptide comprises an amino acid sequence having at least 95% sequence identity to SEQ ID NO: 11 and said guide RNA comprises a CRISPR RNA comprising a CRISPR repeat sequence having at least 95% sequence identity to SEQ ID NO: 116.
332. The method of any one of embodiments 303-329, wherein said RGN polypeptide comprises an amino acid sequence having 100% sequence identity to SEQ ID NO: 11 and said guide RNA comprises a CRISPR RNA comprising a CRISPR repeat sequence having 100% sequence identity to SEQ ID NO: 116.
333. The method of any one of embodiments 303-329, wherein said guide RNA comprises a tracrRNA.
334. The method of embodiment 333, wherein said tracrRNA is selected from the group consisting of:
   a) a tracrRNA having at least 90% sequence identity to SEQ ID NO: 120, wherein said guide RNA further comprises a CRISPR RNA comprising a CRISPR repeat sequence having at least 90% sequence identity to SEQ ID NO: 110, and wherein said RGN polypeptide comprises an amino acid sequence having at least 90% sequence identity to SEQ ID NO: 1;
   b) a tracrRNA having at least 90% sequence identity to SEQ ID NO: 121, wherein said guide RNA further comprises a CRISPR RNA comprising a CRISPR repeat sequence having at least 90% sequence identity to SEQ ID NO: 111, and wherein said RGN polypeptide comprises an amino acid sequence having at least 90% sequence identity to SEQ ID NO: 2;
   c) a tracrRNA having at least 90% sequence identity to SEQ ID NO: 122, wherein said guide RNA further comprises a CRISPR RNA comprising a CRISPR repeat sequence having at least 90% sequence identity to SEQ ID NO: 112, and wherein said RGN polypeptide comprises an amino acid sequence having at least 90% sequence identity to SEQ ID NO: 3;
   d) a tracrRNA having at least 90% sequence identity to SEQ ID NO: 123, wherein said guide RNA further comprises a CRISPR RNA comprising a CRISPR repeat sequence having at least 90% sequence identity to SEQ ID NO: 113, and wherein said RGN polypeptide comprises an amino acid sequence having at least 90% sequence identity to SEQ ID NO: 4;
   e) a tracrRNA having at least 90% sequence identity to SEQ ID NO: 124, wherein said guide RNA further comprises a CRISPR RNA comprising a CRISPR repeat sequence having at least 90% sequence identity to SEQ ID NO: 114, and wherein said RGN polypeptide comprises an amino acid sequence having at least 90% sequence identity to SEQ ID NO: 5;
   f) a tracrRNA having at least 90% sequence identity to SEQ ID NO: 125, wherein said guide RNA further comprises a CRISPR RNA comprising a CRISPR repeat sequence having at least 90% sequence identity to SEQ ID NO: 115, and wherein said RGN polypeptide comprises an amino acid sequence having at least 90% sequence identity to SEQ ID NO: 6;
   g) a tracrRNA having at least 90% sequence identity to SEQ ID NO: 126, wherein said guide RNA further comprises a CRISPR RNA comprising a CRISPR repeat sequence having at least 90% sequence identity to SEQ ID NO: 117, and wherein said RGN polypeptide comprises an amino acid sequence having at least 90% sequence identity to SEQ ID NO: 12;
   h) a tracrRNA having at least 90% sequence identity to SEQ ID NO: 127, wherein said guide RNA further comprises a CRISPR RNA comprising a CRISPR repeat sequence having at least 90% sequence identity to SEQ ID NO: 118, and wherein said RGN polypeptide comprises an amino acid sequence having at least 90% sequence identity to SEQ ID NO: 13; and
   i) a tracrRNA having at least 90% sequence identity to SEQ ID NO: 128, wherein said guide RNA further comprises a CRISPR RNA comprising a CRISPR repeat sequence having at least 90% sequence identity to SEQ ID NO: 119, and wherein said RGN polypeptide comprises an amino acid sequence having at least 90% sequence identity to SEQ ID NO: 16.
335. The method of embodiment 333, wherein said tracrRNA is selected from the group consisting of:
   a) a tracrRNA having at least 95% sequence identity to SEQ ID NO: 120, wherein said guide RNA further comprises a CRISPR RNA comprising a CRISPR repeat sequence having at least 95% sequence identity to SEQ ID NO: 110, and wherein said RGN polypeptide comprises an amino acid sequence having at least 95% sequence identity to SEQ ID NO: 1;
   b) a tracrRNA having at least 95% sequence identity to SEQ ID NO: 121, wherein said guide RNA further comprises a CRISPR RNA comprising a CRISPR repeat sequence having at least 95% sequence identity to SEQ ID NO: 111, and wherein said RGN polypeptide comprises an amino acid sequence having at least 95% sequence identity to SEQ ID NO: 2;
   c) a tracrRNA having at least 95% sequence identity to SEQ ID NO: 122, wherein said guide RNA further comprises a CRISPR RNA comprising a CRISPR repeat sequence having at least 95% sequence identity to SEQ ID NO: 112, and wherein said RGN polypeptide comprises an amino acid sequence having at least 95% sequence identity to SEQ ID NO: 3;
   d) a tracrRNA having at least 95% sequence identity to SEQ ID NO: 123, wherein said guide RNA further comprises a CRISPR RNA comprising a CRISPR repeat sequence having at least 95% sequence identity to SEQ ID NO: 113, and wherein said RGN polypeptide comprises an amino acid sequence having at least 95% sequence identity to SEQ ID NO: 4;
   e) a tracrRNA having at least 95% sequence identity to SEQ ID NO: 124, wherein said guide RNA further comprises a CRISPR RNA comprising a CRISPR repeat sequence having at least 95% sequence identity to SEQ ID NO: 114, and wherein said RGN polypeptide comprises an amino acid sequence having at least 95% sequence identity to SEQ ID NO: 5;
   f) a tracrRNA having at least 95% sequence identity to SEQ ID NO: 125, wherein said guide RNA further comprises a CRISPR RNA comprising a CRISPR repeat sequence having at least 95% sequence identity to SEQ ID NO: 115, and wherein said RGN polypeptide comprises an amino acid sequence having at least 95% sequence identity to SEQ ID NO: 6;
   g) a tracrRNA having at least 95% sequence identity to SEQ ID NO: 126, wherein said guide RNA further comprises a CRISPR RNA comprising a CRISPR repeat sequence having at least 95% sequence identity to SEQ ID NO: 117, and wherein said RGN polypeptide comprises an amino acid sequence having at least 95% sequence identity to SEQ ID NO: 12;
   h) a tracrRNA having at least 95% sequence identity to SEQ ID NO: 127, wherein said guide RNA further comprises a CRISPR RNA comprising a CRISPR repeat sequence having at least 95% sequence identity to SEQ ID NO: 118, and wherein said RGN polypeptide comprises an amino acid sequence having at least 95% sequence identity to SEQ ID NO: 13; and
   i) a tracrRNA having at least 95% sequence identity to SEQ ID NO: 128, wherein said guide RNA further comprises a CRISPR RNA comprising a CRISPR repeat sequence having at least 95% sequence identity to SEQ ID NO: 119, and wherein said RGN polypeptide comprises an amino acid sequence having at least 95% sequence identity to SEQ ID NO: 16.
336. The method of embodiment 333, wherein said tracrRNA is selected from the group consisting of:
   a) a tracrRNA having 100% sequence identity to SEQ ID NO: 120, wherein said guide RNA further comprises a CRISPR RNA comprising a CRISPR repeat sequence having 100% sequence identity to SEQ ID NO: 110, and wherein said RGN polypeptide comprises an amino acid sequence having 100% sequence identity to SEQ ID NO: 1;
   b) a tracrRNA having 100% sequence identity to SEQ ID NO: 121, wherein said guide RNA further comprises a CRISPR RNA comprising a CRISPR repeat sequence having 100% sequence identity to SEQ ID NO: 111, and wherein said RGN polypeptide comprises an amino acid sequence having 100% sequence identity to SEQ ID NO: 2;
   c) a tracrRNA having 100% sequence identity to SEQ ID NO: 122, wherein said guide RNA further comprises a CRISPR RNA comprising a CRISPR repeat sequence having 100% sequence identity to SEQ ID NO: 112, and wherein said RGN polypeptide comprises an amino acid sequence having 100% sequence identity to SEQ ID NO: 3;
   d) a tracrRNA having 100% sequence identity to SEQ ID NO: 123, wherein said guide RNA further comprises a CRISPR RNA comprising a CRISPR repeat sequence having 100% sequence identity to SEQ ID NO: 113, and wherein said RGN polypeptide comprises an amino acid sequence having 100% sequence identity to SEQ ID NO: 4;
   e) a tracrRNA having 100% sequence identity to SEQ ID NO: 124, wherein said guide RNA further comprises a CRISPR RNA comprising a CRISPR repeat sequence having 100% sequence identity to SEQ ID NO: 114, and wherein said RGN polypeptide comprises an amino acid sequence having 100% sequence identity to SEQ ID NO: 5;
   f) a tracrRNA having 100% sequence identity to SEQ ID NO: 125, wherein said guide RNA further comprises a CRISPR RNA comprising a CRISPR repeat sequence having 100% sequence identity to SEQ ID NO: 115, and wherein said RGN polypeptide comprises an amino acid sequence having 100% sequence identity to SEQ ID NO: 6;
   g) a tracrRNA having 100% sequence identity to SEQ ID NO: 126, wherein said guide RNA further comprises a CRISPR RNA comprising a CRISPR repeat sequence having 100% sequence identity to SEQ ID NO: 117, and wherein said RGN polypeptide comprises an amino acid sequence having 100% sequence identity to SEQ ID NO: 12;
   h) a tracrRNA having 100% sequence identity to SEQ ID NO: 127, wherein said guide RNA further comprises a CRISPR RNA comprising a CRISPR repeat sequence having 100% sequence identity to SEQ ID NO: 118, and wherein said RGN polypeptide comprises an amino acid sequence having 100% sequence identity to SEQ ID NO: 13; and
   i) a tracrRNA having 100% sequence identity to SEQ ID NO: 128, wherein said guide RNA further comprises a CRISPR RNA comprising a CRISPR repeat sequence having 100% sequence identity to SEQ ID NO: 119, and wherein said RGN polypeptide comprises an amino acid sequence having 100% sequence identity to SEQ ID NO: 16.
337. The method of any one of embodiments 333-336, wherein said guide RNA is a single guide RNA (sgRNA).
338. The method of any one of embodiments 333-336, wherein said guide RNA is a dual-guide RNA.
339. The method of any one of embodiments 303-338, wherein said target DNA sequence is within a region of said DNA molecule that is single-stranded.
340. The method of any one of embodiments 303-338, wherein said target DNA sequence is within a region of said DNA molecule that is double-stranded.
341. The method of embodiment 340, wherein cleavage of said target DNA sequence by said RGN polypeptide generates a double-stranded break.
342. The method of embodiment 340, wherein cleavage of said target DNA sequence by said RGN polypeptide generates a single-stranded break.
343. The method of any one of embodiments 303-342, wherein said target DNA sequence is located adjacent to a protospacer adjacent motif (PAM).
344. A method for producing a genetically modified cell with a correction in a causal mutation for a genetically inherited disease, the method comprising introducing into the cell:
   a) an RNA-guided nuclease (RGN) polypeptide, wherein the RGN polypeptide comprises an amino acid sequence having at least 90% sequence identity to any one of SEQ ID NOs: 1 to 109, or a polynucleotide encoding said RGN polypeptide, wherein said polynucleotide encoding the RGN polypeptide is operably linked to a promoter to enable expression of the RGN polypeptide in the cell; and
   b) a guide RNA (gRNA), wherein the gRNA comprises a CRISPR repeat sequence comprising a nucleotide sequence having at least 90% sequence identity to any one of SEQ ID NOs: 110 to 119, or a polynucleotide encoding said gRNA, wherein said polynucleotide encoding the gRNA is operably linked to a promoter to enable expression of the gRNA in the cell; whereby the RGN and gRNA target to the genomic location of the causal mutation and modify the genomic sequence to remove the causal mutation.
345. The method of embodiment 344, wherein said RGN polypeptide comprises an amino acid sequence having at least 95% sequence identity to any one of SEQ ID NOs: 1 to 109 and said CRISPR repeat sequence comprises a nucleotide sequence having at least 95% sequence identity to any one of SEQ ID NOs: 110 to 119.
346. The method of embodiment 344, wherein said RGN polypeptide comprises an amino acid sequence having 100% sequence identity to any one of SEQ ID NOs: 1 to 109 and said CRISPR repeat sequence comprises a nucleotide sequence having 100% sequence identity to any one of SEQ ID NOs: 110 to 119.
347. The method of any one of embodiments 344-346, wherein the RGN is operably linked to a base-editing polypeptide.
348. The method of embodiment 347, wherein the base-editing polypeptide is a deaminase.
349. The method of any one of embodiments 344-348, wherein the cell is an animal cell.
350. The method of any one of embodiments 344-348, wherein the cell is a mammalian cell.
351. The method of embodiment 349, wherein the cell is derived from a dog, cat, mouse, rat, rabbit, horse, cow, pig, or human.
352. The method of embodiment 349, wherein the genetically inherited disease is caused by a single nucleotide polymorphism.
353. The method of embodiment 351, wherein the genetically inherited disease is Hurler syndrome.
354. The method of embodiment 353, wherein the gRNA further comprises a spacer sequence that targets a region proximal to the causal single nucleotide polymorphism.
355. A method for producing a genetically modified cell with a deletion in a disease-causing genomic region of instability, the method comprising introducing into the cell:
   a) an RNA-guided nuclease (RGN) polypeptide, wherein the RGN polypeptide comprises an amino acid sequence having at least 90% sequence identity to any one of SEQ ID NOs: 1 to 109, or a polynucleotide encoding said RGN polypeptide, wherein said polynucleotide encoding the RGN polypeptide is operably linked to a promoter to enable expression of the RGN polypeptide in the cell; and
   b) a first guide RNA (gRNA), wherein the first gRNA comprises a CRISPR repeat sequence comprising a nucleotide sequence having at least 90% sequence identity to any one of SEQ ID NOs: 110 to 119, or a polynucleotide encoding said first gRNA, wherein said polynucleotide encoding the first gRNA is operably linked to a promoter to enable expression of the first gRNA in the cell, and further wherein the first gRNA comprises a spacer sequence that targets the 5’flank of the genomic region of instability; and
   c) a second guide RNA (gRNA), wherein the second gRNA comprises a CRISPR repeat sequence comprising a nucleotide sequence having at least 90% sequence identity to any one of SEQ ID NOs: 110 to 119, or a polynucleotide encoding said second gRNA, wherein said polynucleotide encoding the second gRNA is operably linked to a promoter to enable expression of the second gRNA in the cell, and further wherein said second gRNA comprises a spacer sequence that targets the 3’flank of the genomic region of instability;
   whereby the RGN and the first and second gRNAs target to the genomic region of instability and atleast a portion of the genomic region of instability is removed.
356. The method of embodiment 355, wherein said RGN polypeptide comprises an amino acid sequence having at least 95% sequence identity to any one of SEQ ID NOs: 1 to 109 and said CRISPR repeat sequence of said first gRNA and said second gRNA comprises a nucleotide sequence having at least 95% sequence identity to any one of SEQ ID NOs: 110 to 119.
357. The method of embodiment 355, wherein said RGN polypeptide comprises an amino acid sequence having 100% sequence identity to any one of SEQ ID NOs: 1 to 109 and said CRISPR repeat sequence of said first gRNA and said second gRNA comprises a nucleotide sequence having 100% sequence identity to any one of SEQ ID NOs: 110 to 119.
358. The method of any one of embodiments 355-357, wherein the cell is an animal cell.
359. The method of any one of embodiments 355-357, wherein the cell is a mammalian cell.
360. The method of embodiment 359, wherein the cell is derived from a dog, cat, mouse, rat, rabbit, horse, cow, pig, or human.
361. The method of embodiment 358, wherein the genetically inherited disease is Friedrich’s Ataxia or Huntington’s Disease.
362. The method of embodiment 358, wherein the spacer sequence of the first gRNA further targets a region within or proximal to the genomic region of instability.
363. The method of embodiment 362, wherein the spacer sequence of the second gRNA further targets a region within or proximal to the genomic region of instability.
364. A method for producing a genetically modified mammalian hematopoietic progenitor cell having decreased BCL11A mRNA and protein expression, the method comprising introducing into an isolated human hematopoietic progenitor cell:
   a) an RNA-guided nuclease (RGN) polypeptide, wherein the RGN polypeptide comprises an amino acid sequence having at least 90% sequence identity to any one of SEQ ID NOs: 1-109, or a polynucleotide encoding said RGN polypeptide, wherein said polynucleotide encoding the RGN polypeptide is operably linked to a promoter to enable expression of the RGN polypeptide in the cell; and
   b) a guide RNA (gRNA), wherein the gRNA comprises a CRISPR repeat sequence comprising a nucleotide sequence having at least 90% sequence identity to any one of SEQ ID NOs: 110-119, or a polynucleotide encoding said gRNA, wherein said polynucleotide encoding the gRNA is operably linked to a promoter to enable expression of the gRNA in the cell,
   whereby the RGN and gRNA are expressed in the cell and cleave at the BCL11A enhancer region, resulting in genetic modification of the human hematopoietic progenitor cell and reducing the mRNA and/or protein expression of BCL11A.
365. The method of embodiment 364, wherein said RGN polypeptide comprises an amino acid sequence having at least 95% sequence identity to any one of SEQ ID NOs: 1-109 and said CRISPR repeat sequence comprises a nucleotide sequence having at least 95% sequence identity to any one of SEQ ID NOs: 110-119.
366. The method of embodiment 364, wherein said RGN polypeptide comprises an amino acid sequence having 100% sequence identity to any one of SEQ ID NOs: 1-109 and said CRISPR repeat sequence comprises a nucleotide sequence having 100% sequence identity to any one of SEQ ID NOs: 110-119.
367. The method of any one of embodiments 364-366, wherein the gRNA further comprises a spacer sequence that targets a region within or proximal to the BCL11A enhancer region.
368. The method of any one of embodiments 344-367, wherein the guide RNA, first guide RNA, and second guide RNA comprises a tracrRNA.
369. The method of embodiment 368, wherein said tracrRNA comprises a nucleotide sequence having at least 90% sequence identity to any one of SEQ ID NOs: 120 to 128.
370. The method of embodiment 368, wherein said tracrRNA comprises a nucleotide sequence having at least 95% sequence identity to any one of SEQ ID NOs: 120 to 128.
371. The method of embodiment 368, wherein said tracrRNA comprises a nucleotide sequence having 100% sequence identity to any one of SEQ ID NOs: 120 to 128.
372. A method of treating a disease, said method comprising administering to a subject in need thereof an effective amount of a pharmaceutical composition of embodiment 214 or 277.
373. The method of embodiment 372, wherein said disease is associated with a causal mutation and said effective amount of said pharmaceutical composition corrects said causal mutation. 374. Use of the nucleic acid molecule of any one of embodiments 1-14, 50-52, and 111-113, the vector of any one of embodiments 15-28, 53-110, and 114-171, the cell of any one of embodiments 29, 266, and 270-274, the isolated RGN polypeptide of any one of embodiments 37-49, or the system of any one of embodiments 172-213 for the treatment of a disease in a subject.
375. The use of embodiment 374, wherein said disease is associated with a causal mutation and said treating comprises correcting said causal mutation.
376. Use of the nucleic acid molecule of any one of embodiments 1-14, 50-52, and 111-113, the vector of any one of embodiments 15-28, 53-110, and 114-171, the cell of any one of embodiments 29, 266, and 270-274, the isolated RGN polypeptide of any one of embodiments 37-49, or the system of any one of embodiments 172-213 for the manufacture of a medicament useful for treating a disease.
377. The use of embodiment 376, wherein said disease is associated with a causal mutation and an effective amount of said medicament corrects said causal mutation.

The following examples are offered by way of illustration and not by way of limitation.

### EXPERIMENTAL

### Example 1. Identification of RNA-Guided Nucleases

CRISPR-associated sequences with sequence similarity to transposases were identified in genomes of interest. CRISPR repeats were identified by minCED (mining CRISPRs in Environmental Datasets) with the minimum number of repeats in array set to two. Only putative RNA-guided nucleases that co-localize with repeats on the same contig were considered for further investigation. Several increasingly stringent cutoffs for distance between repeats and the putative cas gene on a contig (100 kb, 50 kb, 20 kb, 10 kb) were used. The final filter of 5 kb was selected.

In the literature, CRISPR repeats in active systems are between 27 and 47 nucleotides in length. This feature was used to filter and remove non-CRISPR repetitive features. Part of the acquisition of new spacer sequences in a CRISPR array requires that the first nucleotide of the repeat be a G in order to provide the proper chemistry for array expansion. As a part of this step, the consensus repeat sequence was predicted, as well as the orientation of the repeat-spacer array. This filter was included to prioritize likely functional RGNs. The minimum number of repeats required in an array was increased to three.

Some proteins, mainly DNA-binding proteins, have repeating amino acids in their primary structure that can be falsely detected as CRISPR loci. Putative RGNs whose repetitive features occurred internally in a protein were discarded. Only intergenic repeats were considered for further analysis.

To determine clusters of homologs, the proteins and consensus repeat sequences were aligned and categorized into clusters based on their phylogeny. Repeats and proteins tend to co-cluster phylogenetically, lending weight to the concept of clusters of homologs. The protein cluster information is also displayed in Table 1. Proteins were clustered at 95% identity using cdhit.

The relatedness of strains can be traced by comparing their spacer content. If systems have the same repeat sequence and similar protein sequences, they are said to be related and their spacer content can provide information about their shared history. Homologs in the same clusters tend to share conserved ancestral spacers. Divergent strains have entirely unique spacer content from one another. Clonal isolates will completely share the exact same spacer content. Systems whose repeat sequences are conserved, but whose spacer content varies between genome can promote systems that are more likely to be active and these were prioritized.

109 distinct CRISPR-associated RNA-guided nucleases (RGNs) were identified and are described in Table 1 below. Table 1 provides the name of each RGN, its amino acid sequence, the source from which it was derived, and processed crRNA and tracrRNA sequences.

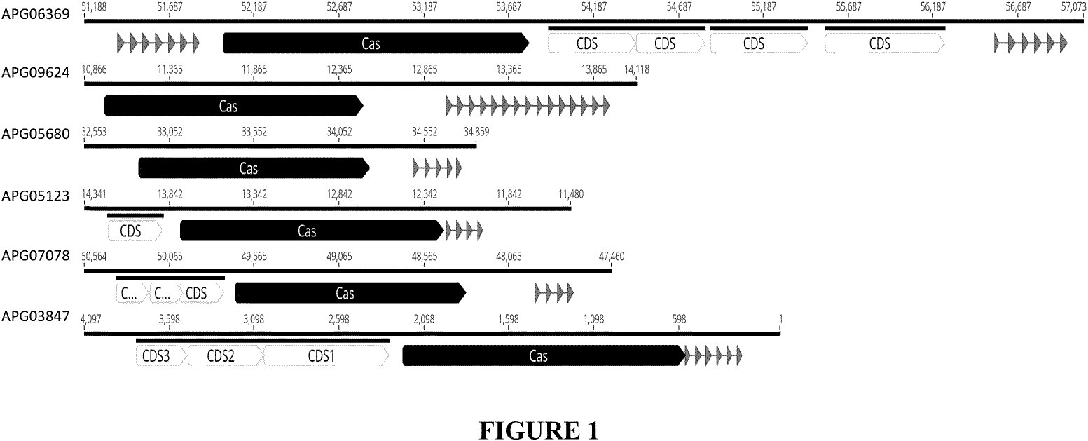

The locus surrounding each putative nuclease was searched for potential accessory genes that might be needed for CRISPR immunity. Several putative nucleases appeared to be in operonic structures with potential accessory genes, but no loci contained homologs to cas1, cas2, cas4 or other known cas genes (FIG. 1). Additionally, several systems contained repeats that were flush with the end of the nuclease and lacked the expected leader sequence upstream of the CRISPR repeats, suggesting a novel mechanism of CRISPR RNA expression.

TABLE 1

RGN system information

| RGN ID | SEQ ID NO. | Source | Prot Cluster | crRNA repeat seq (SEQ ID NO.) | tracrRNA (SEQ ID NO.) | sgRNA (SEQ ID NO) | Consensus Repeat (SEQ ID NO.) |
|---|---|---|---|---|---|---|---|
| APG07339 | 1 | Bacillus sp. | 4 | 110 | 120 | 129 | 201 |
| APG09624 | 2 | Bacillus sp. | 4 | 111 | 121 | 130 | 202 |
| APG03003 | 3 | Bacillus sp. | 4 | 112 | 122 | | 203 |
| APG05405 | 4 | Bacillus sp. | 4 | 113 | 123 | 131 | 204 |
| APG09777 | 5 | Bacillus sp. | 4 | 114 | 124 | 132 | 205 |
| APG05680 | 6 | Bacillus sp. | 16 | 115 | 125 | 310 | 206 |
| APG02119 | 7 | Bacillus sp. | 4 | | | | 207 |
| APG03285 | 8 | Bacillus sp. | 15 | | | | 208 |
| APG04998 | 9 | Bacillus sp. | 11 | | | | 209 |
| APG07078 | 10 | Bacillus sp. | 13 | | | | 210 |
| APG06369 | 11 | Gordoniasp. | 0 | 116 | | | 211 |
| APG03847 | 12 | Micrococcussp. | 2 | 117 | 126 | 133 | 212 |
| APG05625 | 13 | Micrococcussp. | 2 | 118 | 127 | 134 | 213 |
| APG03759 | 14 | Paeniglutamicibacter sp. | 5 | | | | 214 |
| APG05123 | 15 | Streptomycessp. | 3 | | | | 215 |
| APG03524 | 16 | Micrococcussp. | 2 | 119 | 128 | 135 | 216 |
| APG05361 | 17 | Bacillus sp. | 1 | | | | 217 |
| APG04303 | 18 | Bacillus sp. | 14 | | | | 218 |
| APG04291 | 19 | Bacillus sp. | 14 | | | | 219 |
| APG01006 | 20 | Bacillus sp. | 14 | | | | 220 |
| APG06547 | 21 | Bacillus sp. | 22 | | | | 221 |
| APG01699 | 22 | Bacillus sp. | 14 | | | | 222 |
| APG06155 | 23 | Bacillus sp. | 14 | | | | 223 |
| APG09116 | 24 | Bacillus sp. | 4 | | | | 224 |
| APG09403 | 25 | Bacillus sp. | 23 | | | | 225 |
| APG08954 | 26 | Bacillus sp. | 14 | | | | 226 |
| APG02589 | 27 | Bacillus sp. | 14 | | | | 227 |
| APG04061 | 28 | Bacillus sp. | 14 | | | | 228 |
| APG08773 | 29 | Bacillus sp. | 12 | | | | 229 |
| APG02836 | 30 | Bacillus sp. | 14 | | | | 230 |
| APG09123 | 31 | Bacillus sp. | 14 | | | | 231 |
| APG04288 | 32 | Bacillus sp. | 14 | | | | 232 |
| APG06873 | 33 | Bacillus sp. | 12 | | | | 233 |
| APG02381 | 34 | Bacillus sp. | 14 | | | | 234 |
| APG06947 | 35 | Bacillus sp. | 27 | | | | ND |
| APG04677 | 36 | Bacillus sp. | 14 | | | | 236 |
| APG07253 | 37 | Bacillus sp. | 14 | | | | 237 |
| APG08319 | 38 | Bacillus sp. | 24 | | | | ND |
| APG04362 | 39 | Bacillus sp. | 14 | | | | 239 |
| APG00992 | 40 | Bacillus sp. | 14 | | | | 240 |
| APG04193 | 41 | Bacillus sp. | 17 | | | | 241 |
| APG08201 | 42 | Bacillus sp. | 17 | | | | 242 |
| APG01031 | 43 | Bacillus sp. | 14 | | | | 243 |
| APG06773 | 44 | Bacillus sp. | 14 | | | | 244 |
| APG08945 | 45 | Bacillus sp. | 14 | | | | 245 |
| APG03214 | 46 | Bacillus sp. | 4 | | | | 246 |
| APG09942 | 47 | Bacillus sp. | 4 | | | | 247 |
| APG01836 | 48 | Bacillus sp. | 14 | | | | 248 |
| APG06336 | 49 | Bacillus sp. | 14 | | | | 249 |
| APG08839 | 50 | Bacillus sp. | 4 | | | | 250 |
| APG02684 | 51 | Bacillus sp. | 20 | | | | ND |
| APG05281 | 52 | Bacillus sp. | 14 | | | | 252 |
| APG01046 | 53 | Bacillus sp. | 14 | | | | 253 |
| APG01240 | 54 | Bacillus sp. | 14 | | | | 254 |
| APG05981 | 55 | Bacillus sp. | 14 | | | | 255 |
| APG04054 | 56 | Bacillus sp. | 14 | | | | 256 |
| APG07301 | 57 | Bacillus sp. | 14 | | | | 257 |
| APG07284 | 58 | Bacillus sp. | 14 | | | | 258 |
| APG06812 | 59 | Bacillus sp. | 14 | | | | 259 |
| APG08143 | 60 | Bacillus sp. | 14 | | | | 260 |
| APG08031 | 61 | Bacillus sp. | 14 | | | | 261 |
| APG03966 | 62 | Bacillus sp. | 14 | | | | 262 |
| APG05371 | 63 | Bacillus sp. | 14 | | | | 263 |
| APG04324 | 64 | Bacillus sp. | 14 | | | | 264 |
| APG01233 | 65 | Bacillus sp. | 14 | | | | 265 |
| APG00823 | 66 | Bacillus sp. | 14 | | | | 266 |
| APG06704 | 67 | Bacillus sp. | 14 | | | | 267 |
| APG02228 | 68 | Bacillus sp. | 14 | | | | 268 |
| APG08636 | 69 | Bacillus sp. | 6 | | | | 269 |
| APG02665 | 70 | Bacillus sp. | 6 | | | | 270 |
| APG01832 | 71 | Bacillus sp. | 6 | | | | 271 |
| APG03625 | 72 | Bacillus sp. | 14 | | | | 272 |
| APG07479 | 73 | Bacillus sp. | 14 | | | | 273 |
| APG02608 | 74 | Bacillus sp. | 14 | | | | 274 |
| APG04337 | 75 | Bacillus sp. | 12 | | | | 275 |
| APG01431 | 76 | Bacillus sp. | 14 | | | | 276 |
| APG05423 | 77 | Bacillus sp. | 14 | | | | 277 |
| APG01452 | 78 | Bacillus sp. | 14 | | | | 278 |
| APG05729 | 79 | Bacillus sp. | 19 | | | | 279 |
| APG01946 | 80 | Bacillus sp. | 14 | | | | 280 |
| APG02414 | 81 | Bacillus sp. | 14 | | | | 281 |
| APG01839 | 82 | Bacillus sp. | 14 | | | | 282 |
| APG00752 | 83 | Bacillus sp. | 14 | | | | 283 |
| APG02156 | 84 | Bacillus sp. | 14 | | | | 284 |
| APG08789 | 85 | Bacillus sp. | 10 | | | | 285 |
| APG07736 | 86 | Bacillus sp. | 26 | | | | 286 |
| APG01573 | 87 | Bacillus sp. | 14 | | | | 287 |
| APG07722 | 88 | Bacillus sp. | 18 | | | | 288 |
| APG08071 | 89 | Bacillus sp. | 8 | | | | 289 |
| APG01280 | 90 | Bacillus sp. | 14 | | | | 290 |
| APG07455 | 91 | Bacillus sp. | 14 | | | | 291 |
| APG05150 | 92 | Bacillus sp. | 7 | | | | 292 |
| APG09405 | 93 | Bacillus sp. | 14 | | | | 293 |
| APG09583 | 94 | Bacillus sp. | 4 | | | | 294 |
| APG09909 | 95 | Bacillus sp. | 14 | | | | 295 |
| APG07075 | 96 | Bacillus sp. | 14 | | | | 296 |
| APG05892 | 97 | Bacillus sp. | 14 | | | | 297 |
| APG09648 | 98 | Bacillus sp. | 25 | | | | 298 |
| APG02311 | 99 | Bacillus sp. | 21 | | | | 299 |
| APG03906 | 100 | Bacillus sp. | 14 | | | | 300 |
| APG01953 | 101 | Bacillus sp. | 9 | | | | 301 |
| APG00903 | 102 | Bacillus sp. | 14 | | | | 302 |
| APG01543 | 103 | Bacillus sp. | 22 | | | | 303 |
| APG07261 | 104 | Bacillus sp. | 15 | | | | ND |
| APG05635 | 105 | Bacillus sp. | 13 | | | | 305 |
| APG02962 | 106 | Bacillus sp. | 15 | | | | 306 |
| APG07448 | 107 | Bacillus sp. | 15 | | | | 307 |
| APG05378 | 108 | Bacillus sp. | 16 | | | | 308 |
| APG01852 | 109 | Bacillus sp. | 16 | | | | 309 |

ND=Not Determined

### Example 2: Protein Analysis

Nuclease domains were predicted by searching for domains in the interpro database. Split RuvC nuclease domains were predicted using hmm nuclease domain profiles built on split RuvC domains from known Cas proteins. The predicted nuclease residues can be found in Table 2 (ND = Not Determined).

TABLE 2

Nuclease residues

| | | | |
|---|---|---|---|
| RGN ID | RuvC-I | RuvC-II | RuvC-III |
| APG07339 | D297 | E395 | D477 |
| APG09624 | D297 | E395 | D477 |
| APG03003 | D297 | E395 | D477 |
| APG05405 | D297 | E395 | D477 |
| APG09777 | D297 | E395 | D477 |
| APG05680 | D232 | E331 | D407 |
| APG02119 | D297 | E395 | D477 |
| APG03285 | D232 | E331 | D407 |
| APG04998 | D281 | E379 | D456 |
| APG07078 | D233 | E332 | D408 |
| APG06369 | ND | ND | ND |
| APG03847 | D282 | E382 | D463 |
| APG05625 | D282 | E382 | D463 |
| APG03759 | D257 | E357 | D438 |
| APG05123 | D248 | E356 | D437 |
| APG03524 | D282 | E382 | D463 |
| APG05361 | D244 | D362 | D443 |
| APG04303 | D233 | E332 | D408 |
| APG04291 | D233 | E332 | D408 |
| APG01006 | D233 | E332 | D408 |
| APG06547 | D183 | E277 | D359 |
| APG01699 | D233 | E326 | D402 |
| APG06155 | D233 | E332 | D408 |
| APG09116 | D297 | E395 | D477 |
| APG09403 | D183 | E277 | D359 |
| APG08954 | D233 | E326 | D402 |
| APG02589 | D236 | E329 | D405 |
| APG04061 | D233 | E332 | D408 |
| APG08773 | D267 | E363 | D440 |
| APG02836 | D233 | E332 | D408 |
| APG09123 | D233 | E332 | D408 |
| APG04288 | D233 | E332 | D408 |
| APG06873 | D267 | E363 | D440 |
| APG02381 | D233 | E332 | D408 |
| APG06947 | D2 | E95 | D184 |
| APG04677 | D233 | E326 | D402 |
| APG07253 | D233 | E332 | D408 |
| APG08319 | D183 | E267 | D350 |
| APG04362 | D233 | E332 | D408 |
| APG00992 | D233 | E332 | D408 |
| APG04193 | D232 | E331 | D407 |
| APG08201 | D232 | E331 | D407 |
| APG01031 | D226 | E325 | D401 |
| APG06773 | D233 | E332 | D408 |
| APG08945 | D233 | E332 | D408 |
| APG03214 | D297 | E395 | D477 |
| APG09942 | D297 | E395 | D477 |
| APG01836 | D233 | E332 | D408 |
| APG06336 | D233 | E332 | D408 |
| APG08839 | D297 | E395 | D477 |
| APG02684 | D221 | E305 | D387 |
| APG05281 | D34 | E133 | D209 |
| APG01046 | D233 | E332 | D408 |
| APG01240 | D233 | E326 | D402 |
| APG05981 | D233 | E332 | D408 |
| APG04054 | D233 | E332 | D408 |
| APG07301 | D233 | E332 | D408 |
| APG07284 | D233 | E326 | D402 |
| APG06812 | D233 | E326 | D402 |
| APG08143 | D233 | E332 | D408 |
| APG08031 | D233 | E332 | D408 |
| APG03966 | D233 | E332 | D408 |
| APG05371 | D233 | E332 | D408 |
| APG04324 | D233 | E326 | D402 |
| APG01233 | D233 | E332 | D408 |
| APG00823 | D233 | E332 | D408 |
| APG06704 | D233 | E332 | D408 |
| APG02228 | D233 | E332 | D408 |
| APG08636 | D267 | E365 | D442 |
| APG02665 | D267 | E365 | D442 |
| APG01832 | D267 | E365 | D442 |
| APG03625 | D233 | E332 | D408 |
| APG07479 | D233 | E332 | D408 |
| APG02608 | D233 | E332 | D408 |
| APG04337 | D267 | E365 | D442 |
| APG01431 | D233 | E332 | D408 |
| APG05423 | D233 | E332 | D408 |
| APG01452 | D233 | E332 | D408 |
| APG05729 | D231 | E330 | D409 |
| APG01946 | D233 | E332 | D408 |
| APG02414 | D226 | E325 | D401 |
| APG01839 | D236 | E329 | D405 |
| APG00752 | D226 | E325 | D401 |
| APG02156 | D236 | E329 | D405 |
| APG08789 | D280 | E378 | D455 |
| APG07736 | D52 | E145 | D221 |
| APG01573 | D226 | E319 | D395 |
| APG07722 | D232 | E331 | D407 |
| APG08071 | D281 | E379 | D456 |
| APG01280 | D233 | E332 | D408 |
| APG07455 | D233 | E332 | D408 |
| APG05150 | D267 | E365 | D442 |
| APG09405 | D233 | E326 | D402 |
| APG09583 | D297 | E395 | D477 |
| APG09909 | D233 | E332 | D408 |
| APG07075 | D233 | E326 | D402 |
| APG05892 | D226 | E319 | D395 |
| APG09648 | D86 | E170 | D253 |
| APG02311 | D221 | E305 | D387 |
| APG03906 | D233 | E332 | D408 |
| APG01953 | D281 | E379 | D456 |
| APG00903 | D233 | E332 | D408 |
| APG01543 | D183 | E277 | D359 |
| APG07261 | D232 | E331 | D407 |
| APG05635 | D233 | E332 | D408 |
| APG02962 | D232 | E331 | D407 |
| APG07448 | D232 | E331 | D407 |
| APG05378 | D232 | E331 | D407 |
| APG01852 | D232 | E331 | D407 |

### Example 3: Guide RNA Prediction and Confirmation

Cultures of bacteria that natively express the RNA-guided nuclease system under investigation were grown to mid-log phase (OD600 of ~0.600), pelleted, and flash frozen. RNA was isolated from the pellets using a mirVANA miRNA Isolation Kit (Life Technologies, Carlsbad, CA), and sequencing libraries were prepared from the isolated RNA using an NEBNext Small RNA Library Prep kit (NEB, Beverly, MA). The library prep was fractionated on a 6% polyacrylamide gel to capture the RNA species less than 200 nt to detect crRNAs and tracrRNAs, respectively. Deep sequencing (75 bp paired-end) was performed on a Next Seq 500 (High Output kit) by a service provider (MoGene, St. Louis, MO). Reads were quality trimmed using Cutadapt and mapped to reference genomes using Bowtie2. A custom RNAseq pipeline was written in python to detect the crRNA and tracrRNA transcripts. Processed crRNA boundaries were determined by sequence coverage of the native repeat spacer array. The anti-repeat portion of the tracrRNA was identified using permissive BLASTn parameters. RNA sequencing depth confirmed the boundaries of the processed tracrRNA by identifying the transcript containing the anti-repeat. Manual curation of RNAs was performed using secondary structure prediction by RNAfold, an RNA folding software. sgRNA cassettes were prepared by DNA synthesis and were generally designed as follows (5′->3′): the processed tracrRNA, operably linked at its 3′ end to a 4 bp noncomplementary linker (AAAG; SEQ ID NO: 136), operably linked at its 3′ end to the processed repeat portion of the crRNA, operably linked at its 3′ end to a 20-30 bp spacer sequence. Other 4 bp noncomplementary linkers may also be used.

For in vitro assays, sgRNAs and some tracrRNAs were synthesized by in vitro transcription of the sgRNA cassettes with a TranscriptAid T7 High Yield Transcription Kit (ThermoFisher). crRNAs and some tracrRNAs were produced synthetically.

For protein expression and purification, plasmids containing the putative RGNs fused to a C terminal His 10 tag were constructed and transformed into BL21 (DE3) strains of E. coli. Expression was performed using Magic Media self-inducing media supplemented with kanamycin. After lysis and clarification, the proteins were purified by immobilized metal affinity chromatography. Further purification of APG05405 was performed using Heparin chromatography.

The longer of the tracrRNAs (SEQ ID NOs: 140, 145, and 147) were produced by in vitro transcription (IVT) using a dsDNA template with T7 promoter upstream of the tracrRNA sequence. The template for IVT was amplified by PCR from a synthesized gBlock template (Integrated DNA Technologies). Shorter tracr and crRNAs were produced synthetically.

RNA binding was confirmed by differential scanning fluorimetry (Niesen, F.H., H. Berglund, and M. Vedadi. 2007. Nat. Protoc. 2: 2212-2221). Dual RNA complexes were produced by mixing an excess of crRNA with tracrRNA in Annealing Buffer (Synthego, 60 mM KCl 6 mM HEPES pH 7). The candidate effector protein and guide RNA (either dual RNA complex or sgRNA) were incubated at final concentrations of 0.5 µM effector protein and 1 µM guide RNA in phosphate buffered saline (PBS, Thermo Fisher). These were incubated for 20 minutes at room temperature and then mixed 1:1 with Sypro Orange dye solution that had been diluted in PBS. A melt curve was obtained measuring fluorescence intensity (FI) as a function of temperature and the first derivative of the melt curve (dFI/dT) was calculated. A shift in the plot of dFI/dT as a function of temperature of the putative RNP relative to the original nuclease indicates RNA binding and was used to evaluate putative guide RNA combinations. Of the original guide combinations identified, only the full length putative tracrRNA and crRNA were observed to induce a significant shift in the dFI/dT vs. temperature functions for RGNs APG09624 and APG05405. Small shifts in this function were observed for other protein/RNA combinations but were smaller in magnitude than previously observed for functional RNP formation and were not interpreted as indicative of the formation of a functional complex. Peak 1 refers to the temperature associated with the largest observed peak for the given sample. If a second peak is observed, it is indicated in the Peak 2 column. Interpretation of the data regarding the formation of a complex is indicated in the “Binding?” column Table 3. “Yes” indicates binding was observed. “N/A” indicates sufficient data was not available to determine if binding could occur.

TABLE 3

Binding of RGNs to guide RNA

| RGN | crRNA (SEQ ID NO.) | tracrRNA (SEQ ID NO.) | Peak 1 (°C) | Peak 2 (°C) | Binding? |
|---|---|---|---|---|---|
| APG03003 | None | None | 38 | Not obs | N/A |
| APG03003 | 139 | 140 | 38 | Not obs | N/A |
| APG03003 | 141 | 142 | 37 | Not obs | N/A |
| APG09777 | None | None | 39 | Not obs | N/A |
| APG09777 | 139 | 140 | 40 | Not obs | N/A |
| APG09777 | 141 | 142 | 39 | Not obs | N/A |
| APG07339 | None | None | 37 | Not obs | N/A |
| APG07339 | 143 | 144 | 37 | Not obs | N/A |
| APG07339 | 143 | 145 | 38 | Not obs | N/A |
| APG09624 | None | None | 38 | Not obs | N/A |
| APG09624 | 143 | 144 | 37 | Not obs | N/A |
| APG09624 | 143 | 145 | 39 | 44 | Yes |
| APG05405 | None | None | 36 | Not obs | N/A |
| APG05405 | 143 | 144 | 37 | Not obs | N/A |
| APG05405 | 143 | 145 | 43 | Not obs | Yes |
| APG09777 | None | None | 38 | Not obs | N/A |
| APG09777 | 146 | 148 | 39 | Not obs | N/A |
| APG09777 | 146 | 147 | 39 | Not obs | N/A |

### Example 4: Direct ssDNA Target Cleavage

Purified APG09624, APG05405, and catalytically inactivated APG05405 (dAPG05405 set forth as SEQ ID NO: 173) were incubated with single guide RNA (sgRNA) Gs.2 (set forth as SEQ ID NO: 194) in Cutsmart buffer (New England Biolabs B7204S) at a final concentration of 200 nM nuclease and 400 nM sgRNA for 20 minutes. These were then added at a final concentration of 100 nM nuclease to solutions of 5′ Cy5 labeled ssDNA referred to herein as LE111 (set forth as SEQ ID NO: 195) or LE113 (set forth as SEQ ID NO: 196) at 10 nM in 1.5X Cutsmart buffer (New England Biolabs B7204S).

Samples were quenched by adding RNase and EDTA at a final concentration of 0.1 mg/mL and 45 mM, respectively, and placed on ice at the following timepoints: 0, 40, 80, and 120 min. After quenching all samples, they were incubated at 50° C. for 30 min, then 95° C. for 5 min. One-fifth volume of loading buffer (1x TBE, 12% Ficoll, 7 M urea) was added to each reaction and incubated at 95° C. for 15 min, and 5 µl of each reaction were analyzed on 15% TBE-urea acrylamide gel (Bio-Rad 3450092).

Quantitation of cleaved product as a function of time, nuclease, and guide RNA is shown below in Table 4. The sequence of LE111 (SEQ ID NO: 195) was used as a negative control, while the sequence of LE113 (SEQ ID NO: 196) bears a target sequence for the sgRNA that was loaded onto the nuclease.

TABLE 4

Cleavage of Target Oligonucleotide

| Nuclease | Target oligonucleotide | Time (min) | Cleavage % |
|---|---|---|---|
| dAPG05405 | LE111 | 0 | 0 |
| dAPG05405 | LE111 | 0 | 0 |
| dAPG05405 | LE111 | 40 | 0 |
| dAPG05405 | LE111 | 40 | 0 |
| dAPG05405 | LE111 | 80 | 0 |
| dAPG05405 | LE111 | 80 | 0 |
| dAPG05405 | LE111 | 120 | 0 |
| dAPG05405 | LE111 | 120 | 0 |
| dAPG05405 | LE113 | 0 | 0 |
| dAPG05405 | LE113 | 0 | 0 |
| dAPG05405 | LE113 | 40 | 0 |
| dAPG05405 | LE113 | 40 | 0 |
| dAPG05405 | LE113 | 80 | 0 |
| dAPG05405 | LE113 | 80 | 0 |
| dAPG05405 | LE113 | 120 | 0 |
| dAPG05405 | LE113 | 120 | 0 |
| APG09624 | LE111 | 0 | 0 |
| APG09624 | LE111 | 0 | 0 |
| APG09624 | LE111 | 40 | 0 |
| APG09624 | LE111 | 40 | 0 |
| APG09624 | LE111 | 80 | 24 |
| APG09624 | LE111 | 80 | 24 |
| APG09624 | LE111 | 120 | 30 |
| APG09624 | LE111 | 120 | 26 |
| APG09624 | LE113 | 0 | 0 |
| APG09624 | LE113 | 0 | 0 |
| APG09624 | LE113 | 40 | 59 |
| APG09624 | LE113 | 40 | 65 |
| APG09624 | LE113 | 80 | 80 |
| APG09624 | LE113 | 80 | 84 |
| APG09624 | LE113 | 120 | 92 |
| APG09624 | LE113 | 120 | 93 |
| APG05405 | LE111 | 0 | 0 |
| APG05405 | LE111 | 0 | 0 |
| APG05405 | LE111 | 40 | 0 |
| APG05405 | LE111 | 40 | 0 |
| APG05405 | LE111 | 80 | 0 |
| APG05405 | LE111 | 80 | 0 |
| APG05405 | LE111 | 120 | 0 |
| APG05405 | LE111 | 120 | 0 |
| APG05405 | LE113 | 0 | 0 |
| APG05405 | LE113 | 0 | 0 |
| APG05405 | LE113 | 40 | 58 |
| APG05405 | LE113 | 40 | 49 |
| APG05405 | LE113 | 80 | 80 |
| APG05405 | LE113 | 80 | 85 |
| APG05405 | LE113 | 120 | 73 |
| APG05405 | LE113 | 120 | 88 |

Gel analysis revealed time-dependent formation of cleavage products primarily in the samples targeted by the sgRNA and with catalytically active nuclease, demonstrating the RNA-guided nuclease activity of these proteins and providing evidence that the critical catalytic residues are correctly defined. Some non-specific activity is observed when the APG09624 RNP is incubated with LE111, which may be due to the fact that the batches of dAPG05405 and APG05405 were purified by an additional chromatography step, and thus the batch of APG09624 may have some level of background activity due to incomplete removal of nucleases that derived from the expression host.

### Example 5: Programmable DNA Binding and Gene Activation

### Construction of RGN Gene Activation and gRNA Mammalian Expression Plasmids

Nuclease constructs for mammalian expression were synthesized. Human codon-optimized APG05405 with N-terminal SV40 (SEQ ID NO: 149) and C-terminal nucleoplasmin NLS sequences (SEQ ID NOs: 149 and 150, respectively), N-terminal 3xFLAG tag (SEQ ID NO: 151), and a C- or N-terminal VPR activation domain (SEQ ID NO: 154; Chavez, et al. 2015, Nature Methods, 12(4): 326-328) under the control of a cytomegalovirus (CMV) promoter (SEQ ID NO: 152) was produced and introduced into a mammalian expression vector. Both presumed catalytically active and catalytically inactivated (“dAPG5405”) versions of APG05405 were used. Guide RNA expression constructs encoding a single gRNA, each under the control of a human RNA polymerase III U6 promoter (SEQ ID NO: 153), were also produced. Nuclease constructs are indicated in Table 5 below.

TABLE 5

RGN constructs

| Nuclease construct | SEQ ID NO. |
|---|---|
| APG05405 | 155 |
| dAPG05405 | 156 |
| APG05405-VPR | 157 |
| dAPG05405-VPR | 158 |
| VPR-APG05405 | 159 |
| VPR-dAPG05405 | 160 |

### Transfection and Expression in Mammalian Cells

One day prior to transfection, 1x10⁴ HEK293T cells (Sigma) were plated in 96-well plates in Dulbecco’s modified Eagle medium (DMEM) plus 10% (vol/vol) fetal bovine serum (Gibco) and 1% Penicillin-Streptomycin (Gibco). The next day when the cells were at 50-60% confluency, 100 ng of an RGN expression plasmid plus 100 ng of a single gRNA expression plasmid were co-transfected using 0.3 µL of Lipofectamine 3000 (Thermo Scientific) per well, following the manufacturer’s instructions. After 48 hours of growth, total RNA was harvested using the Cells-to-Ct One Step kit (ThermoFisher).

### Taqman Assay for Target Gene Expression

Endogenous genes were chosen which normally have low expression in HEK cells, but which can be induced upon CRISPR activation. RHOXF2 and CD2 were chosen for this purpose. TaqMan gene expression assays are performed using FAM labelled probes for RHOXF2 and CD2, and a VIC labelled probe for ACTB (all probes from ThermoFisher) as a normalization control. TaqMan assays are performed following the manufacturer’s instructions in the Cells-to-CT™ One Step kit (Thermofisher) in a BioRad CFX96 Real Time thermocycler. Background is measured in similar experiments where no gRNA is present. Fold changes in gene expression relative to background are calculated using the 2^{-ΔΔCt} method (Livak et al. 2001, Methods, 25(4):402-8), normalizing expression to ACTB transcript levels.

TABLE 6

Guide RNAs for target gene expression

| Nuclease | Gene | Guide (SEQ ID NO) |
|---|---|---|
| APG05405-VPR | RHOXF2 | 167 |
| APG05405-VPR | RHOXF2 | 168 |
| APG05405-VPR | RHOXF2 | 169 |
| APG05405-VPR | RHOXF2 | 170 |
| APG05405-VPR | RHOXF2 | 171 |
| APG05405-VPR | RHOXF2 | 172 |
| dAPG05405-VPR | RHOXF2 | 167 |
| dAPG05405-VPR | RHOXF2 | 168 |
| dAPG05405-VPR | RHOXF2 | 169 |
| dAPG05405-VPR | RHOXF2 | 170 |
| dAPG05405-VPR | RHOXF2 | 171 |
| dAPG05405-VPR | RHOXF2 | 172 |
| VPR-APG05405 | RHOXF2 | 167 |
| VPR-APG05405 | RHOXF2 | 168 |
| VPR-APG05405 | RHOXF2 | 169 |
| VPR-APG05405 | RHOXF2 | 170 |
| VPR-APG05405 | RHOXF2 | 171 |
| VPR-APG05405 | RHOXF2 | 172 |
| VPR-dAPG05405 | RHOXF2 | 167 |
| VPR-dAPG05405 | RHOXF2 | 168 |
| VPR-dAPG05405 | RHOXF2 | 169 |
| VPR-dAPG05405 | RHOXF2 | 170 |
| VPR-dAPG05405 | RHOXF2 | 171 |
| VPR-dAPG05405 | RHOXF2 | 172 |
| APG05405-VPR | CD2 | 161 |
| APG05405-VPR | CD2 | 162 |
| APG05405-VPR | CD2 | 163 |
| APG05405-VPR | CD2 | 164 |
| APG05405-VPR | CD2 | 165 |
| APG05405-VPR | CD2 | 166 |
| dAPG05405-VPR | CD2 | 161 |
| dAPG05405-VPR | CD2 | 162 |
| dAPG05405-VPR | CD2 | 163 |
| dAPG05405-VPR | CD2 | 164 |
| dAPG05405-VPR | CD2 | 165 |
| dAPG05405-VPR | CD2 | 166 |
| VPR-APG05405 | CD2 | 161 |
| VPR-APG05405 | CD2 | 162 |
| VPR-APG05405 | CD2 | 163 |
| VPR-APG05405 | CD2 | 164 |
| VPR-APG05405 | CD2 | 165 |
| VPR-APG05405 | CD2 | 166 |
| VPR-dAPG05405 | CD2 | 161 |
| VPR-dAPG05405 | CD2 | 162 |
| VPR-dAPG05405 | CD2 | 163 |
| VPR-dAPG05405 | CD2 | 164 |
| VPR-dAPG05405 | CD2 | 165 |
| VPR-dAPG05405 | CD2 | 166 |

### Example 6: Programmable DNA Binding and Base Editing

### Oligonucleotides and PCR

All PCR reactions described below are performed using 10 µl of 2X Master Mix Phusion High-Fidelity DNA polymerase (Thermo Scientific) in a 20 µl reaction including 0.5 uM of each primer. Large genomic regions encompassing each target gene are first amplified using PCR# 1 primers, using a program of 98° C., 1 min; 30 cycles of [98° C., 10 sec; 62° C., 15 sec; 72° C., 5 min]; 72° C., 5 min; 12° C., forever. One microliter of this PCR reaction is then further amplified using primers specific for each guide (PCR#2 primers), using a program of 98° C., 1 min; 35 cycles of [98° C., 10 sec; 67° C., 15 sec; 72° C., 30 sec]; 72° C., 5 min; 12° C., forever. Primers for PCR#2 include Nextera Read 1 and Read 2 Transposase Adapter overhang sequences for Illumina sequencing.

### Construction of RGN Base Editing and gRNA Mammalian Expression Plasmids

Nuclease constructs for mammalian expression are synthesized. Human codon-optimized APG05405 with N-terminal SV40 (SEQ ID NO: 149) and C-terminal nucleoplasmin NLS sequences (SEQ ID NOs: 149 and 150, respectively), N-terminal 3xFLAG tag (SEQ ID NO: 151) and an N-terminal deaminase (for example, hAPOBEC3A; SEQ ID NO: 177) under the control of a cytomegalovirus (CMV) promoter (SEQ ID NO: 152) is produced and introduced into a mammalian expression vector. A catalytically inactivated version of APG05405 (“dAPG5405”) is used. Guide RNA expression constructs encoding a single gRNA each under the control of a human RNA polymerase III U6 promoter (SEQ ID NO: 153) are also produced.

### Transfection and Expression in Mammalian Cells

One day prior to transfection, 1x10⁵ HEK293T cells (Sigma) are plated in 24-well dishes in Dulbecco’s modified Eagle medium (DMEM) plus 10% (vol/vol) fetal bovine serum (Gibco) and 1% Penicillin-Streptomycin (Gibco). When the cells are at 50-60% confluency, 500 ng of a APG05405 expression plasmid plus 500 ng of a single gRNA expression plasmid are co-transfected using 1.5 uL of Lipofectamine 3000 (Thermo Scientific) per well, following the manufacturer’s instructions. After 48 hours of growth, total genomic DNA is harvested using a genomic DNA isolation kit (Machery-Nagel) according to the manufacturer’s instructions.

### Next Generation Sequencing

Products from PCR#2 containing Illumina overhang sequences underwent library preparation following the Illumina 16S Metagenomic Sequencing Library protocol. Deep sequencing is performed on an Illumina Mi-Seq platform by a service provider (MOGene). Typically, 200,000 250 bp paired-end reads (2 x 100,000 reads) are generated per amplicon. The reads are analyzed using CRISPResso (Pinello, et al. 2016 Nature Biotech, 34:695-697) to calculate the rates of base editing. Output alignments are hand-curated to confirm base editing windows as well as identify insertions or deletions. Each position across the target is analyzed to determine the editing rate and specific nucleotide changes that occur at each position.

### Example 7: Trans ssDNA Cleavage

### 7.1 Determining Assay Conditions for Trans DNA Cleavage

Purified APG05405 was incubated with single guide RNA (sgRNA) in Cutsmart buffer (New England Biolabs B7204S) at a final concentration of either 50 nM nuclease and 100 nM sgRNA or 200 nM nuclease and 400 nM sgRNA for 10 min. These RNP solutions were added to solutions of ssDNA – a target or mismatched negative control ssDNA – at a final concentration of 10 nM and a reporter ssDNA probe at a final concentration of 250 nM in 1.5X Cutsmart buffer (New England Biolabs B7204S). The reporter probes (TB0125 and TB0089 set forth as SEQ ID NOs: 197 and 198, respectively) contain a fluorescent dye at the 5′ end (56-FAM for TB0125 and Cy5 for TB0089), a quencher at the 3′ end (3IABkFQ for TB0125 and 3IAbRQSp for TB0089), and optionally an internal quencher (the internal quencher ZEN is only present on TB0125). Cleavage of the reporter probe results in dequenching of the fluorescent dye and thus an increase in fluorescence signal. To monitor fluorescence intensity, 10 µL of each reaction was incubated in a Corning low volume 384-well microplate at 30° C. in a microplate reader (CLARIOstar Plus).

A number of conditions were scouted in order to determine suitable parameters for this assay. In order to determine if there are effects of quenched probe design or fluorophore characteristics, two such reporters were included as a mixture in each reaction. They were at the same concentration as each other in any given reaction. In all cases, the control or target ssDNA concentration (LE201 or LE205 set forth as SEQ ID NOs: 199 and 200, respectively) was 10 nM. The RNP names signify the nuclease and the target as indicated in Table 7 below.

TABLE 7

Ribonucleoprotein complexes

| | | | |
|---|---|---|---|
| Nuclease | sgRNA (SEQ ID NO) | Intended Target | RNP Name |
| APG05405 | Gsg.1 (193) | LE201 | APG05405.1 |
| APG05405 | Gsg.2 (194) | LE205 | APG05405.2 |

The results are shown in Table 8 below.

TABLE 8

Results of trans DNA cleavage assays

| | | | | Slope (Arbitrary Units / min) | |
|---|---|---|---|---|---|
| RNP | [RNP] (nM) | [reporters] (nM) | Target | Cy5 channel | FAM channel |
| APG05405.1 | 25 nM | 0 | LE201 | -0.60 | -0.60 |
| APG05405.1 | 25 nM | 50 | LE201 | 72.60 | 4.20 |
| APG05405.1 | 25 nM | 250 | LE201 | 198.60 | -1.20 |
| APG05405.1 | 25 nM | 500 | LE201 | 294.00 | -3.00 |
| APG05405.1 | 25 nM | 750 | LE201 | 428.40 | -16.80 |
| APG05405.1 | 25 nM | 1000 | LE201 | 509.40 | -19.80 |
| APG05405.1 | 25 nM | 0 | LE205 | -0.48 | -0.42 |
| APG05405.1 | 25 nM | 50 | LE205 | 6.60 | -1.80 |
| APG05405.1 | 25 nM | 250 | LE205 | 57.00 | 0.60 |
| APG05405.1 | 25 nM | 500 | LE205 | 185.40 | -6.60 |
| APG05405.1 | 25 nM | 750 | LE205 | 318.60 | -14.40 |
| APG05405.1 | 25 nM | 1000 | LE205 | 483.60 | 3.60 |
| APG05405.2 | 25 nM | 0 | LE201 | 0.00 | -2.40 |
| APG05405.2 | 25 nM | 50 | LE201 | 12.00 | -3.00 |
| APG05405.2 | 25 nM | 250 | LE201 | 81.00 | 3.60 |
| APG05405.2 | 25 nM | 500 | LE201 | 232.20 | 7.80 |
| APG05405.2 | 25 nM | 750 | LE201 | 423.60 | 7.80 |
| APG05405.2 | 25 nM | 1000 | LE201 | 684.60 | 9.60 |
| APG05405.2 | 25 nM | 0 | LE205 | -0.60 | -2.40 |
| APG05405.2 | 25 nM | 50 | LE205 | 173.40 | 44.40 |
| APG05405.2 | 25 nM | 250 | LE205 | 262.80 | 49.20 |
| APG05405.2 | 25 nM | 500 | LE205 | 381.00 | 44.40 |
| APG05405.2 | 25 nM | 750 | LE205 | 530.40 | 31.80 |
| APG05405.2 | 25 nM | 1000 | LE205 | 678.00 | 21.00 |
| APG05405.1 | 100 nM | 0 | LE201 | 0.36 | -3.00 |
| APG05405.1 | 100 nM | 50 | LE201 | 97.20 | 5.40 |
| APG05405.1 | 100 nM | 250 | LE201 | 429.00 | 19.20 |
| APG05405.1 | 100 nM | 500 | LE201 | 651.00 | 26.40 |
| APG05405.1 | 100 nM | 750 | LE201 | 1176.00 | 29.40 |
| APG05405.1 | 100 nM | 1000 | LE201 | 1601.40 | 29.40 |
| APG05405.1 | 100 nM | 0 | LE205 | -1.80 | -0.36 |
| APG05405.1 | 100 nM | 50 | LE205 | 24.00 | 0.24 |
| APG05405.1 | 100 nM | 250 | LE205 | 142.20 | 3.60 |
| APG05405.1 | 100 nM | 500 | LE205 | 331.80 | 5.40 |
| APG05405.1 | 100 nM | 750 | LE205 | 590.40 | 10.80 |
| APG05405.1 | 100 nM | 1000 | LE205 | 952.80 | 9.00 |
| APG05405.2 | 100 nM | 0 | LE201 | -0.36 | -3.00 |
| APG05405.2 | 100 nM | 50 | LE201 | 30.60 | -4.20 |
| APG05405.2 | 100 nM | 250 | LE201 | 213.60 | 4.80 |
| APG05405.2 | 100 nM | 500 | LE201 | 499.20 | 6.00 |
| APG05405.2 | 100 nM | 750 | LE201 | 1128.00 | -1.80 |
| APG05405.2 | 100 nM | 1000 | LE201 | 1682.40 | 7.20 |
| APG05405.2 | 100 nM | 0 | LE205 | 0.06 | -1.80 |
| APG05405.2 | 100 nM | 50 | LE205 | 389.40 | 72.60 |
| APG05405.2 | 100 nM | 250 | LE205 | 1123.80 | 197.40 |
| APG05405.2 | 100 nM | 500 | LE205 | 1510.20 | 235.20 |
| APG05405.2 | 100 nM | 750 | LE205 | 2048.40 | 256.20 |
| APG05405.2 | 100 nM | 1000 | LE205 | 2393.40 | 234.00 |

From this experiment, it was concluded that the 100 nM concentration of RNP results generally in a higher cleavage rate of the reporter probe than the 25 nM RNP concentration. In general, reporter cleavage rates are higher at higher concentrations of the reporter oligonucleotides up to 250 nM reporter concentration, with little benefit observed from further increase in reporter concentrations. Notably, for the TB0089 reporter (detected in the Cy5 channel), there are substantially higher levels of background activity that interfere with target differentiation, especially at reporter concentrations higher than 250 nM. Therefore, it was concluded that reporter concentrations higher than 250 nM would not be beneficial and that in general, the doubly quenched TB0125 probe (detected in the FAM channel) is more suitable for future experiments since it provides a higher ratio of specific to background activity at a wide range of reporter concentrations.

### 7.2 APG09624 Trans DNA Cleavage and Effect of Purification on Non-Specific Activity

Purified APG05405 and APG09624 were incubated with single guide RNA (sgRNA) as indicated below in 1X Cutsmart buffer (New England Biolabs B7204S) at a final concentration of 200 nM nuclease and 400 nM sgRNA for 10 min at 37° C.

TABLE 9

Ribonucleoprotein complexes

| | | | | |
|---|---|---|---|---|
| Nuclease | Nuclease batch | sgRNA | Intended Target | RNP Name |
| APG09624 | N/A | Gsg.2 | LE113 | APG09624.2 |
| APG05405 | A | Gsg.2 | LE113 | APG05405.2A |
| APG05405 | B | Gsg.2 | LE113 | APG05405.2B |

These RNP solutions were then added to solutions of ssDNA – a target or mismatched negative control ssDNA – at a final concentration of 10 nM and a reporter probe (TP0003; set forth as SEQ ID NO: 314) at a final concentration of 250 nM in 1.5X Cutsmart buffer (New England Biolabs B7204S). Thereporter probe contains a fluorescent dye at the 5′ end and a quencher at the 3′ end. Cleavage of the reporter probe results in dequenching of the fluorescent dye and thus an increase in fluorescence signal. To monitor fluorescence intensity, 10 µl of each reaction was incubated in a Corning low volume 384-well microplate at 37° C. in a microplate reader (CLARIOstar Plus).

Incubation with target sequences resulted in a substantial increase in fluorescence intensity as a function of time relative to the negative control. The rate of cleavage is expediently summarized as the slope of the linear portion of the fluorescence vs. time function as shown in Table 10.

TABLE 10

Results of trans DNA cleavage assay

| RNP | ssDNA Target Sequence | Slope (Arb. Units / min) |
|---|---|---|
| APG09624.2 | LE111 | 182 |
| APG09624.2 | LE113 | 561 |
| APG05405.2A | LE111 | 197 |
| APG05405.2A | LE113 | 1625 |
| APG05405.2B | LE111 | 73 |
| APG05405.2B | LE113 | 1054 |

These data show differentiation of the target sequences by the RNPs formed from both APG05405 and APG09624.

### 7.3 Trans DNA Cleavage Activation by PCR Products

Oligonucleotides containing degenerate nucleotides 5′ of the target were PCR amplified to produce target sequences. Target dsPCR2 and dsPCR3 contain target sequences ACTACAACAGCCACAACGTCTATATCATGG (dsPCR2) and TGGAATGGGAACTAAAGTAATGG (dsPCR3) set forth as SEQ ID NOs: 311 and 312, respectively) contained an 8 bp and 5 bp degenerate region, respectively, on the 5′ side of the target encoded by the guide RNA. The oligo pairs were annealed and PCR amplified with appropriate primers. In addition, ssDNA targets were included in the experiment -oligonucleotides containing the reverse complement of the target sequences described above
CCATGATATAGACGTTGTGGCTGTTGTAGT (LE205; SEQ ID NO: 200) and
CCATTACTTTAGTTCCCATTCCA (LE501; SEQ ID NO: 174).

RNP solutions were formed by incubation of nuclease and sgRNA at 0.5 µM and 1 µM, respectively in 1X NEBuffer 2 (New England Biolabs) and incubated at room temperature for 20 minutes.

TABLE 11

Ribonucleoprotein complexes

| | | |
|---|---|---|
| sgRNA | Intended Targets | RNP Name |
| Gsg.3 (SEQ ID NO: 175) | dsPCR3, LE501 | APG05405.3 |
| Gsg.2 (SEQ ID NO: 194) | dsPCR2, LE205 | APG05405.2 |

The cleavage reaction was performed in 1.5X NEBuffer 2 with 1.5 µM reporter with a 5′ TEX615 label and a 3′ Iowa Black FQ quencher and 100 nM of the respective PCR product or ssDNA oligonucleotide LE501 or LE205 (set forth as SEQ ID NOs: 174 and 200, respectively; LE501 comprised a 5′ FAM fluorophore). Cleavage of the reporter probe results in dequenching of the fluorescent dye and thus an increase in fluorescence signal. To monitor fluorescence intensity, 10 µl of each reaction was incubated in a Corning low volume 384-well microplate at 37° C. in a microplate reader (CLARIOstar Plus). The results of kinetic analysis are shown in Table 12.

TABLE 12

Results of trans DNA cleavage assay

| Target | RNP | Slope (Arb. Units / min) |
|---|---|---|
| dsPCR3 | APG05405.3 | 780 |
| | APG05405.2 | 102 |
| dsPCR2 | APG05405.3 | 78 |
| | APG05405.2 | 768 |
| LE501 | APG05405.3 | 624 |
| | APG05405.2 | 120 |
| LE205 | APG05405.3 | 90 |
| | APG05405.2 | 1434 |

These results demonstrate target sequence specific activation of non-sequence specific cleavage of ssDNA. Both dsDNA PCR products and target ssDNA oligonucleotides were able to induce this activity.

### 7.4 PAM Determination by Induced Non-Specific ssDNA Cleavage

Isolation of the ternary complex containing a DNA target (including a PAM library) with the RNP and sequencing of the DNA recovered from it can be used to identify the PAM sequence, if the given system requires a PAM for DNA binding, modification or cleavage. The complex can be captured by a number of methods, such as immuno-pulldown, capture with immobilized metal affinity resin (such as Ni-NTA agarose), or isolation by size exclusion chromatography.

Alternatively, a parallel library of DNA fragments with distinct PAM sequences adjacent to a fixed target is produced. RNPs containing the putative nuclease and a suitable guide RNA (single guide or dual-RNA guide targeting the fixed target) are incubated with each of the fragments in the library and assessed for binding using a shift in electrophoretic mobility of the DNA fragment, size exclusion liquid chromatography, or co-precipitation using a solid support with affinity for either component.

### 7.5 PAM Determination Using a Parallel Plasmid DNA Library

A plasmid library is produced containing a target sequence, ACTACAACAGCCACAACGTCTATATCATGG (set forth as SEQ ID NO: 313), preceded by an 8 bp degenerate sequence (NNNNNNNN set forth as SEQ ID NO: 176). Upon transformation into competent cells and plating onto agar plates with selective media, each colony resulting from the transformation of this reaction corresponds in principle to a clonal plasmid DNA sequence and thus preparations of plasmid DNA from cultures deriving from single colonies are unique plasmid preparations sampled from the original library. Plasmid preparations are obtained from a sampling of 96 colonies. These preparations are individually subjected to Sanger sequencing to verify their PAM sequence.

Purified APG05405 is incubated with single guide RNA (sgRNA) Gsg.2 (set forth as SEQ ID NO: 194) in 1X Cutsmart buffer (New England Biolabs B7204S) at a final concentration of 200 nM nuclease and 400 nM sgRNA for 20 minutes at room temperature.

These RNP solutions were added at a final concentration of 100 nM to solutions of plasmid DNA and ssDNA reporter strand comprising a fluorophore and a quencher at 50 nM in 1.5X Cutsmart buffer (New England Biolabs B7204S). To monitor fluorescence intensity as a function of time, 10 µl of each reaction is incubated in a Corning low volume 384-well microplate at 37° C. in a microplate reader (CLARIOstar Plus). Each well corresponds to an individual digestion reaction with a specific PAM sequence. Upon completion of data collection, the rate of fluorescence increase will be determined. A consensus PAM sequence will be built by analyzing the sequences that correspond to wells with high rates of fluorescence increase. If inconclusive, an additional library can be produced and evaluated.

### Example 8: Use of ssDNA Cleavage as a Diagnostic

Due to the capability of these nucleases to generate an optically detectable signal in the presence of a target DNA sequence, they promise utility for implementation into diagnostic devices for the detection of genetic diseases or agents of infectious disease, such as bacteria, viruses, or fungi.

A diagnostic procedure may include isolation or amplification of nucleic acids from a sample to be tested. It may also be suitable to use some samples without performing any isolation or purification of nucleic acids, as they may be present in the sample at high enough quantities to be detectable without amplification (such as PCR) or free of materials that interfere with detection or signal production.

RNPs formed as described in the other examples could then be exposed to the sample (or processed sample as described in the preceding paragraph) along with a reporter, such as the fluorophore and quencher modified ssDNA oligonucleotides used in previous examples, or some other sort of ssDNA substrate that produces a visible or otherwise easily detectable signal when cleaved. If using fluorophore-quencher conjugated DNA oligonucleotides (as in the previously described examples), these can be detected using a fluorimeter as described in previous examples. To simplify detection, an endpoint assay can be performed instead of the kinetic assays described above, meaning that the assays can be performed for a fixed time and read out at the end of this elapsed time, relative to positive and negative controls.

These reagents may also be integrated into a lateral flow testing device which allows for the detection of a given disease-causing agent or specific nucleic acid sequence (such as a diseased allele in an individual) with very little instrumentation. In this assay, the ssDNA reporter would be conjugated to multiple molecules suitable for antibody or affinity reagent capture, such as fluorescein, biotin, and/or digoxigenin.

### Example 8.1 - COVID19 Diagnostic Assay

Samples are collected from patients using nasopharyngeal swabs in universal transport medium (UTM) according to standard practice, and RNA is extracted. Reverse Transcription Loop-Mediated Isothermal Amplification (RT-LAMP) is used to amplify the genetic material, similar to Broughton et al 2020 (Nat. Biotechnol. 38: 870-874). In some embodiments, single-stranded DNA (ssDNA) produced by RT-LAMP may be sufficient for PAM-independent detection by an RGN disclosed herein. In some embodiments, the RT-LAMP produces ssDNA using amplification by a phosphorothioate primer only on the target strand, allowing for T7 exonuclease digestion of the non-target strand.

RT-LAMP amplification is performed with appropriate primers to amplify the N gene and the E gene of the SARS-CoV2 genome, as well as human RNase P as a quality control check for sample collection and preparation, similar to Broughton et al 2020. One of the two LAMP internal primers (commonly called FIP or BIP) would contain phosphorothioate groups. Upon treatment of the completed PCR reaction with T7 exonuclease, the ssDNA extended from the phosphorothioate primer is the major species present in the solution. Guides against this sequence are evaluated for specific and efficient activation using the fluorescence assays described above. For specificity, the detection scheme can be tested against homologous genes in other coronaviruses, to ensure lack of cross-reactivity, such as HCoV-OC43, HCoV-HKU1, HCoV-229E, HCoV-NL63, MERS-CoV, and/or SARS-CoV. The assay may be converted into a lateral flow assay by utilizing an oligonucleotide containing FAM and biotin.

That which is claimed:

1. A nucleic acid molecule comprising a polynucleotide encoding an RNA-guided nuclease (RGN) polypeptide, wherein said polynucleotide comprises a nucleotide sequence encoding an RGN polypeptide comprising an amino acid sequence having at least 90% sequence identity to any one of SEQ ID NOs: 2, 4, 1, 3, and 5 to 109;
wherein said RGN polypeptide is capable of binding a target DNA sequence of a DNA molecule in an RNA-guided sequence specific manner when bound to a guide RNA (gRNA) capable of hybridizing to said target DNA sequence, and
wherein said polynucleotide encoding an RGN polypeptide is operably linked to a promoter heterologous to said polynucleotide.

2. The nucleic acid molecule of claim 1, wherein said RGN polypeptide comprises an amino acid sequence having at least 95% sequence identity to any one of SEQ ID NOs: 2, 4, 1, 3, and 5 to 109.

3. The nucleic acid molecule of claim 1, wherein said RGN polypeptide comprises an amino acid sequence having 100% sequence identity to any one of SEQ ID NOs: 2, 4, 1, 3, and 5 to 109.

4. The nucleic acid molecule of any one of claims 1-3, wherein said target DNA sequence is within a region of said DNA molecule that is single-stranded.

5. The nucleic acid molecule of claim 4, wherein said RGN polypeptide is capable of cleaving said target DNA sequence upon binding.

6. The nucleic acid molecule of any one of claims 1-3, wherein said target DNA sequence is within a region of said DNA molecule that is double-stranded.

7. The nucleic acid molecule of claim 6, wherein said RGN polypeptide is capable of cleaving said target DNA sequence upon binding.

8. The nucleic acid molecule of claim 7, wherein cleavage by said RGN polypeptide generates a double-stranded break.

9. The nucleic acid molecule of claim 7, wherein cleavage by said RGN polypeptide generates a single-stranded break.

10. The nucleic acid molecule of any one of claims 6-9, wherein the RGN polypeptide is operably fused to a base-editing polypeptide.

11. The nucleic acid molecule of claim 10, wherein the base-editing polypeptide is a deaminase.

12. The nucleic acid molecule of any one of claims 6-11, wherein said target DNA sequence is located adjacent to a protospacer adjacent motif (PAM).

13. The nucleic acid molecule of any one of claims 1-12, wherein the RGN polypeptide comprises one or more nuclear localization signals.

14. The nucleic acid molecule of any one of claims 1-13, wherein the RGN polypeptide is codon optimized for expression in a eukaryotic cell.

15. A vector comprising the nucleic acid molecule of any one of claims 1-14.

16. The vector of claim 15, further comprising at least one nucleotide sequence encoding said gRNA capable of hybridizing to said target DNA sequence.

17. The vector of claim 16, wherein said RGN polypeptide comprises an amino acid sequence having at least 90% sequence identity to SEQ ID NO: 11 and said gRNA comprises a CRISPR RNA comprising a CRISPR repeat sequence having at least 90% sequence identity to SEQ ID NO: 116.

18. The vector of claim 16, wherein said RGN polypeptide comprises an amino acid sequence having at least 95% sequence identity to SEQ ID NO: 11 and said gRNA comprises a CRISPR RNA comprising a CRISPR repeat sequence having at least 95% sequence identity to SEQ ID NO: 116.

19. The vector of claim 16, wherein said RGN polypeptide comprises an amino acid sequence having 100% sequence identity to SEQ ID NO: 11 and said gRNA comprises a CRISPR RNA comprising a CRISPR repeat sequence having 100% sequence identity to SEQ ID NO: 116.

20. The vector of claim 16, wherein said gRNA comprises a tracrRNA.

21. The vector of claim 20, wherein said tracrRNA is selected from the group consisting of
a) a tracrRNA having at least 90% sequence identity to SEQ ID NO: 121, wherein said gRNA further comprises a CRISPR RNA comprising a CRISPR repeat sequence having at least 90% sequence identity to SEQ ID NO: 111, and wherein said RGN polypeptide comprises an amino acid sequence having at least 90% sequence identity to SEQ ID NO: 2;
b) a tracrRNA having at least 90% sequence identity to SEQ ID NO: 123, wherein said gRNA further comprises a CRISPR RNA comprising a CRISPR repeat sequence having at least 90% sequence identity to SEQ ID NO: 113, and wherein said RGN polypeptide comprises an amino acid sequence having at least 90% sequence identity to SEQ ID NO: 4;
c) a tracrRNA having at least 90% sequence identity to SEQ ID NO: 120, wherein said gRNA further comprises a CRISPR RNA comprising a CRISPR repeat sequence having at least 90% sequence identity to SEQ ID NO: 110, and wherein said RGN polypeptide comprises an amino acid sequence having at least 90% sequence identity to SEQ ID NO: 1;
d) a tracrRNA having at least 90% sequence identity to SEQ ID NO: 122, wherein said gRNA further comprises a CRISPR RNA comprising a CRISPR repeat sequence having at least 90% sequence identity to SEQ ID NO: 112, and wherein said RGN polypeptide comprises an amino acid sequence having at least 90% sequence identity to SEQ ID NO: 3;
e) a tracrRNA having at least 90% sequence identity to SEQ ID NO: 124, wherein said gRNA further comprises a CRISPR RNA comprising a CRISPR repeat sequence having at least 90% sequence identity to SEQ ID NO: 114, and wherein said RGN polypeptide comprises an amino acid sequence having at least 90% sequence identity to SEQ ID NO: 5;
f) a tracrRNA having at least 90% sequence identity to SEQ ID NO: 125, wherein said gRNA further comprises a CRISPR RNA comprising a CRISPR repeat sequence having at least 90% sequence identity to SEQ ID NO: 115, and wherein said RGN polypeptide comprises an amino acid sequence having at least 90% sequence identity to SEQ ID NO: 6;
g) a tracrRNA having at least 90% sequence identity to SEQ ID NO: 126, wherein said gRNA further comprises a CRISPR RNA comprising a CRISPR repeat sequence having at least 90% sequence identity to SEQ ID NO: 117, and wherein said RGN polypeptide comprises an amino acid sequence having at least 90% sequence identity to SEQ ID NO: 12;
h) a tracrRNA having at least 90% sequence identity to SEQ ID NO: 127, wherein said gRNA further comprises a CRISPR RNA comprising a CRISPR repeat sequence having at least 90% sequence identity to SEQ ID NO: 118, and wherein said RGN polypeptide comprises an amino acid sequence having at least 90% sequence identity to SEQ ID NO: 13; and
i) a tracrRNA having at least 90% sequence identity to SEQ ID NO: 128, wherein said gRNA further comprises a CRISPR RNA comprising a CRISPR repeat sequence having at least 90% sequence identity to SEQ ID NO: 119, and wherein said RGN polypeptide comprises an amino acid sequence having at least 90% sequence identity to SEQ ID NO: 16.

22. The vector of claim 20, wherein said tracrRNA is selected from the group consisting of:
a) a tracrRNA having at least 95% sequence identity to SEQ ID NO: 121, wherein said gRNA further comprises a CRISPR RNA comprising a CRISPR repeat sequence having at least 95% sequence identity to SEQ ID NO: 111, and wherein said RGN polypeptide comprises an amino acid sequence having at least 95% sequence identity to SEQ ID NO: 2;
b) a tracrRNA having at least 95% sequence identity to SEQ ID NO: 123, wherein said gRNA further comprises a CRISPR RNA comprising a CRISPR repeat sequence having at least 95% sequence identity to SEQ ID NO: 113, and wherein said RGN polypeptide comprises an amino acid sequence having at least 95% sequence identity to SEQ ID NO: 4;
c) a tracrRNA having at least 95% sequence identity to SEQ ID NO: 120, wherein said gRNA further comprises a CRISPR RNA comprising a CRISPR repeat sequence having at least 95% sequence identity to SEQ ID NO: 110, and wherein said RGN polypeptide comprises an amino acid sequence having at least 95% sequence identity to SEQ ID NO: 1;
d) a tracrRNA having at least 95% sequence identity to SEQ ID NO: 122, wherein said gRNA further comprises a CRISPR RNA comprising a CRISPR repeat sequence having at least 95% sequence identity to SEQ ID NO: 112, and wherein said RGN polypeptide comprises an amino acid sequence having at least 95% sequence identity to SEQ ID NO: 3;
e) a tracrRNA having at least 95% sequence identity to SEQ ID NO: 124, wherein said gRNA further comprises a CRISPR RNA comprising a CRISPR repeat sequence having at least 95% sequence identity to SEQ ID NO: 114, and wherein said RGN polypeptide comprises an amino acid sequence having at least 95% sequence identity to SEQ ID NO: 5;
f) a tracrRNA having at least 95% sequence identity to SEQ ID NO: 125, wherein said gRNA further comprises a CRISPR RNA comprising a CRISPR repeat sequence having at least 95% sequence identity to SEQ ID NO: 115, and wherein said RGN polypeptide comprises an amino acid sequence having at least 95% sequence identity to SEQ ID NO: 6;
g) a tracrRNA having at least 95% sequence identity to SEQ ID NO: 126, wherein said gRNA further comprises a CRISPR RNA comprising a CRISPR repeat sequence having at least 95% sequence identity to SEQ ID NO: 117, and wherein said RGN polypeptide comprises an amino acid sequence having at least 95% sequence identity to SEQ ID NO: 12;
h) a tracrRNA having at least 95% sequence identity to SEQ ID NO: 127, wherein said gRNA further comprises a CRISPR RNA comprising a CRISPR repeat sequence having at least 95% sequence identity to SEQ ID NO: 118, and wherein said RGN polypeptide comprises an amino acid sequence having at least 95% sequence identity to SEQ ID NO: 13; and
i) a tracrRNA having at least 95% sequence identity to SEQ ID NO: 128, wherein said gRNA further comprises a CRISPR RNA comprising a CRISPR repeat sequence having at least 95% sequence identity to SEQ ID NO: 119, and wherein said RGN polypeptide comprises an amino acid sequence having at least 95% sequence identity to SEQ ID NO: 16.

23. The vector of claim 20, wherein said tracrRNA is selected from the group consisting of:
a) a tracrRNA having 100% sequence identity to SEQ ID NO: 121, wherein said gRNA further comprises a CRISPR RNA comprising a CRISPR repeat sequence having 100% sequence identity to SEQ ID NO: 111, and wherein said RGN polypeptide comprises an amino acid sequence having 100% sequence identity to SEQ ID NO: 2;
b) a tracrRNA having 100% sequence identity to SEQ ID NO: 123, wherein said gRNA further comprises a CRISPR RNA comprising a CRISPR repeat sequence having 100% sequence identity to SEQ ID NO: 113, and wherein said RGN polypeptide comprises an amino acid sequence having 100% sequence identity to SEQ ID NO: 4;
c) a tracrRNA having 100% sequence identity to SEQ ID NO: 120, wherein said gRNA further comprises a CRISPR RNA comprising a CRISPR repeat sequence having 100% sequence identity to SEQ ID NO: 110, and wherein said RGN polypeptide comprises an amino acid sequence having 100% sequence identity to SEQ ID NO: 1;
d) a tracrRNA having 100% sequence identity to SEQ ID NO: 122, wherein said gRNA further comprises a CRISPR RNA comprising a CRISPR repeat sequence having 100% sequence identity to SEQ ID NO: 112, and wherein said RGN polypeptide comprises an amino acid sequence having 100% sequence identity to SEQ ID NO: 3;
e) a tracrRNA having 100% sequence identity to SEQ ID NO: 124, wherein said gRNA further comprises a CRISPR RNA comprising a CRISPR repeat sequence having 100% sequence identity to SEQ ID NO: 114, and wherein said RGN polypeptide comprises an amino acid sequence having 100% sequence identity to SEQ ID NO: 5;
f) a tracrRNA having 100% sequence identity to SEQ ID NO: 125, wherein said gRNA further comprises a CRISPR RNA comprising a CRISPR repeat sequence having 100% sequence identity to SEQ ID NO: 115, and wherein said RGN polypeptide comprises an amino acid sequence having 100% sequence identity to SEQ ID NO: 6;
g) a tracrRNA having 100% sequence identity to SEQ ID NO: 126, wherein said gRNA further comprises a CRISPR RNA comprising a CRISPR repeat sequence having 100% sequence identity to SEQ ID NO: 117, and wherein said RGN polypeptide comprises an amino acid sequence having 100% sequence identity to SEQ ID NO: 12;
h) a tracrRNA having 100% sequence identity to SEQ ID NO: 127, wherein said gRNA further comprises a CRISPR RNA comprising a CRISPR repeat sequence having 100% sequence identity to SEQ ID NO: 118, and wherein said RGN polypeptide comprises an amino acid sequence having 100% sequence identity to SEQ ID NO: 13; and
i) a tracrRNA having 100% sequence identity to SEQ ID NO: 128, wherein said gRNA further comprises a CRISPR RNA comprising a CRISPR repeat sequence having 100% sequence identity to SEQ ID NO: 119, and wherein said RGN polypeptide comprises an amino acid sequence having 100% sequence identity to SEQ ID NO: 16.

24. The vector of any one of claims 20-23, where said gRNA is a single guide RNA.

25. The vector of any one of claims 20-23, wherein said gRNA is a dual-guide RNA.

26. A cell comprising the nucleic acid molecule of any one of claims 1-14 or the vector of any one of claims 15-25.

27. A method for making an RGN polypeptide comprising culturing the cell of claim 26 under conditions in which the RGN polypeptide is expressed.

28. A method for making an RGN polypeptide comprising introducing into a cell a heterologous nucleic acid molecule comprising a nucleotide sequence encoding an RNA-guided nuclease (RGN) polypeptide comprising an amino acid sequence having at least 90% sequence identity to any one of SEQ ID NOs: 2, 4, 1, 3 and 5 to 109;
wherein said RGN polypeptide binds a target DNA sequence of a DNA molecule in an RNA-guided sequence specific manner when bound to a guide RNA (gRNA) capable of hybridizing to said target DNA sequence;
and culturing said cell under conditions in which the RGN polypeptide is expressed.

29. The method of claim 28, wherein said RGN polypeptide comprises an amino acid sequence having at least 95% sequence identity to any one of SEQ ID NOs: 2, 4, 1, 3, and 5 to 109.

30. The method of claim 28, wherein said RGN polypeptide comprises an amino acid sequence having 100% sequence identity to any one of SEQ ID NOs: 2, 4, 1, 3, and 5 to 109.

31. The method of any one of claims 28-30, further comprising purifying said RGN polypeptide.

32. The method of any one of claims 28-30, wherein said cell further expresses one or more guide RNAs that binds to said RGN polypeptide to form an RGN ribonucleoprotein complex.

33. The method of claim 32, further comprising purifying said RGN ribonucleoprotein complex.

34. An isolated RNA-guided nuclease (RGN) polypeptide, wherein said RGN polypeptide comprises an amino acid sequence having at least 90% sequence identity to any one of SEQ ID NOs: 2, 4, 1, 3, and 5 to 109; and
wherein said RGN polypeptide is capable of binding a target DNA sequence of a DNA molecule in an RNA-guided sequence specific manner when bound to a guide RNA (gRNA) capable of hybridizing to said target DNA sequence.

35. The isolated RGN polypeptide of claim 34, wherein said RGN polypeptide comprises an amino acid sequence having at least 95% sequence identity to any one of SEQ ID NOs: 2, 4, 1, 3, and 5 to 109.

36. The isolated RGN polypeptide of claim 34, wherein said RGN polypeptide comprises an amino acid sequence having 100% sequence identity to any one of SEQ ID NOs: 2, 4, 1, 3, and 5 to 109.

37. The isolated RGN polypeptide of any one of claims 34-36, wherein said target DNA sequence is within a region of said DNA molecule that is single-stranded.

38. The isolated RGN polypeptide of claim 37, wherein said RGN polypeptide is capable of cleaving said target DNA sequence upon binding.

39. The isolated RGN polypeptide of any one of claims 34-36, wherein said target DNA sequence is within a region of said DNA molecule that is double-stranded.

40. The isolated RGN polypeptide of claim 39, wherein said RGN polypeptide is capable of cleaving said target DNA sequence upon binding.

41. The isolated RGN polypeptide of claim 40, wherein cleavage by said RGN polypeptide generates a double-stranded break.

42. The isolated RGN polypeptide of claim 40, wherein cleavage by said RGN polypeptide generates a single-stranded break.

43. The isolated RGN polypeptide of any one of claims 39-42, wherein the RGN polypeptide is operably fused to a base-editing polypeptide.

44. The isolated RGN polypeptide of claim 43, wherein the base-editing polypeptide is a deaminase.

45. The isolated RGN polypeptide of any one of claims 34-44, wherein said target DNA sequence is located adjacent to a protospacer adjacent motif (PAM).

46. The isolated RGN polypeptide of any one of claims 34-45, wherein the RGN polypeptide comprises one or more nuclear localization signals.

47. A system for binding a target DNA sequence of a DNA molecule, said system comprising:
a) one or more guide RNAs capable of hybridizing to said target DNA sequence or one or more polynucleotides comprising one or more nucleotide sequences encoding the one or more guide RNAs (gRNAs); and
b) an RNA-guided nuclease (RGN) polypeptide comprising an amino acid sequence having at least 90% sequence identity to any one of SEQ ID NOs: 2, 4, 1, 3, and 5 to 109 or a polynucleotide comprising a nucleotide sequence encoding the RGN polypeptide;
wherein at least one of said nucleotide sequence encoding the one or more guide RNAs and said nucleotide sequence encoding the RGN polypeptide is operably linked to a promoter heterologous to said nucleotide sequence; and
wherein the one or more guide RNAs are capable of forming a complex with the RGN polypeptide in order to direct said RGN polypeptide to bind to said target DNA sequence of the DNA molecule.

48. A system for binding a target DNA sequence of a DNA molecule, said system comprising:
a) one or more guide RNAs capable of hybridizing to said target DNA sequence or one or more polynucleotides comprising one or more nucleotide sequences encoding the one or more guide RNAs (gRNAs); and
b) an RNA-guided nuclease (RGN) polypeptide comprising an amino acid sequence having at least 90% sequence identity to any one of SEQ ID NOs: 2, 4, 1, 3, and 5 to 109;
wherein the one or more guide RNAs are capable of hybridizing to the target DNA sequence, and wherein the one or more guide RNAs are capable of forming a complex with the RGN polypeptide in order to direct said RGN polypeptide to bind to said target DNA sequence of the DNA molecule.

49. The system of claim 48, wherein at least one of said nucleotides sequences encoding the one or more guide RNAs is operably linked to a promoter heterologous to said nucleotide sequence.

50. The system of any one of claims 47-49, wherein said RGN polypeptide comprises an amino acid sequence having at least 95% sequence identity to any one of SEQ ID NOs: 2, 4, 1, 3, and 5 to 109.

51. The system of any one of claims 47-49, wherein said RGN polypeptide comprises an amino acid sequence having 100% sequence identity to any one of SEQ ID NOs: 2, 4, 1, 3, and 5 to 109.

52. The system of any one of claims 47-51, wherein said RGN polypeptide and said one or more guide RNAs are not found complexed to one another in nature.

53. The system of any one of claims 47-51, wherein said target DNA sequence is a eukaryotic target DNA sequence.

54. The system of any one of claims 47-53, wherein said RGN polypeptide comprises an amino acid sequence having at least 90% sequence identity to SEQ ID NO: 11 and said one or more guide RNAs comprise a CRISPR RNA comprising a CRISPR repeat sequence having at least 90% sequence identity to SEQ ID NO: 116.

55. The system of any one of claims 47-53, wherein said RGN polypeptide comprises an amino acid sequence having at least 95% sequence identity to SEQ ID NO: 11 and said one or more guide RNAs comprise a CRISPR RNA comprising a CRISPR repeat sequence having at least 95% sequence identity to SEQ ID NO: 116.

56. The system of any one of claims 47-53, wherein said RGN polypeptide comprises an amino acid sequence having 100% sequence identity to SEQ ID NO: 11 and said one or more guide RNAs comprise a CRISPR RNA comprising a CRISPR repeat sequence having 100% sequence identity to SEQ ID NO: 116.

57. The system of any one of claims 47-53, wherein said one or more guide RNAs comprise a tracrRNA.

58. The system of claim 57, wherein said tracrRNA is selected from the group consisting of:
a) a tracrRNA having at least 90% sequence identity to SEQ ID NO: 121, wherein said one or more guide RNAs further comprise a CRISPR RNA comprising a CRISPR repeat sequence having at least 90% sequence identity to SEQ ID NO: 111, and wherein said RGN polypeptide comprises an amino acid sequence having at least 90% sequence identity to SEQ ID NO: 2;
b) a tracrRNA having at least 90% sequence identity to SEQ ID NO: 123, wherein said one or more guide RNAs further comprise a CRISPR RNA comprising a CRISPR repeat sequence having at least 90% sequence identity to SEQ ID NO: 113, and wherein said RGN polypeptide comprises an amino acid sequence having at least 90% sequence identity to SEQ ID NO: 4;
c) a tracrRNA having at least 90% sequence identity to SEQ ID NO: 120, wherein said one or more guide RNAs further comprise a CRISPR RNA comprising a CRISPR repeat sequence having at least 90% sequence identity to SEQ ID NO: 110, and wherein said RGN polypeptide comprises an amino acid sequence having at least 90% sequence identity to SEQ ID NO: 1;
d) a tracrRNA having at least 90% sequence identity to SEQ ID NO: 122, wherein said one or more guide RNAs further comprise a CRISPR RNA comprising a CRISPR repeat sequence having at least 90% sequence identity to SEQ ID NO: 112, and wherein said RGN polypeptide comprises an amino acid sequence having at least 90% sequence identity to SEQ ID NO: 3;
e) a tracrRNA having at least 90% sequence identity to SEQ ID NO: 124, wherein said one or more guide RNAs further comprise a CRISPR RNA comprising a CRISPR repeat sequence having at least 90% sequence identity to SEQ ID NO: 114, and wherein said RGN polypeptide comprises an amino acid sequence having at least 90% sequence identity to SEQ ID NO: 5;
f) a tracrRNA having at least 90% sequence identity to SEQ ID NO: 125, wherein said one or more guide RNAs further comprise a CRISPR RNA comprising a CRISPR repeat sequence having at least 90% sequence identity to SEQ ID NO: 115, and wherein said RGN polypeptide comprises an amino acid sequence having at least 90% sequence identity to SEQ ID NO: 6;
g) a tracrRNA having at least 90% sequence identity to SEQ ID NO: 126, wherein said one or more guide RNAs further comprise a CRISPR RNA comprising a CRISPR repeat sequence having at least 90% sequence identity to SEQ ID NO: 117, and wherein said RGN polypeptide comprises an amino acid sequence having at least 90% sequence identity to SEQ ID NO: 12;
h) a tracrRNA having at least 90% sequence identity to SEQ ID NO: 127, wherein said one or more guide RNAs further comprise a CRISPR RNA comprising a CRISPR repeat sequence having at least 90% sequence identity to SEQ ID NO: 118, and wherein said RGN polypeptide comprises an amino acid sequence having at least 90% sequence identity to SEQ ID NO: 13; and
i) a tracrRNA having at least 90% sequence identity to SEQ ID NO: 128, wherein said one or more guide RNAs further comprise a CRISPR RNA comprising a CRISPR repeat sequence having at least 90% sequence identity to SEQ ID NO: 119, and wherein said RGN polypeptide comprises an amino acid sequence having at least 90% sequence identity to SEQ ID NO: 16.

59. The system of claim 57, wherein said tracrRNA is selected from the group consisting of:
a) a tracrRNA having at least 95% sequence identity to SEQ ID NO: 121, wherein said one or more guide RNAs further comprise a CRISPR RNA comprising a CRISPR repeat sequence having at least 95% sequence identity to SEQ ID NO: 111, and wherein said RGN polypeptide comprises an amino acid sequence having at least 95% sequence identity to SEQ ID NO: 2;
b) a tracrRNA having at least 95% sequence identity to SEQ ID NO: 123, wherein said one or more guide RNAs further comprise a CRISPR RNA comprising a CRISPR repeat sequence having at least 95% sequence identity to SEQ ID NO: 113, and wherein said RGN polypeptide comprises an amino acid sequence having at least 95% sequence identity to SEQ ID NO: 4;
c) a tracrRNA having at least 95% sequence identity to SEQ ID NO: 120, wherein said one or more guide RNAs further comprise a CRISPR RNA comprising a CRISPR repeat sequence having at least 95% sequence identity to SEQ ID NO: 110, and wherein said RGN polypeptide comprises an amino acid sequence having at least 95% sequence identity to SEQ ID NO: 1;
d) a tracrRNA having at least 95% sequence identity to SEQ ID NO: 122, wherein said one or more guide RNAs further comprise a CRISPR RNA comprising a CRISPR repeat sequence having at least 95% sequence identity to SEQ ID NO: 112, and wherein said RGN polypeptide comprises an amino acid sequence having at least 95% sequence identity to SEQ ID NO: 3;
e) a tracrRNA having at least 95% sequence identity to SEQ ID NO: 124, wherein said one or more guide RNAs further comprise a CRISPR RNA comprising a CRISPR repeat sequence having at least 95% sequence identity to SEQ ID NO: 114, and wherein said RGN polypeptide comprises an amino acid sequence having at least 95% sequence identity to SEQ ID NO: 5;
f) a tracrRNA having at least 95% sequence identity to SEQ ID NO: 125, wherein said one or more guide RNAs further comprise a CRISPR RNA comprising a CRISPR repeat sequence having at least 95% sequence identity to SEQ ID NO: 115, and wherein said RGN polypeptide comprises an amino acid sequence having at least 95% sequence identity to SEQ ID NO: 6;
g) a tracrRNA having at least 95% sequence identity to SEQ ID NO: 126, wherein said one or more guide RNAs further comprise a CRISPR RNA comprising a CRISPR repeat sequence having at least 95% sequence identity to SEQ ID NO: 117, and wherein said RGN polypeptide comprises an amino acid sequence having at least 95% sequence identity to SEQ ID NO: 12;
h) a tracrRNA having at least 95% sequence identity to SEQ ID NO: 127, wherein said one or more guide RNAs further comprise a CRISPR RNA comprising a CRISPR repeat sequence having at least 95% sequence identity to SEQ ID NO: 118, and wherein said RGN polypeptide comprises an amino acid sequence having at least 95% sequence identity to SEQ ID NO: 13; and
i) a tracrRNA having at least 95% sequence identity to SEQ ID NO: 128, wherein said one or more guide RNAs further comprise a CRISPR RNA comprising a CRISPR repeat sequence having at least 95% sequence identity to SEQ ID NO: 119, and wherein said RGN polypeptide comprises an amino acid sequence having at least 95% sequence identity to SEQ ID NO: 16.

60. The system of claim 57, wherein said tracrRNA is selected from the group consisting of:
a) a tracrRNA having 100% sequence identity to SEQ ID NO: 121, wherein said one or more guide RNAs further comprise a CRISPR RNA comprising a CRISPR repeat sequence having 100% sequence identity to SEQ ID NO: 111, and wherein said RGN polypeptide comprises an amino acid sequence having 100% sequence identity to SEQ ID NO: 2;
b) a tracrRNA having 100% sequence identity to SEQ ID NO: 123, wherein said one or more guide RNAs further comprise a CRISPR RNA comprising a CRISPR repeat sequence having 100% sequence identity to SEQ ID NO: 113, and wherein said RGN polypeptide comprises an amino acid sequence having 100% sequence identity to SEQ ID NO: 4;
c) a tracrRNA having 100% sequence identity to SEQ ID NO: 120, wherein said one or more guide RNAs further comprise a CRISPR RNA comprising a CRISPR repeat sequence having 100% sequence identity to SEQ ID NO: 110, and wherein said RGN polypeptide comprises an amino acid sequence having 100% sequence identity to SEQ ID NO: 1;
d) a tracrRNA having 100% sequence identity to SEQ ID NO: 122, wherein said one or more guide RNAs further comprise a CRISPR RNA comprising a CRISPR repeat sequence having 100% sequence identity to SEQ ID NO: 112, and wherein said RGN polypeptide comprises an amino acid sequence having 100% sequence identity to SEQ ID NO: 3;
e) a tracrRNA having 100% sequence identity to SEQ ID NO: 124, wherein said one or more guide RNAs further comprise a CRISPR RNA comprising a CRISPR repeat sequence having 100% sequence identity to SEQ ID NO: 114, and wherein said RGN polypeptide comprises an amino acid sequence having 100% sequence identity to SEQ ID NO: 5;
f) a tracrRNA having 100% sequence identity to SEQ ID NO: 125, wherein said one or more guide RNAs further comprise a CRISPR RNA comprising a CRISPR repeat sequence having 100% sequence identity to SEQ ID NO: 115, and wherein said RGN polypeptide comprises an amino acid sequence having 100% sequence identity to SEQ ID NO: 6;
g) a tracrRNA having 100% sequence identity to SEQ ID NO: 126, wherein said one or more guide RNAs further comprise a CRISPR RNA comprising a CRISPR repeat sequence having 100% sequence identity to SEQ ID NO: 117, and wherein said RGN polypeptide comprises an amino acid sequence having 100% sequence identity to SEQ ID NO: 12;
h) a tracrRNA having 100% sequence identity to SEQ ID NO: 127, wherein said one or more guide RNAs further comprise a CRISPR RNA comprising a CRISPR repeat sequence having 100% sequence identity to SEQ ID NO: 118, and wherein said RGN polypeptide comprises an amino acid sequence having 100% sequence identity to SEQ ID NO: 13; and
i) a tracrRNA having 100% sequence identity to SEQ ID NO: 128, wherein said one or more guide RNAs further comprise a CRISPR RNA comprising a CRISPR repeat sequence having 100% sequence identity to SEQ ID NO: 119, and wherein said RGN polypeptide comprises an amino acid sequence having 100% sequence identity to SEQ ID NO: 16.

61. The system of any one of claims 57-60, wherein said one or more guide RNAs are a single guide RNA (sgRNA).

62. The system of any one of claims 57-60, wherein said one or more guide RNAs are a dual-guide RNA.

63. The system of any one of claims 47-62, wherein the target DNA sequence is within a cell.

64. The system of claim 63, wherein the cell is a eukaryotic cell.

65. The system of claim 64, wherein the eukaryotic cell is a plant cell.

66. The system of claim 64, wherein the eukaryotic cell is a mammalian cell.

67. The system of claim 64, wherein the eukaryotic cell is an insect cell.

68. The system of claim 63, wherein the cell is a prokaryotic cell.

69. The system of any one of claims 47-68, wherein said target DNA sequence is within a region of said DNA molecule that is single-stranded.

70. The system of claim 69, wherein when transcribed the one or more guide RNAs is capable of hybridizing to the target DNA sequence and the guide RNA is capable of forming a complex with the RGN polypeptide to direct cleavage of the target DNA sequence.

71. The system of any one of claims 47-68, wherein said target DNA sequence is within a region of said DNA molecule that is double-stranded.

72. The system of claim 71, wherein when transcribed the one or more guide RNAs is capable of hybridizing to the target DNA sequence and the guide RNA is capable of forming a complex with the RGN polypeptide to direct cleavage of the target DNA sequence.

73. The system of claim 72, wherein said RGN polypeptide is capable of generating a double-stranded break.

74. The system of claim 72, wherein said RGN polypeptide is capable of generating a single-stranded break.

75. The system of any one of claims 71-74, wherein the RGN polypeptide is operably linked to a base-editing polypeptide.

76. The system of claim 75, wherein said base-editing polypeptide is a deaminase.

77. The system of any one of claims 71-76, wherein said target DNA sequence is located adjacent to a protospacer adjacent motif (PAM).

78. The system of any one of claims 47-77, wherein the RGN polypeptide comprises one or more nuclear localization signals.

79. The system of any one of claims 47-78, wherein the RGN polypeptide is codon optimized for expression in a eukaryotic cell.

80. The system of any one of claims 47-79, wherein polynucleotides comprising the nucleotide sequences encoding the one or more guide RNAs and the polynucleotide comprising the nucleotide sequence encoding an RGN polypeptide are located on one vector.

81. The system of any one of claims 47-80, wherein said system further comprises one or more donor polynucleotides or one or more polynucleotides comprising one or more nucleotide sequences encoding the one or more donor polynucleotides.

82. A pharmaceutical composition comprising the nucleic acid molecule of any one of claims 1-14, the vector of any one of claims 15-25, the cell of claim 26, the isolated RGN polypeptide of any one of claims 34-46, and the system of any one of claims 47-81, and a pharmaceutically acceptable carrier.

83. A method for binding a target DNA sequence of a DNA molecule comprising delivering a system according to any one of claims 47-81, to said target DNA sequence or a cell comprising the target DNA sequence.

84. The method of claim 83, wherein said RGN polypeptide or said guide RNA further comprises a detectable label, thereby allowing for detection of said target DNA sequence.

85. The method of claim 83, wherein said guide RNA or said RGN polypeptide further comprises an expression modulator, thereby modulating expression of said target DNA sequence or a gene under transcriptional control by said target DNA sequence.

86. A method for cleaving a target DNA sequence of a DNA molecule comprising delivering a system according to any one of claims 47-81, to said target DNA sequence or a cell comprising the target DNA sequence.

87. The method of claim 86, wherein said modified target DNA sequence comprises insertion of heterologous DNA into the target DNA sequence.

88. The method of claim 86, wherein said modified target DNA sequence comprises deletion of at least one nucleotide from the target DNA sequence.

89. The method of claim 86, wherein said modified target DNA sequence comprises mutation of at least one nucleotide in the target DNA sequence.

90. A method for binding a target DNA sequence of a DNA molecule, said method comprising:
a) assembling an RNA-guided nuclease (RGN) ribonucleotide complex in vitro by combining:
i) one or more guide RNAs capable of hybridizing to the target DNA sequence; and
ii) an RGN polypeptide comprising an amino acid sequence having at least 90% sequence identity to any one of SEQ ID NOs: 2, 4, 1, 3, and 5 to 109;
under conditions suitable for formation of the RGN ribonucleotide complex; and
b) contacting said target DNA sequence or a cell comprising said target DNA sequence with the in vitro-assembled RGN ribonucleotide complex;
wherein the one or more guide RNAs hybridize to the target DNA sequence, thereby directing said RGN polypeptide to bind to said target DNA sequence.

91. The method of claim 90, wherein said target DNA sequence is within a region of said DNA molecule that is single-stranded.

92. The method of claim 90, wherein said target DNA sequence is within a region of said DNA molecule that is double-stranded.

93. The method of claim 92, wherein said target DNA sequence is located adjacent to a protospacer adjacent motif (PAM).

94. The method of any one of claims 90-93, wherein said RGN polypeptide or said guide RNA further comprises a detectable label, thereby allowing for detection of said target DNA sequence.

95. The method of any one of claims 90-93, wherein said guide RNA or said RGN polypeptide further comprises an expression modulator, thereby allowing for the modulation of expression of said target DNA sequence.

96. A method for cleaving and/or modifying a target DNA sequence of a DNA molecule, comprising contacting the DNA molecule with:
a) an RNA-guided nuclease (RGN) polypeptide, wherein said RGN comprises an amino acid sequence having at least 90% sequence identity to any one of SEQ ID NOs: 2, 4, 1, 3, and 5 to 109; and
b) one or more guide RNAs capable of targeting the RGN of (a) to the target DNA sequence;
wherein the one or more guide RNAs hybridize to the target DNA sequence, thereby directing said RGN polypeptide to bind to said target DNA sequence and cleavage and/or modification of said target DNA sequence occurs.

97. The method of claim 96, wherein said target DNA sequence is within a region of said DNA molecule that is single-stranded.

98. The method of claim 96, wherein said target DNA sequence is within a region of said DNA molecule that is double-stranded.

99. The method of claim 98, wherein cleavage by said RGN polypeptide generates a double-stranded break.

100. The method of claim 98, wherein cleavage by said RGN polypeptide generates a single-stranded break.

101. The method of any one of claims 98-100, wherein said RGN polypeptide is operably linked to a base-editing polypeptide.

102. The method of claim 101, wherein said base-editing polypeptide comprises a deaminase.

103. The method of any one of claims 98-102, wherein said target DNA sequence is located adjacent to a protospacer adjacent motif (PAM).

104. The method of any one of claims 96-103, wherein said modified target DNA sequence comprises insertion of heterologous DNA into the target DNA sequence.

105. The method of any one of claims 96-103, wherein said modified target DNA sequence comprises deletion of at least one nucleotide from the target DNA sequence.

106. The method of any one of claims 96-103, wherein said modified target DNA sequence comprises mutation of at least one nucleotide in the target DNA sequence.

107. The method of any one of claims 90-106, wherein said RGN comprises an amino acid sequence having at least 95% sequence identity to any one of SEQ ID NOs: 2, 4, 1, 3, and 5 to 109.

108. The method of any one of claims 90-106, wherein said RGN comprises an amino acid sequence having 100% sequence identity to any one of SEQ ID NOs: 2, 4, 1, 3, and 5 to 109.

109. The method of any one of claims 90-108, wherein said RGN polypeptide and said one or more guide RNAs are not found complexed to one another in nature.

110. The method of any one of claims 90-109, wherein said target DNA sequence is a eukaryotic target DNA sequence.

111. The method of any one of claims 90-110, wherein said RGN polypeptide comprises an amino acid sequence having at least 90% sequence identity to SEQ ID NO: 11 and said one or more guide RNAs comprise a CRISPR RNA comprising a CRISPR repeat sequence having at least 90% sequence identity to SEQ ID NO: 116.

112. The method of any one of claims 90-110, wherein said RGN polypeptide comprises an amino acid sequence having at least 95% sequence identity to SEQ ID NO: 11 and said one or more guide RNAs comprise a CRISPR RNA comprising a CRISPR repeat sequence having at least 95% sequence identity to SEQ ID NO: 116.

113. The method of any one of claims 90-110, wherein said RGN polypeptide comprises an amino acid sequence having 100% sequence identity to SEQ ID NO: 11 and said one or more guide RNAs comprise a CRISPR RNA comprising a CRISPR repeat sequence having 100% sequence identity to SEQ ID NO: 116.

114. The method of any one of claims 90-110, wherein said one or more guide RNAs comprise a tracrRNA.

115. The method of claim 114, wherein said tracrRNA is selected from the group consisting of:
a) a tracrRNA having at least 90% sequence identity to SEQ ID NO: 121, wherein said one or more guide RNAs further comprise a CRISPR RNA comprising a CRISPR repeat sequence having at least 90% sequence identity to SEQ ID NO: 111, and wherein said RGN polypeptide comprises an amino acid sequence having at least 90% sequence identity to SEQ ID NO: 2;
b) a tracrRNA having at least 90% sequence identity to SEQ ID NO: 123, wherein said one or more guide RNAs further comprise a CRISPR RNA comprising a CRISPR repeat sequence having at least 90% sequence identity to SEQ ID NO: 113, and wherein said RGN polypeptide comprises an amino acid sequence having at least 90% sequence identity to SEQ ID NO: 4;
c) a tracrRNA having at least 90% sequence identity to SEQ ID NO: 120, wherein said one or more guide RNAs further comprise a CRISPR RNA comprising a CRISPR repeat sequence having at least 90% sequence identity to SEQ ID NO: 110, and wherein said RGN polypeptide comprises an amino acid sequence having at least 90% sequence identity to SEQ ID NO: 1;
d) a tracrRNA having at least 90% sequence identity to SEQ ID NO: 122, wherein said one or more guide RNAs further comprise a CRISPR RNA comprising a CRISPR repeat sequence having at least 90% sequence identity to SEQ ID NO: 112, and wherein said RGN polypeptide comprises an amino acid sequence having at least 90% sequence identity to SEQ ID NO: 3;
e) a tracrRNA having at least 90% sequence identity to SEQ ID NO: 124, wherein said one or more guide RNAs further comprise a CRISPR RNA comprising a CRISPR repeat sequence having at least 90% sequence identity to SEQ ID NO: 114, and wherein said RGN polypeptide comprises an amino acid sequence having at least 90% sequence identity to SEQ ID NO: 5;
f) a tracrRNA having at least 90% sequence identity to SEQ ID NO: 125, wherein said one or more guide RNAs further comprise a CRISPR RNA comprising a CRISPR repeat sequence having at least 90% sequence identity to SEQ ID NO: 115, and wherein said RGN polypeptide comprises an amino acid sequence having at least 90% sequence identity to SEQ ID NO: 6;
g) a tracrRNA having at least 90% sequence identity to SEQ ID NO: 126, wherein said one or more guide RNAs further comprise a CRISPR RNA comprising a CRISPR repeat sequence having at least 90% sequence identity to SEQ ID NO: 117, and wherein said RGN polypeptide comprises an amino acid sequence having at least 90% sequence identity to SEQ ID NO: 12;
h) a tracrRNA having at least 90% sequence identity to SEQ ID NO: 127, wherein said one or more guide RNAs further comprise a CRISPR RNA comprising a CRISPR repeat sequence having at least 90% sequence identity to SEQ ID NO: 118, and wherein said RGN polypeptide comprises an amino acid sequence having at least 90% sequence identity to SEQ ID NO: 13; and
i) a tracrRNA having at least 90% sequence identity to SEQ ID NO: 128, wherein said one or more guide RNAs further comprise a CRISPR RNA comprising a CRISPR repeat sequence having at least 90% sequence identity to SEQ ID NO: 119, and wherein said RGN polypeptide comprises an amino acid sequence having at least 90% sequence identity to SEQ ID NO: 16.

116. The method of claim 114, wherein said tracrRNA is selected from the group consisting of:
a) a tracrRNA having at least 95% sequence identity to SEQ ID NO: 121, wherein said one or more guide RNAs further comprise a CRISPR RNA comprising a CRISPR repeat sequence having at least 95% sequence identity to SEQ ID NO: 111, and wherein said RGN polypeptide comprises an amino acid sequence having at least 95% sequence identity to SEQ ID NO: 2;
b) a tracrRNA having at least 95% sequence identity to SEQ ID NO: 123, wherein said one or more guide RNAs further comprise a CRISPR RNA comprising a CRISPR repeat sequence having at least 95% sequence identity to SEQ ID NO: 113, and wherein said RGN polypeptide comprises an amino acid sequence having at least 95% sequence identity to SEQ ID NO: 4;
c) a tracrRNA having at least 95% sequence identity to SEQ ID NO: 120, wherein said one or more guide RNAs further comprise a CRISPR RNA comprising a CRISPR repeat sequence having at least 95% sequence identity to SEQ ID NO: 110, and wherein said RGN polypeptide comprises an amino acid sequence having at least 95% sequence identity to SEQ ID NO: 1;
d) a tracrRNA having at least 95% sequence identity to SEQ ID NO: 122, wherein said one or more guide RNAs further comprise a CRISPR RNA comprising a CRISPR repeat sequence having at least 95% sequence identity to SEQ ID NO: 112, and wherein said RGN polypeptide comprises an amino acid sequence having at least 95% sequence identity to SEQ ID NO: 3;
e) a tracrRNA having at least 95% sequence identity to SEQ ID NO: 124, wherein said one or more guide RNAs further comprise a CRISPR RNA comprising a CRISPR repeat sequence having at least 95% sequence identity to SEQ ID NO: 114, and wherein said RGN polypeptide comprises an amino acid sequence having at least 95% sequence identity to SEQ ID NO: 5;
f) a tracrRNA having at least 95% sequence identity to SEQ ID NO: 125, wherein said one or more guide RNAs further comprise a CRISPR RNA comprising a CRISPR repeat sequence having at least 95% sequence identity to SEQ ID NO: 115, and wherein said RGN polypeptide comprises an amino acid sequence having at least 95% sequence identity to SEQ ID NO: 6;
g) a tracrRNA having at least 95% sequence identity to SEQ ID NO: 126, wherein said one or more guide RNAs further comprise a CRISPR RNA comprising a CRISPR repeat sequence having at least 95% sequence identity to SEQ ID NO: 117, and wherein said RGN polypeptide comprises an amino acid sequence having at least 95% sequence identity to SEQ ID NO: 12;
h) a tracrRNA having at least 95% sequence identity to SEQ ID NO: 127, wherein said one or more guide RNAs further comprise a CRISPR RNA comprising a CRISPR repeat sequence having at least 95% sequence identity to SEQ ID NO: 118, and wherein said RGN polypeptide comprises an amino acid sequence having at least 95% sequence identity to SEQ ID NO: 13; and
i) a tracrRNA having at least 95% sequence identity to SEQ ID NO: 128, wherein said one or more guide RNAs further comprise a CRISPR RNA comprising a CRISPR repeat sequence having at least 95% sequence identity to SEQ ID NO: 119, and wherein said RGN polypeptide comprises an amino acid sequence having at least 95% sequence identity to SEQ ID NO: 16.

117. The method of claim 114, wherein said tracrRNA is selected from the group consisting of:
a) a tracrRNA having 100% sequence identity to SEQ ID NO: 121, wherein said one or more guide RNAs further comprise a CRISPR RNA comprising a CRISPR repeat sequence having 100% sequence identity to SEQ ID NO: 111, and wherein said RGN polypeptide comprises an amino acid sequence having 100% sequence identity to SEQ ID NO: 2;
b) a tracrRNA having 100% sequence identity to SEQ ID NO: 123, wherein said one or more guide RNAs further comprise a CRISPR RNA comprising a CRISPR repeat sequence having 100% sequence identity to SEQ ID NO: 113, and wherein said RGN polypeptide comprises an amino acid sequence having 100% sequence identity to SEQ ID NO: 4;
c) a tracrRNA having 100% sequence identity to SEQ ID NO: 120, wherein said one or more guide RNAs further comprise a CRISPR RNA comprising a CRISPR repeat sequence having 100% sequence identity to SEQ ID NO: 110, and wherein said RGN polypeptide comprises an amino acid sequence having 100% sequence identity to SEQ ID NO: 1;
d) a tracrRNA having 100% sequence identity to SEQ ID NO: 122, wherein said one or more guide RNAs further comprise a CRISPR RNA comprising a CRISPR repeat sequence having 100% sequence identity to SEQ ID NO: 112, and wherein said RGN polypeptide comprises an amino acid sequence having 100% sequence identity to SEQ ID NO: 3;
e) a tracrRNA having 100% sequence identity to SEQ ID NO: 124, wherein said one or more guide RNAs further comprise a CRISPR RNA comprising a CRISPR repeat sequence having 100% sequence identity to SEQ ID NO: 114, and wherein said RGN polypeptide comprises an amino acid sequence having 100% sequence identity to SEQ ID NO: 5;
f) a tracrRNA having 100% sequence identity to SEQ ID NO: 125, wherein said one or more guide RNAs further comprise a CRISPR RNA comprising a CRISPR repeat sequence having 100% sequence identity to SEQ ID NO: 115, and wherein said RGN polypeptide comprises an amino acid sequence having 100% sequence identity to SEQ ID NO: 6;
g) a tracrRNA having 100% sequence identity to SEQ ID NO: 126, wherein said one or more guide RNAs further comprise a CRISPR RNA comprising a CRISPR repeat sequence having 100% sequence identity to SEQ ID NO: 117, and wherein said RGN polypeptide comprises an amino acid sequence having 100% sequence identity to SEQ ID NO: 12;
h) a tracrRNA having 100% sequence identity to SEQ ID NO: 127, wherein said one or more guide RNAs further comprise a CRISPR RNA comprising a CRISPR repeat sequence having 100% sequence identity to SEQ ID NO: 118, and wherein said RGN polypeptide comprises an amino acid sequence having 100% sequence identity to SEQ ID NO: 13; and
i) a tracrRNA having 100% sequence identity to SEQ ID NO: 128, wherein said one or more guide RNAs further comprise a CRISPR RNA comprising a CRISPR repeat sequence having 100% sequence identity to SEQ ID NO: 119, and wherein said RGN polypeptide comprises an amino acid sequence having 100% sequence identity to SEQ ID NO: 16.

118. The method of any one of claims 114-117, wherein said one or more guide RNAs are a single guide RNA (sgRNA).

119. The method of any one of claims 114-117, wherein said one or more guide RNAs are a dual-guide RNA.

120. The method of any one of claims 83-119, wherein the target DNA sequence is within a cell.

121. The method of claim 120, wherein the cell is a eukaryotic cell.

122. The method of claim 121, wherein the eukaryotic cell is a plant cell.

123. The method of claim 121, wherein the eukaryotic cell is a mammalian cell.

124. The method of claim 121, wherein the eukaryotic cell is an insect cell.

125. The method of claim 120, wherein the cell is a prokaryotic cell.

126. A cell comprising a modified target DNA sequence according to the method of any one of claims 96-119.

127. The cell of claim 126, wherein the cell is a eukaryotic cell.

128. The cell of claim 127, wherein the eukaryotic cell is a plant cell.

129. A plant comprising the cell of claim 128.

130. A seed comprising the cell of claim 128.

131. The cell of claim 127, wherein the eukaryotic cell is a mammalian cell.

132. The cell of claim 131, wherein the mammalian cell is a human cell.

133. The cell of claim 132, wherein the human cell is an immune cell.

134. The cell of claim 133, wherein the human cell is a stem cell.

135. The cell of claim 134, wherein the stem cell is an induced pluripotent stem cell.

136. The cell of claim 127, wherein the eukaryotic cell is an insect cell.

137. The cell of claim 126, wherein the cell is a prokaryotic cell.

138. A pharmaceutical composition comprising the cell of any one of embodiments 127 and 131-135, and a pharmaceutically acceptable carrier.

139. A kit for detecting a target DNA sequence of a DNA molecule in a sample, the kit comprising:
a) an RNA-guided nuclease (RGN) polypeptide comprising an amino acid sequence having at least 90% sequence identity to any one of SEQ ID NOs: 2, 4, 1, 3, and 5 to 109 or a polynucleotide comprising a nucleotide sequence encoding the RGN polypeptide, wherein said RGN polypeptide is capable of binding and cleaving said target DNA sequence of a DNA molecule in an RNA-guided sequence specific manner when bound to a guide RNA capable of hybridizing to said target DNA sequence;
b) said guide RNA or a polynucleotide comprising a nucleotide sequence encoding said guide RNA; and
c) a detector single-stranded DNA (ssDNA) that does not hybridize with the guide RNA.

140. The kit of claim 139, wherein said RGN polypeptide comprises an amino acid sequence having at least 95% sequence identity to any one of SEQ ID NOs: 2, 4, 1, 3, and 5 to 109.

141. The kit of claim 139, wherein said RGN polypeptide comprises an amino acid sequence having 100% sequence identity to any one of SEQ ID NOs: 2, 4, 1, 3, and 5 to 109.

142. The kit of any one of claims 139-141, wherein at least one of said nucleotide sequence encoding the guide RNA and said nucleotide sequence encoding the RGN polypeptide is operably linked to a promoter heterologous to said nucleotide sequence.

143. The kit of any one of claims 139-142, wherein said RGN polypeptide and said one or more guide RNAs are not found complexed to one another in nature.

144. The kit of any one of claims 139-142, wherein said target DNA sequence is a eukaryotic target DNA sequence.

145. The kit of any one of claims 139-144, wherein said detector ssDNA comprises a fluorophore/quencher pair.

146. The kit of any one of claims 139-144, wherein said detector ssDNA comprises a fluorescence resonance energy transfer (FRET) pair.

147. The kit of any one of claims 139-146, wherein said RGN polypeptide comprises an amino acid sequence having at least 90% sequence identity to SEQ ID NO: 11 and said one or more guide RNAs comprise a CRISPR RNA comprising a CRISPR repeat sequence having at least 90% sequence identity to SEQ ID NO: 116.

148. The kit of any one of claims 139-146, wherein said RGN polypeptide comprises an amino acid sequence having at least 95% sequence identity to SEQ ID NO: 11 and said one or more guide RNAs comprise a CRISPR RNA comprising a CRISPR repeat sequence having at least 95% sequence identity to SEQ ID NO: 116.

149. The kit of any one of claims 139-146, wherein said RGN polypeptide comprises an amino acid sequence having 100% sequence identity to SEQ ID NO: 11 and said one or more guide RNAs comprise a CRISPR RNA comprising a CRISPR repeat sequence having 100% sequence identity to SEQ ID NO: 116.

150. The kit of any one of claims 139-146, wherein said one or more guide RNAs comprise a tracrRNA.

151. The kit of claim 150, wherein said tracrRNA is selected from the group consisting of:
a) a tracrRNA having at least 90% sequence identity to SEQ ID NO: 121, wherein said one or more guide RNAs further comprise a CRISPR RNA comprising a CRISPR repeat sequence having at least 90% sequence identity to SEQ ID NO: 111, and wherein said RGN polypeptide comprises an amino acid sequence having at least 90% sequence identity to SEQ ID NO: 2;
b) a tracrRNA having at least 90% sequence identity to SEQ ID NO: 123, wherein said one or more guide RNAs further comprise a CRISPR RNA comprising a CRISPR repeat sequence having at least 90% sequence identity to SEQ ID NO: 113, and wherein said RGN polypeptide comprises an amino acid sequence having at least 90% sequence identity to SEQ ID NO: 4;
c) a tracrRNA having at least 90% sequence identity to SEQ ID NO: 120, wherein said one or more guide RNAs further comprise a CRISPR RNA comprising a CRISPR repeat sequence having at least 90% sequence identity to SEQ ID NO: 110, and wherein said RGN polypeptide comprises an amino acid sequence having at least 90% sequence identity to SEQ ID NO: 1;
d) a tracrRNA having at least 90% sequence identity to SEQ ID NO: 122, wherein said one or more guide RNAs further comprise a CRISPR RNA comprising a CRISPR repeat sequence having at least 90% sequence identity to SEQ ID NO: 112, and wherein said RGN polypeptide comprises an amino acid sequence having at least 90% sequence identity to SEQ ID NO: 3;
e) a tracrRNA having at least 90% sequence identity to SEQ ID NO: 124, wherein said one or more guide RNAs further comprise a CRISPR RNA comprising a CRISPR repeat sequence having at least 90% sequence identity to SEQ ID NO: 114, and wherein said RGN polypeptide comprises an amino acid sequence having at least 90% sequence identity to SEQ ID NO: 5;
f) a tracrRNA having at least 90% sequence identity to SEQ ID NO: 125, wherein said one or more guide RNAs further comprise a CRISPR RNA comprising a CRISPR repeat sequence having at least 90% sequence identity to SEQ ID NO: 115, and wherein said RGN polypeptide comprises an amino acid sequence having at least 90% sequence identity to SEQ ID NO: 6;
g) a tracrRNA having at least 90% sequence identity to SEQ ID NO: 126, wherein said one or more guide RNAs further comprise a CRISPR RNA comprising a CRISPR repeat sequence having at least 90% sequence identity to SEQ ID NO: 117, and wherein said RGN polypeptide comprises an amino acid sequence having at least 90% sequence identity to SEQ ID NO: 12;
h) a tracrRNA having at least 90% sequence identity to SEQ ID NO: 127, wherein said one or more guide RNAs further comprise a CRISPR RNA comprising a CRISPR repeat sequence having at least 90% sequence identity to SEQ ID NO: 118, and wherein said RGN polypeptide comprises an amino acid sequence having at least 90% sequence identity to SEQ ID NO: 13; and
i) a tracrRNA having at least 90% sequence identity to SEQ ID NO: 128, wherein said one or more guide RNAs further comprise a CRISPR RNA comprising a CRISPR repeat sequence having at least 90% sequence identity to SEQ ID NO: 119, and wherein said RGN polypeptide comprises an amino acid sequence having at least 90% sequence identity to SEQ ID NO: 16.

152. The kit of claim 150, wherein said tracrRNA is selected from the group consisting of:
a) a tracrRNA having at least 95% sequence identity to SEQ ID NO: 121, wherein said one or more guide RNAs further comprise a CRISPR RNA comprising a CRISPR repeat sequence having at least 95% sequence identity to SEQ ID NO: 111, and wherein said RGN polypeptide comprises an amino acid sequence having at least 95% sequence identity to SEQ ID NO: 2;
b) a tracrRNA having at least 95% sequence identity to SEQ ID NO: 123, wherein said one or more guide RNAs further comprise a CRISPR RNA comprising a CRISPR repeat sequence having at least 95% sequence identity to SEQ ID NO: 113, and wherein said RGN polypeptide comprises an amino acid sequence having at least 95% sequence identity to SEQ ID NO: 4;
c) a tracrRNA having at least 95% sequence identity to SEQ ID NO: 120, wherein said one or more guide RNAs further comprise a CRISPR RNA comprising a CRISPR repeat sequence having at least 95% sequence identity to SEQ ID NO: 110, and wherein said RGN polypeptide comprises an amino acid sequence having at least 95% sequence identity to SEQ ID NO: 1;
d) a tracrRNA having at least 95% sequence identity to SEQ ID NO: 122, wherein said one or more guide RNAs further comprise a CRISPR RNA comprising a CRISPR repeat sequence having at least 95% sequence identity to SEQ ID NO: 112, and wherein said RGN polypeptide comprises an amino acid sequence having at least 95% sequence identity to SEQ ID NO: 3;
e) a tracrRNA having at least 95% sequence identity to SEQ ID NO: 124, wherein said one or more guide RNAs further comprise a CRISPR RNA comprising a CRISPR repeat sequence having at least 95% sequence identity to SEQ ID NO: 114, and wherein said RGN polypeptide comprises an amino acid sequence having at least 95% sequence identity to SEQ ID NO: 5;
f) a tracrRNA having at least 95% sequence identity to SEQ ID NO: 125, wherein said one or more guide RNAs further comprise a CRISPR RNA comprising a CRISPR repeat sequence having at least 95% sequence identity to SEQ ID NO: 115, and wherein said RGN polypeptide comprises an amino acid sequence having at least 95% sequence identity to SEQ ID NO: 6;
g) a tracrRNA having at least 95% sequence identity to SEQ ID NO: 126, wherein said one or more guide RNAs further comprise a CRISPR RNA comprising a CRISPR repeat sequence having at least 95% sequence identity to SEQ ID NO: 117, and wherein said RGN polypeptide comprises an amino acid sequence having at least 95% sequence identity to SEQ ID NO: 12;
h) a tracrRNA having at least 95% sequence identity to SEQ ID NO: 127, wherein said one or more guide RNAs further comprise a CRISPR RNA comprising a CRISPR repeat sequence having at least 95% sequence identity to SEQ ID NO: 118, and wherein said RGN polypeptide comprises an amino acid sequence having at least 95% sequence identity to SEQ ID NO: 13; and
i) a tracrRNA having at least 95% sequence identity to SEQ ID NO: 128, wherein said one or more guide RNAs further comprise a CRISPR RNA comprising a CRISPR repeat sequence having at least 95% sequence identity to SEQ ID NO: 119, and wherein said RGN polypeptide comprises an amino acid sequence having at least 95% sequence identity to SEQ ID NO: 16.

153. The kit of claim 150, wherein said tracrRNA is selected from the group consisting of:
a) a tracrRNA having 100% sequence identity to SEQ ID NO: 121, wherein said one or more guide RNAs further comprise a CRISPR RNA comprising a CRISPR repeat sequence having 100% sequence identity to SEQ ID NO: 111, and wherein said RGN polypeptide comprises an amino acid sequence having 100% sequence identity to SEQ ID NO: 2;
b) a tracrRNA having 100% sequence identity to SEQ ID NO: 123, wherein said one or more guide RNAs further comprise a CRISPR RNA comprising a CRISPR repeat sequence having 100% sequence identity to SEQ ID NO: 113, and wherein said RGN polypeptide comprises an amino acid sequence having 100% sequence identity to SEQ ID NO: 4;
c) a tracrRNA having 100% sequence identity to SEQ ID NO: 120, wherein said one or more guide RNAs further comprise a CRISPR RNA comprising a CRISPR repeat sequence having 100% sequence identity to SEQ ID NO: 110, and wherein said RGN polypeptide comprises an amino acid sequence having 100% sequence identity to SEQ ID NO: 1;
d) a tracrRNA having 100% sequence identity to SEQ ID NO: 122, wherein said one or more guide RNAs further comprise a CRISPR RNA comprising a CRISPR repeat sequence having 100% sequence identity to SEQ ID NO: 112, and wherein said RGN polypeptide comprises an amino acid sequence having 100% sequence identity to SEQ ID NO: 3;
e) a tracrRNA having 100% sequence identity to SEQ ID NO: 124, wherein said one or more guide RNAs further comprise a CRISPR RNA comprising a CRISPR repeat sequence having 100% sequence identity to SEQ ID NO: 114, and wherein said RGN polypeptide comprises an amino acid sequence having 100% sequence identity to SEQ ID NO: 5;
f) a tracrRNA having 100% sequence identity to SEQ ID NO: 125, wherein said one or more guide RNAs further comprise a CRISPR RNA comprising a CRISPR repeat sequence having 100% sequence identity to SEQ ID NO: 115, and wherein said RGN polypeptide comprises an amino acid sequence having 100% sequence identity to SEQ ID NO: 6;
g) a tracrRNA having 100% sequence identity to SEQ ID NO: 126, wherein said one or more guide RNAs further comprise a CRISPR RNA comprising a CRISPR repeat sequence having 100% sequence identity to SEQ ID NO: 117, and wherein said RGN polypeptide comprises an amino acid sequence having 100% sequence identity to SEQ ID NO: 12;
h) a tracrRNA having 100% sequence identity to SEQ ID NO: 127, wherein said one or more guide RNAs further comprise a CRISPR RNA comprising a CRISPR repeat sequence having 100% sequence identity to SEQ ID NO: 118, and wherein said RGN polypeptide comprises an amino acid sequence having 100% sequence identity to SEQ ID NO: 13; and
i) a tracrRNA having 100% sequence identity to SEQ ID NO: 128, wherein said one or more guide RNAs further comprise a CRISPR RNA comprising a CRISPR repeat sequence having 100% sequence identity to SEQ ID NO: 119, and wherein said RGN polypeptide comprises an amino acid sequence having 100% sequence identity to SEQ ID NO: 16.

154. The kit of any one of claims 150-153, wherein said one or more guide RNAs are a single guide RNA (sgRNA).

155. The kit of any one of claims 150-153, wherein said one or more guide RNAs are a dual-guide RNA.

156. The kit of any one of claims 139-155, wherein said target DNA sequence is within a region of said DNA molecule that is single-stranded.

157. The kit of any one of claims 139-155, wherein said target DNA sequence is within a region of said DNA molecule that is double-stranded.

158. The kit of claim 157, wherein cleavage by said RGN polypeptide generates a double-stranded break.

159. The kit of claim 157, wherein cleavage by said RGN polypeptide generates a single-stranded break.

160. The kit of any one of claims 157-159, wherein said target DNA sequence is located adjacent to a protospacer adjacent motif (PAM).

161. A method of detecting a target DNA sequence of a DNA molecule in a sample, the method comprising:
a) contacting the sample with:
i) an RNA-guided nuclease (RGN) polypeptide comprising an amino acid sequence having at least 90% sequence identity to any one of SEQ ID NOs: 2, 4, 1, 3, and 5 to 109, wherein said RGN polypeptide is capable of binding and cleaving said target DNA sequence of a DNA molecule in an RNA-guided sequence specific manner when bound to a guide RNA capable of hybridizing to said target DNA sequence;
ii) said guide RNA; and
iii) a detector single-stranded DNA (ssDNA) that does not hybridize with the guide RNA; and
b) measuring a detectable signal produced by cleavage of the detector ssDNA by the RGN, thereby detecting the target DNA sequence.

162. The method of claim 161, wherein said RGN polypeptide comprises an amino acid sequence having at least 95% sequence identity to any one of SEQ ID NOs: 2, 4, 1, 3, and 5 to 109.

163. The method of claim 161, wherein said RGN polypeptide comprises an amino acid sequence having 100% sequence identity to any one of SEQ ID NOs: 2, 4, 1, 3, and 5 to 109.

164. The method of any one of claims 161-163, wherein said sample comprises DNA molecules from a cell lysate.

165. The method of any one of claims 161-163, wherein said sample comprises cells.

166. The method of claim 165, wherein said cells are eukaryotic cells.

167. The method of any one of claims 161-163, wherein the DNA molecule comprising the target DNA sequence is produced by reverse-transcription of an RNA template molecule present in a sample comprising RNA.

168. The method of claim 167, wherein the RNA template molecule is an RNA virus.

169. The method of claim 168, wherein the RNA virus is a coronavirus.

170. The method of claim 169, wherein the coronavirus is a bat SARS-like coronavirus, SARS-CoV, or SARS-CoV-2.

171. The method of any one of claims 167-170, wherein the samples comprising RNA is derived from a sample comprising cells.

172. The method of any one of claims 161-171, wherein said detector ssDNA comprises a fluorophore/quencher pair.

173. The method of any one of claims 161-171, wherein said detector ssDNA comprises a fluorescence resonance energy transfer (FRET) pair.

174. The method of any one of claims 161-173, wherein said method further comprises amplifying nucleic acids in the sample prior to or together with the contacting of step a).

175. A method of cleaving single-stranded DNAs (ssDNAs), the method comprising contacting a population of nucleic acids, wherein said population comprises a DNA molecule comprising a target DNA sequence and a plurality of non-target ssDNAs with:
a) an RNA-guided nuclease (RGN) polypeptide compriing an amino acid sequence having at least 90% sequence identity to any one of SEQ ID NOs: 2, 4, 1, 3, and 5 to 109, wherein said RGN polypeptide is capable of binding and cleaving said target DNA sequence in an RNA-guided sequence specific manner when bound to a guide RNA capable of hybridizing to said target DNA sequence; and
b) said guide RNA;
wherein the RGN polypeptide cleaves non-target ssDNAs of said plurality.

176. The method of claim 175, wherein said RGN polypeptide comprises an amino acid sequence having at least 95% sequence identity to any one of SEQ ID NOs: 2, 4, 1, 3, and 5 to 109.

177. The method of claim 175, wherein said RGN polypeptide comprises an amino acid sequence having 100% sequence identity to any one of SEQ ID NOs: 2, 4, 1, 3, and 5 to 109.

178. The method of any one of claims 175-177, wherein said population of nucleic acids are within a cell lysate.

179. The method of any one of claims 175-178, wherein the DNA molecule comprising the target DNA sequence is produced by reverse-transcription of an RNA template molecule.

180. The method of any one of claims 161-179, wherein said RGN polypeptide and said guide RNA are not found complexed to one another in nature.

181. The method of any one of claims 161-180, wherein said RGN polypeptide comprises an amino acid sequence having at least 90% sequence identity to SEQ ID NO: 11 and said guide RNA comprises a CRISPR RNA comprising a CRISPR repeat sequence having at least 90% sequence identity to SEQ ID NO: 116.

182. The method of any one of claims 161-180, wherein said RGN polypeptide comprises an amino acid sequence having at least 95% sequence identity to SEQ ID NO: 11 and said guide RNA comprises a CRISPR RNA comprising a CRISPR repeat sequence having at least 95% sequence identity to SEQ ID NO: 116.

183. The method of any one of claims 161-180, wherein said RGN polypeptide comprises an amino acid sequence having 100% sequence identity to SEQ ID NO: 11 and said guide RNA comprises a CRISPR RNA comprising a CRISPR repeat sequence having 100% sequence identity to SEQ ID NO: 116.

184. The method of any one of claims 161-180, wherein said guide RNA comprises a tracrRNA.

185. The method of claim 184, wherein said tracrRNA is selected from the group consisting of:
a) a tracrRNA having at least 90% sequence identity to SEQ ID NO: 121, wherein said guide RNA further comprises a CRISPR RNA comprising a CRISPR repeat sequence having at least 90% sequence identity to SEQ ID NO: 111, and wherein said RGN polypeptide comprises an amino acid sequence having at least 90% sequence identity to SEQ ID NO: 2;
b) a tracrRNA having at least 90% sequence identity to SEQ ID NO: 123, wherein said guide RNA further comprises a CRISPR RNA comprising a CRISPR repeat sequence having at least 90% sequence identity to SEQ ID NO: 113, and wherein said RGN polypeptide comprises an amino acid sequence having at least 90% sequence identity to SEQ ID NO: 4;
c) a tracrRNA having at least 90% sequence identity to SEQ ID NO: 120, wherein said guide RNA further comprises a CRISPR RNA comprising a CRISPR repeat sequence having at least 90% sequence identity to SEQ ID NO: 110, and wherein said RGN polypeptide comprises an amino acid sequence having at least 90% sequence identity to SEQ ID NO: 1;
d) a tracrRNA having at least 90% sequence identity to SEQ ID NO: 122, wherein said guide RNA further comprises a CRISPR RNA comprising a CRISPR repeat sequence having at least 90% sequence identity to SEQ ID NO: 112, and wherein said RGN polypeptide comprises an amino acid sequence having at least 90% sequence identity to SEQ ID NO: 3;
e) a tracrRNA having at least 90% sequence identity to SEQ ID NO: 124, wherein said guide RNA further comprises a CRISPR RNA comprising a CRISPR repeat sequence having at least 90% sequence identity to SEQ ID NO: 114, and wherein said RGN polypeptide comprises an amino acid sequence having at least 90% sequence identity to SEQ ID NO: 5;
f) a tracrRNA having at least 90% sequence identity to SEQ ID NO: 125, wherein said guide RNA further comprises a CRISPR RNA comprising a CRISPR repeat sequence having at least 90% sequence identity to SEQ ID NO: 115, and wherein said RGN polypeptide comprises an amino acid sequence having at least 90% sequence identity to SEQ ID NO: 6;
g) a tracrRNA having at least 90% sequence identity to SEQ ID NO: 126, wherein said guide RNA further comprises a CRISPR RNA comprising a CRISPR repeat sequence having at least 90% sequence identity to SEQ ID NO: 117, and wherein said RGN polypeptide comprises an amino acid sequence having at least 90% sequence identity to SEQ ID NO: 12;
h) a tracrRNA having at least 90% sequence identity to SEQ ID NO: 127, wherein said guide RNA further comprises a CRISPR RNA comprising a CRISPR repeat sequence having at least 90% sequence identity to SEQ ID NO: 118, and wherein said RGN polypeptide comprises an amino acid sequence having at least 90% sequence identity to SEQ ID NO: 13; and
i) a tracrRNA having at least 90% sequence identity to SEQ ID NO: 128, wherein said guide RNA further comprises a CRISPR RNA comprising a CRISPR repeat sequence having at least 90% sequence identity to SEQ ID NO: 119, and wherein said RGN polypeptide comprises an amino acid sequence having at least 90% sequence identity to SEQ ID NO: 16.

186. The method of claim 184, wherein said tracrRNA is selected from the group consisting of:
a) a tracrRNA having at least 95% sequence identity to SEQ ID NO: 121, wherein said guide RNA further comprises a CRISPR RNA comprising a CRISPR repeat sequence having at least 95% sequence identity to SEQ ID NO: 111, and wherein said RGN polypeptide comprises an amino acid sequence having at least 95% sequence identity to SEQ ID NO: 2;
b) a tracrRNA having at least 95% sequence identity to SEQ ID NO: 123, wherein said guide RNA further comprises a CRISPR RNA comprising a CRISPR repeat sequence having at least 95% sequence identity to SEQ ID NO: 113, and wherein said RGN polypeptide comprises an amino acid sequence having at least 95% sequence identity to SEQ ID NO: 4;
c) a tracrRNA having at least 95% sequence identity to SEQ ID NO: 120, wherein said guide RNA further comprises a CRISPR RNA comprising a CRISPR repeat sequence having at least 95% sequence identity to SEQ ID NO: 110, and wherein said RGN polypeptide comprises an amino acid sequence having at least 95% sequence identity to SEQ ID NO: 1;
d) a tracrRNA having at least 95% sequence identity to SEQ ID NO: 122, wherein said guide RNA further comprises a CRISPR RNA comprising a CRISPR repeat sequence having at least 95% sequence identity to SEQ ID NO: 112, and wherein said RGN polypeptide comprises an amino acid sequence having at least 95% sequence identity to SEQ ID NO: 3;
e) a tracrRNA having at least 95% sequence identity to SEQ ID NO: 124, wherein said guide RNA further comprises a CRISPR RNA comprising a CRISPR repeat sequence having at least 95% sequence identity to SEQ ID NO: 114, and wherein said RGN polypeptide comprises an amino acid sequence having at least 95% sequence identity to SEQ ID NO: 5;
f) a tracrRNA having at least 95% sequence identity to SEQ ID NO: 125, wherein said guide RNA further comprises a CRISPR RNA comprising a CRISPR repeat sequence having at least 95% sequence identity to SEQ ID NO: 115, and wherein said RGN polypeptide comprises an amino acid sequence having at least 95% sequence identity to SEQ ID NO: 6;
g) a tracrRNA having at least 95% sequence identity to SEQ ID NO: 126, wherein said guide RNA further comprises a CRISPR RNA comprising a CRISPR repeat sequence having at least 95% sequence identity to SEQ ID NO: 117, and wherein said RGN polypeptide comprises an amino acid sequence having at least 95% sequence identity to SEQ ID NO: 12;
h) a tracrRNA having at least 95% sequence identity to SEQ ID NO: 127, wherein said guide RNA further comprises a CRISPR RNA comprising a CRISPR repeat sequence having at least 95% sequence identity to SEQ ID NO: 118, and wherein said RGN polypeptide comprises an amino acid sequence having at least 95% sequence identity to SEQ ID NO: 13; and
i) a tracrRNA having at least 95% sequence identity to SEQ ID NO: 128, wherein said guide RNA further comprises a CRISPR RNA comprising a CRISPR repeat sequence having at least 95% sequence identity to SEQ ID NO: 119, and wherein said RGN polypeptide comprises an amino acid sequence having at least 95% sequence identity to SEQ ID NO: 16.

187. The method of claim 184, wherein said tracrRNA is selected from the group consisting of:
a) a tracrRNA having 100% sequence identity to SEQ ID NO: 121, wherein said guide RNA further comprises a CRISPR RNA comprising a CRISPR repeat sequence having 100% sequence identity to SEQ ID NO: 111, and wherein said RGN polypeptide comprises an amino acid sequence having 100% sequence identity to SEQ ID NO: 2;
b) a tracrRNA having 100% sequence identity to SEQ ID NO: 123, wherein said guide RNA further comprises a CRISPR RNA comprising a CRISPR repeat sequence having 100% sequence identity to SEQ ID NO: 113, and wherein said RGN polypeptide comprises an amino acid sequence having 100% sequence identity to SEQ ID NO: 4;
c) a tracrRNA having 100% sequence identity to SEQ ID NO: 120, wherein said guide RNA further comprises a CRISPR RNA comprising a CRISPR repeat sequence having 100% sequence identity to SEQ ID NO: 110, and wherein said RGN polypeptide comprises an amino acid sequence having 100% sequence identity to SEQ ID NO: 1;
d) a tracrRNA having 100% sequence identity to SEQ ID NO: 122, wherein said guide RNA further comprises a CRISPR RNA comprising a CRISPR repeat sequence having 100% sequence identity to SEQ ID NO: 112, and wherein said RGN polypeptide comprises an amino acid sequence having 100% sequence identity to SEQ ID NO: 3;
e) a tracrRNA having 100% sequence identity to SEQ ID NO: 124, wherein said guide RNA further comprises a CRISPR RNA comprising a CRISPR repeat sequence having 100% sequence identity to SEQ ID NO: 114, and wherein said RGN polypeptide comprises an amino acid sequence having 100% sequence identity to SEQ ID NO: 5;
f) a tracrRNA having 100% sequence identity to SEQ ID NO: 125, wherein said guide RNA further comprises a CRISPR RNA comprising a CRISPR repeat sequence having 100% sequence identity to SEQ ID NO: 115, and wherein said RGN polypeptide comprises an amino acid sequence having 100% sequence identity to SEQ ID NO: 6;
g) a tracrRNA having 100% sequence identity to SEQ ID NO: 126, wherein said guide RNA further comprises a CRISPR RNA comprising a CRISPR repeat sequence having 100% sequence identity to SEQ ID NO: 117, and wherein said RGN polypeptide comprises an amino acid sequence having 100% sequence identity to SEQ ID NO: 12;
h) a tracrRNA having 100% sequence identity to SEQ ID NO: 127, wherein said guide RNA further comprises a CRISPR RNA comprising a CRISPR repeat sequence having 100% sequence identity to SEQ ID NO: 118, and wherein said RGN polypeptide comprises an amino acid sequence having 100% sequence identity to SEQ ID NO: 13; and
i) a tracrRNA having 100% sequence identity to SEQ ID NO: 128, wherein said guide RNA further comprises a CRISPR RNA comprising a CRISPR repeat sequence having 100% sequence identity to SEQ ID NO: 119, and wherein said RGN polypeptide comprises an amino acid sequence having 100% sequence identity to SEQ ID NO: 16.

188. The method of any one of claims 184-187, wherein said guide RNA is a single guide RNA (sgRNA).

189. The method of any one of claims 184-187, wherein said guide RNA is a dual-guide RNA.

190. The method of any one of claims 161-189, wherein said target DNA sequence is within a region of said DNA molecule that is single-stranded.

191. The method of any one of claims 161-189, wherein said target DNA sequence is within a region of said DNA molecule that is double-stranded.

192. The method of claim 191, wherein cleavage of said target DNA sequence by said RGN polypeptide generates a double-stranded break.

193. The method of claim 191, wherein cleavage of said target DNA sequence by said RGN polypeptide generates a single-stranded break.

194. The method of any one of claims 191-193, wherein said target DNA sequence is located adjacent to a protospacer adjacent motif (PAM).

195. A method of treating a disease, said method comprising administering to a subject in need thereof an effective amount of a pharmaceutical composition of embodiment 82 or 138.

196. The method of embodiment 195, wherein said disease is associated with a causal mutation and said effective amount of said pharmaceutical composition corrects said causal mutation.
